(19) United States
(12) Patent Application Publication
(10) Pub. No.: US 2023/0180502 A1
(43) Pub. Date: Jun. 8, 2023

(54) LIGHT-EMITTING DEVICE INCLUDING DIAMINE COMPOUND, ELECTRONIC APPARATUS INCLUDING THE LIGHT-EMITTING DEVICE, AND THE DIAMINE COMPOUND

(71) Applicant: Samsung Display Co., Ltd., Yongin-si (KR)

(72) Inventors: Jeongmin Lee, Yongin-si (KR); Minji Kim, Yongin-si (KR); Hyunbin Park, Yongin-si (KR); Eunjae Jeong, Yongin-si (KR); Jiyong Choi, Yongin-si (KR); Sanghyun Han, Yongin-si (KR)

(21) Appl. No.: 18/060,818

(22) Filed: Dec. 1, 2022

(30) Foreign Application Priority Data
Dec. 6, 2021 (KR) 10-2021-0173133

(51) Int. Cl. H10K 50/15; C07C 211/54; H10K 85/60; C07D 307/91; C07D 333/76; C07D 209/82

(52) U.S. Cl. CPC H01L 51/5056; C07C 211/54; H01L 51/006; H01L 51/0073; C07D 307/91; H01L 51/0074; C07D 333/76; H01L 51/0072; C07D 209/82; H01L 51/0061; C07B 2200/05

(57) ABSTRACT

Provided are a light-emitting device including a diamine compound represented by Formula 1, an electronic apparatus including the light-emitting device, and a diamine compound represented by Formula 1, wherein details of Formula 1 are the same as described in the detailed description:

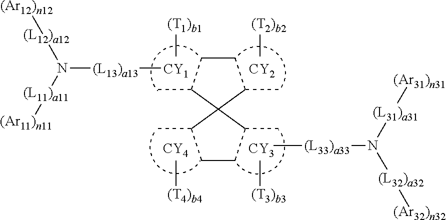

### CROSS-REFERENCE TO RELATED APPLICATION

This application claims priority to and the benefit of Korean Patent Application No. 10-2021-0173133, filed on Dec. 6, 2021, in the Korean Intellectual Property Office, the entire content of which is hereby incorporated by reference.

### BACKGROUND

### 1. Field

One or more embodiments of the present disclosure relate to a light-emitting device including diamine compound, an electronic apparatus including the light-emitting device, and the diamine compound.

### 2. Description of the Related Art

From among light-emitting devices, self-emissive devices have wide viewing angles, high contrast ratios, short response times, and excellent characteristics in terms of luminance, driving voltage, and response speed.

In a light-emitting device, a first electrode is on a substrate, and a hole transport region, an emission layer, an electron transport region, and a second electrode are sequentially on the first electrode. Holes provided from the first electrode move toward the emission layer through the hole transport region, and electrons provided from the second electrode move toward the emission layer through the electron transport region. Carriers, such as holes and electrons, recombine in the emission layer to produce excitons. These excitons transition from an excited state to a ground state to thereby generate light.

### SUMMARY

One or more embodiments of the present disclosure relate to a light-emitting device including diamine compound, an electronic apparatus including the light-emitting device, and the diamine compound.

Additional aspects of embodiments will be set forth in part in the description which follows and, in part, will be apparent from the description, or may be learned by practice of the presented embodiments of the disclosure.

According to one or more embodiments, a light-emitting device includes:

a first electrode,

a second electrode facing the first electrode, and

an interlayer between the first electrode and the second electrode and including an emission layer, and

at least one diamine compound represented by Formula 1.

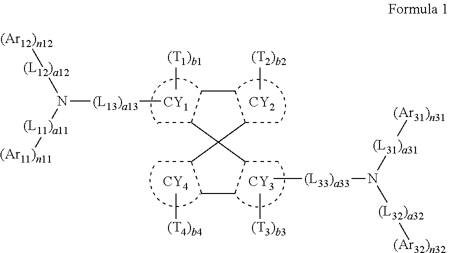

In Formula 1,

rings CY₁ to CY₄ may each independently be a C₃-C₃₀ carbocyclic group or a C₁-C₃₀ heterocyclic group,

T₁ to T₄ are each defined the same as described with respect to R₁₀ₐ,

b1 to b4 may each independently be an integer from 0 to 20,

L₁₁ to L₁₃ and L₃₁ to L₃₃ may each independently be a C₃-C₃₀ carbocyclic group unsubstituted or substituted with at least one R₁₀ₐ or a C₁-C₃₀ heterocyclic group unsubstituted or substituted with at least one R₁₀ₐ,

a11 to a13 and a31 to a33 may each independently be an integer from 0 to 3,

when a11 is 0, *-(L₁₁)ₐ₁₁-*′ may be a single bond,

when a12 is 0, *-(L₁₂)ₐ₁₂-*′ may be a single bond,

when a13 is 0, *-(L₁₃)ₐ₁₃-*′ may be a single bond,

when a31 is 0, *-(L₃₁)ₐ₃₁-*′ may be a single bond,

when a32 is 0, *-(L₃₂)ₐ₃₂-*′ may be a single bond,

when a33 is 0, *-(L₃₃)ₐ₃₃-*′ may be a single bond,

Ar₁₁, Ar₁₂, Ar₃₁, and Ar₃₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

at least one of Ar₁₁, Ar₁₂, Ar₃₁, and Ar₃₂ may be substituted with four or more deuterium atoms,

n11, n12, n31, and n32 may each independently be an integer from 1 to 3,

R₁₀ₐ may be:

deuterium, —F, —Cl, —Br, —I, a hydroxyl group, a cyano group, or a nitro group,

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), B(Q₁₁)(Q₁₂), —C(═O)(Q₁₁), —S(═O)₂(Q₁₁), —P(═O)(Q₁₁)(Q₁₂), or any combination thereof,

a C₃-C₆₀ carbocyclic group, a C₁-C₆₀ heterocyclic group, a C₆-C₆₀ aryloxy group; or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —B(Q₃₁)(Q₃₂), —C(═O)(Q₃₁), —S(═O)₂(Q₃₁), or —P(═O)(Q₃₁)(Q₃₂),

wherein Q₁ to Q₂, Q₁₁ to Q₁₃, Q₂₁ to Q₂₃, and Q₃₁ to Q₃₃ may each independently be hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

According to one or more embodiments, an electronic apparatus includes the light-emitting device.

One or more embodiments include the at least one diamine compound represented by Formula 1.

### BRIEF DESCRIPTION OF THE DRAWINGS

The above and other aspects and features of certain embodiments of the disclosure will be more apparent from the following description taken in conjunction with the accompanying drawings, in which:

FIG. 1 shows a schematic view of a light-emitting device according to an embodiment;

FIG. 2 shows a schematic view of an electronic apparatus according to an embodiment; and

FIG. 3 shows a schematic view of an electronic apparatus according to an embodiment.

### DETAILED DESCRIPTION

Reference will now be made in more detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of embodiments of the present description. As used herein, the term “and/or” includes any and all combinations of one or more of the associated listed items. Throughout the disclosure, the expression “at least one of a, b or c” indicates only a, only b, only c, both a and b, both a and c, both b and c, all of a, b, and c, or variations thereof.

In an embodiment, provided is a light-emitting device including: a first electrode; a second electrode facing the first electrode; an interlayer between the first electrode and the second electrode and including an emission layer; and at least one diamine compound represented by Formula 1:

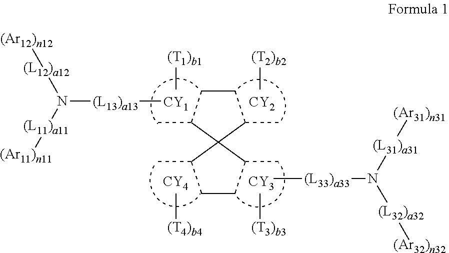

wherein, in Formula 1,

rings CY₁ to CY₄ may each independently be a C₃-C₃₀ carbocyclic group or a C₁-C₃₀ heterocyclic group,

T₁ to T₄ are each defined the same as described with respect to R₁₀ₐ,

b1 to b4 may each independently be an integer from 0 to 20,

L₁₁ to L₁₃ and L₃₁ to L₃₃ may each independently be a C₃-C₃₀ carbocyclic group unsubstituted or substituted with at least one R₁₀ₐ or a C₁-C₃₀ heterocyclic group unsubstituted or substituted with at least one R₁₀ₐ,

a11 to a13 and a31 to a33 may each independently be an integer from 0 to 3, wherein when a11 is 0, *-(L₁₁)ₐ₁₁-*′ may be a single bond, when a12 is 0, *-(L₁₂)ₐ₁₂-*′ may be a single bond, when a13 is 0, *-(L₁₃)ₐ₁₃-*′ may be a single bond, when a31 is 0, *-(L₃₁)ₐ₃₁-*′ may be a single bond, when a32 is 0, *-(L₃₂)ₐ₃₂-*′ may be a single bond, when a33 is 0, *-(L₃₃)ₐ₃₃-*′ may be a single bond,

Ar₁₁, Ar₁₂, Ar₃₁, and Ar₃₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

at least one of Ar₁₁, Ar₁₂, Ar₃₁, and Ar₃₂ may be substituted with four or more deuterium atoms,

n11, n12, n31, and n32 may each independently be an integer from 1 to 3,

R₁₀ₐ may be:

deuterium, —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group; or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —B(Q₃₁)(Q₃₂), —C(═O)(Q₃₁), —S(═O)₂(Q₃₁), or —P(═O)(Q₃₁)(Q₃₂),

wherein Q₁ to Q₂, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

In an embodiment, the interlayer may include the diamine compound represented by Formula 1.

In an embodiment, the first electrode may be an anode, the second electrode may be a cathode, and the interlayer layer further includes a hole transport region between the first electrode and the emission layer and an electron transport region between the emission layer and the second electrode, wherein the hole transport region may include a hole injection layer, a hole transport layer, an emission auxiliary layer, an electron blocking layer, or any combination thereof, and the electron transport region may include a hole blocking layer, an electron transport layer, an electron injection layer, or any combination thereof.

In an embodiment, the hole transport region may include the diamine compound represented by Formula 1.

In an embodiment, the hole transport layer may include the diamine compound represented by Formula 1.

In an embodiment, the hole transport layer may be in direct contact (e.g., physical contact) with the emission layer.

In an embodiment, the light-emitting device may further include a first capping layer and a second capping layer, the first capping layer may be on a surface of the first electrode, and the second capping layer may be on a surface of the second electrode.

In an embodiment, at least one of the first capping layer or the second capping layer may include the diamine compound represented by Formula 1.

In an embodiment, the emission layer may include a fluorescence dopant.

In an embodiment, the emission layer may include a phosphorescent dopant.

In an embodiment, the emission layer may emit blue light.

In one or more embodiments, an electronic apparatus may include the light-emitting device.

In an embodiment, an electronic apparatus may further include a thin-film transistor, the thin-film transistor may include a source electrode and a drain electrode, and the first electrode of the light-emitting device may be electrically connected to at least one selected from the source electrode and the drain electrode of the thin-film transistor.

In an embodiment, the electronic apparatus may further include a color filter, a color conversion layer, a touch screen layer, a polarizing layer, or any combination thereof.

One or more embodiments include the diamine compound represented by Formula 1:

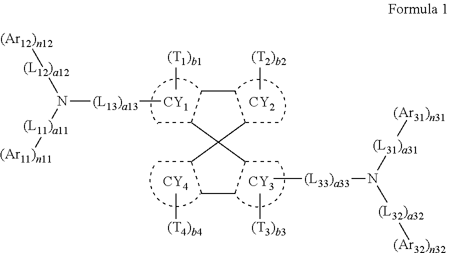

wherein, in Formula 1,

rings CY₁ to CY₄ may each independently be a C₃-C₃₀ carbocyclic group or a C₁-C₃₀ heterocyclic group,

T₁ to T₄ are each defined the same as described with respect to R₁₀ₐ,

b1 to b4 may each independently be an integer from 0 to 20,

L₁₁ to L₁₃ and L₃₁ to L₃₃ may each independently be a C₃-C₃₀ carbocyclic group unsubstituted or substituted with at least one R₁₀ₐ or a C₁-C₃₀ heterocyclic group unsubstituted or substituted with at least one R₁₀ₐ,

a11 to a13 and a31 to a33 may each independently be an integer from 0 to 3, wherein when a11 is 0, *-(L₁₁)ₐ₁₁-*′ may be a single bond, when a12 is 0, *-(L₁₂)ₐ₁₂-*′ may be a single bond, when a13 is 0, *-(L₁₃)ₐ₁₃-*′ may be a single bond, when a31 is 0, *-(L₃₁)ₐ₃₁-*′ may be a single bond, when a32 is 0, *-(L₃₂)ₐ₃₂-*′ may be a single bond, when a33 is 0, *-(L₃₃)ₐ₃₃-′ may be a single bond,

Ar₁₁, Ar₁₂, Ar₃₁, and Ar₃₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

at least one of Ar₁₁, Ar₁₂, Ar₃₁, and Ar₃₂ may be substituted with four or more deuterium atoms,

n11, n12, n31, and n32 may each independently be an integer from 1 to 3,

R₁₀ₐ may be:

deuterium, —F, —Cl, —Br, —I, a hydroxyl group, a cyano group, or a nitro group;

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), B(Q₁₁)(Q₁₂), —C(═O)(Q₁₁), —S(═O)₂(Q₁₁), —P(═O)(Q₁₁)(Q₁₂), or any combination thereof;

a C₃-C₆₀ carbocyclic group, a C₁-C₆₀ heterocyclic group, a C₆-C₆₀ aryloxy group; or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —B(Q₃₁)(Q₃₂), —C(═O)(Q₃₁), —S(═O)₂(Q₃₁), or —P(═O)(Q₃₁)(Q₃₂),

wherein Q₁ to Q₂, Q₁₁ to Q₁₃, Q₂₁ to Q₂₃, and Q₃₁ to Q₃₃ may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof.

In an embodiment, rings CY₁ to CY₄ may each independently be a benzene group or a naphthalene group.

In an embodiment, rings CY₁ and CY₂ may be identical to each other.

In an embodiment, rings CY₃ and CY₄ may be identical to each other.

In an embodiment, rings CY₁ and CY₃ may be identical to each other.

In an embodiment, rings CY₁ and CY₂ may be different from each other.

In an embodiment, rings CY₃ and CY₄ may be different from each other.

In an embodiment, rings CY₁ and CY₃ may be different from each other.

In an embodiment, rings CY₁ to CY₄ may be configured to be the same with or different from each other, and thus, by varying the size of steric hindrance related to a spatial distribution of the diamine compound and/or an electron distribution such as an electron density of the diamine compound, suitable or desired electrochemical properties may be realized. For example, the electrochemical properties of the diamine compound may be controlled by controlling the steric hindrance of the diamine compound resulting from positions of substituents in the diamine compound and/or from distribution of electron density in the diamine compound.

In an embodiment, in Formula 1,

a group represented by

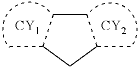

may be a group represented by one of Formulae 1-5-1 to 1-5-4:

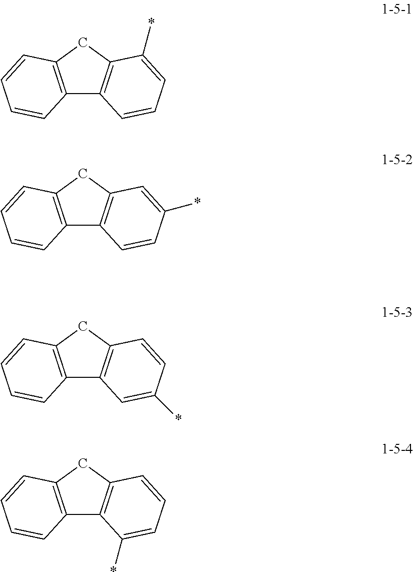 [SMILES: *c1cccc2c1Cc1ccccc1-2 ; *c1ccc2c(c1)Cc1ccccc1-2 ; *c1ccc2c(c1)-c1ccccc1C2 ; *c1cccc2c1-c1ccccc1C2]

In addition, in Formulae 1-5-1 to 1-5-4,

* may be a binding site to an atom included in the group represented by

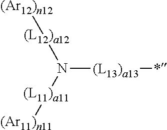

in Formula 1, and *″ may be a binding site to a neighboring atom.

In an embodiment, in Formula 1,

a group represented by

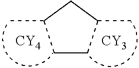

may be a group represented by one of Formulae 1-5-1 to 1-5-4:

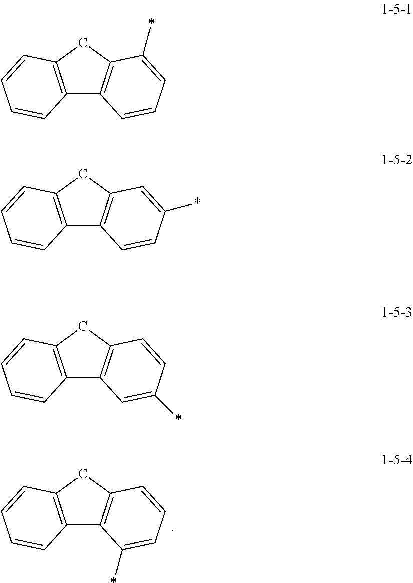 [SMILES: *c1cccc2c1Cc1ccccc1-2 ; *c1ccc2c(c1)Cc1ccccc1-2 ; *c1ccc2c(c1)-c1ccccc1C2 ; *c1cccc2c1-c1ccccc1C2]

In addition, in Formulae 1-5-1 to 1-5-4,

* may be a binding site to an atom included in the group represented by

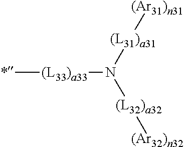

in Formula 1, and *″ may be a binding site to a neighboring atom.

In an embodiment, in Formula 1,

a group represented by

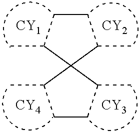

may be a group represented by one of Formulae 1-5-5 to 1-5-20:

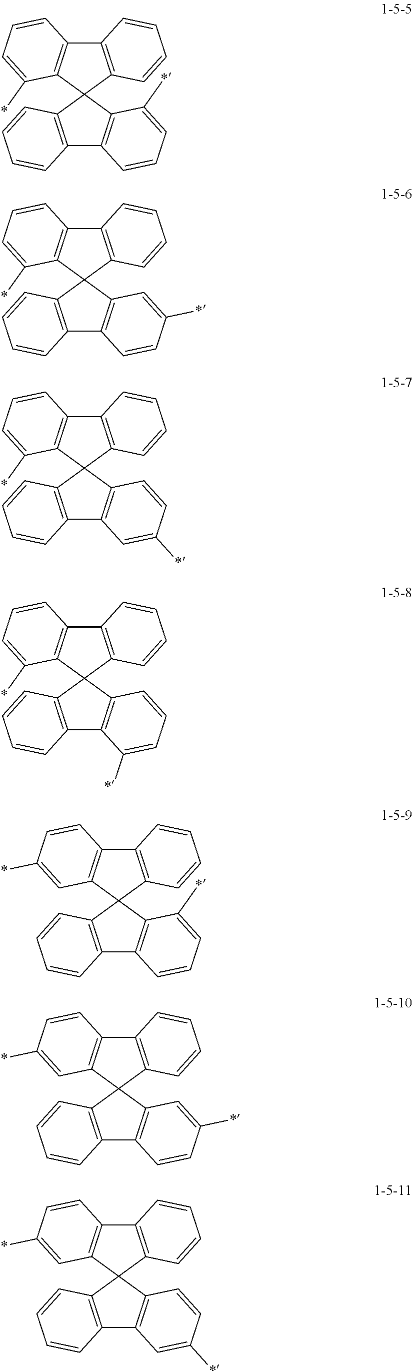

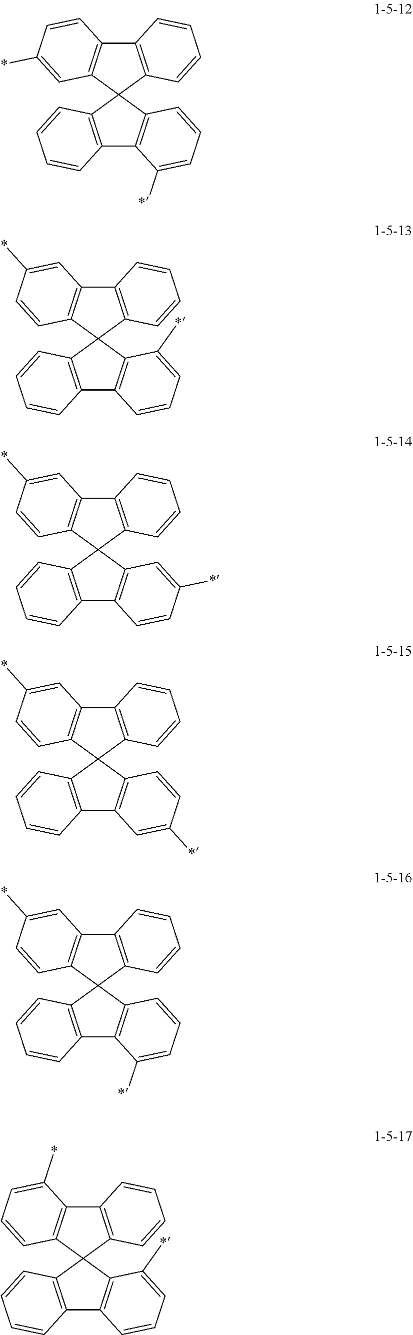

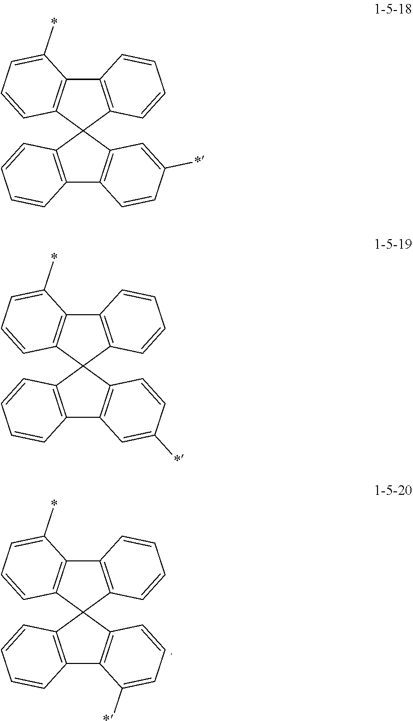

In addition, in Formulae 1-5-5 to 1-5-20,

* may be a binding site to an atom included in the group represented by

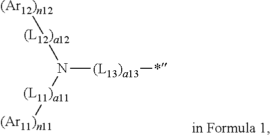

*′ may be a binding site to an atom included in the group represented by

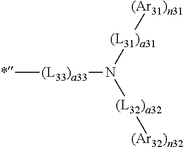

in Formula 1, and *″ may be a binding site to a neighboring atom.

In an embodiment, at least one of L₁₁, L₁₂, L₁₃, L₃₁, L₃₂, and L₃₃ may be a benzene group unsubstituted or substituted with at least one R₁₀ₐ, a naphthalene group unsubstituted or substituted with at least one R₁₀ₐ, a carbazole group unsubstituted or substituted with at least one R₁₀ₐ, a fluorene group unsubstituted or substituted with at least one R₁₀ₐ, a dibenzofuran group unsubstituted or substituted with at least one R₁₀ₐ, or a dibenzothiophene group unsubstituted or substituted with at least one R₁₀ₐ.

In an embodiment, at least one of L₁₁, L₁₂, L₁₃, L₃₁, L₃₂, and L₃₃ may be a group represented by one of Formulae 1-6-1 to 1-6-6.

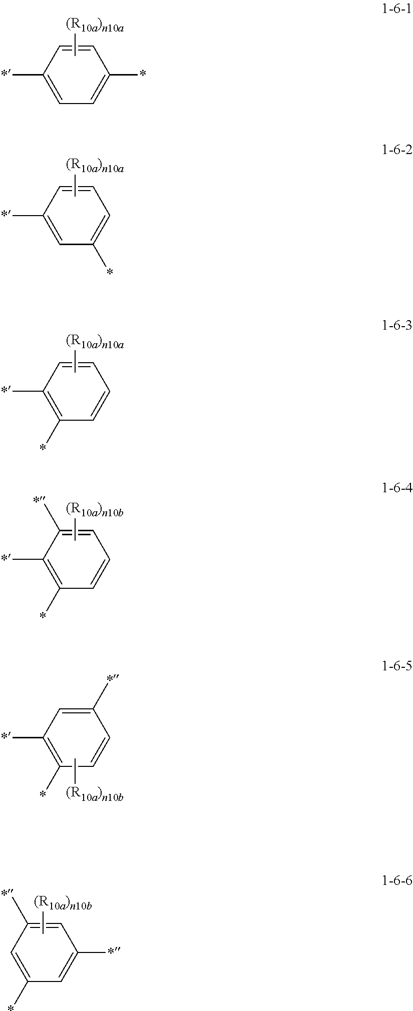

In addition, in Formulae 1-6-1 to 1-6-6, R₁₀ₐ is defined the same as described with respect to Formula 1,

n10a may an integer from 0 to 4, n10b may be an integer from 0 to 3, and *, *′, and *″ may be a binding site to a neighboring atom.

In an embodiment, a13 and a33 may each be 0. Accordingly, the unshared electron pair of the nitrogen atom may be resonance stabilized directly to a spiro non-fluorene moiety.

In an embodiment, at least one of a11, a12, a31, and a32 may be 1. Accordingly, the length of the conjugation system may be increased and the energy level of the highest occupied molecular orbital (HOMO) and/or the energy level of the lowest unoccupied molecular orbital (LUMO) of the diamine compound may be controlled.

In an embodiment, at least one selected from Ar₁₁, Ar₁₂, Ar₃₁, and Ar₃₂ may be a benzene group substituted with four or more deuterium atoms, a naphthalene group substituted with four or more deuterium atoms, an anthracene group substituted with four or more deuterium atoms, a phenanthrene group substituted with four or more deuterium atoms, a pyrene group substituted with four or more deuterium atoms, or a chrysene group substituted with four or more deuterium atoms.

In an embodiment, at least one of Ar₁₁, Ar₁₂, Ar₃₁, and Ar₃₂ may be a partially deuterated group.

In an embodiment, at least one of Ar₁₁, Ar₁₂, Ar₃₁, and Ar₃₂ may be a fully deuterated group.

In an embodiment, at least one of Ar₁₁, Ar₁₂, Ar₃₁, and Ar₃₂ may be a group represented by Formula 1-1 or a group represented by Formula 1-2:

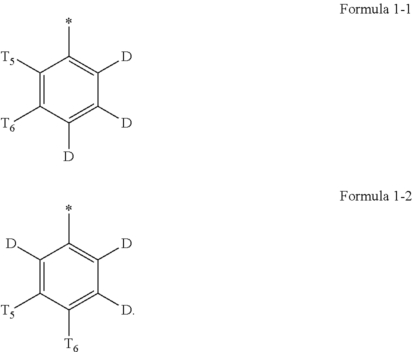

In addition, in Formulae 1-1 and 1-2,

T₅ to T₆ are each defined the same as described with respect to R₁₀ₐ, T₅ and T₆ may be connected to each other to form a C₃-C₃₀ carbocyclic group unsubstituted or substituted with at least one R₁₀ₐ or a C₁-C₃₀ heterocyclic group unsubstituted or substituted with at least one R₁₀ₐ, and * may be a binding site to a neighboring atom.

In an embodiment, a group represented by Formula 1-1 may be a group represented by one of Formulae 1-1-1 to 1-1-3:

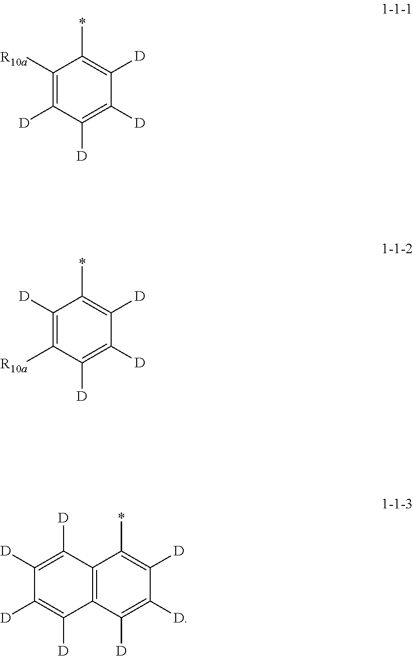

In addition, in Formulae 1-1-1 and 1-1-2,

R₁₀ₐ is defined the same as described with respect to R₁₀ₐ in Formula 1, and * may be a binding site to a neighboring atom.

In an embodiment, a group represented by Formula 1-2 may be a group represented by one of Formulae 1-2-1 to 1-2-3:

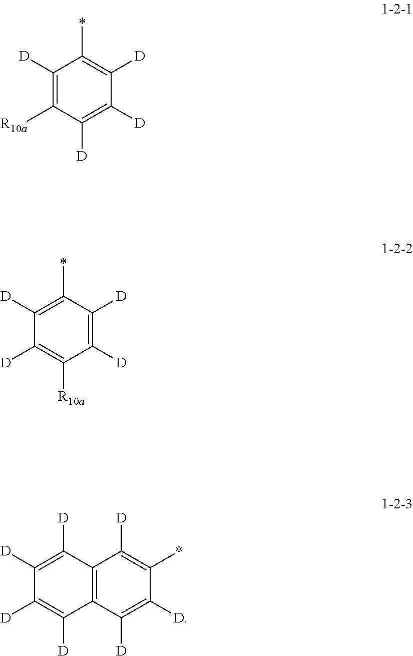

In addition, in Formulae 1-2-1 and 1-2-2,

R₁₀ₐ is defined the same as described with respect to R₁₀ₐ in Formula 1, and * may be a binding site to a neighboring atom.

In an embodiment, at least one selected from Ar₁₁, Ar₁₂, Ar₃₁ and Ar₃₂ may be a group represented by Formula 1-3 or a group represented by Formula 1-4:

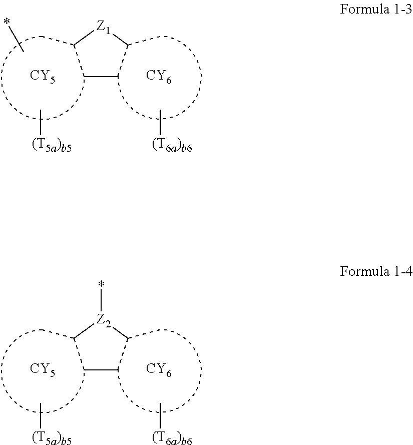

In addition, in Formulae 1-3 and 1-4,

Z₁ may be O, S, N(T₇), P(T₇), C(T₇)(T₈), or Si(T₇)(T₈),

Z₂ may be N, P, C(T₇), or Si(T₇),

CY₅ and CY₆ may each independently be a C₃-C₃₀ carbocyclic group or a C₁-C₃₀ heterocyclic group,

T₅ₐ and T₆ₐ are each defined the same as described with respect to R₁₀ₐ, T₅ₐ and T₆ₐ are connected to each other to form a C₃-C₃₀ carbocyclic group unsubstituted or substituted with at least one R₁₀ₐ or a C₁-C₃₀ heterocyclic group unsubstituted or substituted with at least one R₁₀ₐ,

b5 and b6 may each independently be an integer from 0 to 5,

T₇ to T₈ are each the same as R₁₀ₐ as described herein, T₇ and T₈ are connected to each other to form a C₃-C₃₀ carbocyclic group unsubstituted or substituted with at least one R₁₀ₐ or a C₁-C₃₀ heterocyclic group unsubstituted or substituted with at least one R₁₀ₐ, and

* indicates a binding site to a neighboring atom.

In an embodiment, Ar₁₁ may be a group substituted with at least four deuterium atoms, and Ar₁₂ may be a group represented by Formula 1-3 or a group represented by Formula 1-4.

In an embodiment, Ar₁₁ may be a group substituted with four or more deuterium atoms, and at least one of Ar₃₁ or A₃₂ may be a group represented by Formula 1-3 or a group represented by Formula 1-4.

In an embodiment, in a group represented by Formula 1-3,

a group represented by

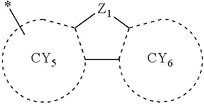

may be one selected from croups represented by Formulae 1-3-1 to 1-3-4:

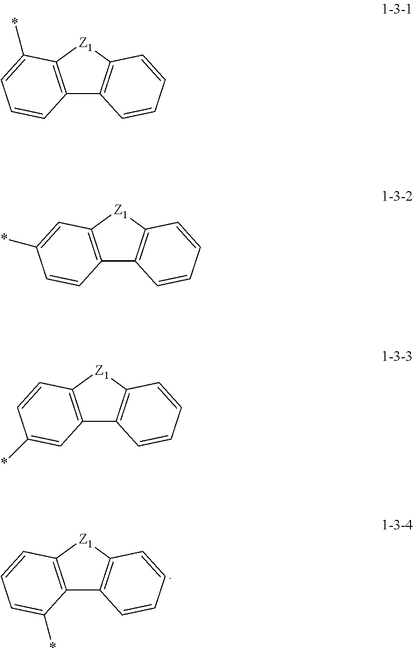

In addition, in Formulae 1-3-1 to 1-3-4, * indicates a binding site to a neighboring atom.

In an embodiment, a C₃-C₃₀ carbocyclic group may be a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indeno phenanthrene group, or an indenoanthracene group, and

a C₁-C₃₀ heterocyclic group may be a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, or an azadibenzofuran group.

In an embodiment, a diamine compound represented by Formula 1 may be one selected from Compounds 1 to 420:

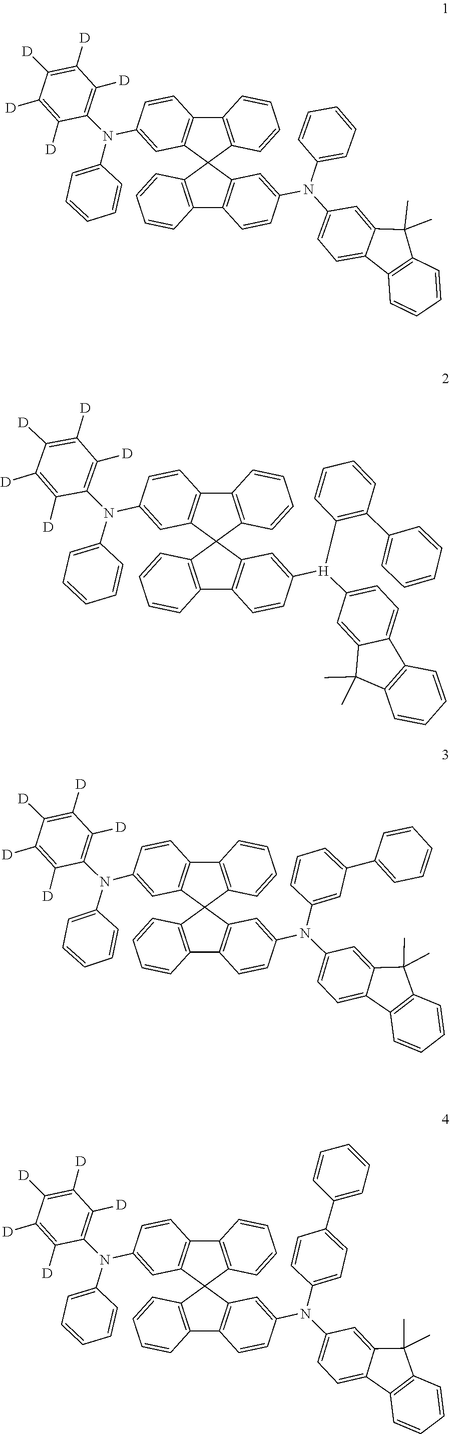

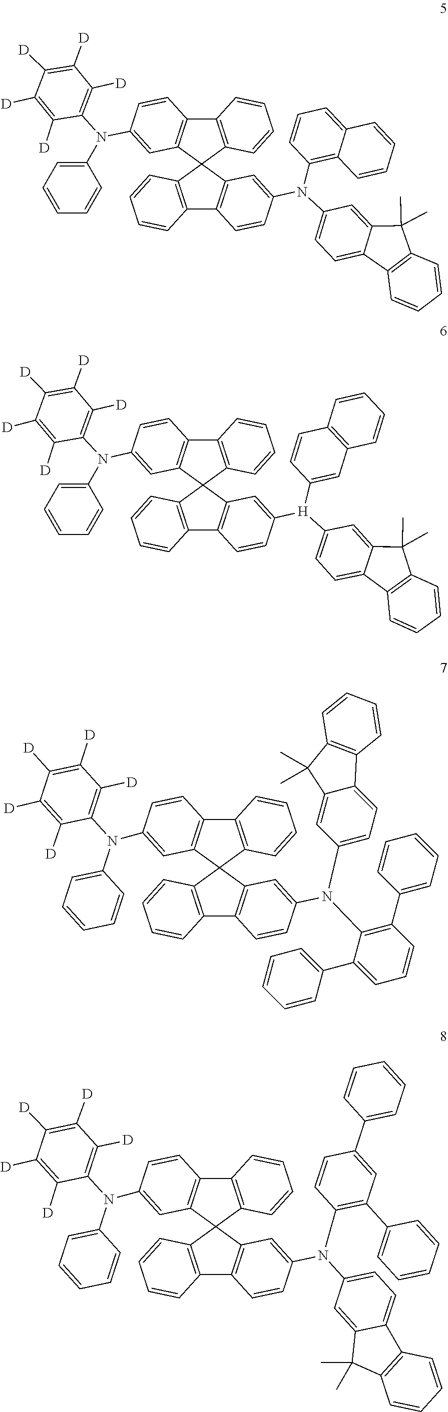

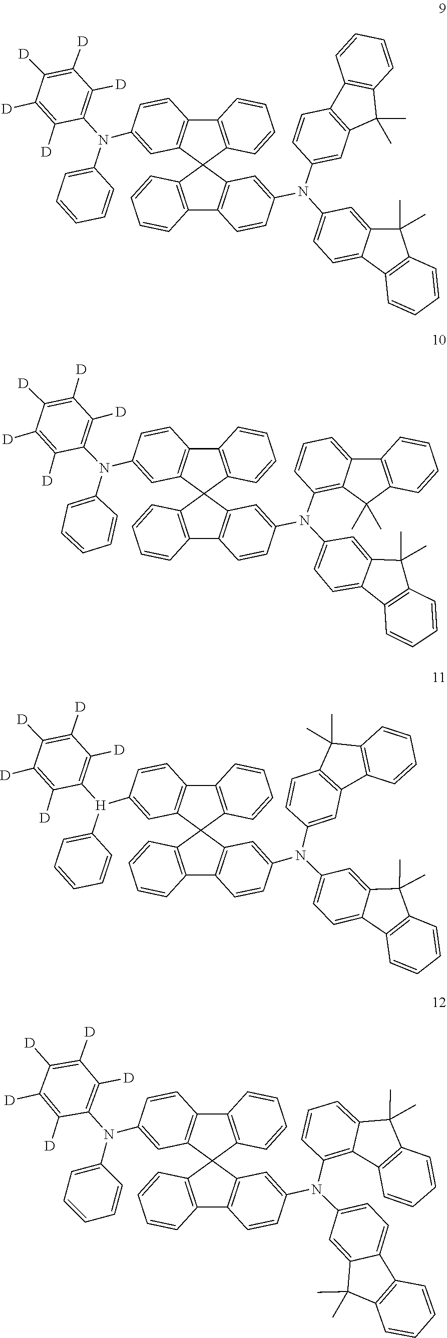

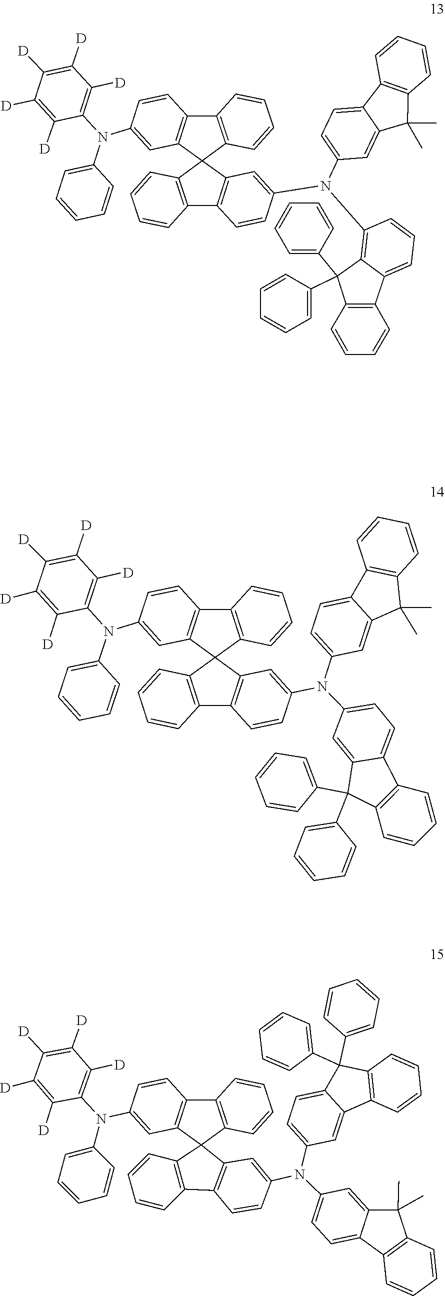 [SMILES: [2H]c1c([2H])c([2H])c(N(c2ccccc2)c2ccc3c(c2)C2(c4ccccc4-c4ccc(N(c5ccc6c(c5)C(C)(C)c5ccccc5-6)c5cccc6c5C(c5ccccc5)(c5ccccc5)c5ccccc5-6)cc42)c2ccccc2-3)c([2H])c1[2H] ; [2H]c1c([2H])c([2H])c(N(c2ccccc2)c2ccc3c(c2)C2(c4ccccc4-c4ccc(N(c5ccc6c(c5)C(C)(C)c5ccccc5-6)c5ccc6c(c5)C(c5ccccc5)(c5ccccc5)c5ccccc5-6)cc42)c2ccccc2-3)c([2H])c1[2H] ; [2H]c1c([2H])c([2H])c(N(c2ccccc2)c2ccc3c(c2)C2(c4ccccc4-c4ccc(N(c5ccc6c(c5)-c5ccccc5C6(c5ccccc5)c5ccccc5)c5ccc6c(c5)C(C)(C)c5ccccc5-6)cc42)c2ccccc2-3)c([2H])c1[2H]]

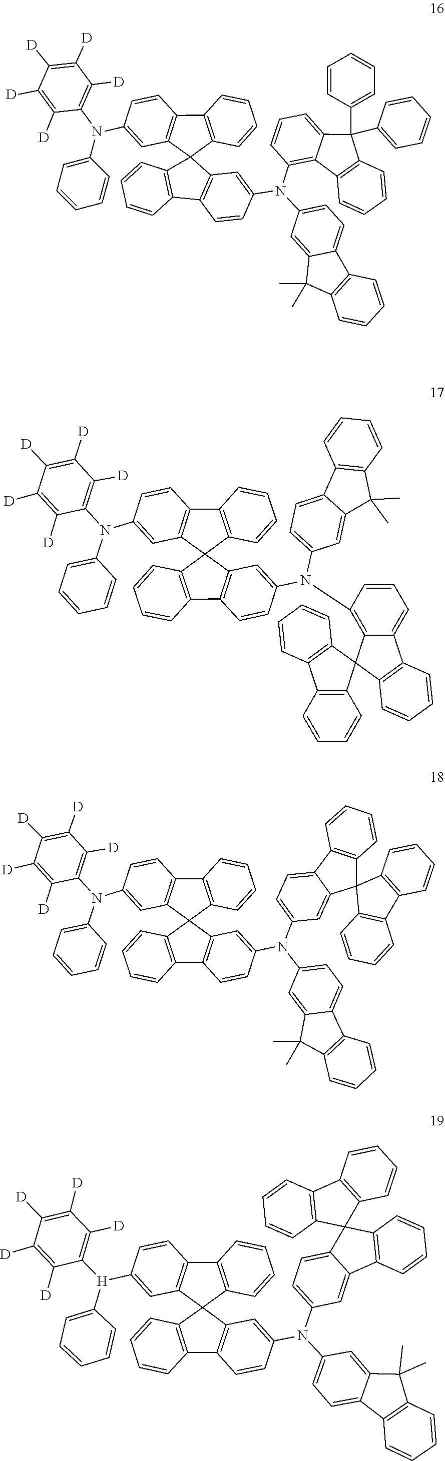

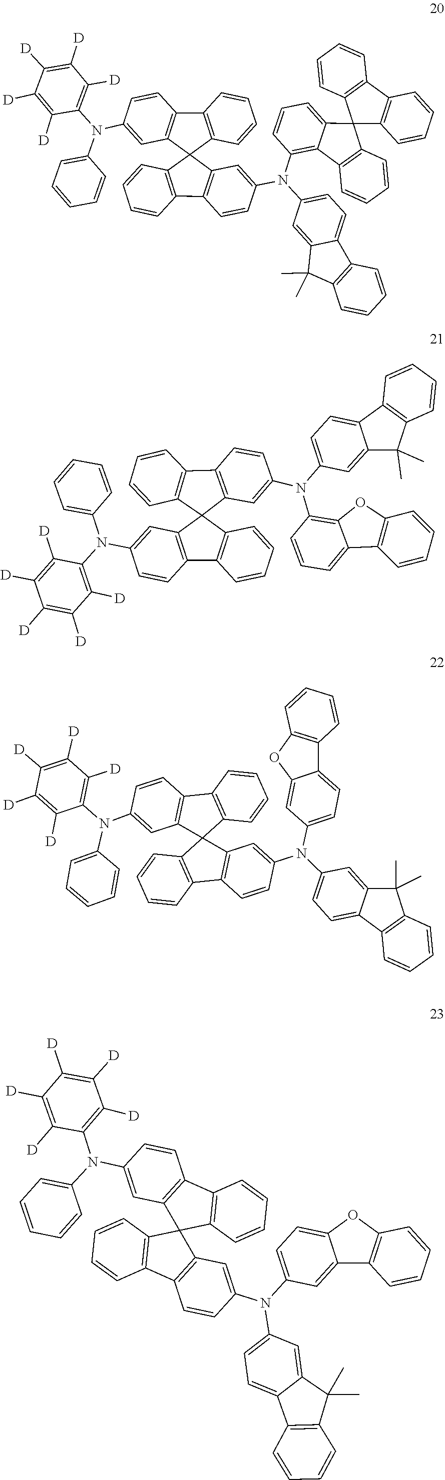

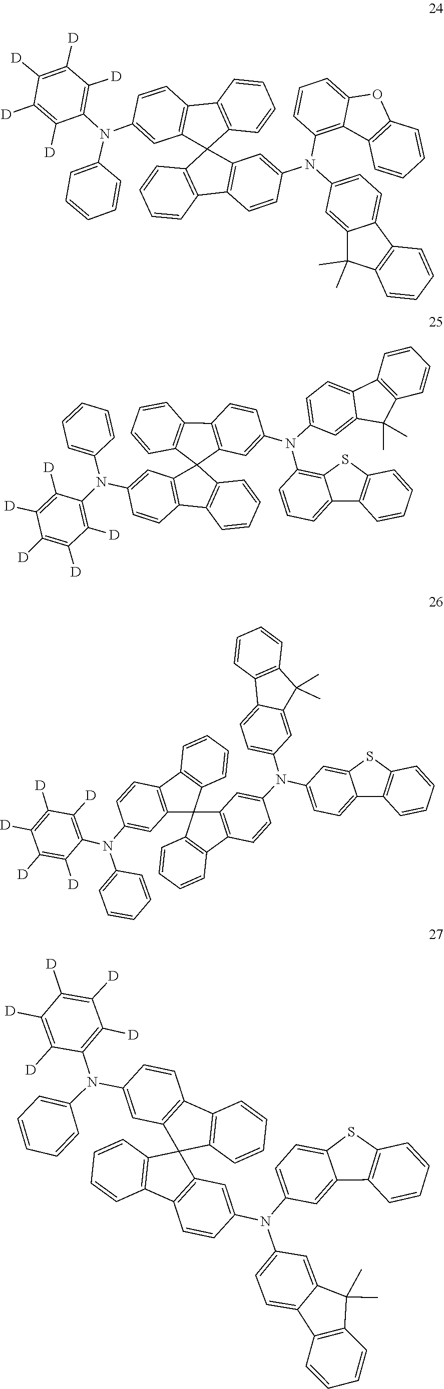

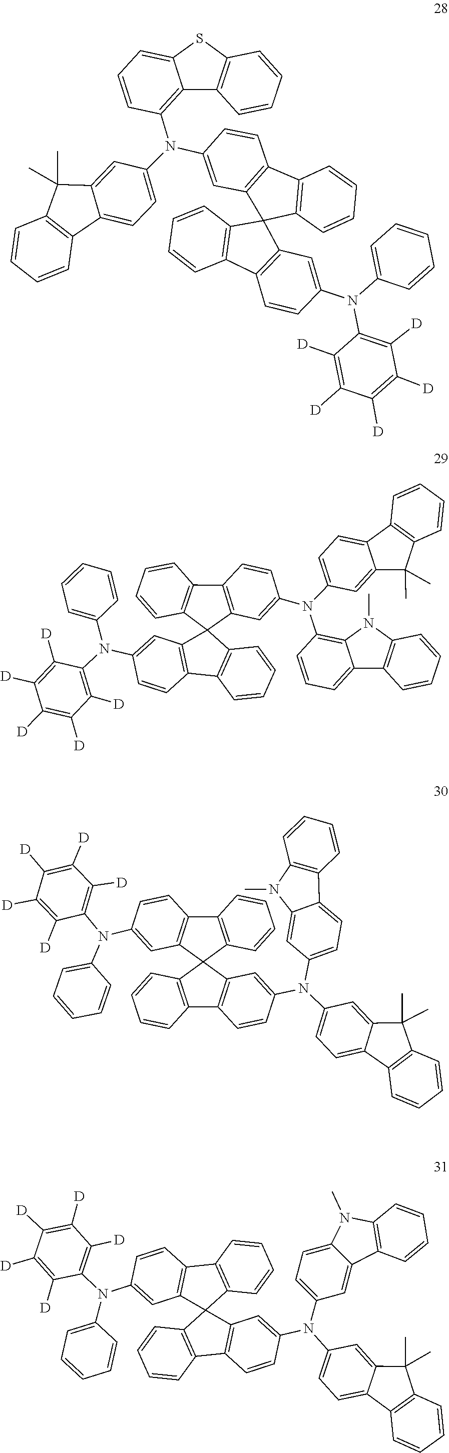

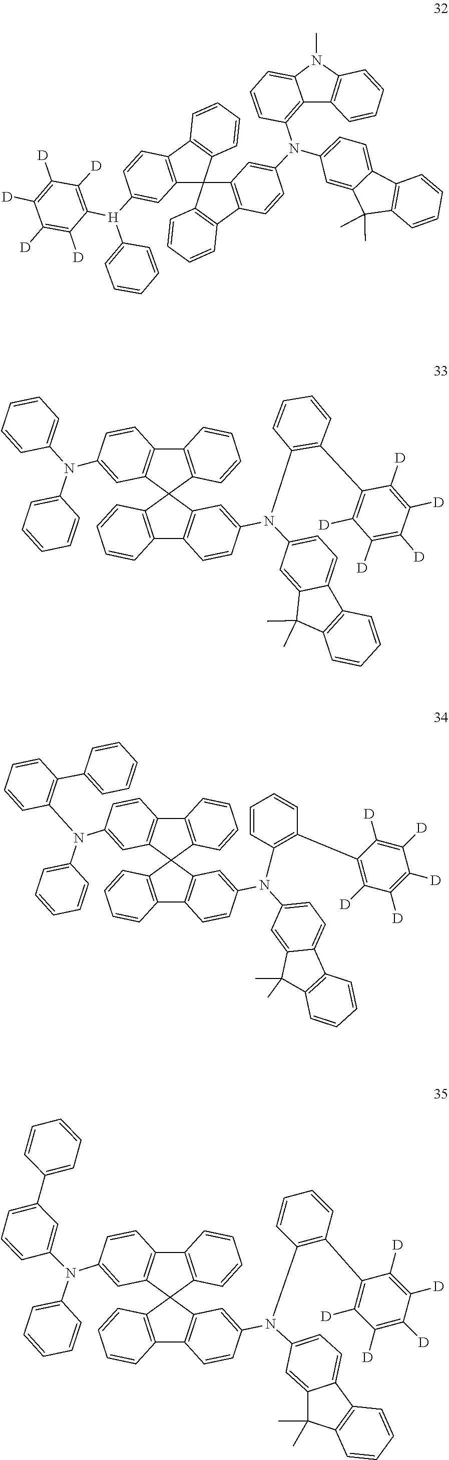

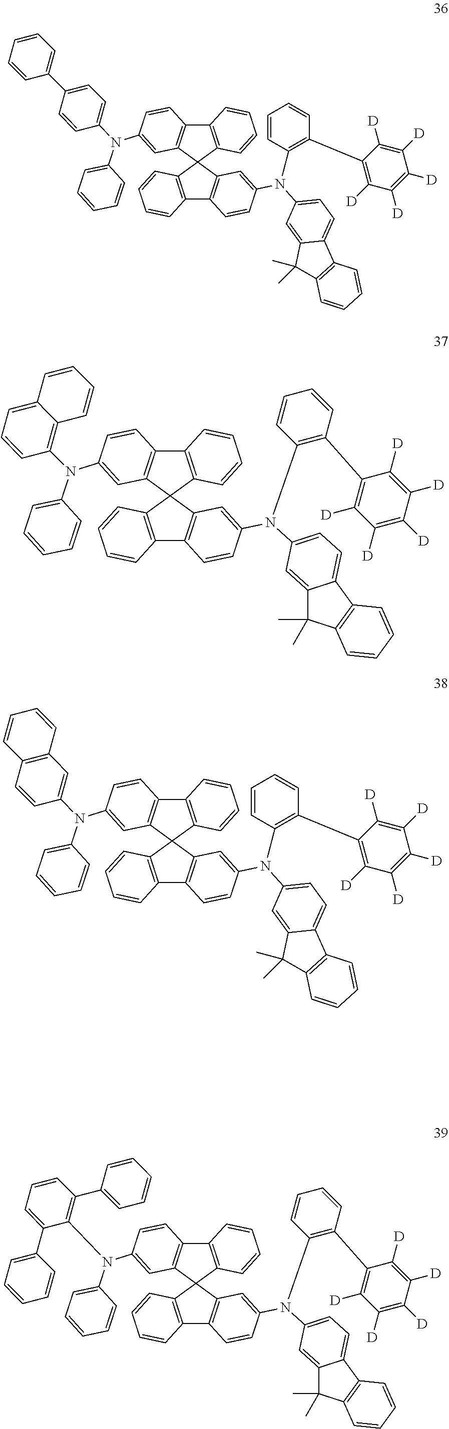

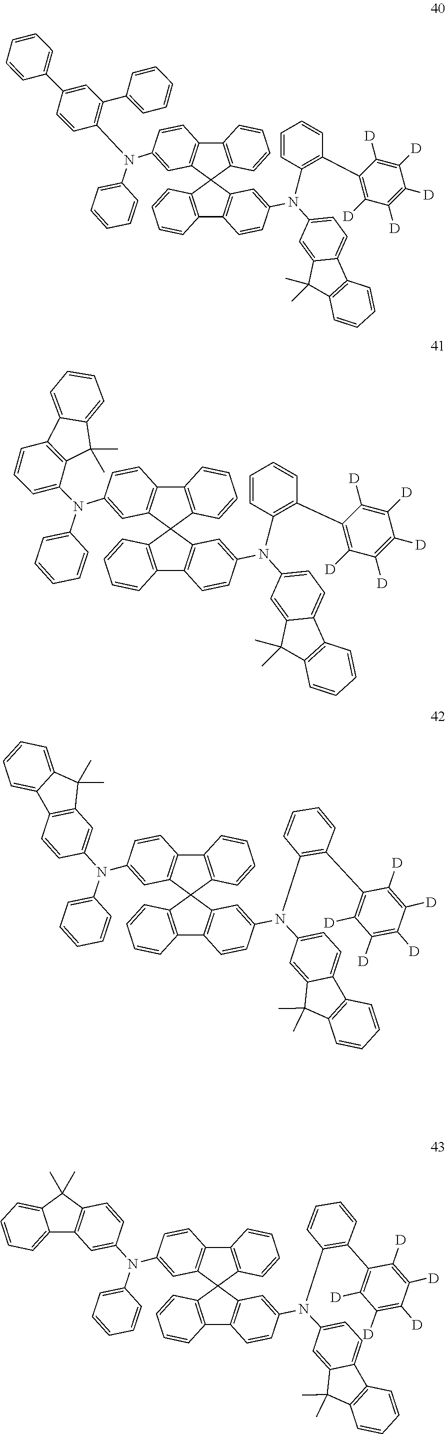

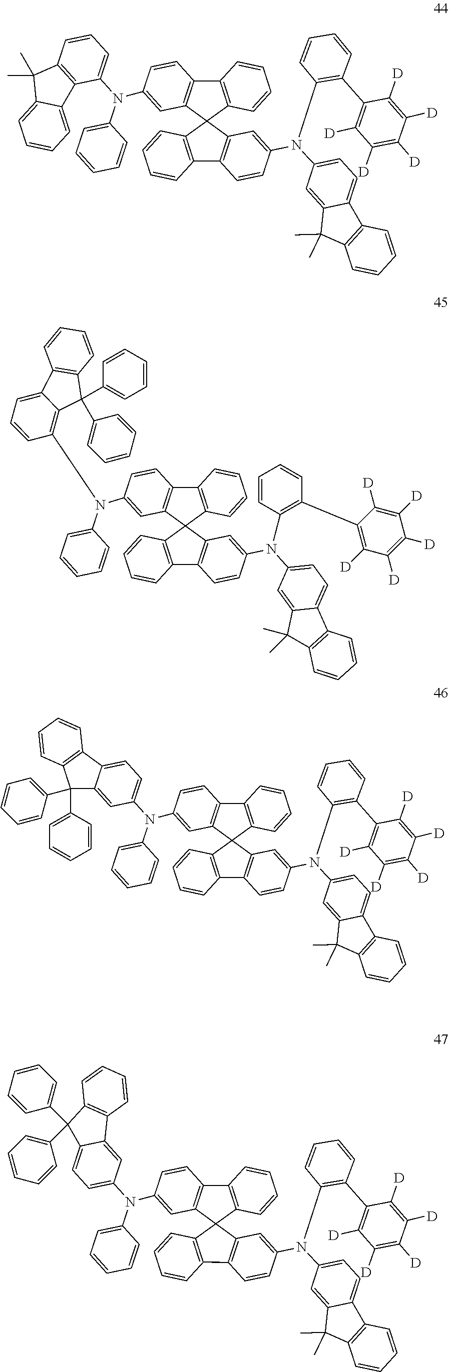

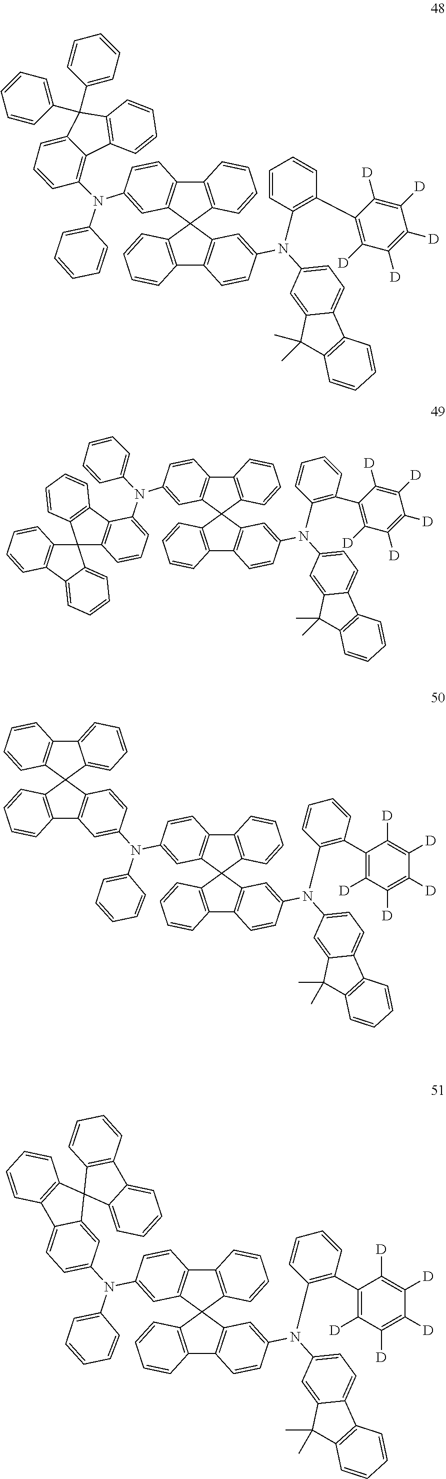

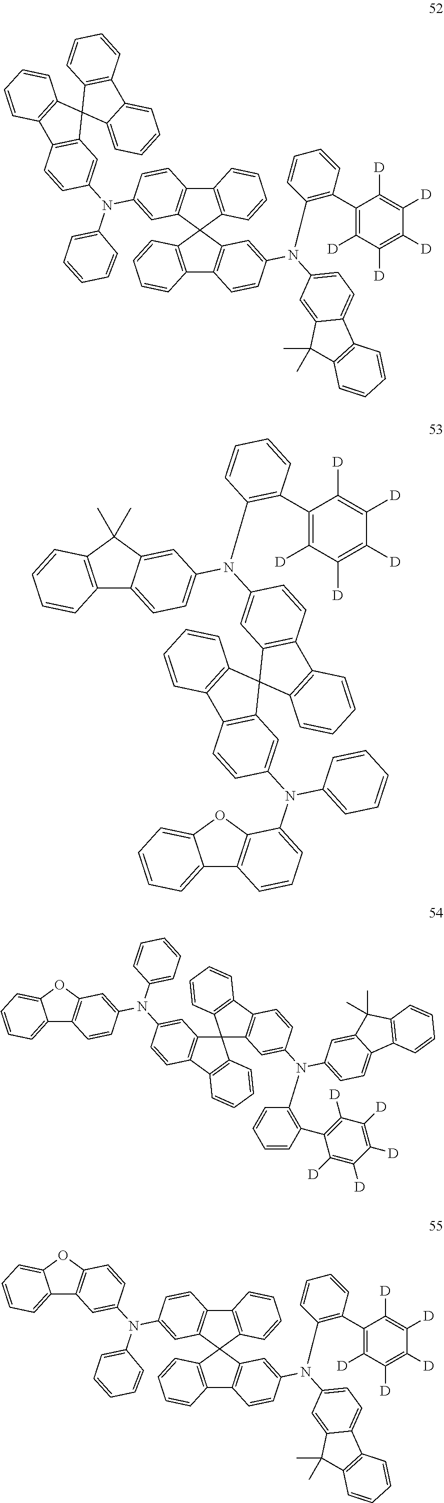

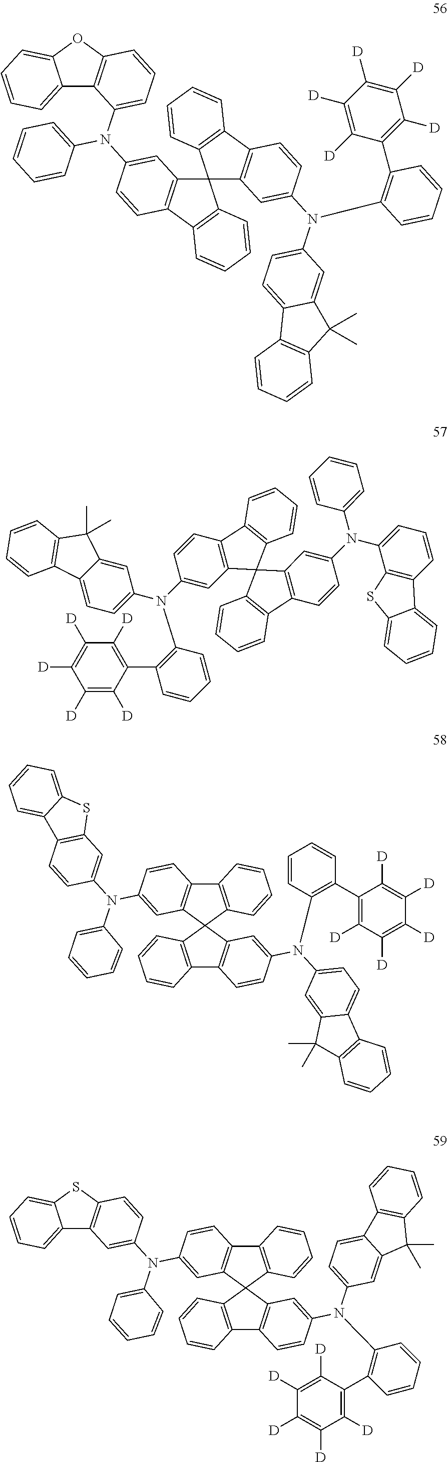

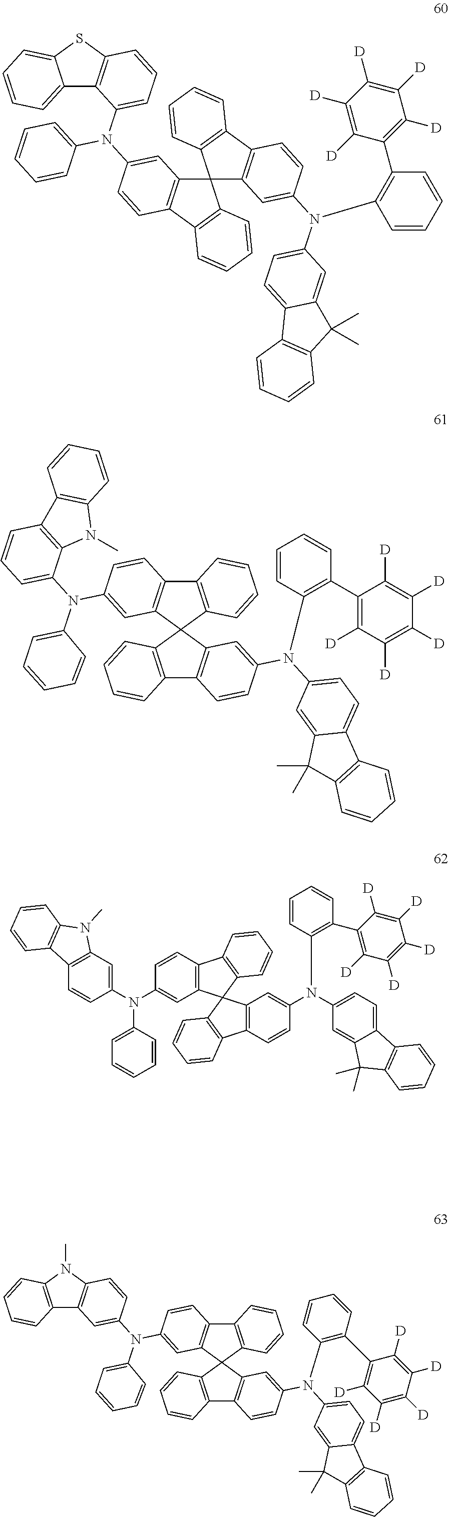

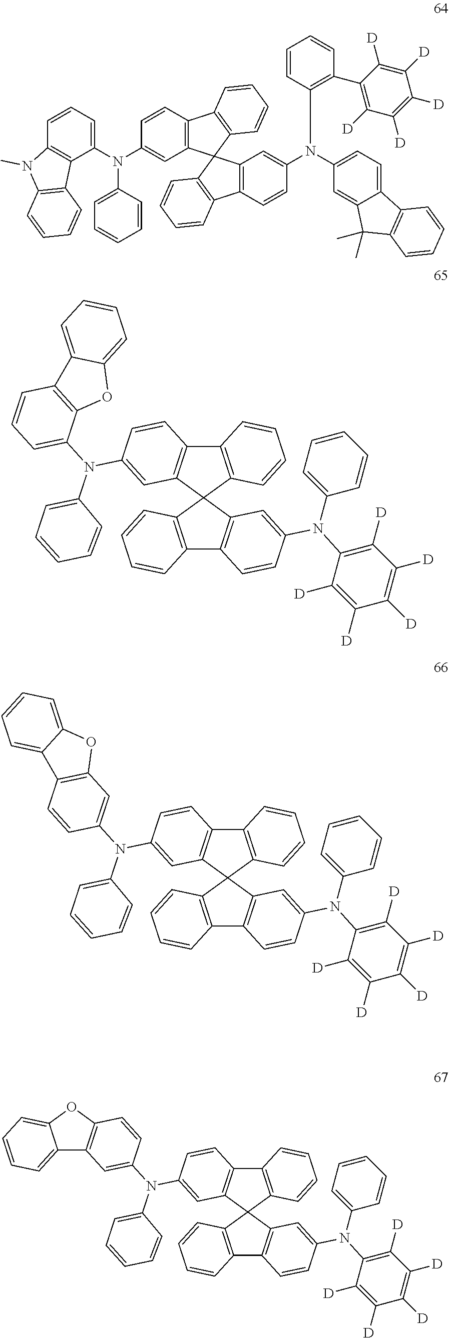

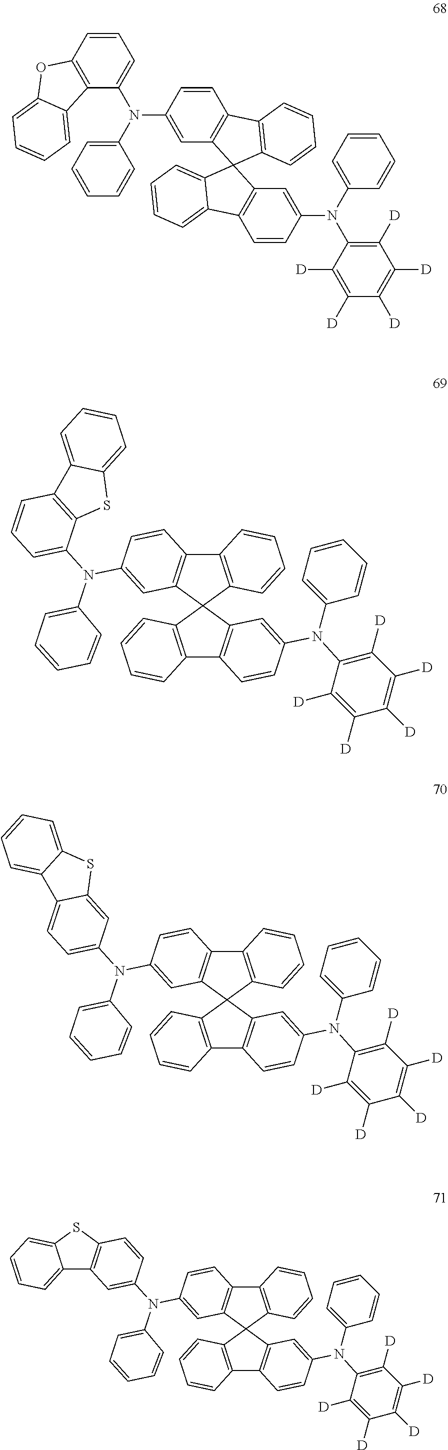 [SMILES: [2H]c1c([2H])c([2H])c(N(c2ccccc2)c2ccc3c(c2)C2(c4ccccc4-3)c3ccccc3-c3ccc(N(c4ccccc4)c4cccc5oc6ccccc6c45)cc32)c([2H])c1[2H] ; [2H]c1c([2H])c([2H])c(N(c2ccccc2)c2ccc3c(c2)C2(c4ccccc4-3)c3ccccc3-c3ccc(N(c4ccccc4)c4cccc5c4sc4ccccc45)cc32)c([2H])c1[2H] ; [2H]c1c([2H])c([2H])c(N(c2ccccc2)c2ccc3c(c2)C2(c4ccccc4-c4ccc(N(c5ccccc5)c5ccc6c(c5)sc5ccccc56)cc42)c2ccccc2-3)c([2H])c1[2H] ; [2H]c1c([2H])c([2H])c(N(c2ccccc2)c2ccc3c(c2)C2(c4ccccc4-c4ccc(N(c5ccccc5)c5ccc6sc7ccccc7c6c5)cc42)c2ccccc2-3)c([2H])c1[2H]]

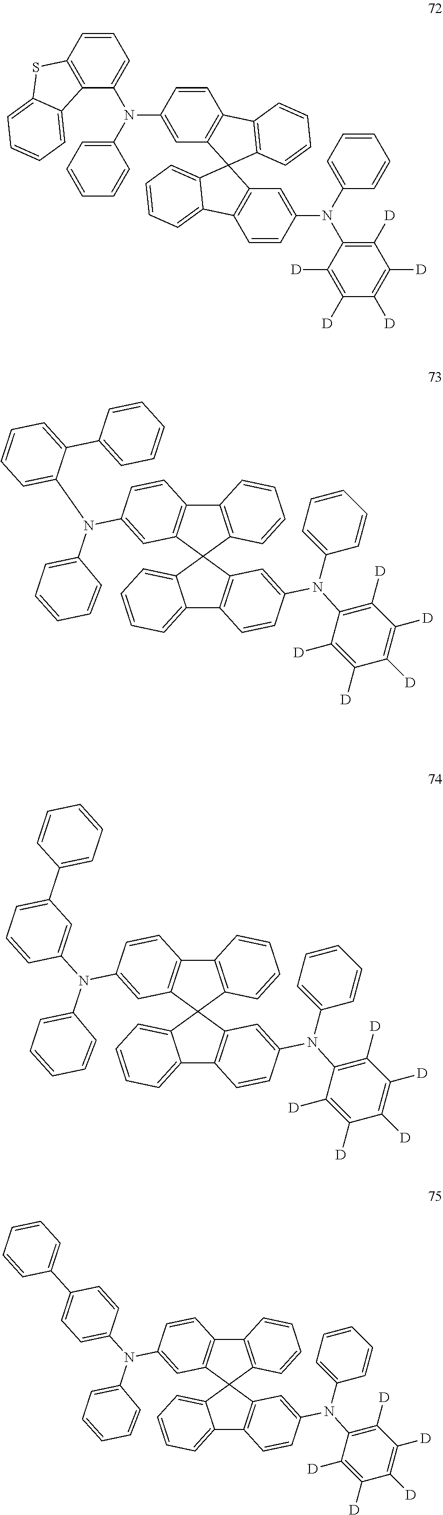 [SMILES: [2H]c1c([2H])c([2H])c(N(c2ccccc2)c2ccc3c(c2)C2(c4ccccc4-3)c3ccccc3-c3ccc(N(c4ccccc4)c4cccc5sc6ccccc6c45)cc32)c([2H])c1[2H] ; [2H]c1c([2H])c([2H])c(N(c2ccccc2)c2ccc3c(c2)C2(c4ccccc4-c4ccc(N(c5ccccc5)c5ccccc5-c5ccccc5)cc42)c2ccccc2-3)c([2H])c1[2H] ; [2H]c1c([2H])c([2H])c(N(c2ccccc2)c2ccc3c(c2)C2(c4ccccc4-c4ccc(N(c5ccccc5)c5cccc(-c6ccccc6)c5)cc42)c2ccccc2-3)c([2H])c1[2H] ; [2H]c1c([2H])c([2H])c(N(c2ccccc2)c2ccc3c(c2)C2(c4ccccc4-c4ccc(N(c5ccccc5)c5ccc(-c6ccccc6)cc5)cc42)c2ccccc2-3)c([2H])c1[2H]]

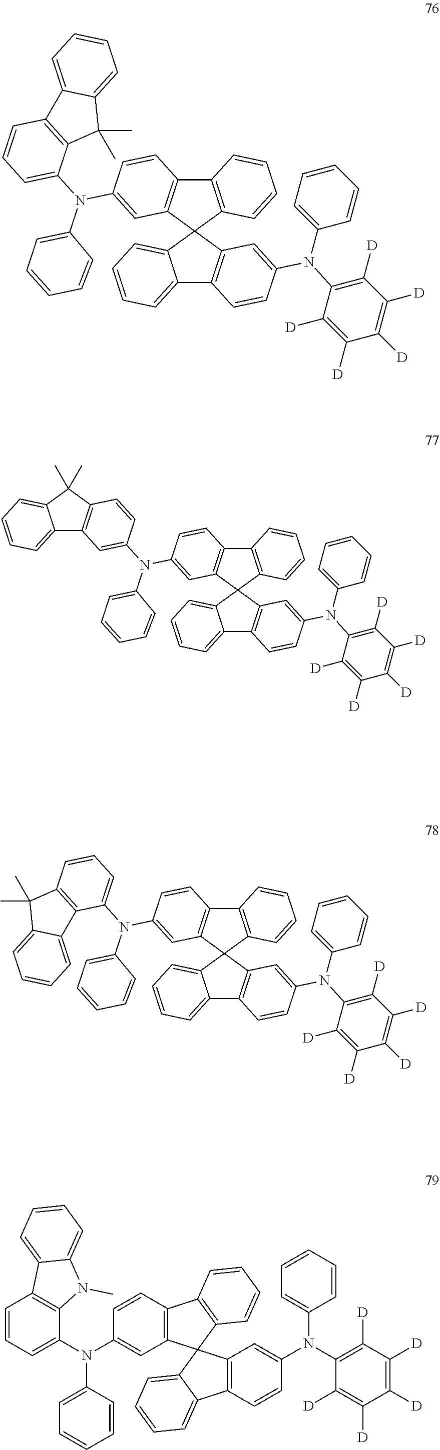

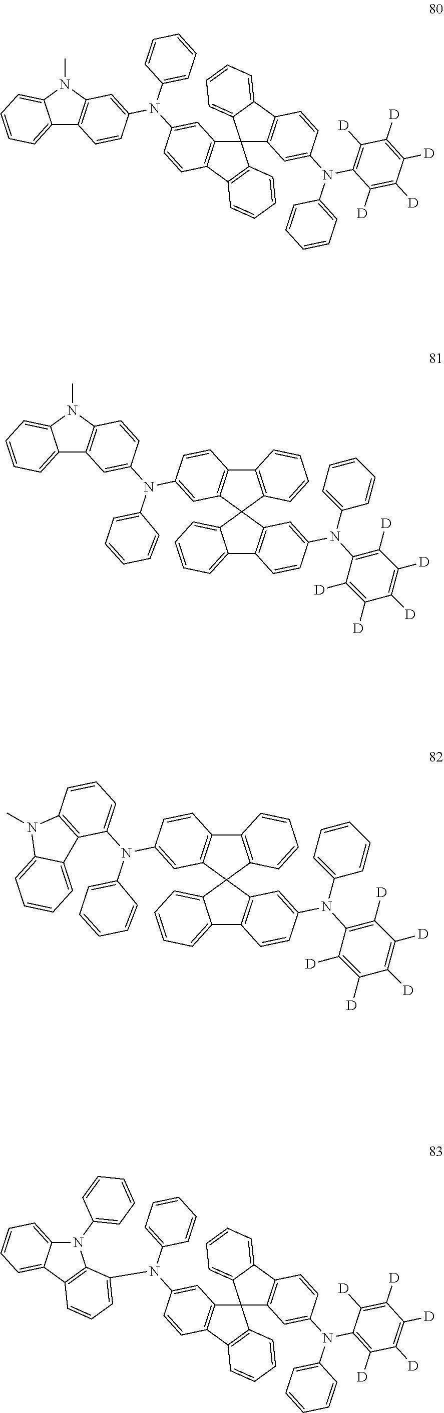

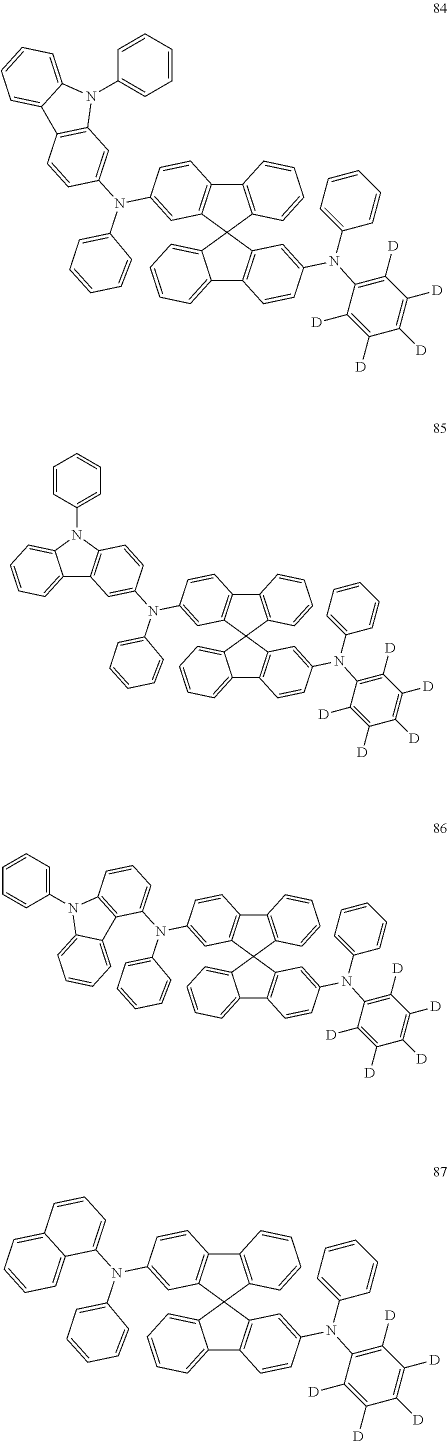

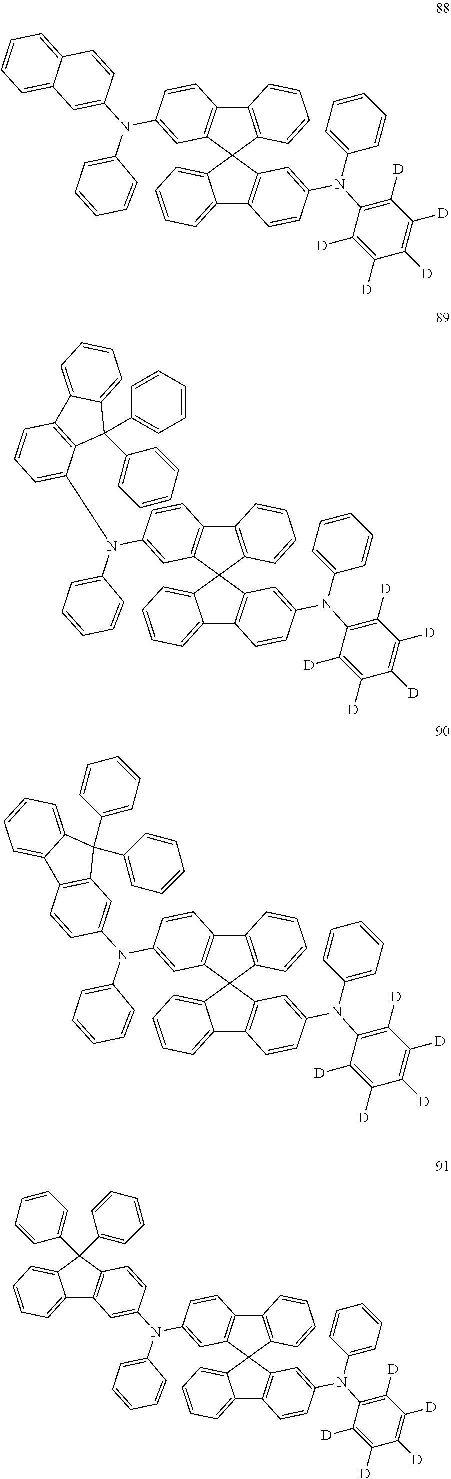

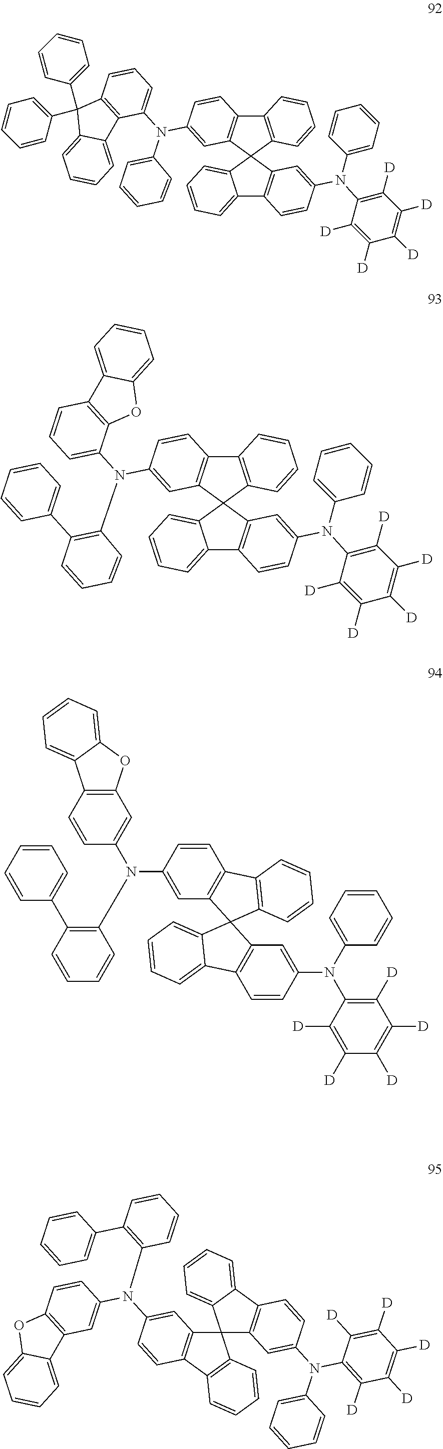

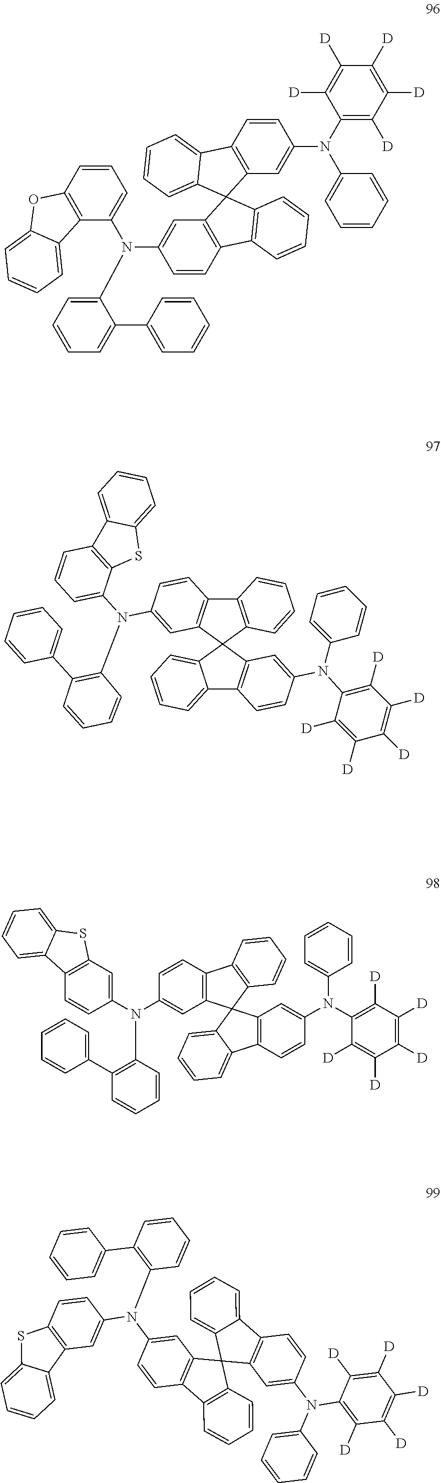

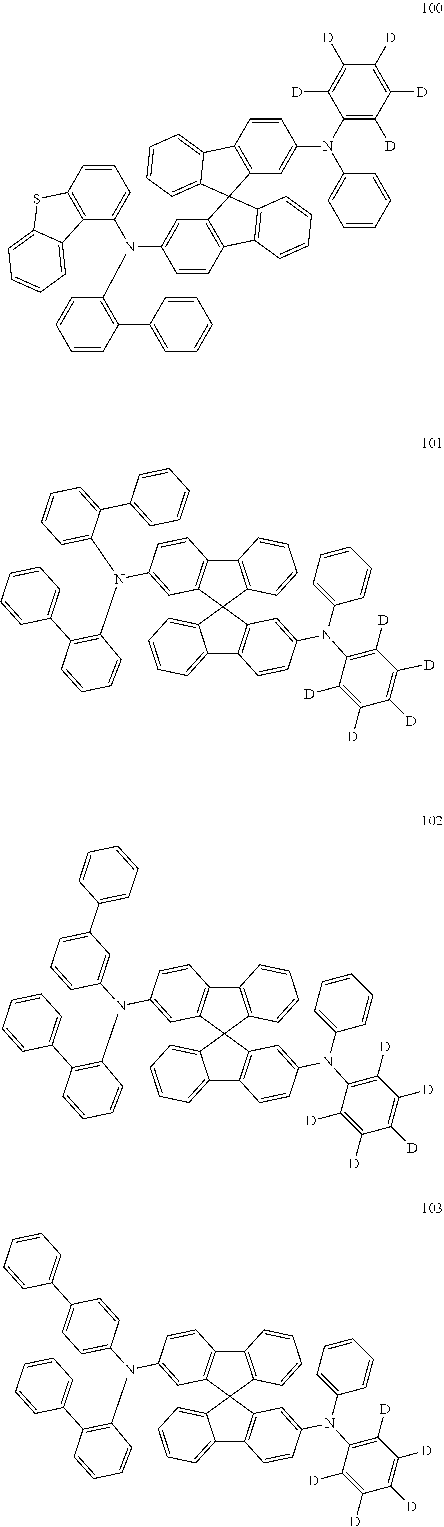

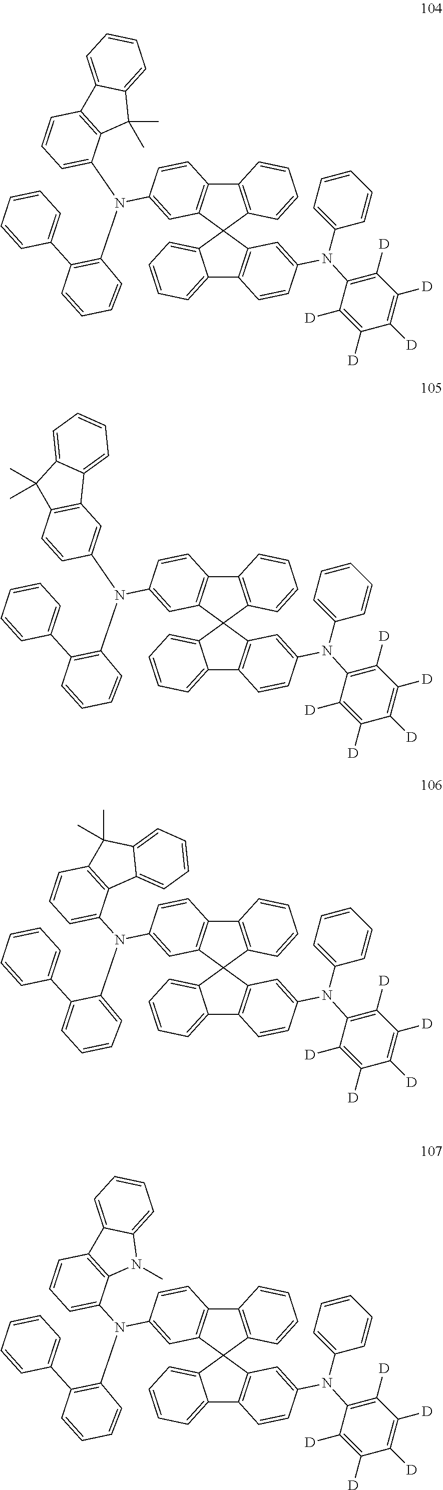

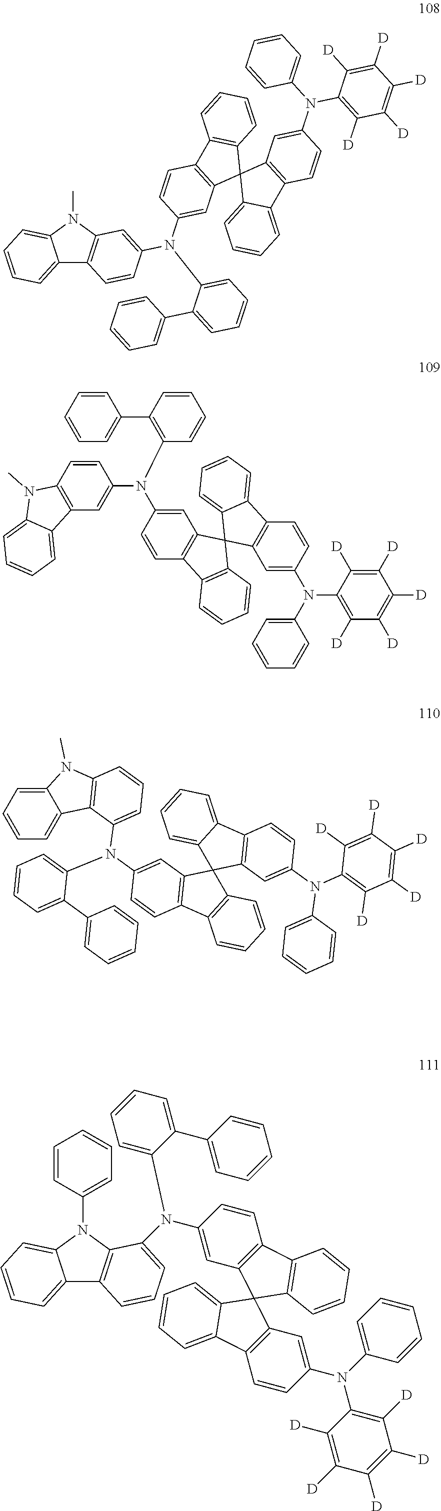

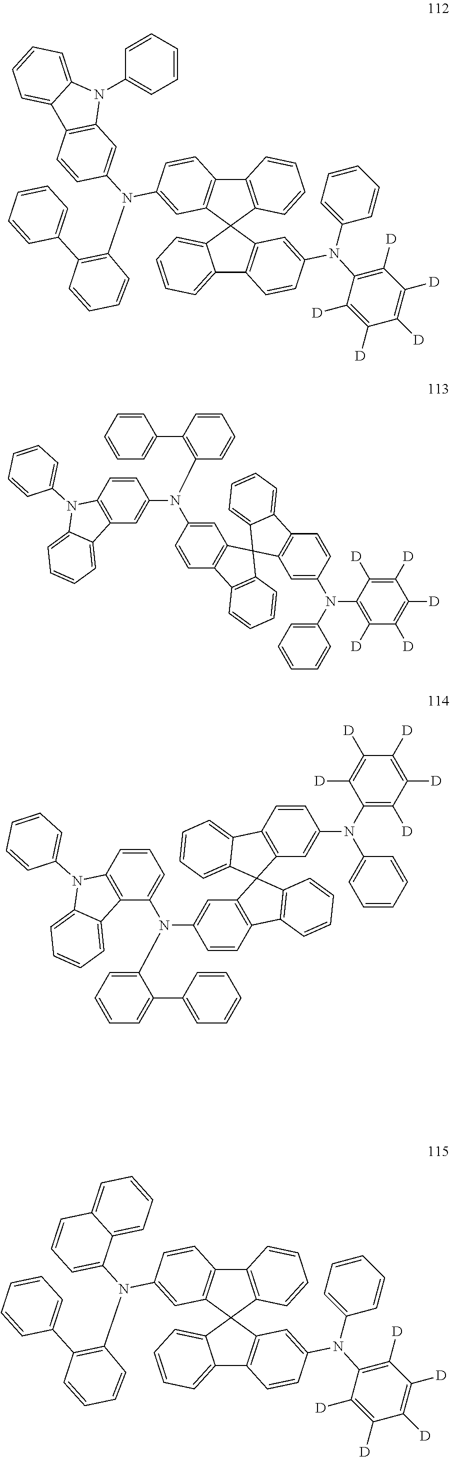

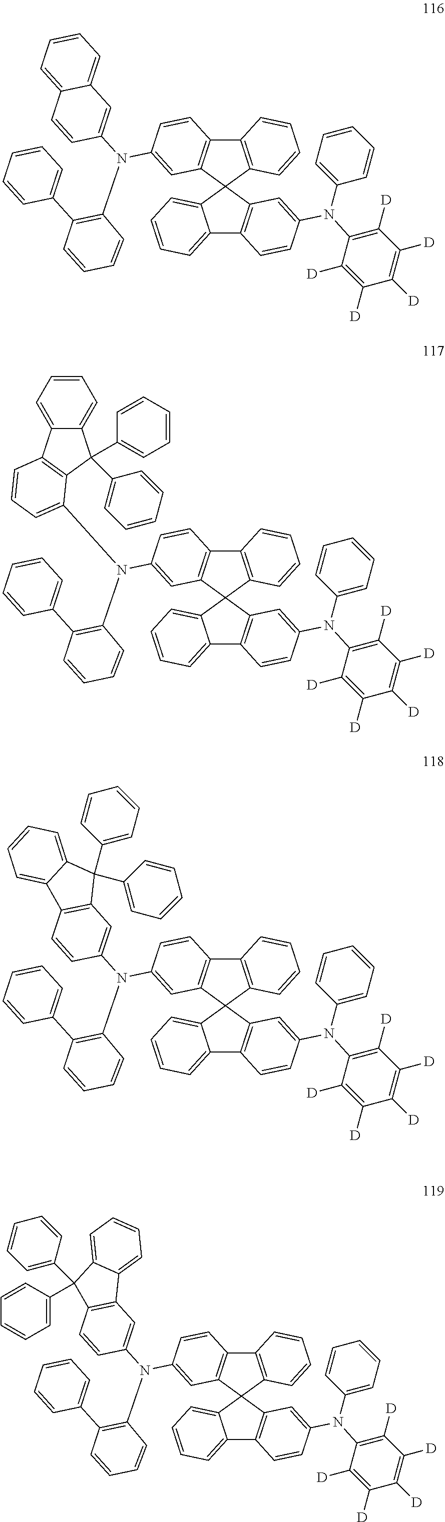

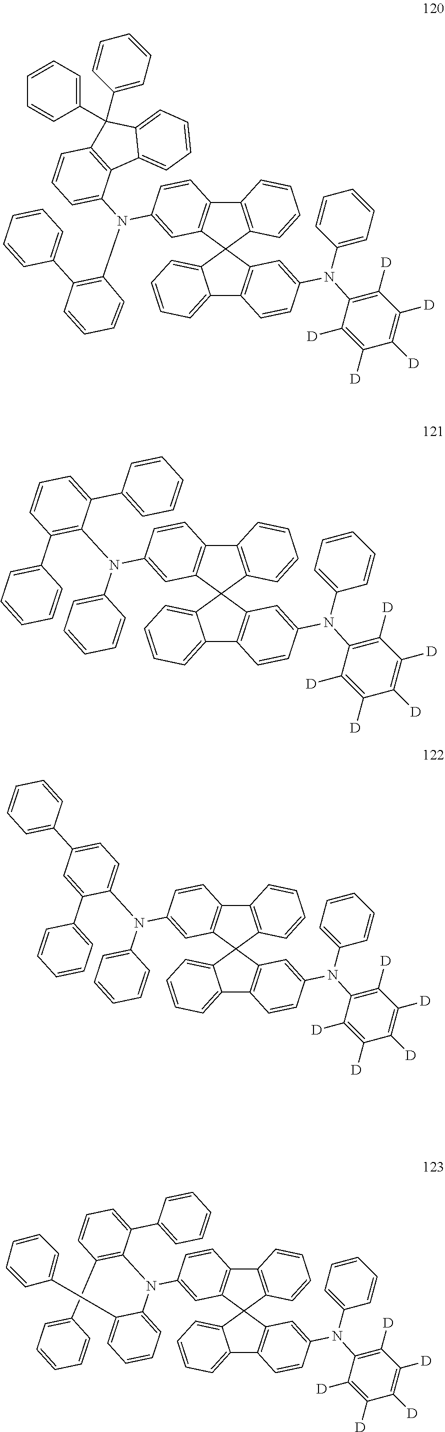

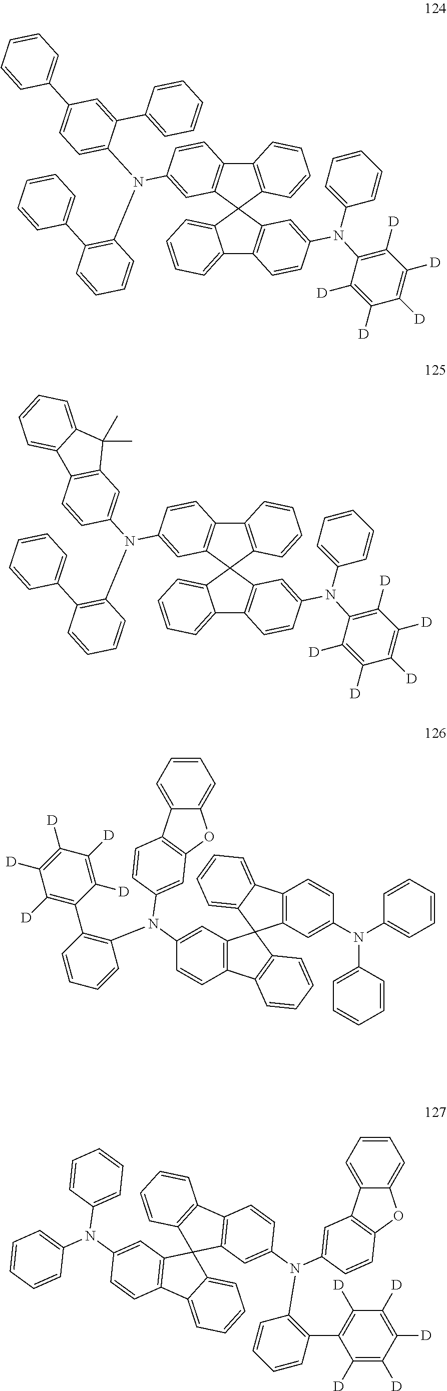

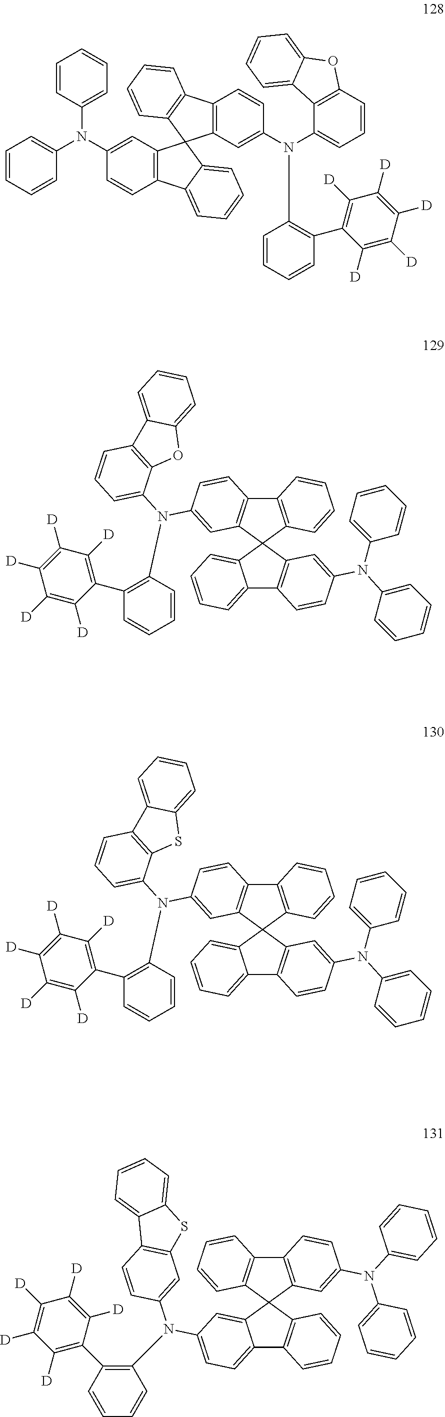

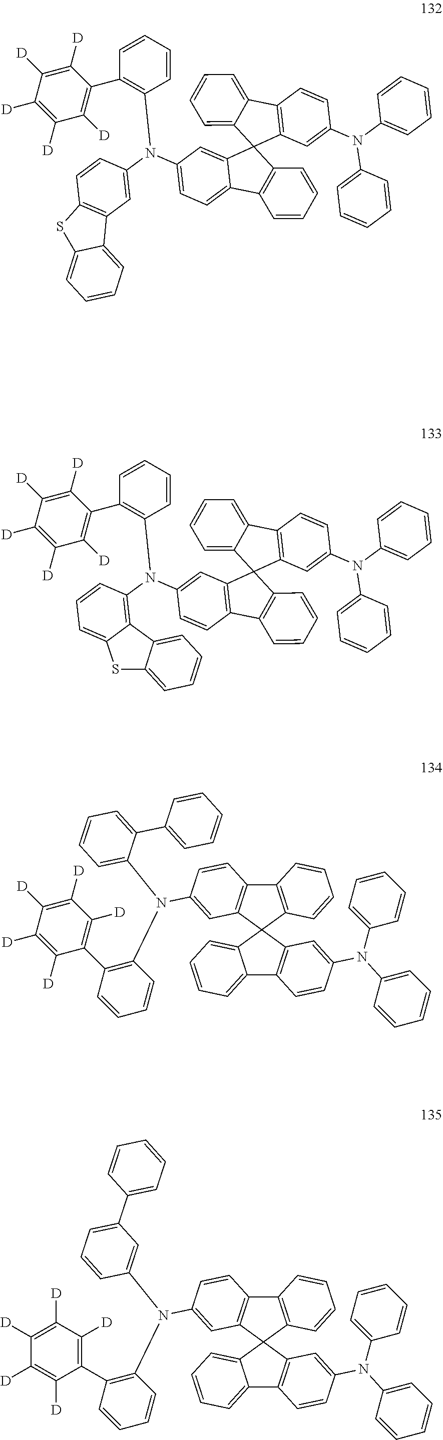

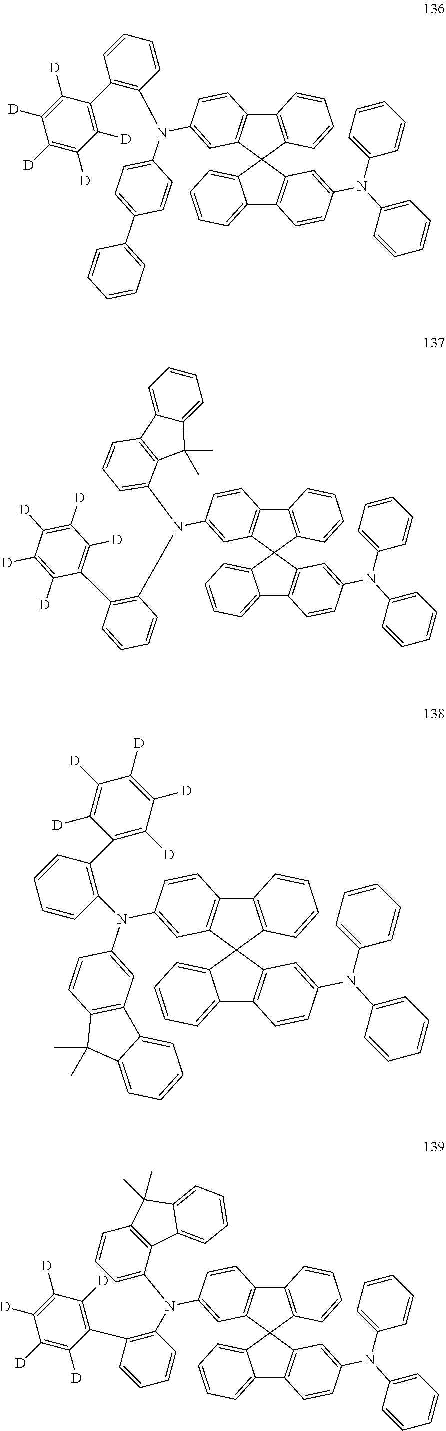

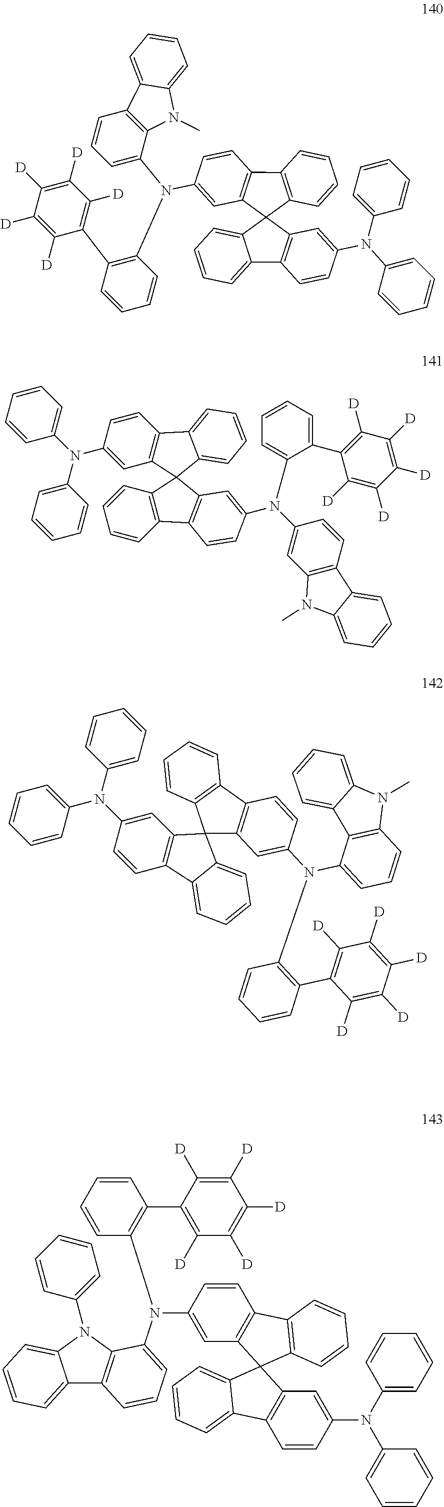

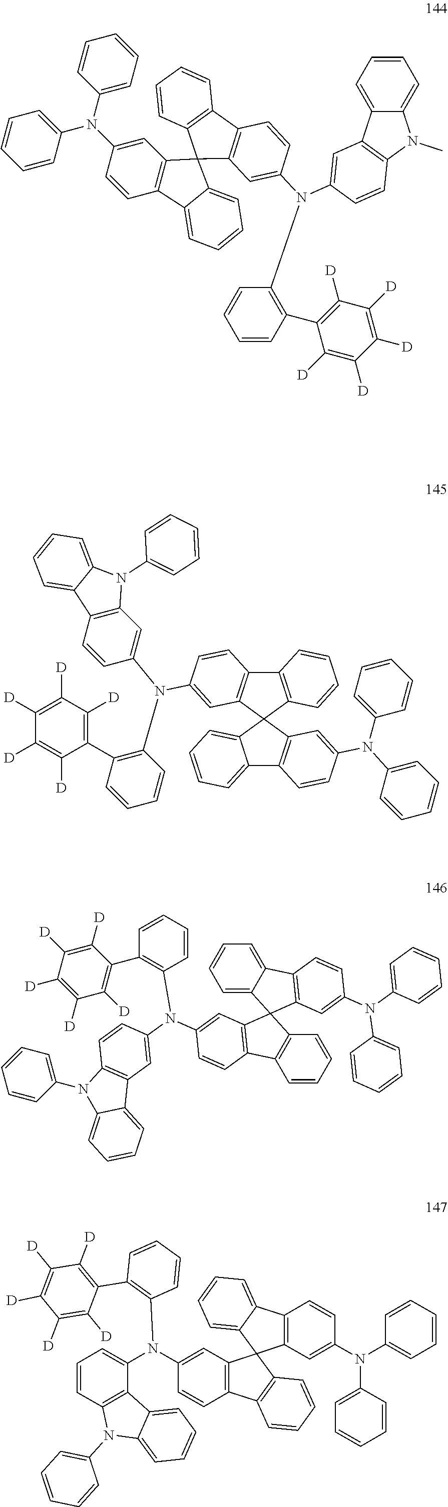

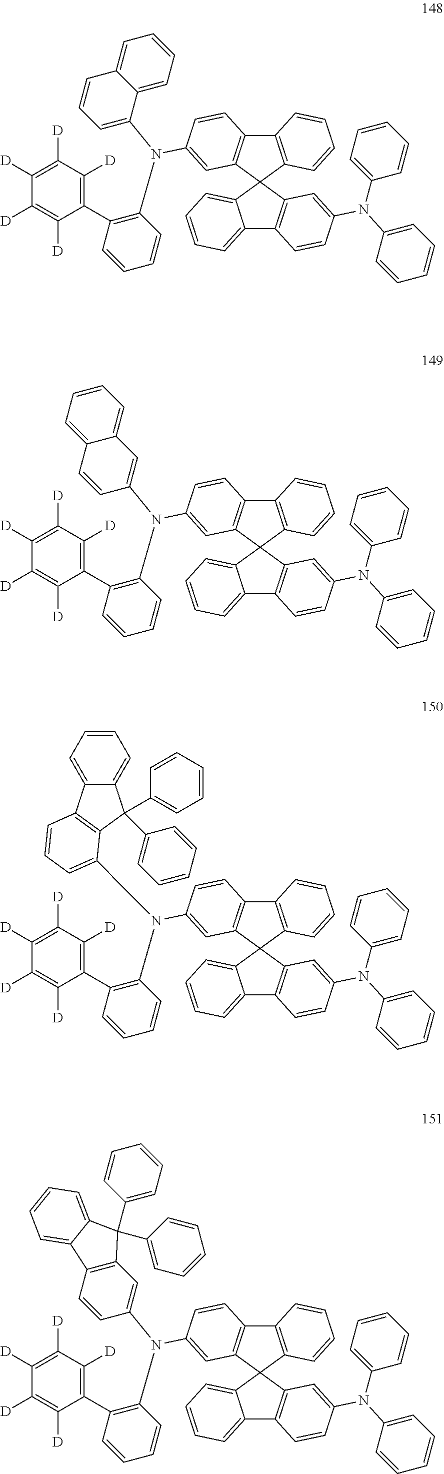

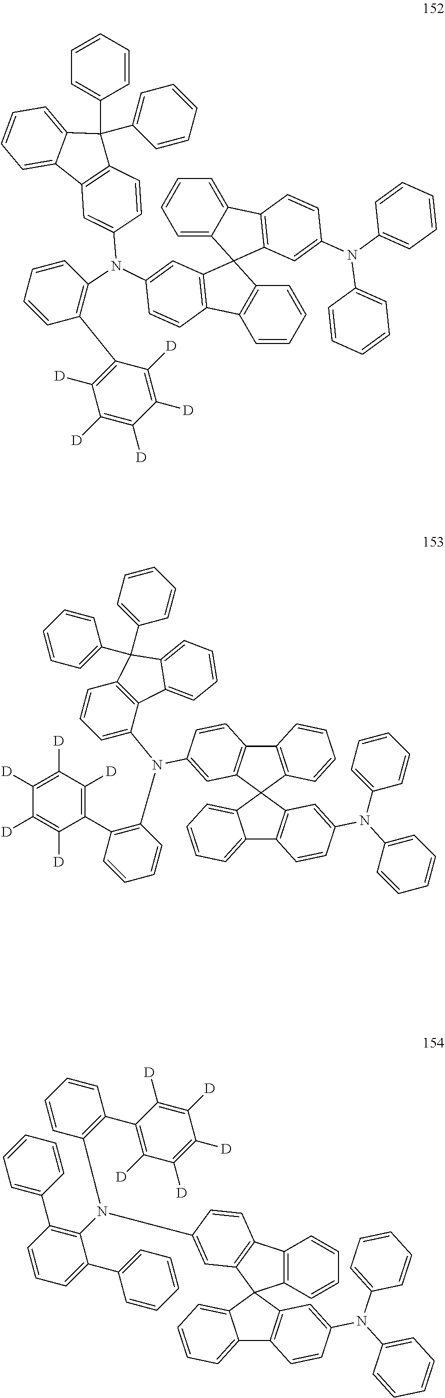 [SMILES: [2H]c1c([2H])c([2H])c(-c2ccccc2N(c2ccc3c(c2)-c2ccccc2C3(c2ccccc2)c2ccccc2)c2ccc3c(c2)C2(c4ccccc4-c4ccc(N(c5ccccc5)c5ccccc5)cc42)c2ccccc2-3)c([2H])c1[2H] ; [2H]c1c([2H])c([2H])c(-c2ccccc2N(c2ccc3c(c2)C2(c4ccccc4-c4ccc(N(c5ccccc5)c5ccccc5)cc42)c2ccccc2-3)c2cccc3c2-c2ccccc2C3(c2ccccc2)c2ccccc2)c([2H])c1[2H] ; [2H]c1c([2H])c([2H])c(-c2ccccc2N(c2ccc3c(c2)C2(c4ccccc4-c4ccc(N(c5ccccc5)c5ccccc5)cc42)c2ccccc2-3)c2c(-c3ccccc3)cccc2-c2ccccc2)c([2H])c1[2H]]

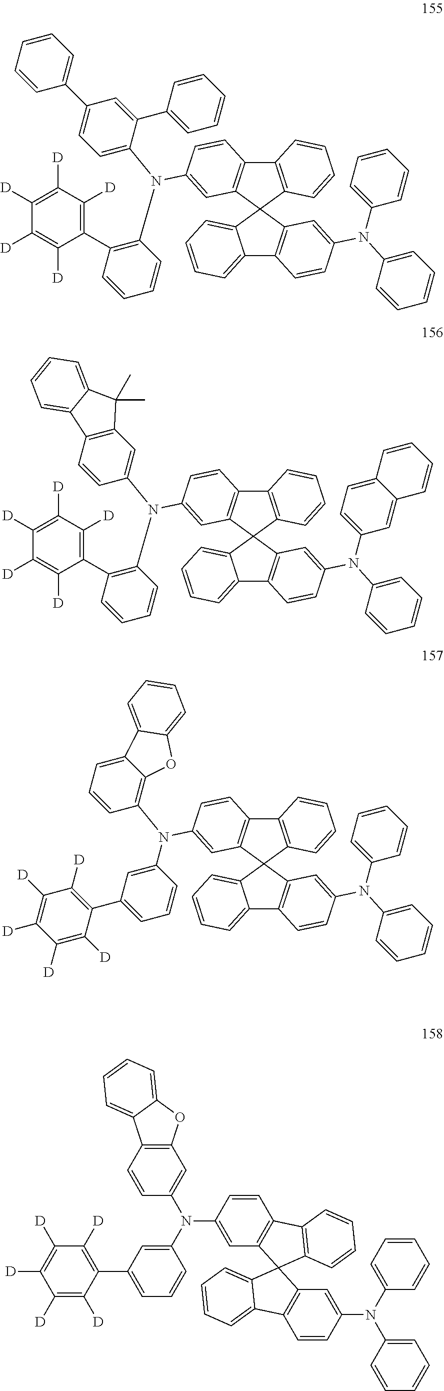

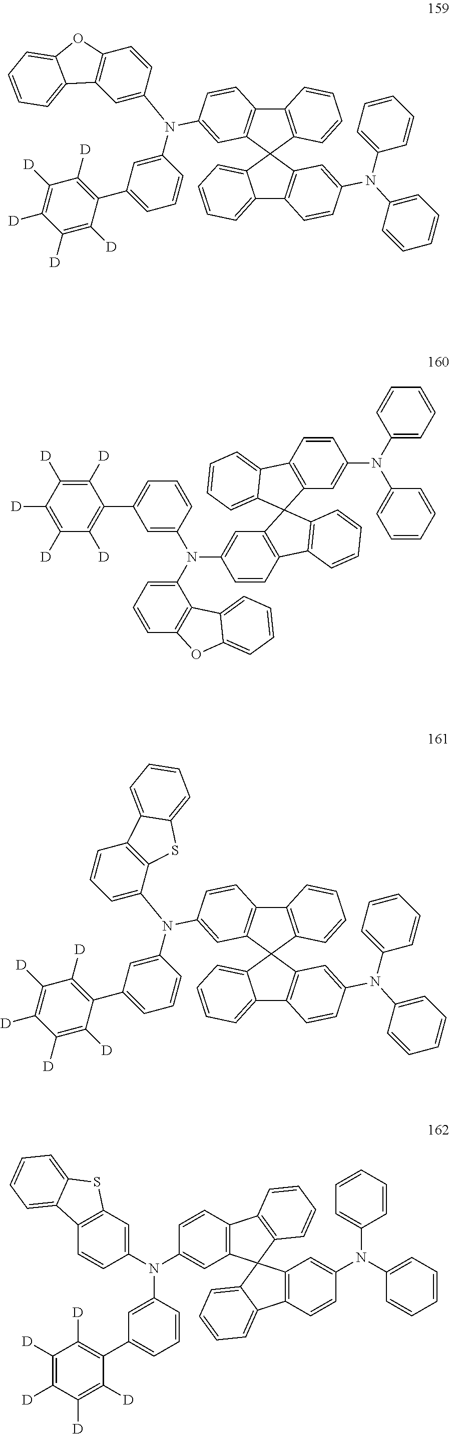

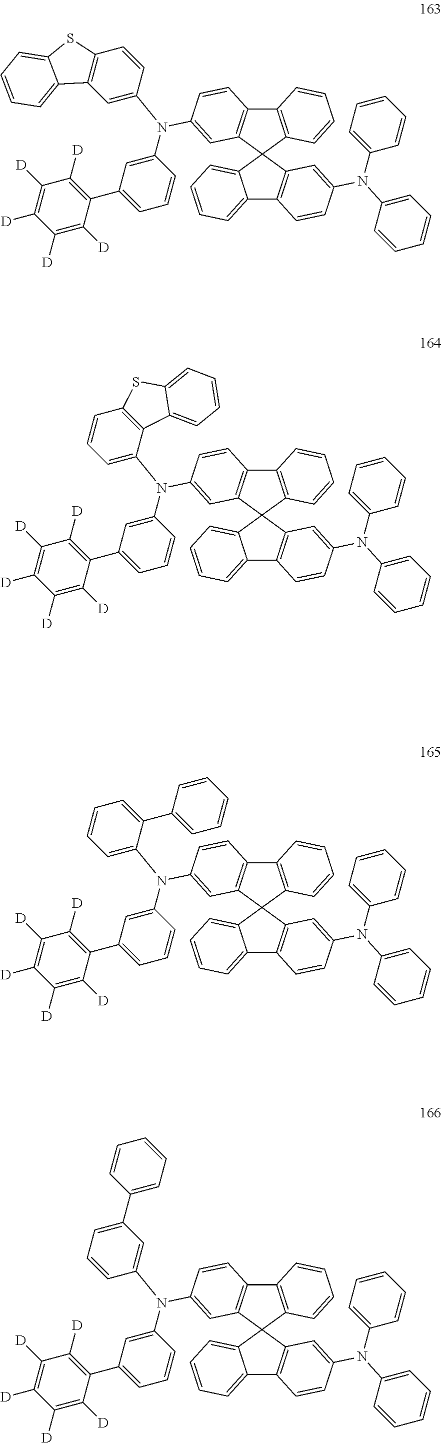

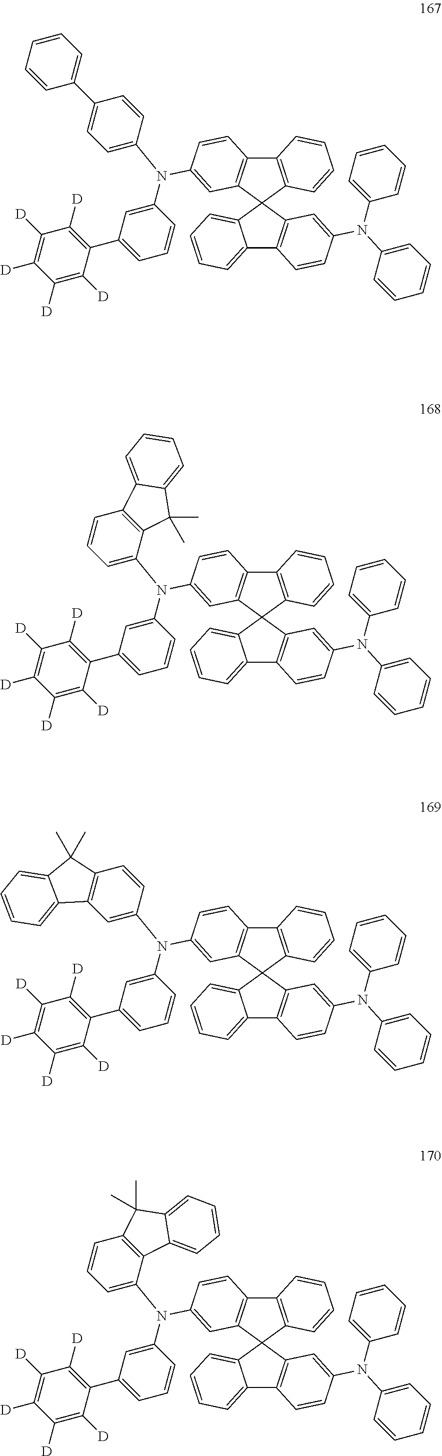

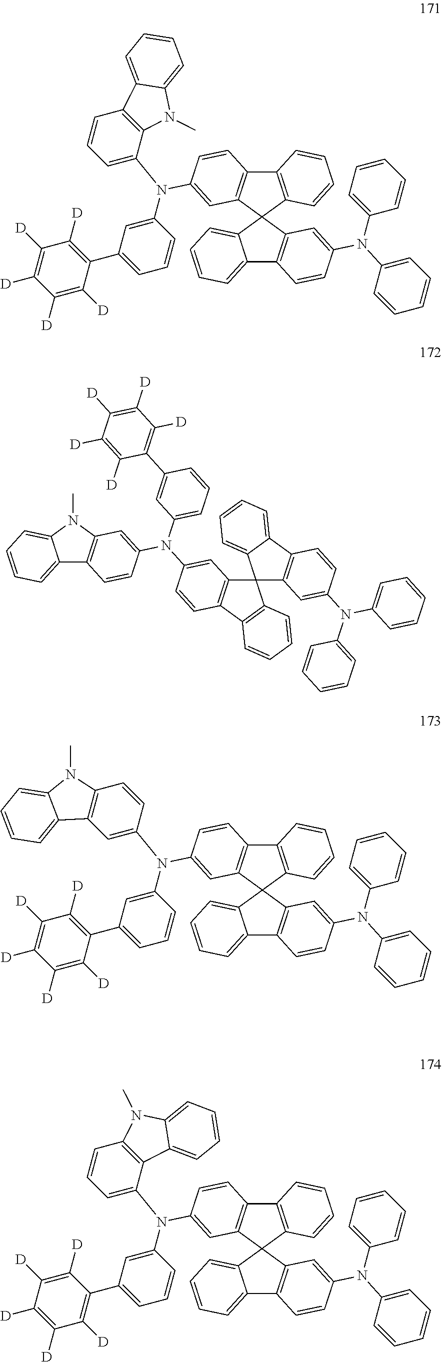

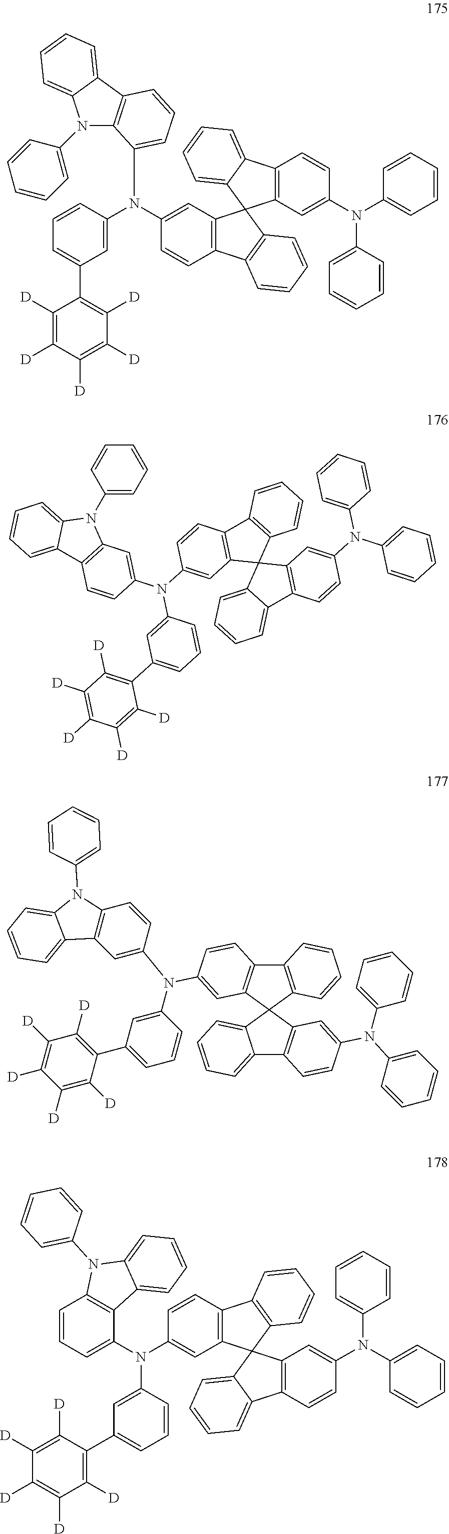

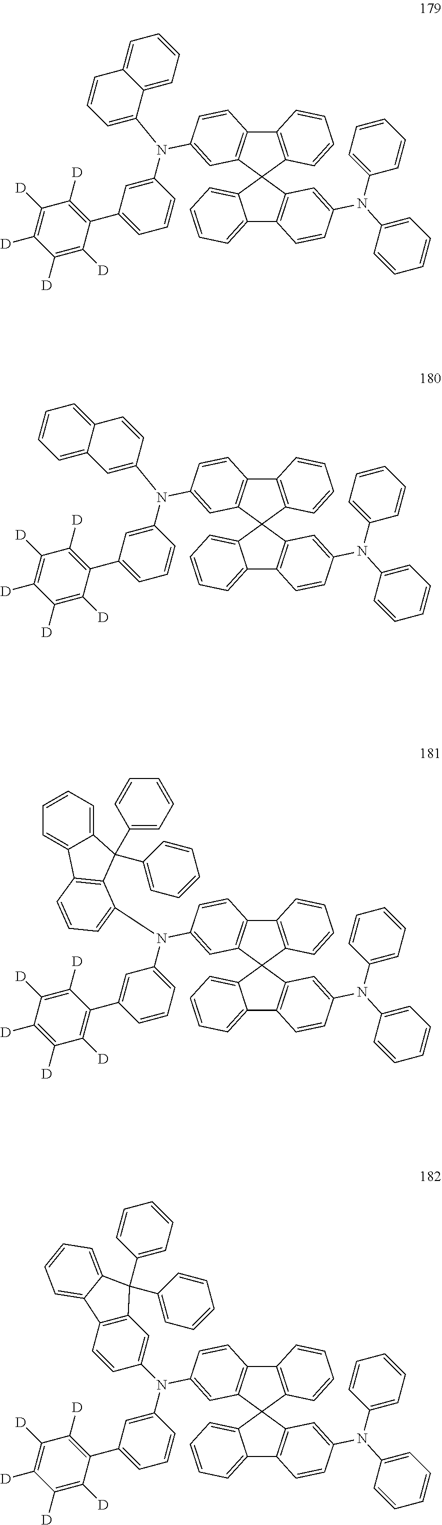

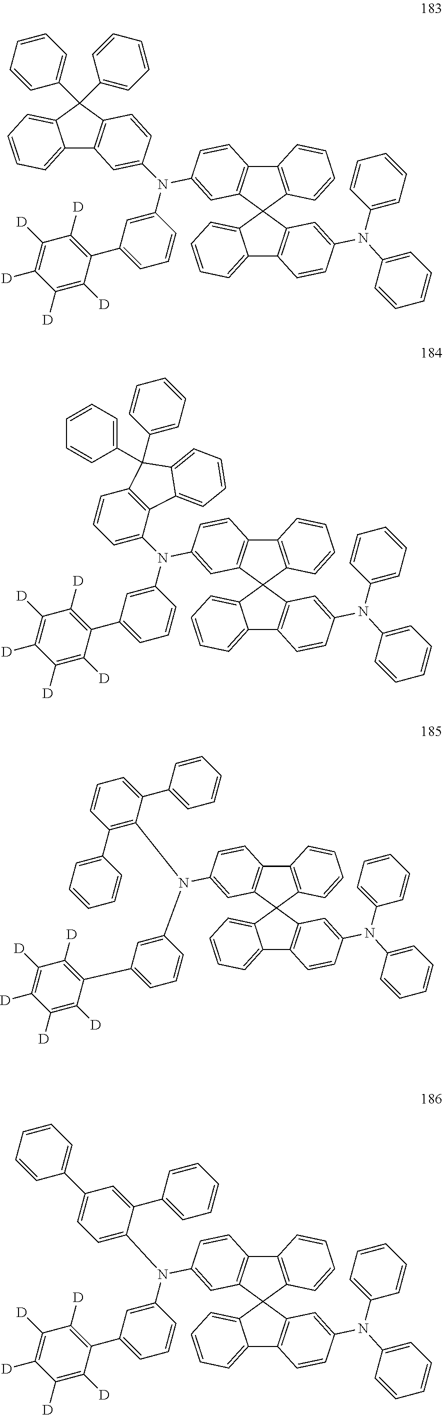

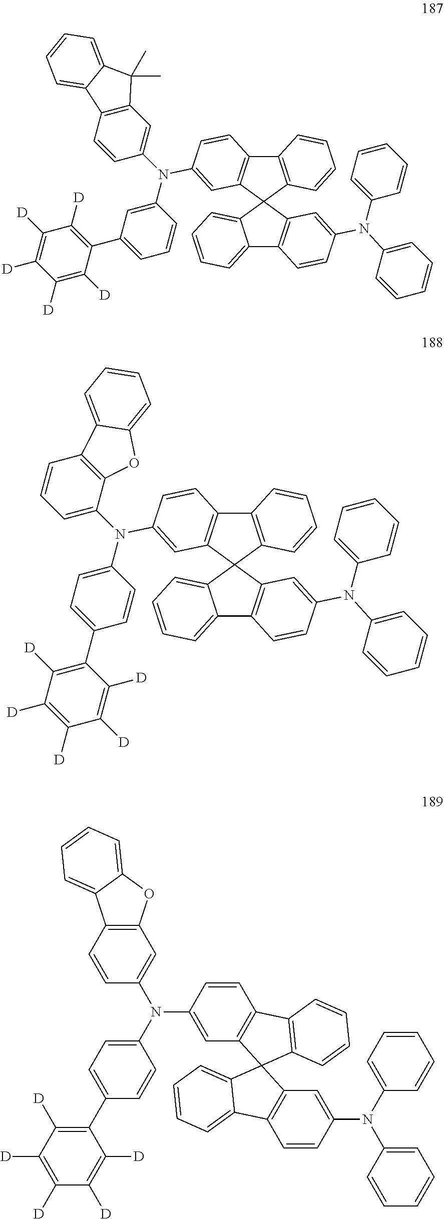 [SMILES: [2H]c1c([2H])c([2H])c(-c2cccc(N(c3ccc4c(c3)C(C)(C)c3ccccc3-4)c3ccc4c(c3)C3(c5ccccc5-c5ccc(N(c6ccccc6)c6ccccc6)cc53)c3ccccc3-4)c2)c([2H])c1[2H] ; [2H]c1c([2H])c([2H])c(-c2ccc(N(c3ccc4c(c3)C3(c5ccccc5-c5ccc(N(c6ccccc6)c6ccccc6)cc53)c3ccccc3-4)c3cccc4c3oc3ccccc34)cc2)c([2H])c1[2H] ; [2H]c1c([2H])c([2H])c(-c2ccc(N(c3ccc4c(c3)C3(c5ccccc5-c5ccc(N(c6ccccc6)c6ccccc6)cc53)c3ccccc3-4)c3ccc4c(c3)oc3ccccc34)cc2)c([2H])c1[2H]]

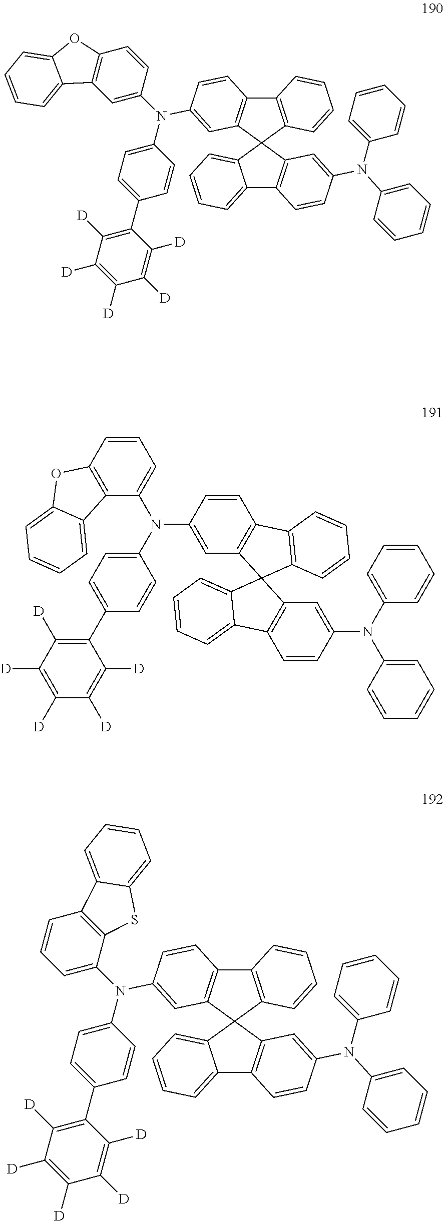 [SMILES: [2H]c1c([2H])c([2H])c(-c2ccc(N(c3ccc4c(c3)C3(c5ccccc5-c5ccc(N(c6ccccc6)c6ccccc6)cc53)c3ccccc3-4)c3ccc4oc5ccccc5c4c3)cc2)c([2H])c1[2H] ; [2H]c1c([2H])c([2H])c(-c2ccc(N(c3ccc4c(c3)C3(c5ccccc5-c5ccc(N(c6ccccc6)c6ccccc6)cc53)c3ccccc3-4)c3cccc4oc5ccccc5c34)cc2)c([2H])c1[2H] ; [2H]c1c([2H])c([2H])c(-c2ccc(N(c3ccc4c(c3)C3(c5ccccc5-c5ccc(N(c6ccccc6)c6ccccc6)cc53)c3ccccc3-4)c3cccc4c3sc3ccccc34)cc2)c([2H])c1[2H]]

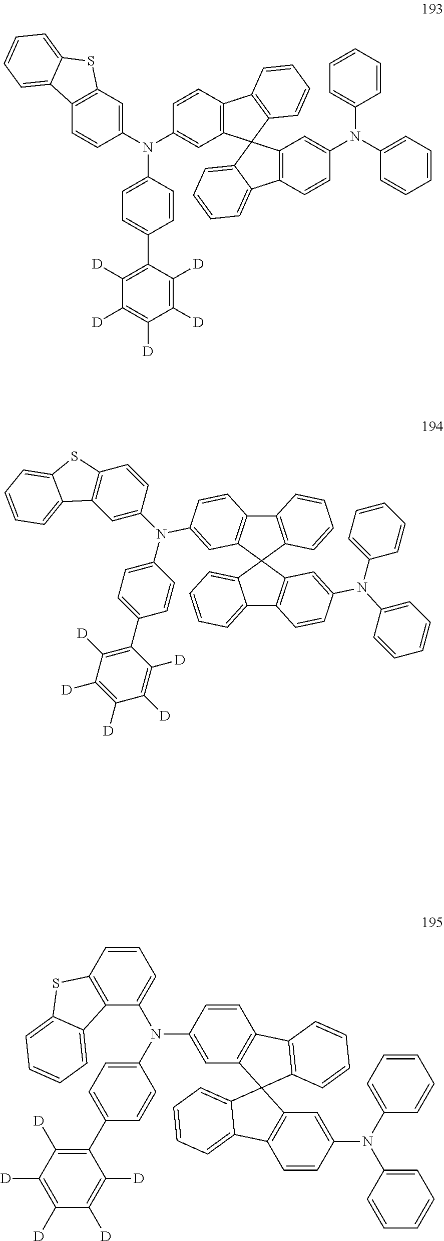 [SMILES: [2H]c1c([2H])c([2H])c(-c2ccc(N(c3ccc4c(c3)C3(c5ccccc5-c5ccc(N(c6ccccc6)c6ccccc6)cc53)c3ccccc3-4)c3ccc4c(c3)sc3ccccc34)cc2)c([2H])c1[2H] ; [2H]c1c([2H])c([2H])c(-c2ccc(N(c3ccc4c(c3)C3(c5ccccc5-c5ccc(N(c6ccccc6)c6ccccc6)cc53)c3ccccc3-4)c3ccc4sc5ccccc5c4c3)cc2)c([2H])c1[2H] ; [2H]c1c([2H])c([2H])c(-c2ccc(N(c3ccc4c(c3)C3(c5ccccc5-c5ccc(N(c6ccccc6)c6ccccc6)cc53)c3ccccc3-4)c3cccc4sc5ccccc5c34)cc2)c([2H])c1[2H]]

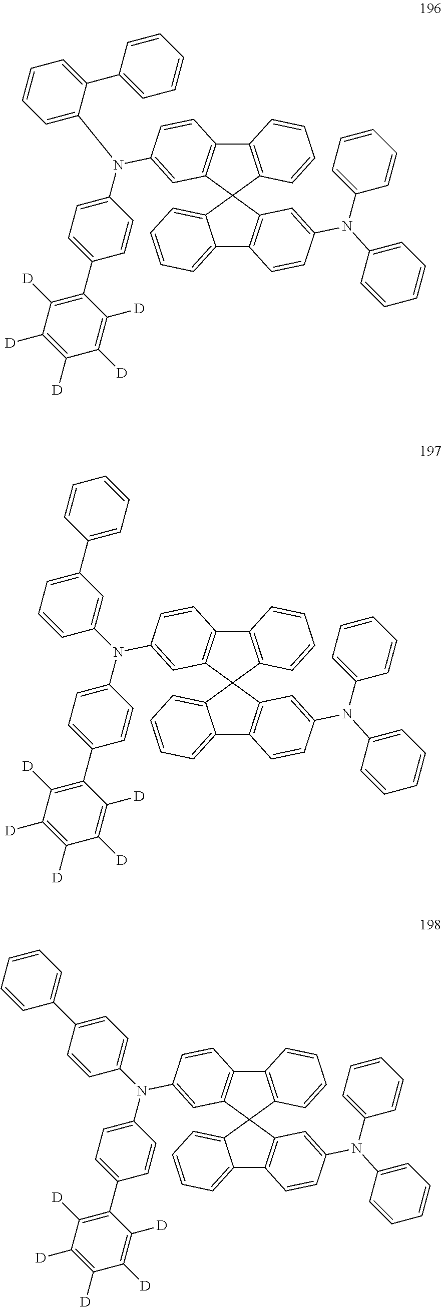 [SMILES: [2H]c1c([2H])c([2H])c(-c2ccc(N(c3ccc4c(c3)C3(c5ccccc5-c5ccc(N(c6ccccc6)c6ccccc6)cc53)c3ccccc3-4)c3ccccc3-c3ccccc3)cc2)c([2H])c1[2H] ; [2H]c1c([2H])c([2H])c(-c2ccc(N(c3cccc(-c4ccccc4)c3)c3ccc4c(c3)C3(c5ccccc5-c5ccc(N(c6ccccc6)c6ccccc6)cc53)c3ccccc3-4)cc2)c([2H])c1[2H] ; [2H]c1c([2H])c([2H])c(-c2ccc(N(c3ccc(-c4ccccc4)cc3)c3ccc4c(c3)C3(c5ccccc5-c5ccc(N(c6ccccc6)c6ccccc6)cc53)c3ccccc3-4)cc2)c([2H])c1[2H]]

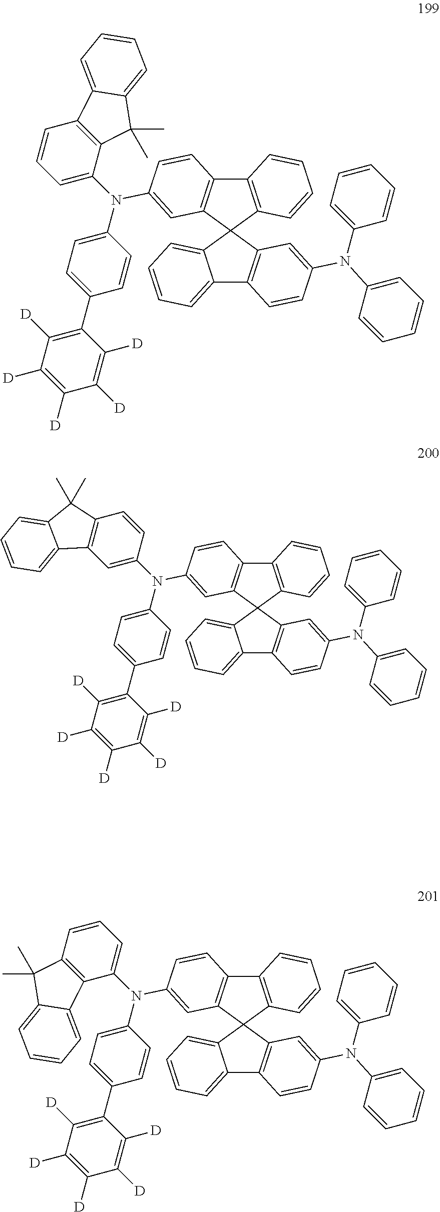 [SMILES: [2H]c1c([2H])c([2H])c(-c2ccc(N(c3ccc4c(c3)C3(c5ccccc5-c5ccc(N(c6ccccc6)c6ccccc6)cc53)c3ccccc3-4)c3cccc4c3C(C)(C)c3ccccc3-4)cc2)c([2H])c1[2H] ; [2H]c1c([2H])c([2H])c(-c2ccc(N(c3ccc4c(c3)-c3ccccc3C4(C)C)c3ccc4c(c3)C3(c5ccccc5-c5ccc(N(c6ccccc6)c6ccccc6)cc53)c3ccccc3-4)cc2)c([2H])c1[2H] ; [2H]c1c([2H])c([2H])c(-c2ccc(N(c3ccc4c(c3)C3(c5ccccc5-c5ccc(N(c6ccccc6)c6ccccc6)cc53)c3ccccc3-4)c3cccc4c3-c3ccccc3C4(C)C)cc2)c([2H])c1[2H]]

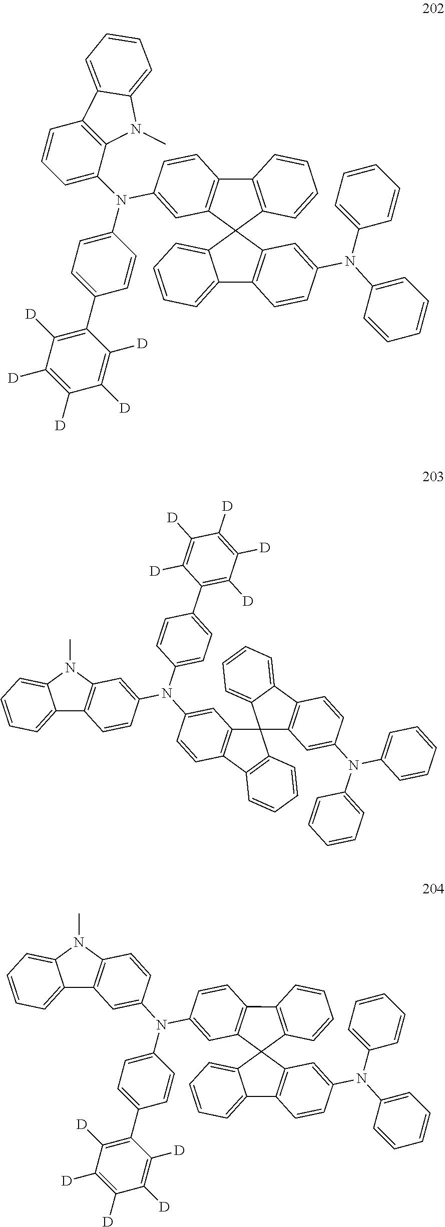 [SMILES: [2H]c1c([2H])c([2H])c(-c2ccc(N(c3ccc4c(c3)C3(c5ccccc5-c5ccc(N(c6ccccc6)c6ccccc6)cc53)c3ccccc3-4)c3cccc4c5ccccc5n(C)c34)cc2)c([2H])c1[2H] ; [2H]c1c([2H])c([2H])c(-c2ccc(N(c3ccc4c(c3)C3(c5ccccc5-c5ccc(N(c6ccccc6)c6ccccc6)cc53)c3ccccc3-4)c3ccc4c5ccccc5n(C)c4c3)cc2)c([2H])c1[2H] ; [2H]c1c([2H])c([2H])c(-c2ccc(N(c3ccc4c(c3)C3(c5ccccc5-c5ccc(N(c6ccccc6)c6ccccc6)cc53)c3ccccc3-4)c3ccc4c(c3)c3ccccc3n4C)cc2)c([2H])c1[2H]]

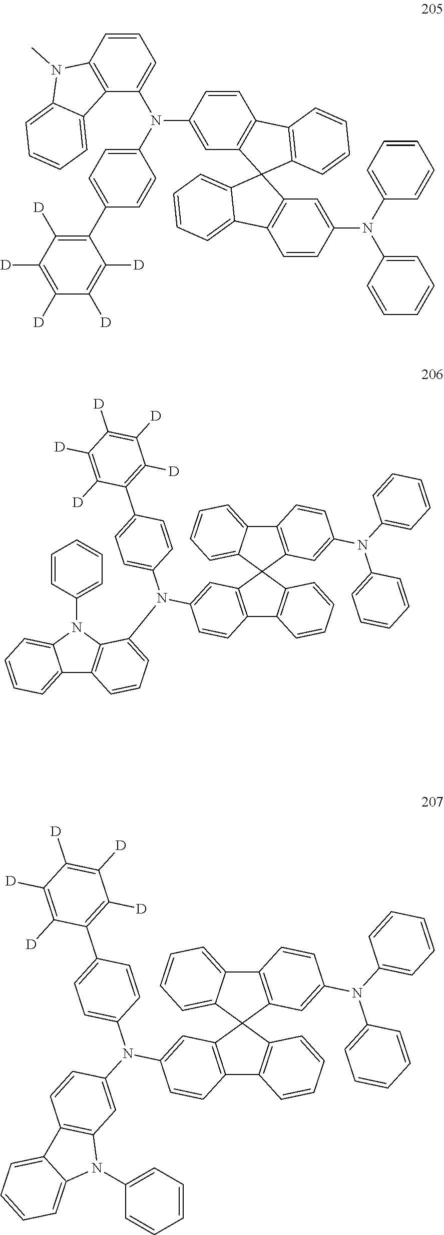 [SMILES: [2H]c1c([2H])c([2H])c(-c2ccc(N(c3ccc4c(c3)C3(c5ccccc5-c5ccc(N(c6ccccc6)c6ccccc6)cc53)c3ccccc3-4)c3cccc4c3c3ccccc3n4C)cc2)c([2H])c1[2H] ; [2H]c1c([2H])c([2H])c(-c2ccc(N(c3ccc4c(c3)C3(c5ccccc5-c5ccc(N(c6ccccc6)c6ccccc6)cc53)c3ccccc3-4)c3cccc4c5ccccc5n(-c5ccccc5)c34)cc2)c([2H])c1[2H] ; [2H]c1c([2H])c([2H])c(-c2ccc(N(c3ccc4c(c3)C3(c5ccccc5-c5ccc(N(c6ccccc6)c6ccccc6)cc53)c3ccccc3-4)c3ccc4c5ccccc5n(-c5ccccc5)c4c3)cc2)c([2H])c1[2H]]

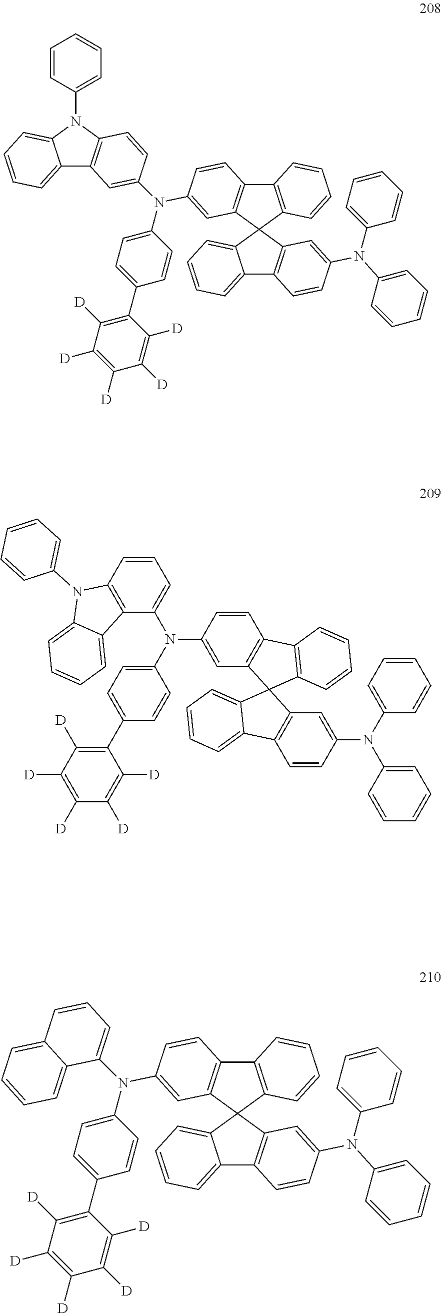 [SMILES: [2H]c1c([2H])c([2H])c(-c2ccc(N(c3ccc4c(c3)C3(c5ccccc5-c5ccc(N(c6ccccc6)c6ccccc6)cc53)c3ccccc3-4)c3ccc4c(c3)c3ccccc3n4-c3ccccc3)cc2)c([2H])c1[2H] ; [2H]c1c([2H])c([2H])c(-c2ccc(N(c3ccc4c(c3)C3(c5ccccc5-c5ccc(N(c6ccccc6)c6ccccc6)cc53)c3ccccc3-4)c3cccc4c3c3ccccc3n4-c3ccccc3)cc2)c([2H])c1[2H] ; [2H]c1c([2H])c([2H])c(-c2ccc(N(c3ccc4c(c3)C3(c5ccccc5-c5ccc(N(c6ccccc6)c6ccccc6)cc53)c3ccccc3-4)c3cccc4ccccc34)cc2)c([2H])c1[2H]]

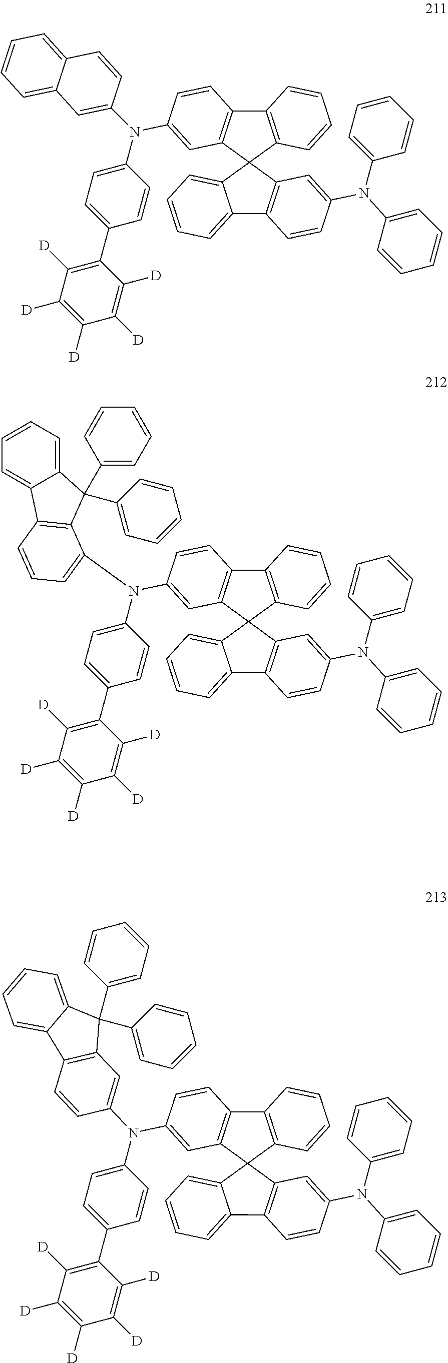 [SMILES: [2H]c1c([2H])c([2H])c(-c2ccc(N(c3ccc4c(c3)C3(c5ccccc5-c5ccc(N(c6ccccc6)c6ccccc6)cc53)c3ccccc3-4)c3ccc4ccccc4c3)cc2)c([2H])c1[2H] ; [2H]c1c([2H])c([2H])c(-c2ccc(N(c3ccc4c(c3)C3(c5ccccc5-c5ccc(N(c6ccccc6)c6ccccc6)cc53)c3ccccc3-4)c3cccc4c3C(c3ccccc3)(c3ccccc3)c3ccccc3-4)cc2)c([2H])c1[2H] ; [2H]c1c([2H])c([2H])c(-c2ccc(N(c3ccc4c(c3)C(c3ccccc3)(c3ccccc3)c3ccccc3-4)c3ccc4c(c3)C3(c5ccccc5-c5ccc(N(c6ccccc6)c6ccccc6)cc53)c3ccccc3-4)cc2)c([2H])c1[2H]]

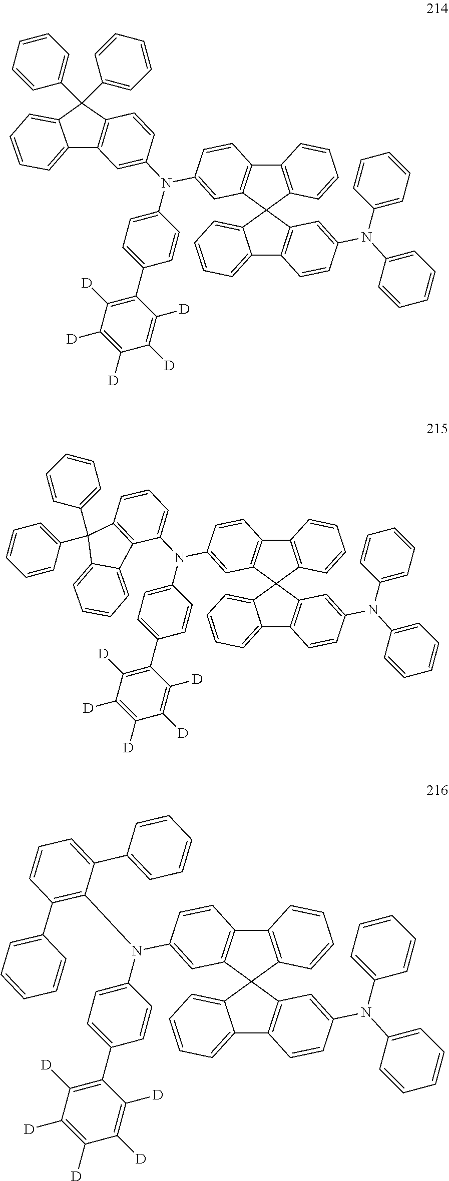 [SMILES: [2H]c1c([2H])c([2H])c(-c2ccc(N(c3ccc4c(c3)-c3ccccc3C4(c3ccccc3)c3ccccc3)c3ccc4c(c3)C3(c5ccccc5-c5ccc(N(c6ccccc6)c6ccccc6)cc53)c3ccccc3-4)cc2)c([2H])c1[2H] ; [2H]c1c([2H])c([2H])c(-c2ccc(N(c3ccc4c(c3)C3(c5ccccc5-c5ccc(N(c6ccccc6)c6ccccc6)cc53)c3ccccc3-4)c3cccc4c3-c3ccccc3C4(c3ccccc3)c3ccccc3)cc2)c([2H])c1[2H] ; [2H]c1c([2H])c([2H])c(-c2ccc(N(c3ccc4c(c3)C3(c5ccccc5-c5ccc(N(c6ccccc6)c6ccccc6)cc53)c3ccccc3-4)c3c(-c4ccccc4)cccc3-c3ccccc3)cc2)c([2H])c1[2H]]

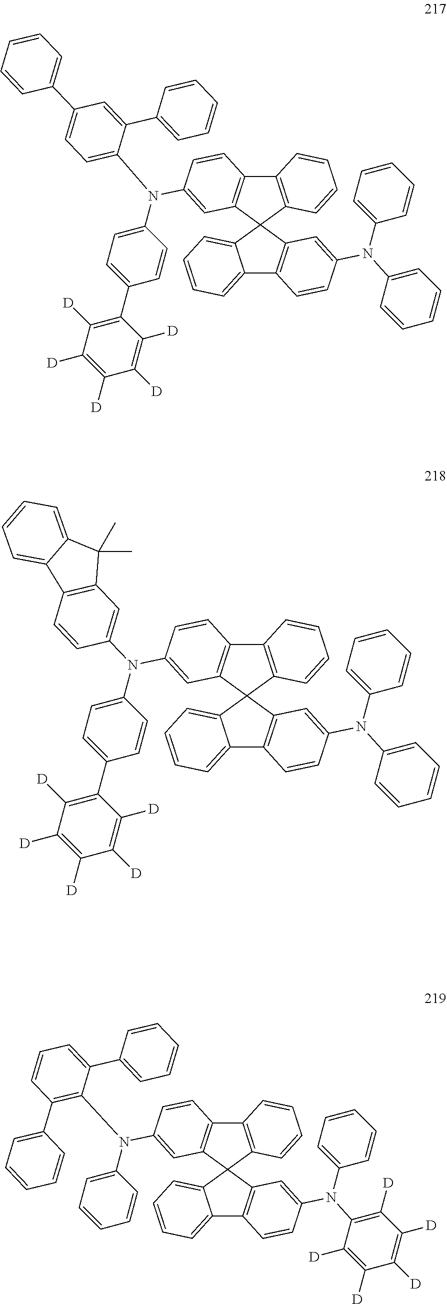 [SMILES: [2H]c1c([2H])c([2H])c(-c2ccc(N(c3ccc4c(c3)C3(c5ccccc5-c5ccc(N(c6ccccc6)c6ccccc6)cc53)c3ccccc3-4)c3ccc(-c4ccccc4)cc3-c3ccccc3)cc2)c([2H])c1[2H] ; [2H]c1c([2H])c([2H])c(-c2ccc(N(c3ccc4c(c3)C(C)(C)c3ccccc3-4)c3ccc4c(c3)C3(c5ccccc5-c5ccc(N(c6ccccc6)c6ccccc6)cc53)c3ccccc3-4)cc2)c([2H])c1[2H] ; [2H]c1c([2H])c([2H])c(N(c2ccccc2)c2ccc3c(c2)C2(c4ccccc4-c4ccc(N(c5ccccc5)c5c(-c6ccccc6)cccc5-c5ccccc5)cc42)c2ccccc2-3)c([2H])c1[2H]]

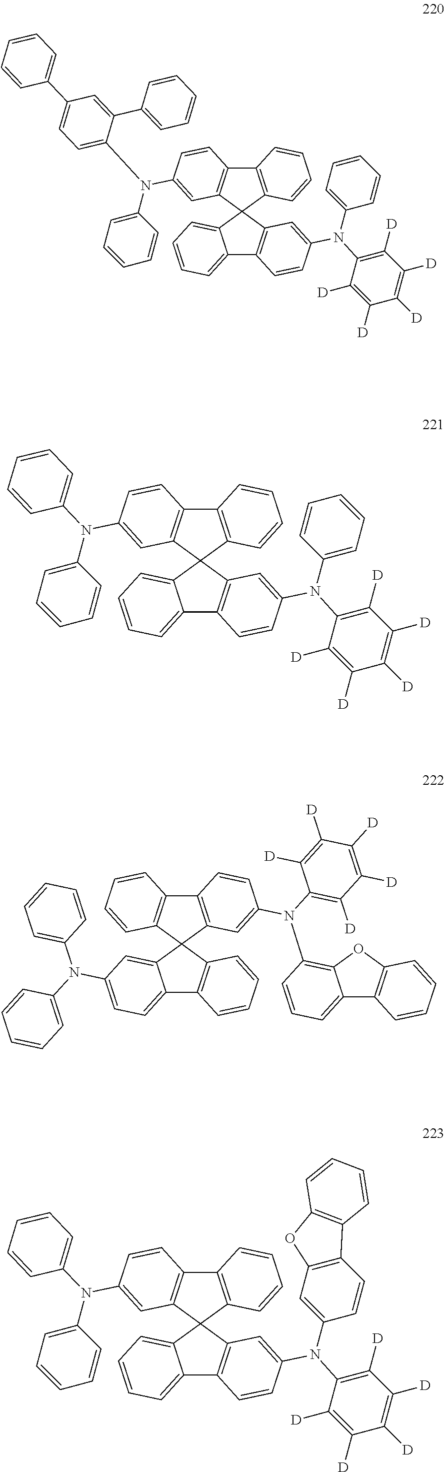 [SMILES: [2H]c1c([2H])c([2H])c(N(c2ccccc2)c2ccc3c(c2)C2(c4ccccc4-c4ccc(N(c5ccccc5)c5ccc(-c6ccccc6)cc5-c5ccccc5)cc42)c2ccccc2-3)c([2H])c1[2H] ; [2H]c1c([2H])c([2H])c(N(c2ccccc2)c2ccc3c(c2)C2(c4ccccc4-c4ccc(N(c5ccccc5)c5ccccc5)cc42)c2ccccc2-3)c([2H])c1[2H] ; [2H]c1c([2H])c([2H])c(N(c2ccc3c(c2)C2(c4ccccc4-c4ccc(N(c5ccccc5)c5ccccc5)cc42)c2ccccc2-3)c2cccc3c2oc2ccccc23)c([2H])c1[2H] ; [2H]c1c([2H])c([2H])c(N(c2ccc3c(c2)C2(c4ccccc4-c4ccc(N(c5ccccc5)c5ccccc5)cc42)c2ccccc2-3)c2ccc3c(c2)oc2ccccc23)c([2H])c1[2H]]

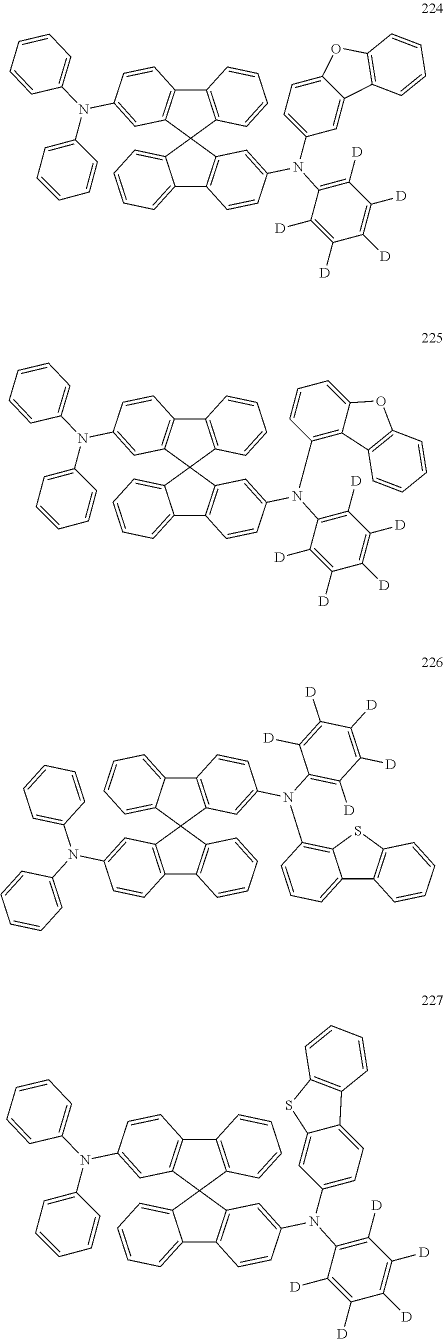 [SMILES: [2H]c1c([2H])c([2H])c(N(c2ccc3c(c2)C2(c4ccccc4-c4ccc(N(c5ccccc5)c5ccccc5)cc42)c2ccccc2-3)c2ccc3oc4ccccc4c3c2)c([2H])c1[2H] ; [2H]c1c([2H])c([2H])c(N(c2ccc3c(c2)C2(c4ccccc4-c4ccc(N(c5ccccc5)c5ccccc5)cc42)c2ccccc2-3)c2cccc3oc4ccccc4c23)c([2H])c1[2H] ; [2H]c1c([2H])c([2H])c(N(c2ccc3c(c2)C2(c4ccccc4-c4ccc(N(c5ccccc5)c5ccccc5)cc42)c2ccccc2-3)c2cccc3c2sc2ccccc23)c([2H])c1[2H] ; [2H]c1c([2H])c([2H])c(N(c2ccc3c(c2)C2(c4ccccc4-c4ccc(N(c5ccccc5)c5ccccc5)cc42)c2ccccc2-3)c2ccc3c(c2)sc2ccccc23)c([2H])c1[2H]]

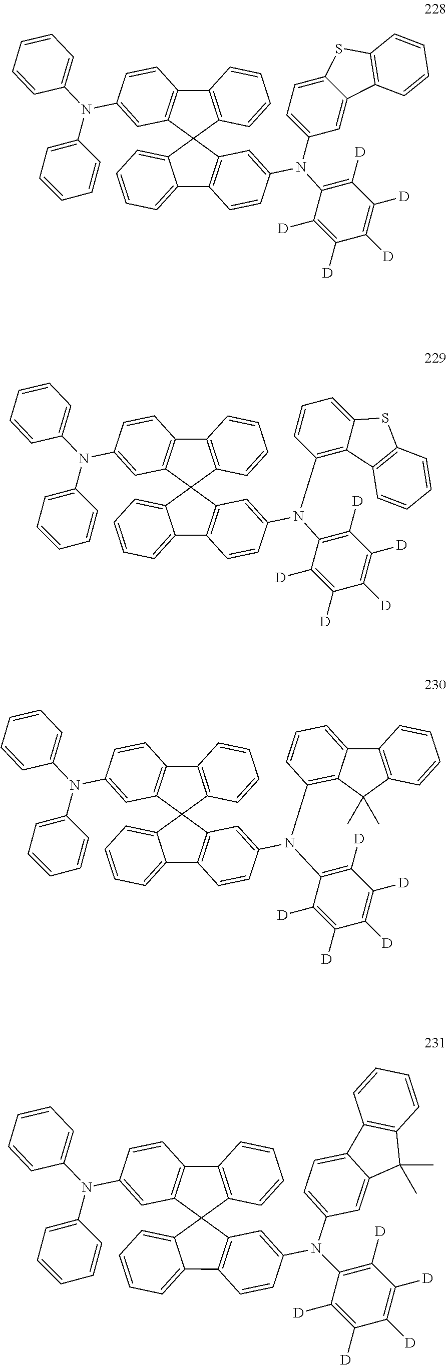

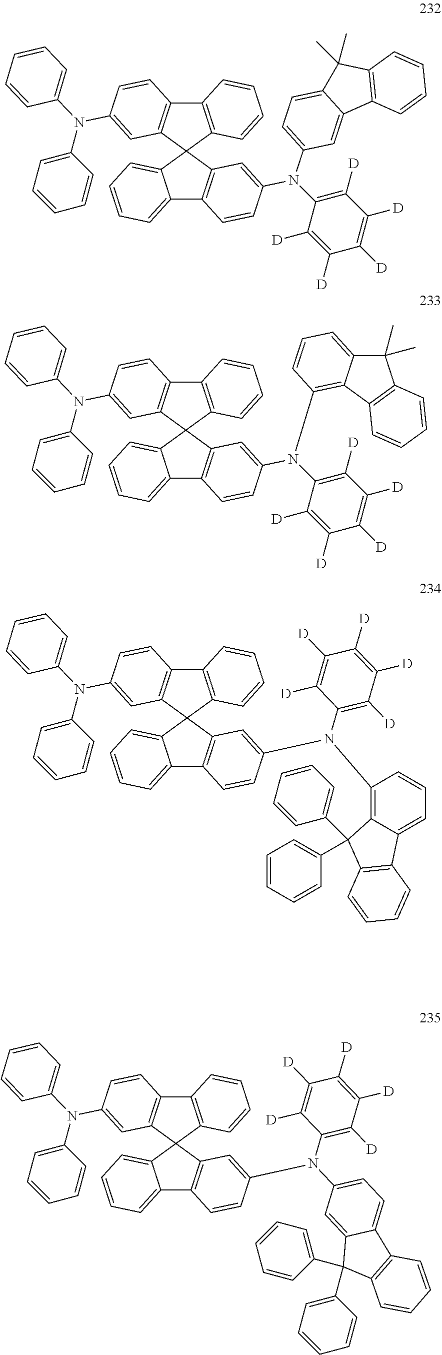

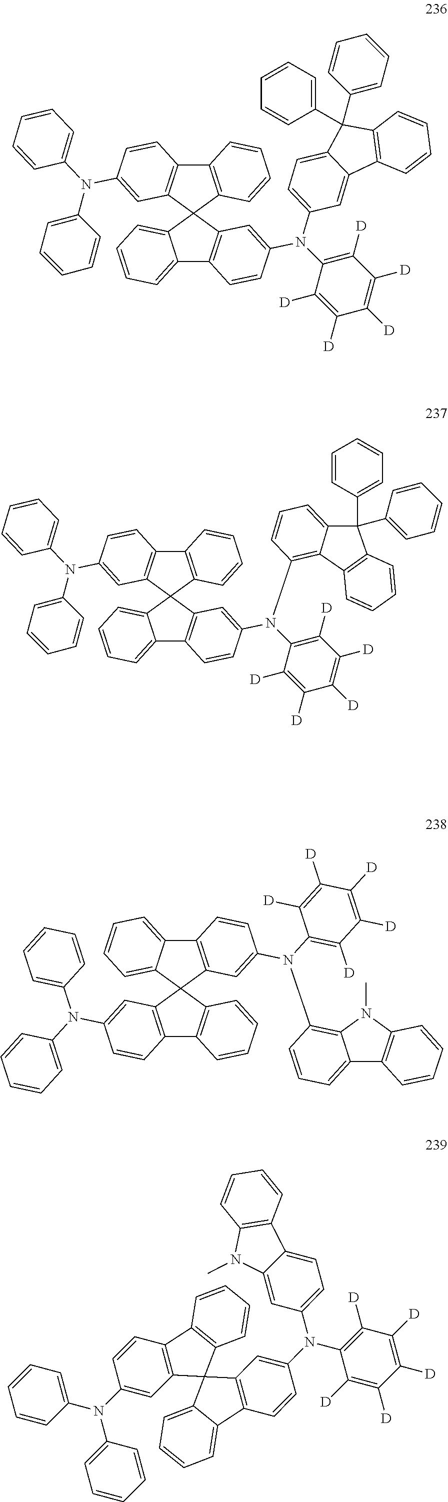

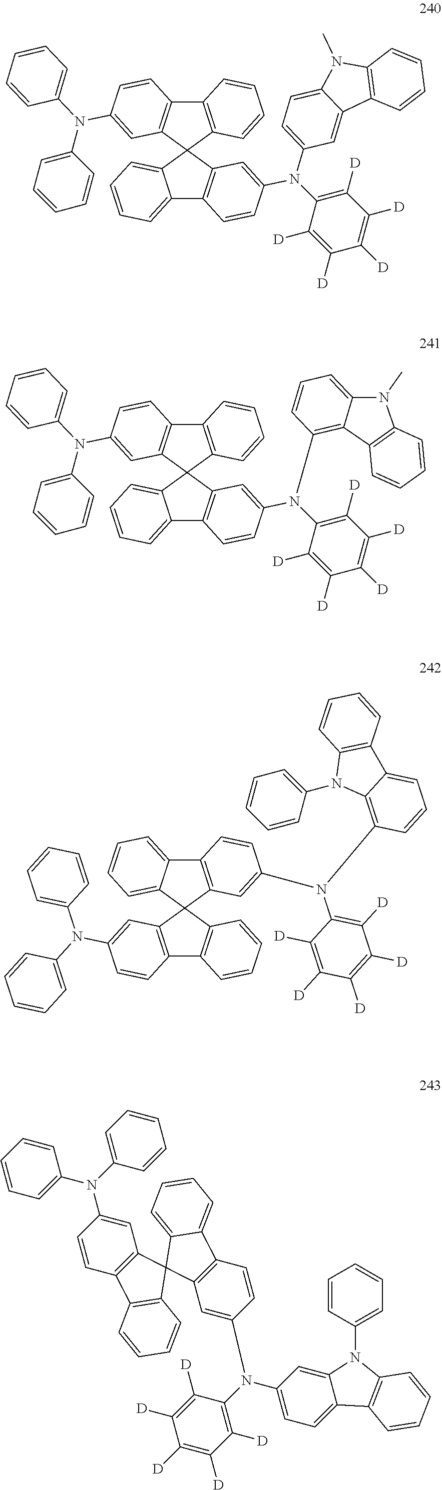

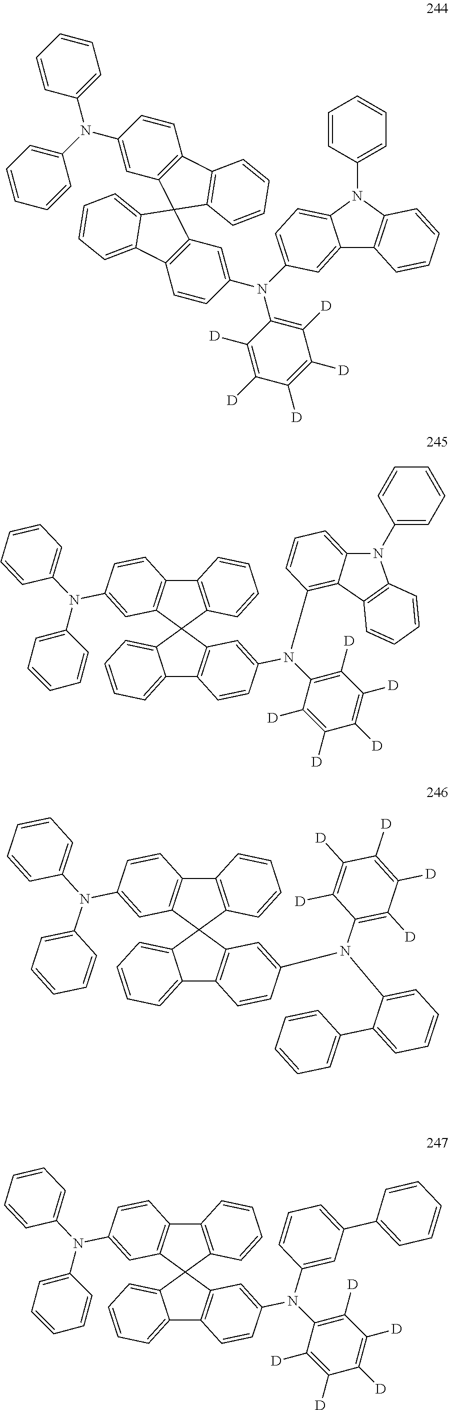

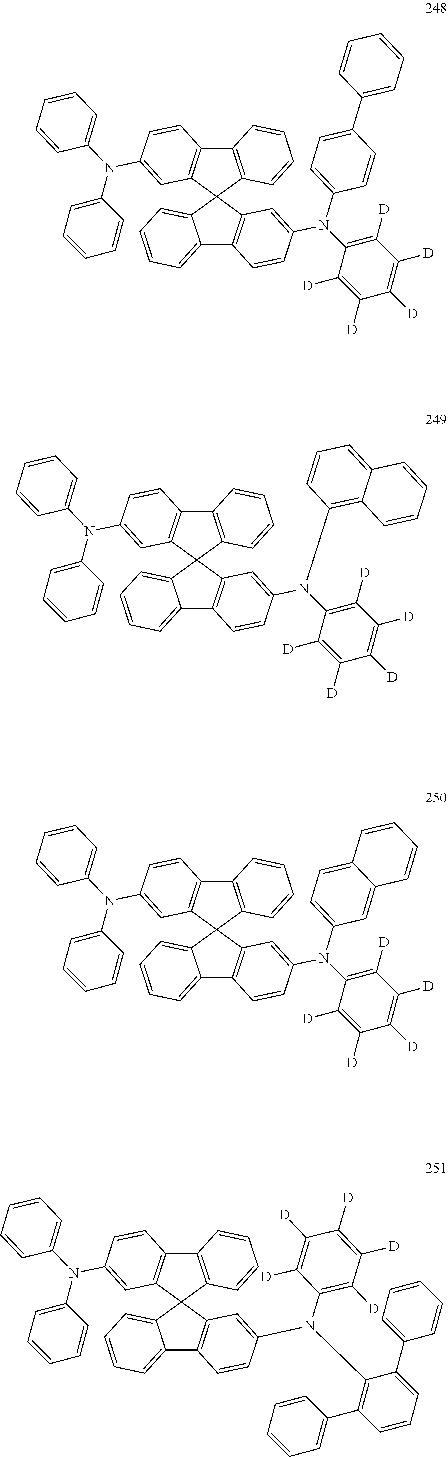 [SMILES: [2H]c1c([2H])c([2H])c(N(c2ccc(-c3ccccc3)cc2)c2ccc3c(c2)C2(c4ccccc4-c4ccc(N(c5ccccc5)c5ccccc5)cc42)c2ccccc2-3)c([2H])c1[2H] ; [2H]c1c([2H])c([2H])c(N(c2ccc3c(c2)C2(c4ccccc4-c4ccc(N(c5ccccc5)c5ccccc5)cc42)c2ccccc2-3)c2cccc3ccccc23)c([2H])c1[2H] ; [2H]c1c([2H])c([2H])c(N(c2ccc3c(c2)C2(c4ccccc4-c4ccc(N(c5ccccc5)c5ccccc5)cc42)c2ccccc2-3)c2ccc3ccccc3c2)c([2H])c1[2H] ; [2H]c1c([2H])c([2H])c(N(c2ccc3c(c2)C2(c4ccccc4-c4ccc(N(c5ccccc5)c5ccccc5)cc42)c2ccccc2-3)c2c(-c3ccccc3)cccc2-c2ccccc2)c([2H])c1[2H]]

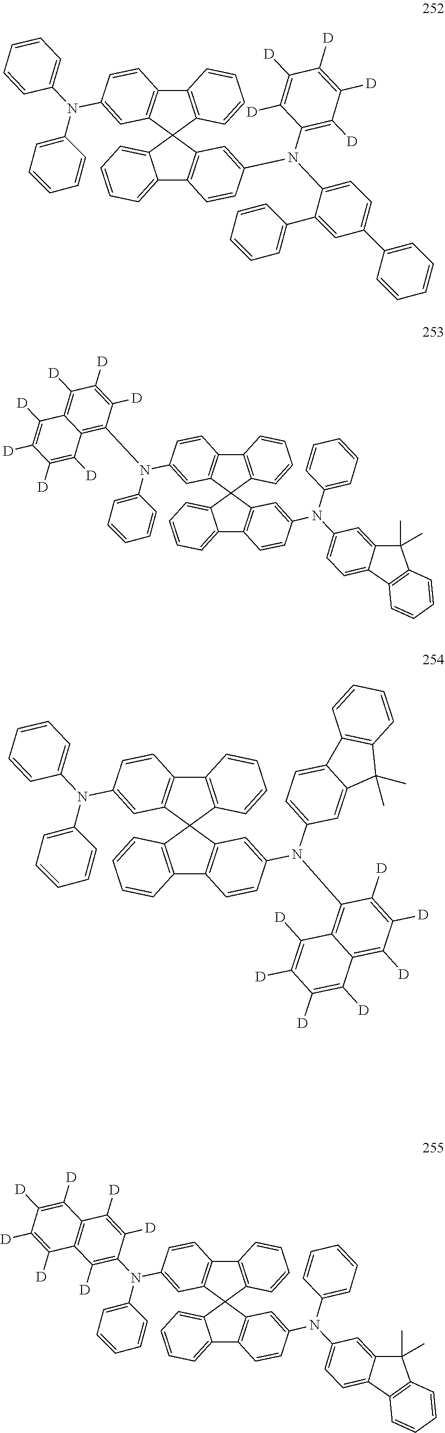

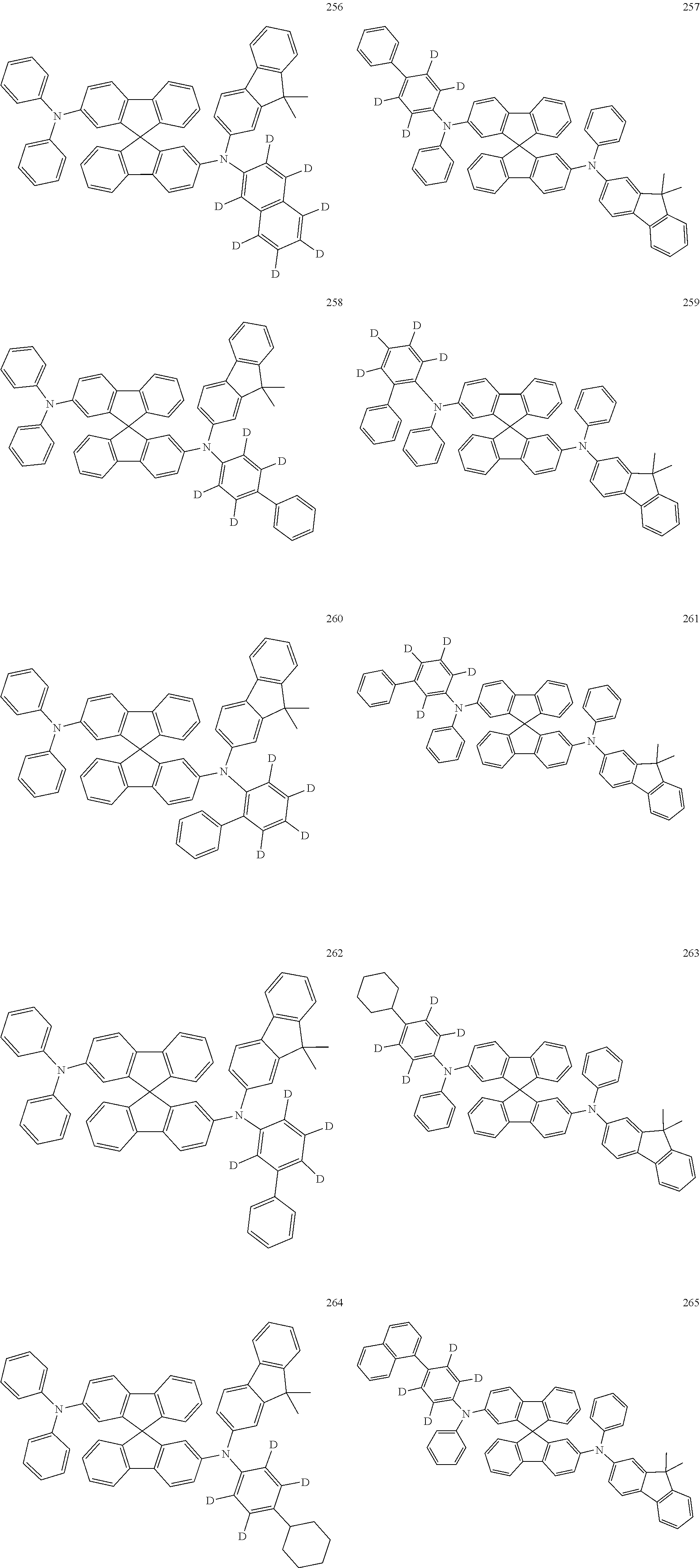

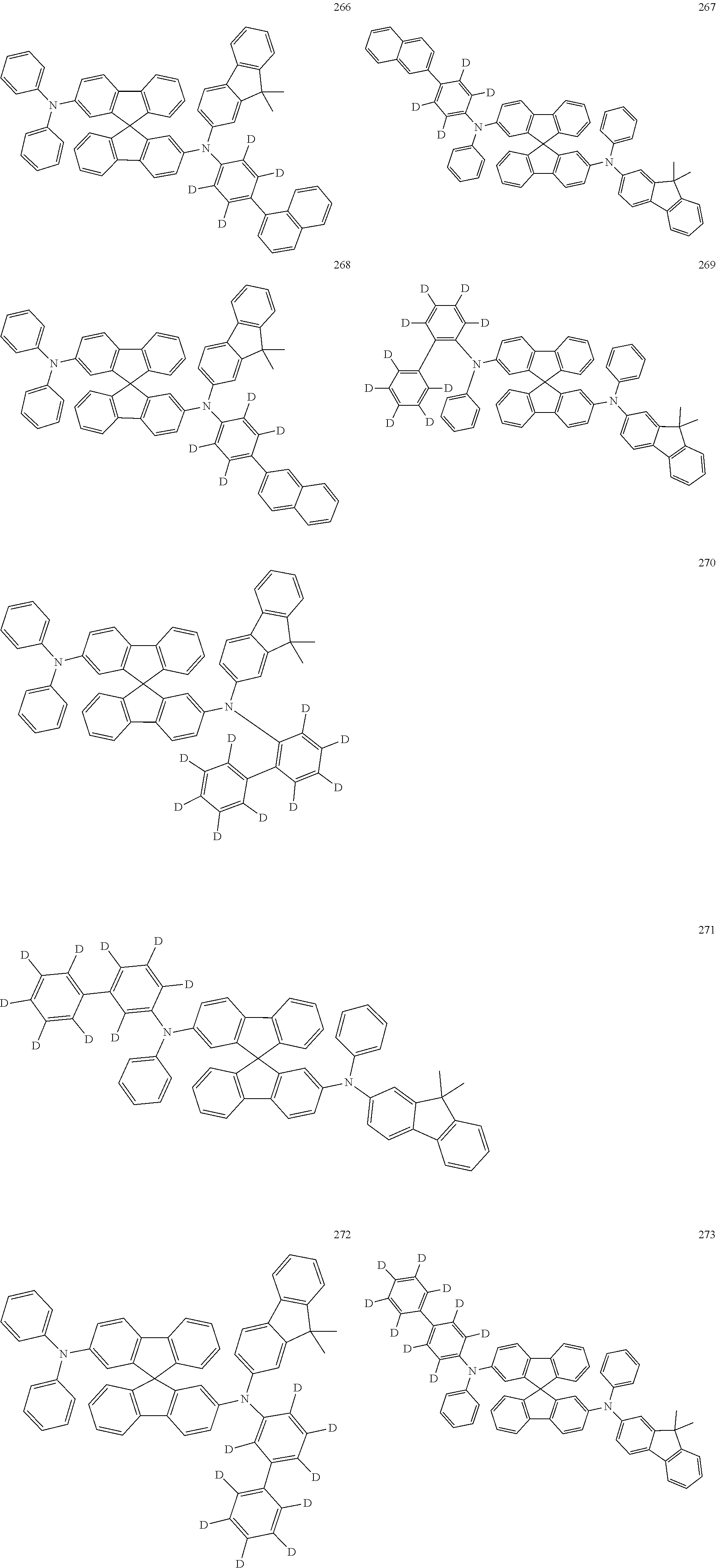

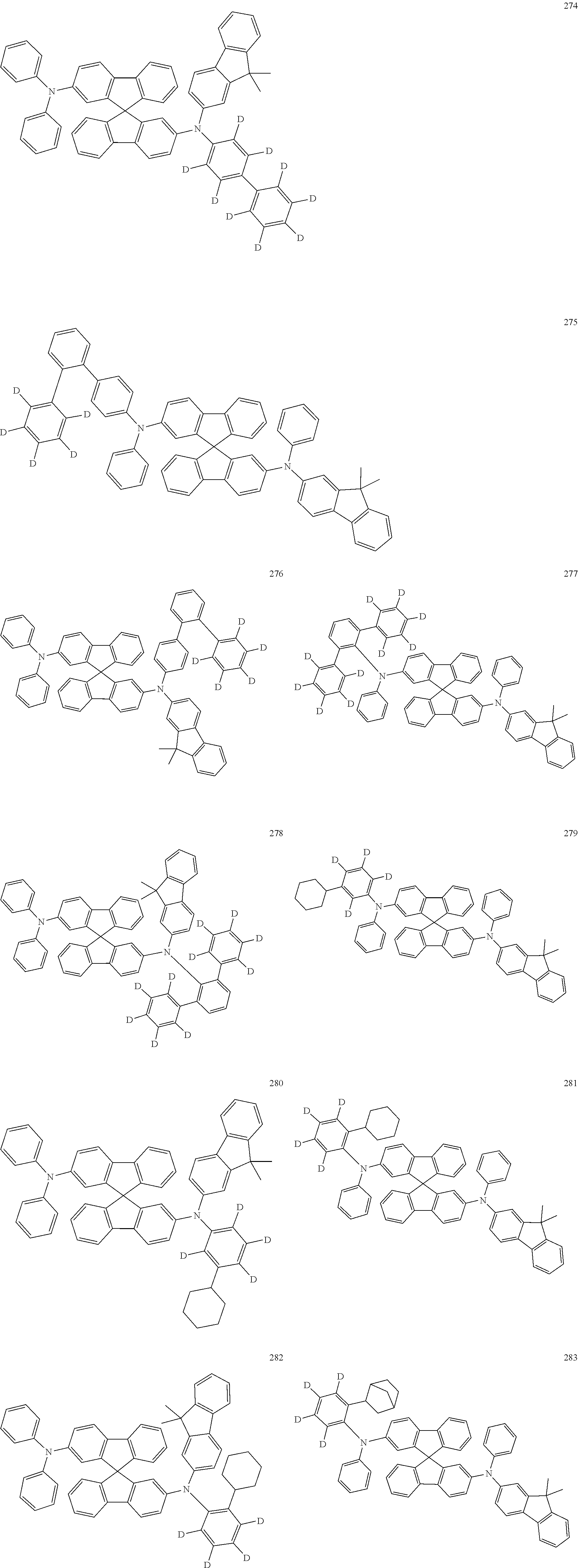

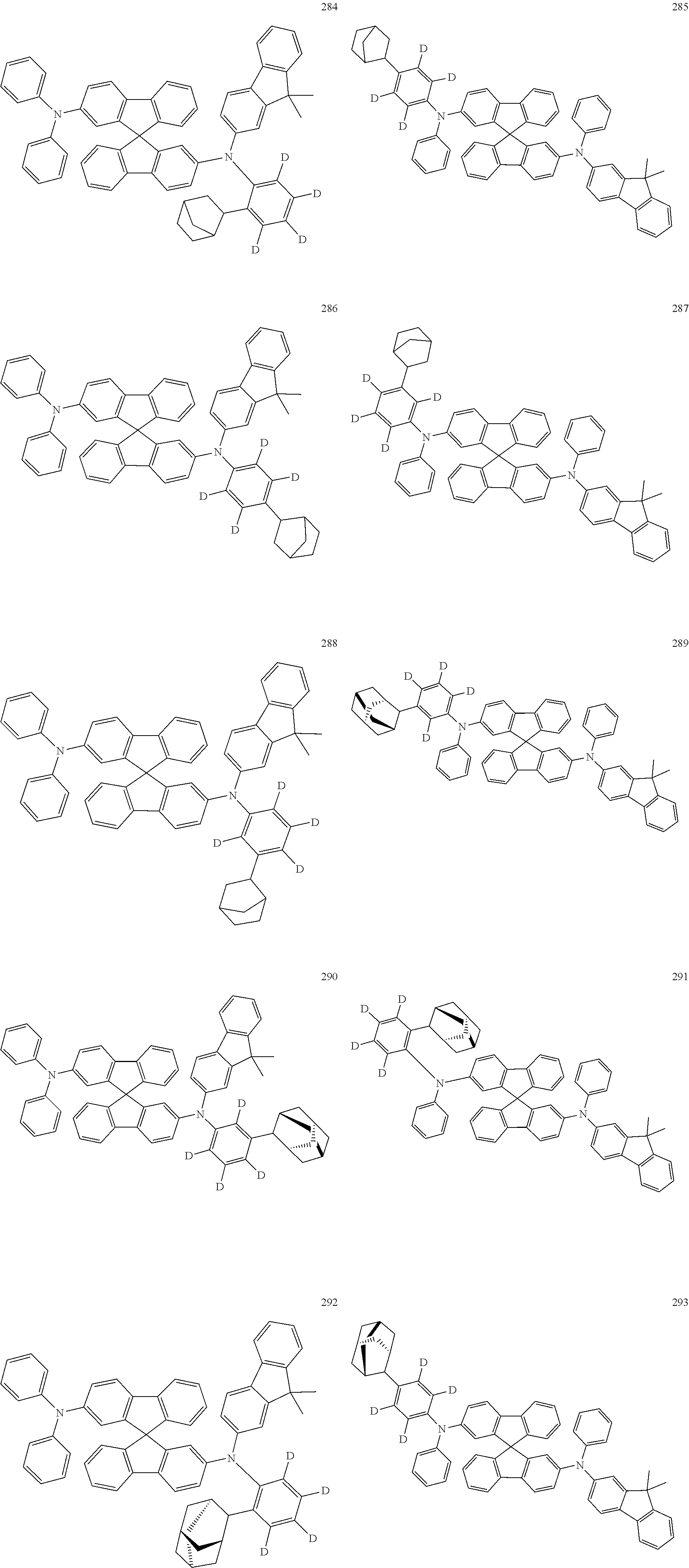

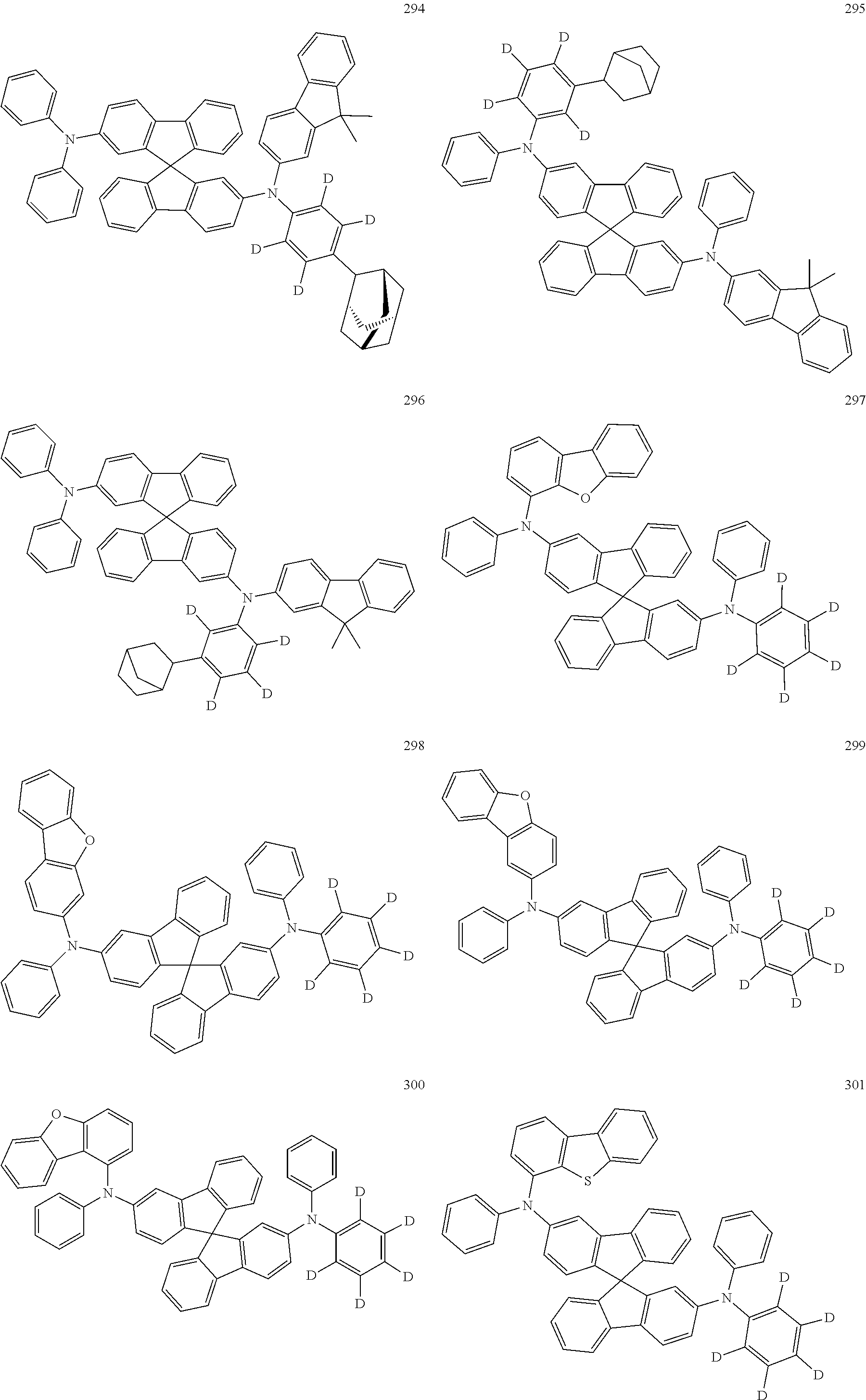

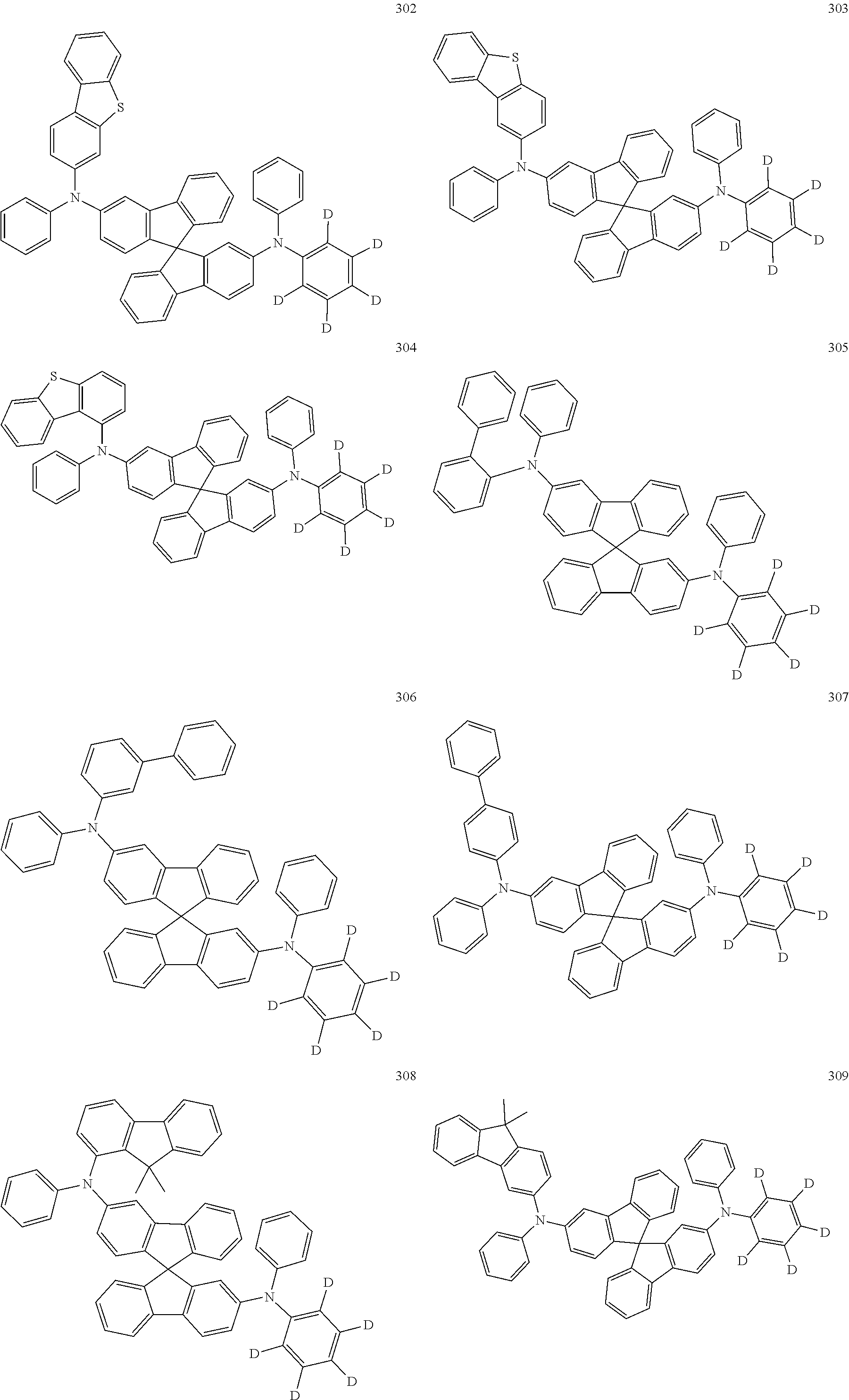

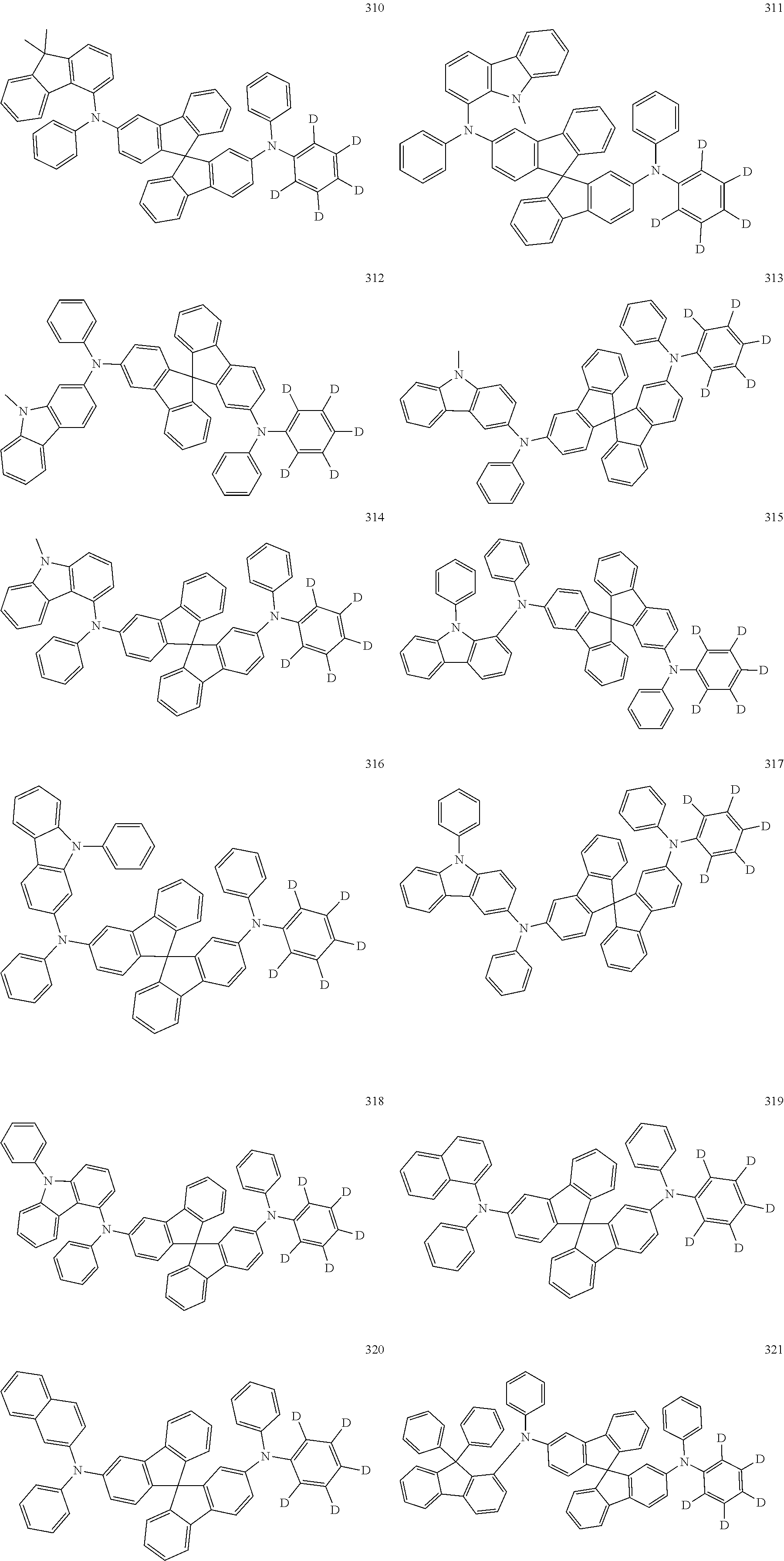

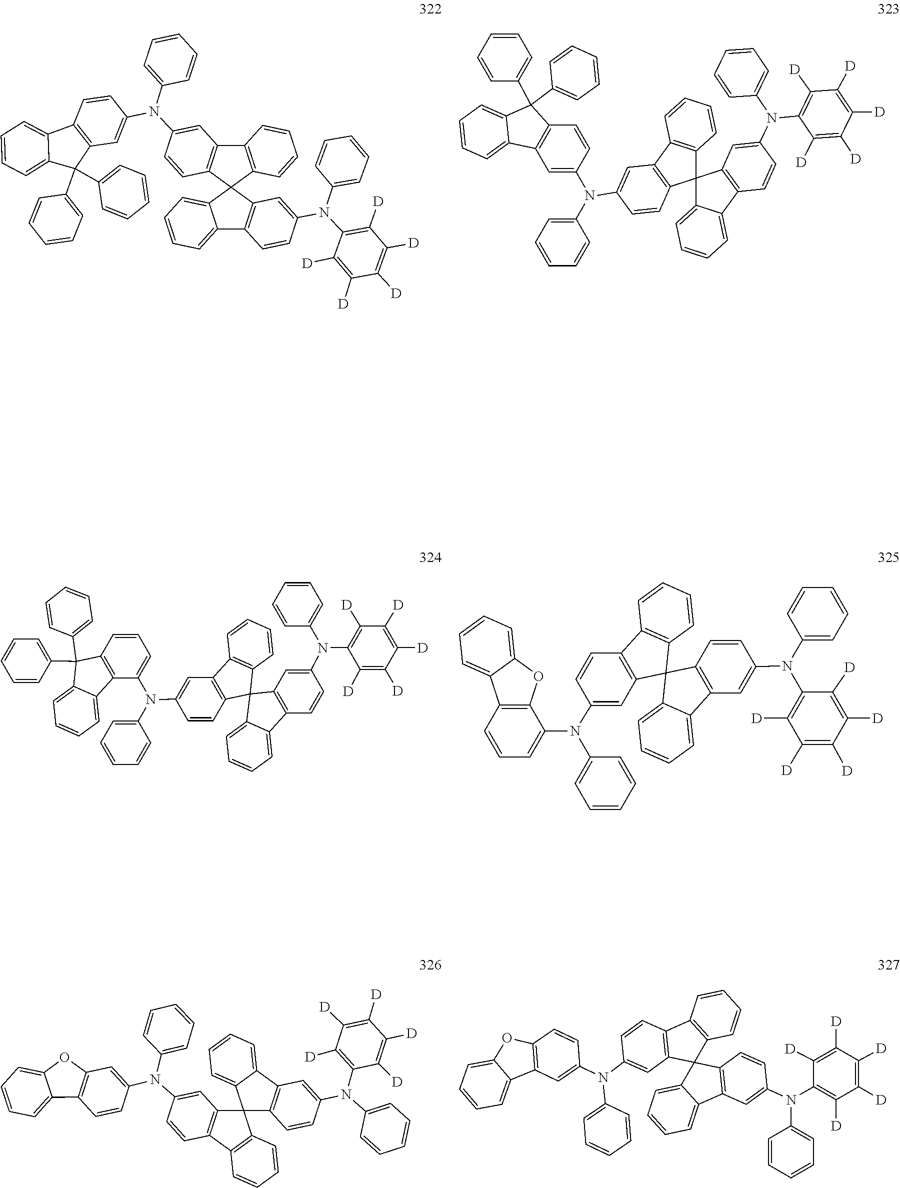

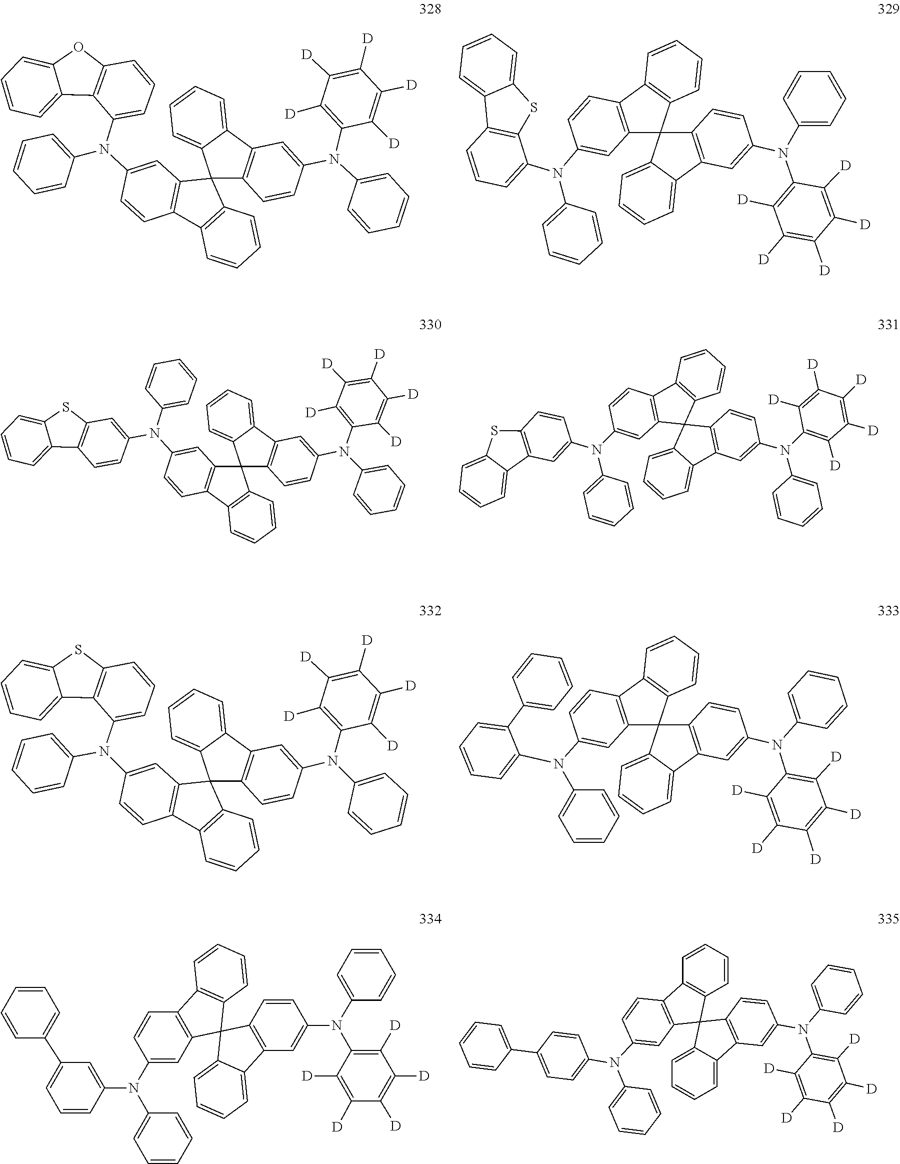

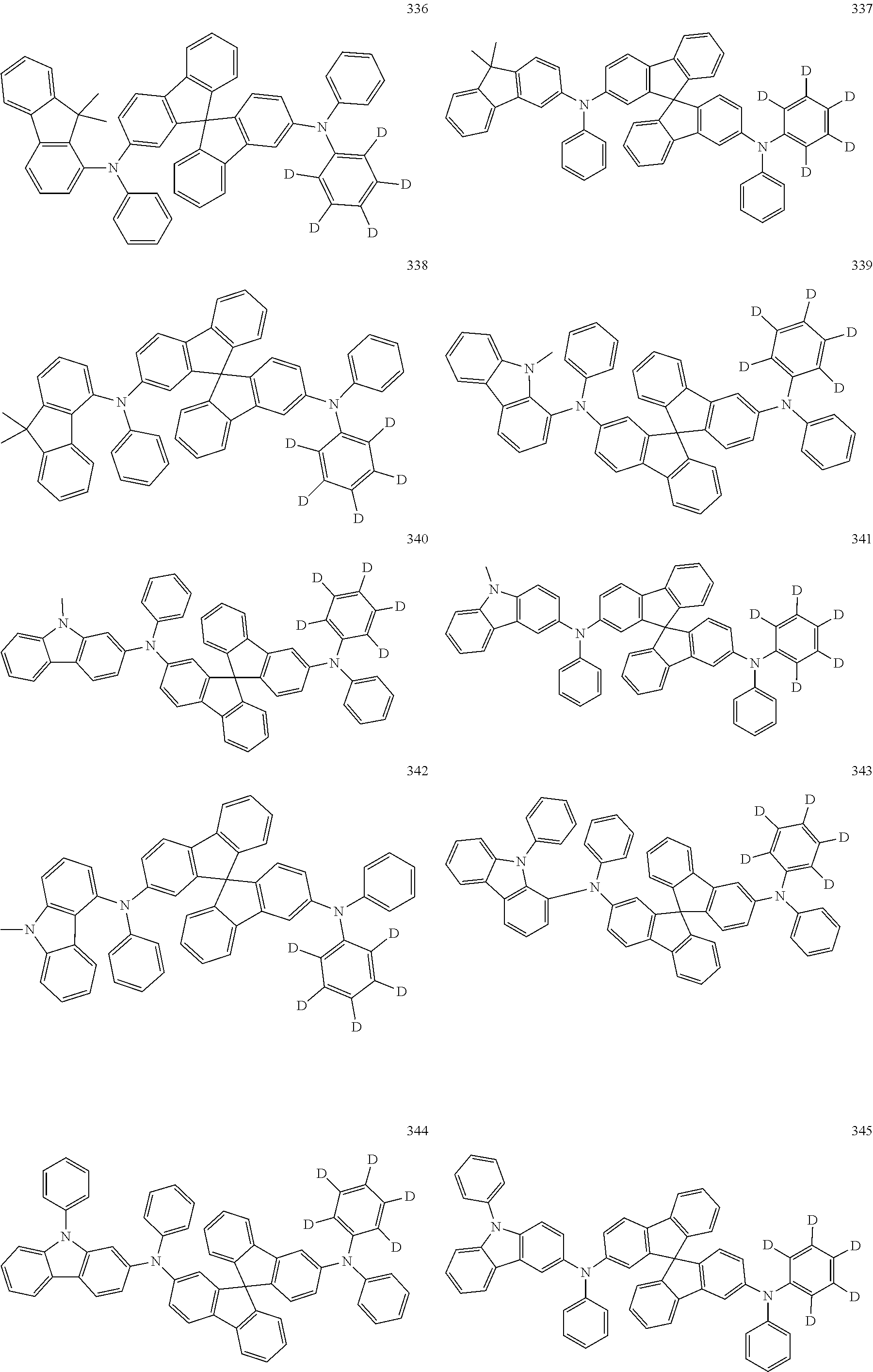

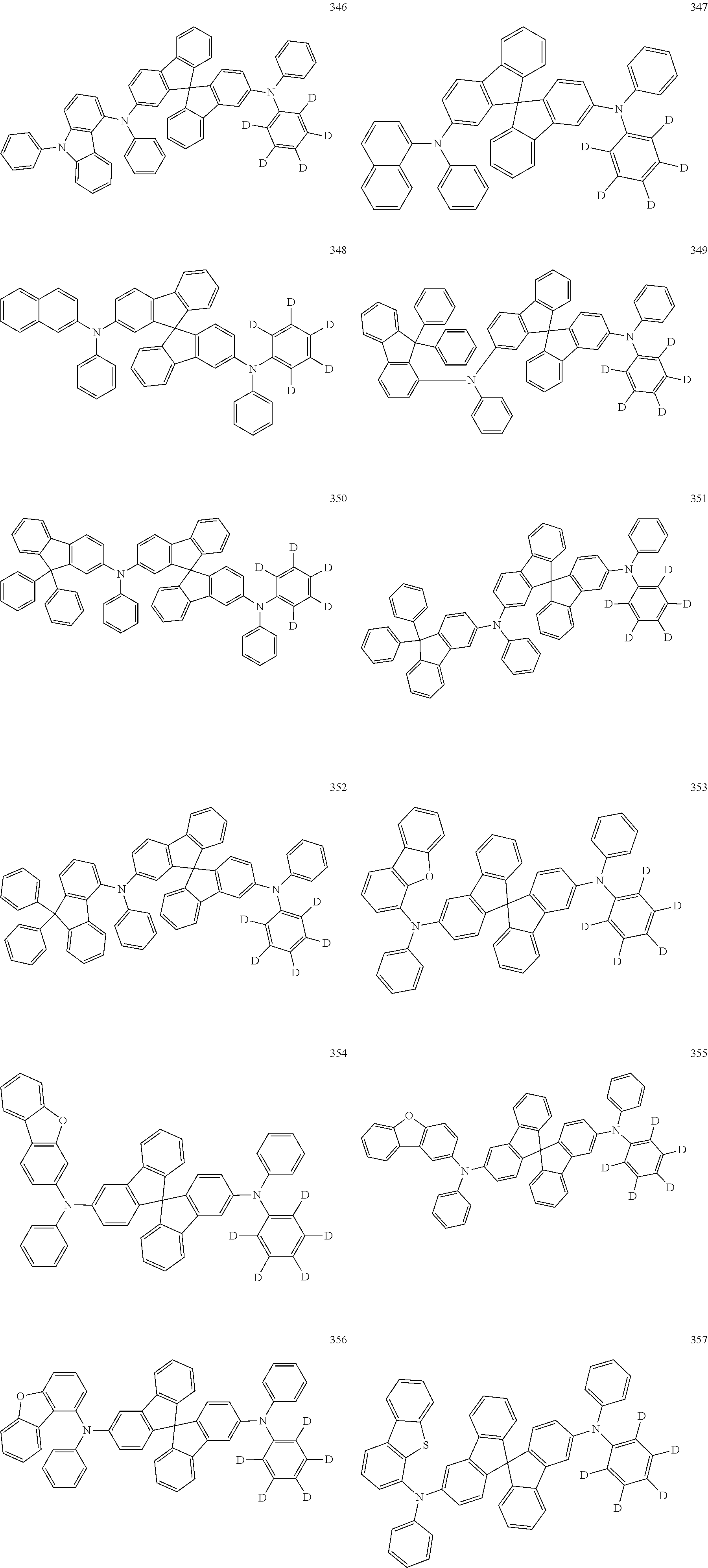

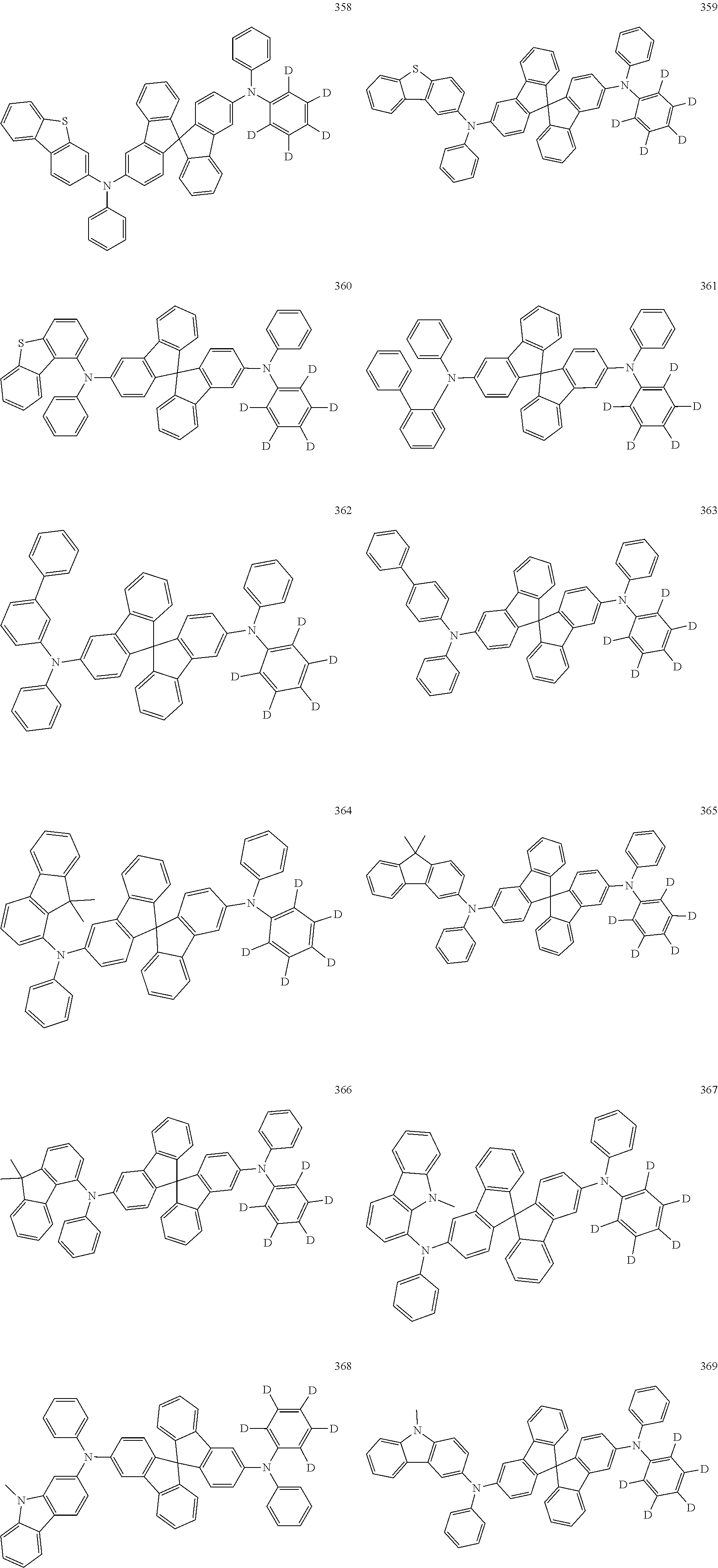

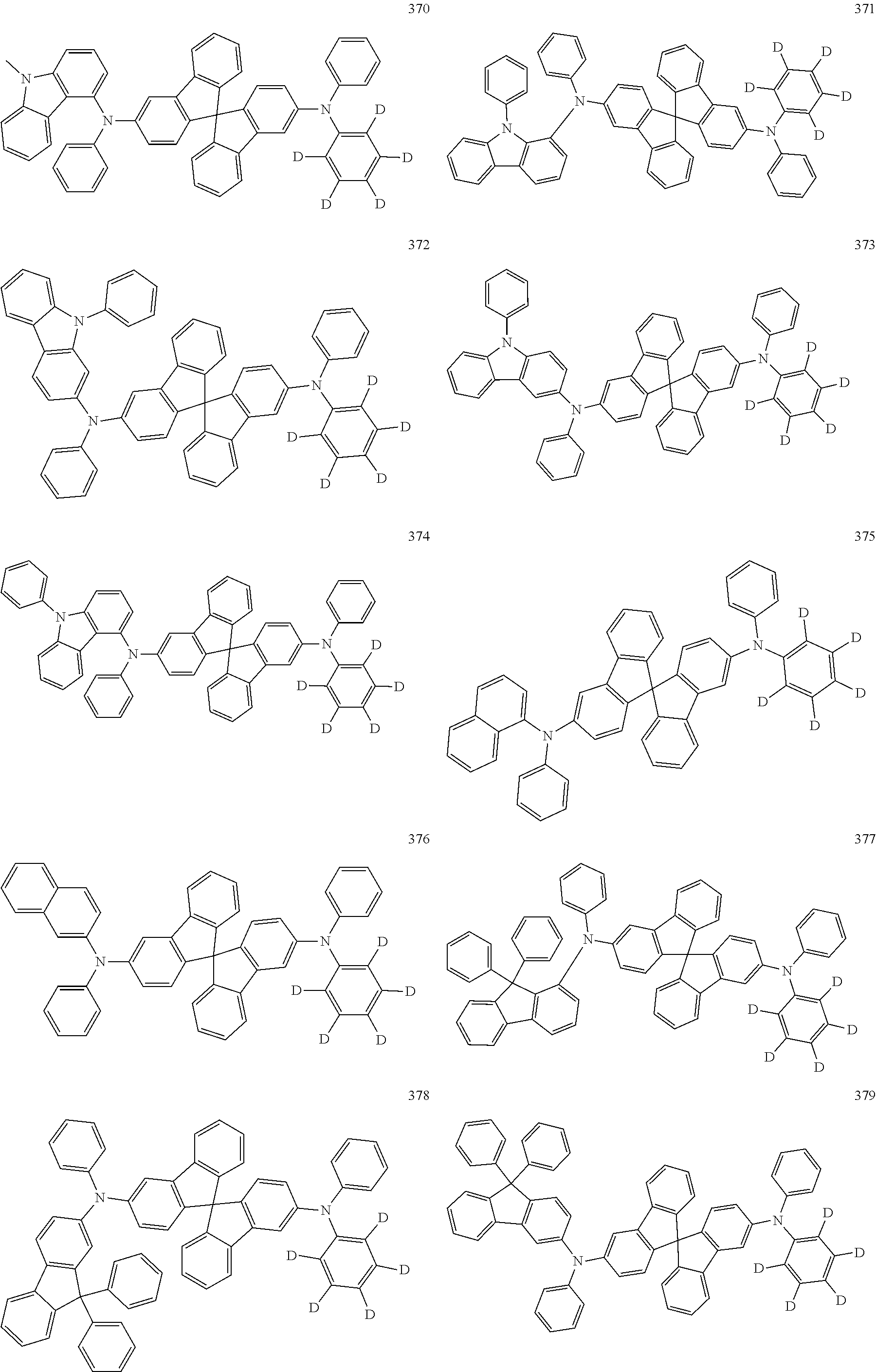

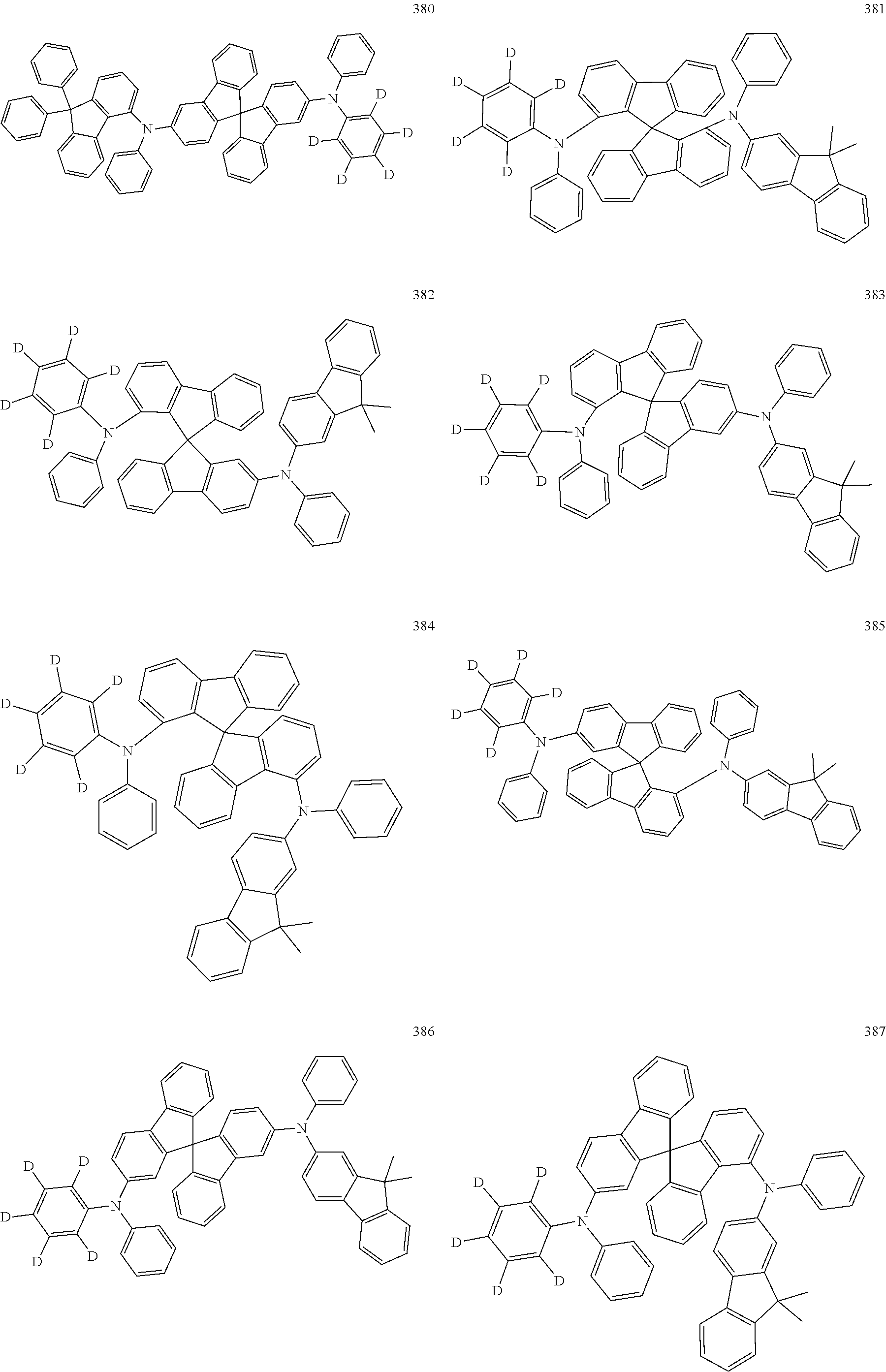

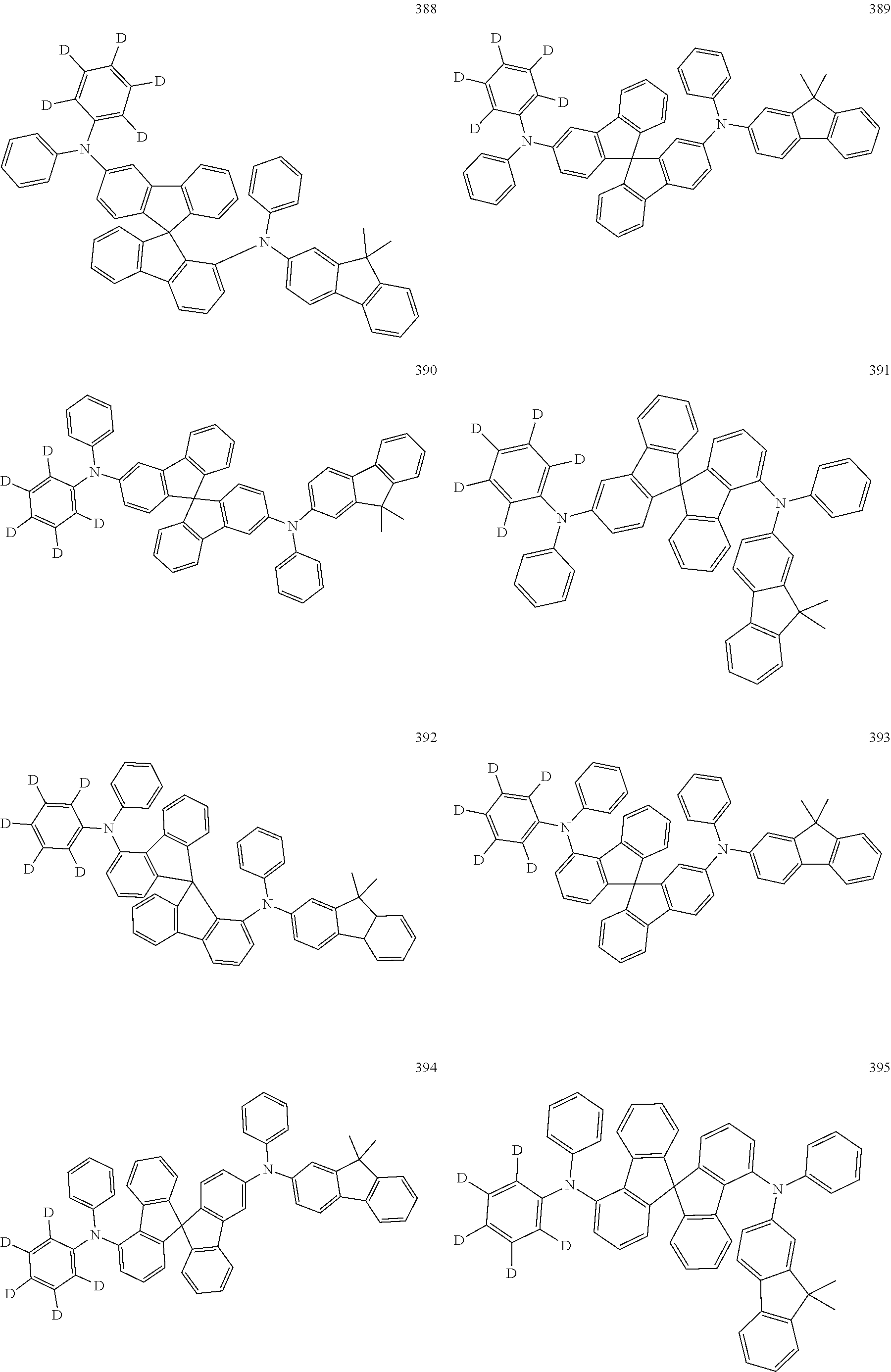

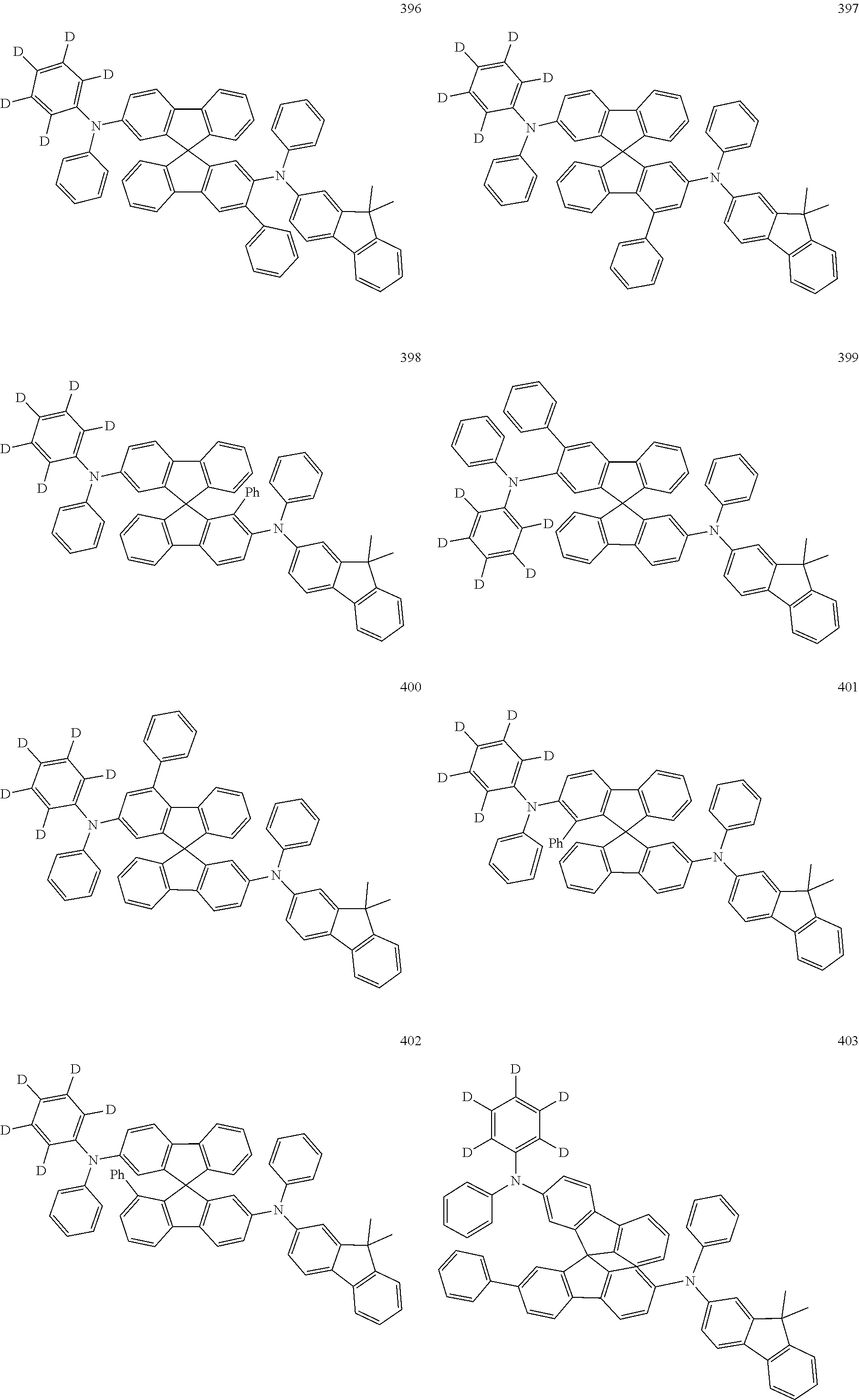

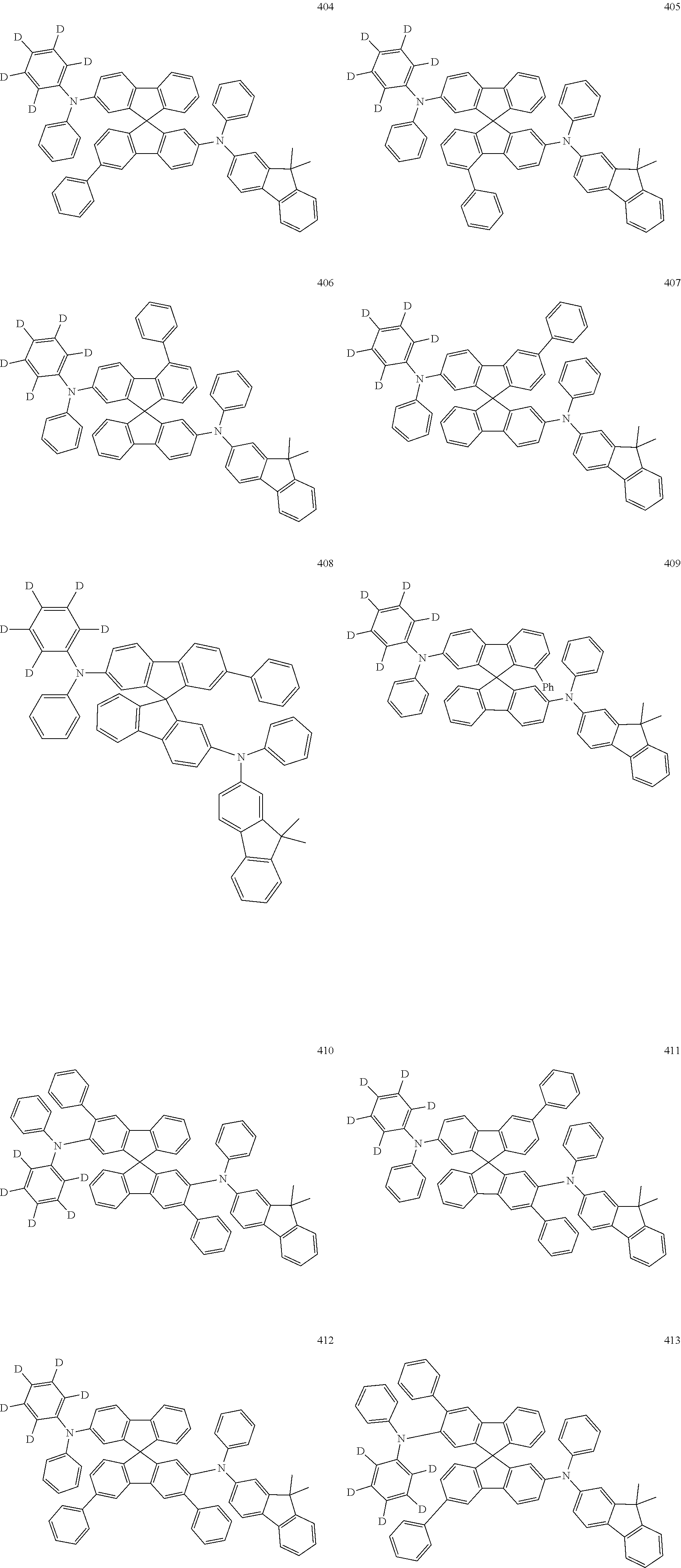

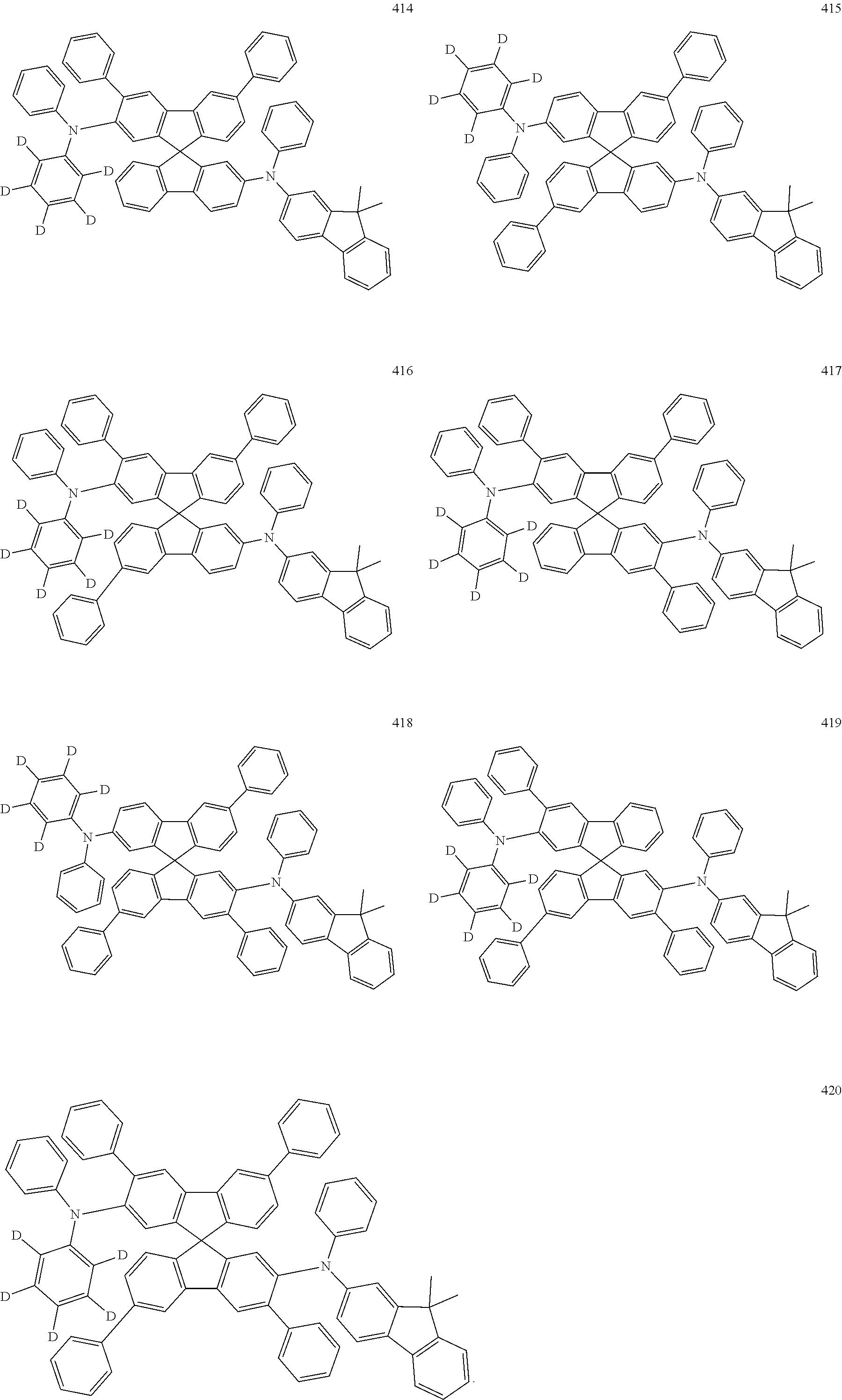

The diamine compound represented by Formula 1 may include at least one amine moiety of which a substituent is substituted with four or more deuterium atoms. Accordingly, an isotope effect caused by deuterium may be identified in the diamine compound according to an embodiment.

For example, because of the isotope effect, the vibrational energy of the amine moiety may be reduced, an interaction between diamine compounds may be reduced, and heat resistance and lifespan of the diamine compound may be improved.

Furthermore, the diamine compound represented by Formula 1 may further include at least one substituent represented by Formula 1-3 or 1-4. Accordingly, the diamine compound according to an embodiment may have a larger structure. Furthermore, the diamine compound may maintain a suitable or optimal intermolecular density.

In addition, amine moieties included in the diamine compound of Formula 1 may have different electrochemical environments from each other. As a result, energy levels such as HOMO, LUMO, T1, S1, etc. may be micro-controlled (e.g., finely controlled) and hole mobility may be easily controlled.

As a result, the hole mobility and thermal resistance of the diamine compound may evenly be improved (e.g., may both be improved, and, for example, may both be improved by similar amounts), and an electronic device including the diamine compound, for example, an organic light-emitting device, may have low driving voltage, high efficiency, and a long lifespan.

Synthesis methods of the diamine compound represented by Formula 1 may be recognizable by one of ordinary skill in the art by referring to Synthesis Examples and/or Examples provided below.

At least one diamine compound represented by Formula 1 may be used in a light-emitting device (for example, an organic light-emitting device). Accordingly, provided is a light-emitting device including: a first electrode; a second electrode facing the first electrode; an interlayer between the first electrode and the second electrode and including an emission layer; and a diamine compound represented by Formula 1 as described herein.

In an embodiment,

the first electrode of the light-emitting device may be an anode,

the second electrode of the light-emitting device may be a cathode,

the interlayer may further include a hole transport region between the first electrode and the emission layer and an electron transport region between the emission layer and the second electrode,

the hole transport region may include a hole injection layer, a hole transport layer, an emission auxiliary layer, an electron blocking layer, or any combination thereof, and

the electron transport region may include a buffer layer, a hole blocking layer, an electron control layer, an electron transport layer, an electron injection layer, or any combination thereof.

In an embodiment, the diamine compound may be included between the first electrode and the second electrode of the light-emitting device. Accordingly, the diamine compound may be included in the interlayer of the light-emitting device, for example, in the emission layer of the interlayer.

In an embodiment, the emission layer of the interlayer of the light-emitting device may include a dopant and a host, and the diamine compound may be included in the host. In other words, the diamine compound may act as a host. The emission layer may emit red light, green light, blue light, and/or white light. For example, the emission layer may emit blue light. The blue light may have a maximum emission wavelength of, for example, about 400 nm to about 490 nm.

In an embodiment, the emission layer of the interlayer of the light-emitting device may include a dopant and a host, the diamine compound may be included in the host, and the dopant may emit blue light. In some embodiments, the dopant may include a transition metal and ligand(s) in the number of m, m may be an integer from 1 to 6, the ligand(s) in the number of m may be identical to or different from each other, at least one of the ligand(s) in the number of m may be bound to the transition metal via a carbon-transition metal bond, and the carbon-transition metal bond may be a coordinate bond (which may also be referred to as a coordinate covalent bond or a dative bond). For example, at least one of the ligand(s) in the number of m may be a carbene ligand (e.g., Ir(pmp)₃ and/or the like). The transition metal may be, for example, iridium, platinum, osmium, palladium, rhodium, or gold. The emission layer and the dopant may be the same as described in the present specification.

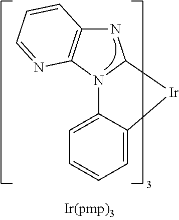

In an embodiment, the light-emitting device may include a capping layer outside the first electrode and/or outside the second electrode.

In an embodiment, the light-emitting device may further include at least one of a first capping layer outside the first electrode and a second capping layer outside the second electrode, and at least one selected from the first capping layer and the second capping layer may include the diamine compound represented by Formula 1. For more details of the first capping layer and/or second capping layer the descriptions of the first capping layer and/or second capping layer in the present specification may be referred to.

In an embodiment, the light-emitting device may further include:

a first capping layer outside the first electrode and including the diamine compound represented by Formula 1;

a second capping layer outside the second electrode and including the diamine compound represented by Formula 1; or

the first capping layer and the second capping layer.

The expression “(an interlayer and/or a capping layer) includes at least one diamine compound,” as used herein may include a case in which “(an interlayer and/or a capping layer) includes identical diamine compounds represented by Formula 1” and a case in which “(an organic layer) includes two or more different diamine compounds represented by Formula 1.”

For example, the interlayer and/or capping layer may include Compound 1 only as the diamine compound. In this regard, Compound 1 may be present in the emission layer of the light-emitting device. In one or more embodiments, the interlayer may include, as the diamine compound, Compound 1 and Compound 2. In this regard, Compound 1 and Compound 2 may be present in the same layer (for example, all of Compound 1 and Compound 2 may be present in the emission layer), or may be present in different layers (for example, Compound 1 may be present in the emission layer, and Compound 2 may be present in the electron transport region).

The term “interlayer,” as used herein, refers to a single layer and/or all of a plurality of layers between the first electrode and the second electrode of the light-emitting device.

Another aspect of embodiments provides an electronic apparatus including the light-emitting device. The electronic apparatus may further include a thin-film transistor. For example, the electronic apparatus may further include a thin-film transistor including a source electrode and a drain electrode, wherein the first electrode of the light-emitting device may be electrically connected to the source electrode or the drain electrode. In an embodiment, the electronic apparatus may further include a color filter, a color conversion layer, a touch screen layer, a polarizing layer, or any combination thereof. For more details of the electronic apparatus, related descriptions provided herein may be referred to.

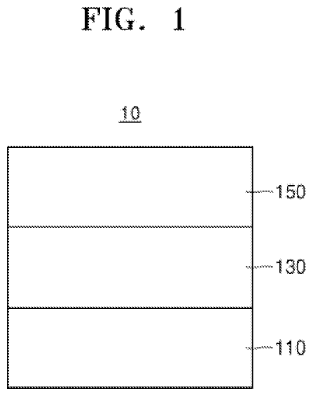

### DESCRIPTION OF FIG. 1

FIG. 1 is a schematic cross-sectional view of a light-emitting device 10 according to an embodiment. The light-emitting device 10 includes a first electrode 110, an interlayer 130, and a second electrode 150.

Hereinafter, the structure of the light-emitting device 10 according to an embodiment and a method of manufacturing the light-emitting device 10 will be described with reference to FIG. 1.

### First Electrode 110

In FIG. 1, a substrate may be additionally under the first electrode 110 and/or on the second electrode 150. As the substrate, a glass substrate and/or a plastic substrate may be used. In one or more embodiments, the substrate may be a flexible substrate, and may include plastics having excellent heat resistance and durability, such as polyimide, polyethylene terephthalate (PET), polycarbonate, polyethylene naphthalate, polyarylate (PAR), polyetherimide, or any combination thereof.

The first electrode 110 may be formed by, for example, depositing and/or sputtering a material for forming the first electrode 110 on the substrate. When the first electrode 110 is an anode, a material for forming the first electrode 110 may be a high-work function material that facilitates injection of holes.

The first electrode 110 may be a reflective electrode, a semi-transmissive electrode, or a transmissive electrode. When the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. In one or more embodiments, when the first electrode 110 is a semi-transmissive electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may have a single-layered structure consisting of a single layer or a multi-layered structure including a plurality of layers. For example, the first electrode 110 may have a three-layered structure of ITO/Ag/ITO.

### Interlayer 130

The interlayer 130 may be on the first electrode 110. The interlayer 130 may include an emission layer.

The interlayer 130 may further include a hole transport region between the first electrode 110 and the emission layer, and an electron transport region between the emission layer and the second electrode 150.

The interlayer 130 may further include, in addition to various suitable organic materials, a metal-containing compound such as an organometallic compound, an inorganic material such as quantum dots, and/or the like.

In one or more embodiments, the interlayer 130 may include, i) two or more emitting units sequentially stacked between the first electrode 110 and the second electrode 150, and ii) a charge generation layer between the two or more emitting units. When the interlayer 130 includes emitting units and a charge generation layer as described above, the light-emitting device 10 may be a tandem light-emitting device.

### Hole Transport Region in Interlayer 130

The hole transport region may have: i) a single-layered structure consisting of a single layer consisting of a single material, ii) a single-layered structure consisting of a single layer consisting of a plurality of different materials, or iii) a multi-layered structure including a plurality of layers including different materials.

The hole transport region may include a hole injection layer, a hole transport layer, an emission auxiliary layer, an electron-blocking layer, or any combination thereof.

For example, the hole transport region may have a multi-layered structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron-blocking layer structure, the layers of each structure being stacked sequentially from the first electrode 110.

The hole transport region may include a compound represented by Formula 201, a compound represented by Formula 202, or any combination thereof:

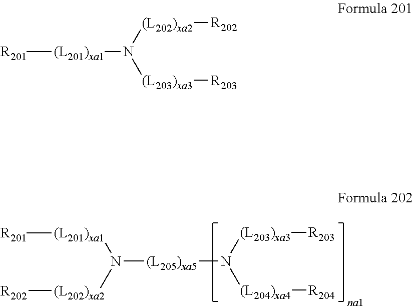

wherein, in Formulae 201 and 202,

L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

L₂₀₅ may be *—O—*′, *—S—*′, *—N(Q₂₀₁)—*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,

xa1 to xa4 may each independently be an integer from 0 to 5,

xa5 may be an integer from 1 to 10,

R₂₀₁ to R₂₀₄ and Q₂₀₁ may each independently be a C₃-C₆₀ carbocyclic group unsubstituted or substituted with at least one R₁₀ₐ, or a C₁-C₆₀ heterocyclic group unsubstituted or substituted with at least one R₁₀ₐ,

R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group (for example, a carbazole group and/or the like) unsubstituted or substituted with at least one R₁₀ₐ (for example, Compound HT16),

R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ, to form a C₈-C₆₀ polycyclic group unsubstituted or substituted with at least one R₁₀ₐ, and

na1 may be an integer from 1 to 4.

For example, each of Formulae 201 and 202 may include at least one selected from croups represented by Formulae CY201 to CY217:

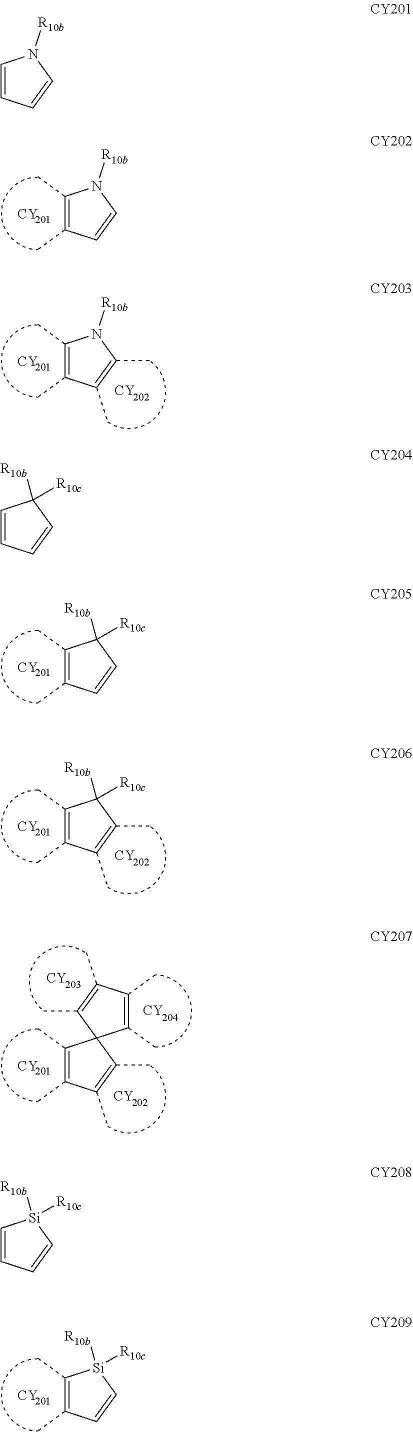

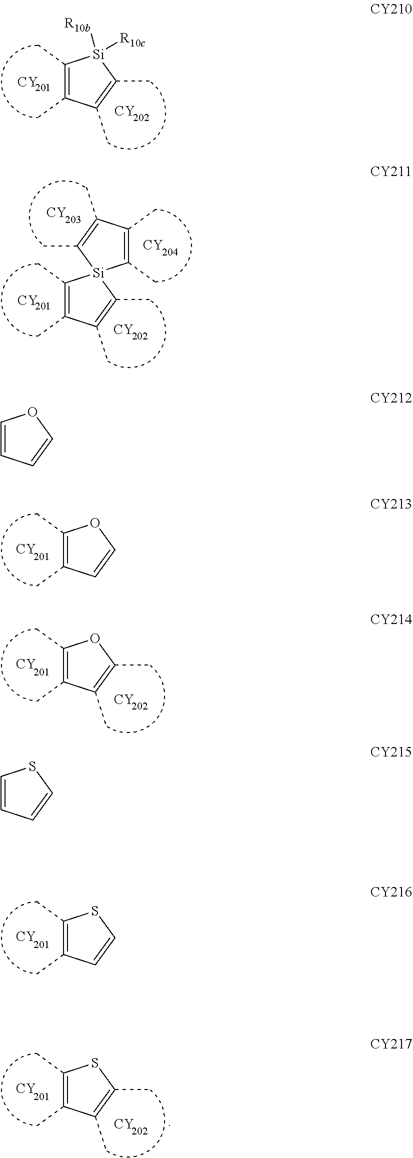

In Formulae CY201 to CY217, R_{10b} and R_{10c} may each be the same as described with respect to R₁₀ₐ, rings CY₂₀₁ to CY₂₀₄ may each independently be a C₃-C₂₀ carbocyclic group or a C₁-C₂₀ heterocyclic group, and at least one hydrogen in Formulae CY201 to CY217 may be unsubstituted or substituted with R₁₀ₐ as described above.

In an embodiment, rings CY201 to CY204 in Formulae CY201 to CY217 may each independently be a benzene group, a naphthalene group, a phenanthrene group, or an anthracene group.

In one or more embodiments, each of Formulae 201 and 202 may include at least one selected from groups represented by Formulae CY201 to CY203.

In one or more embodiments, Formula 201 may include at least one selected from the groups represented by Formulae CY201 to CY203 and at least one selected from the groups represented by Formulae CY204 to CY217.

In one or more embodiments, in Formula 201, xa1 may be 1, R₂₀₁ may be a group represented by one selected from Formulae CY201 to CY203, xa2 may be 0, and R₂₀₂ may be a group represented by one selected from Formulae CY204 to CY207.

In one or more embodiments, each of Formulae 201 and 202 may not include a group represented by one selected from Formulae CY201 to CY203.

In one or more embodiments, each of Formulae 201 and 202 may not include a group represented by one selected from Formulae CY201 to CY203, and may include at least one selected from the groups represented by Formulae CY204 to CY217.

In one or more embodiments, each of Formulae 201 and 202 may not include a group represented by one selected from Formulae CY201 to CY217.

In an embodiment, the hole transport region may include one of Compounds HT1 to HT46, m-MTDATA, TDATA, 2-TNATA, NPB(NPD), β-NPB, TPD, Spiro-TPD, Spiro-NPB, methylated NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), or any combination thereof:

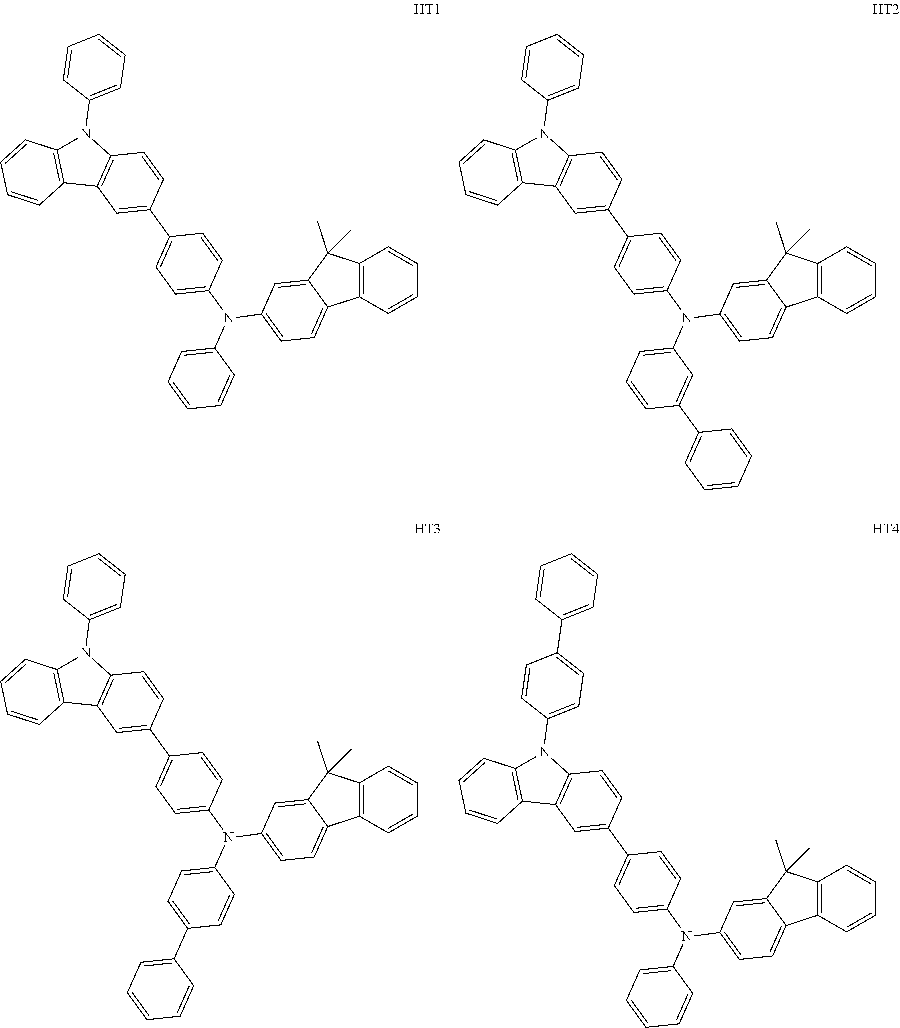

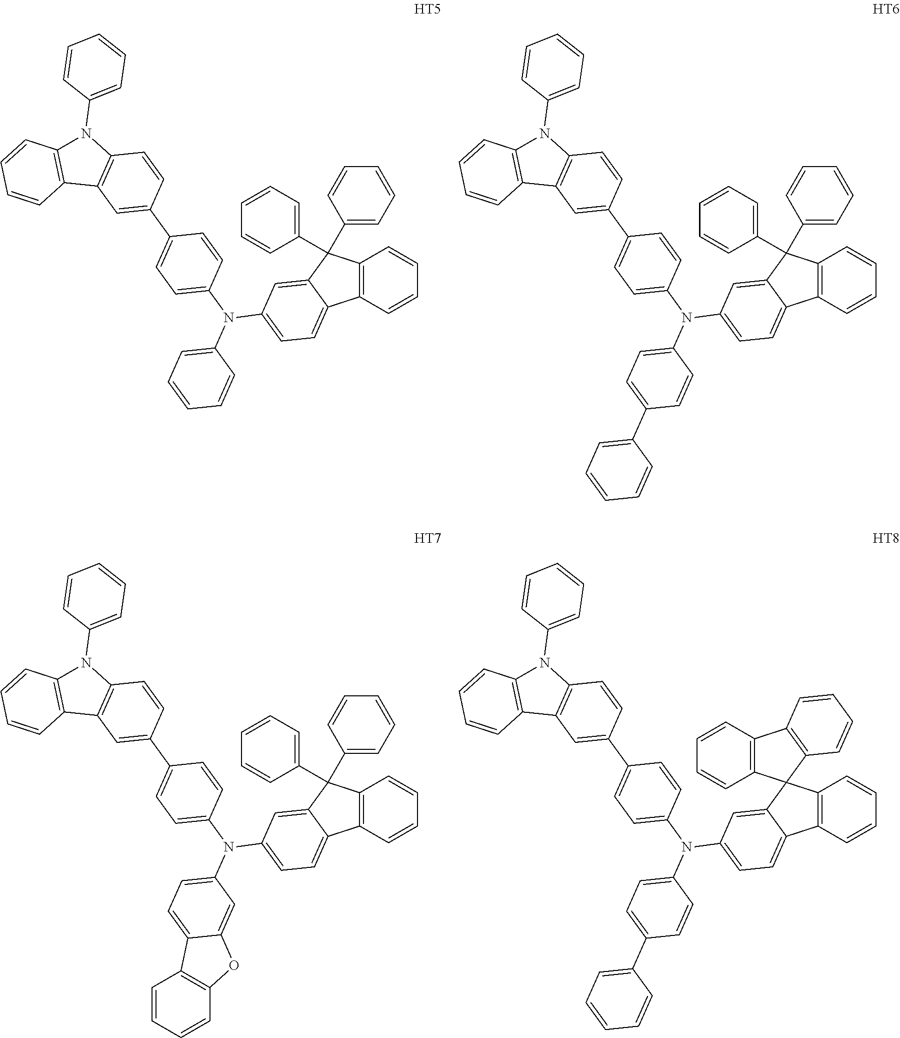 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

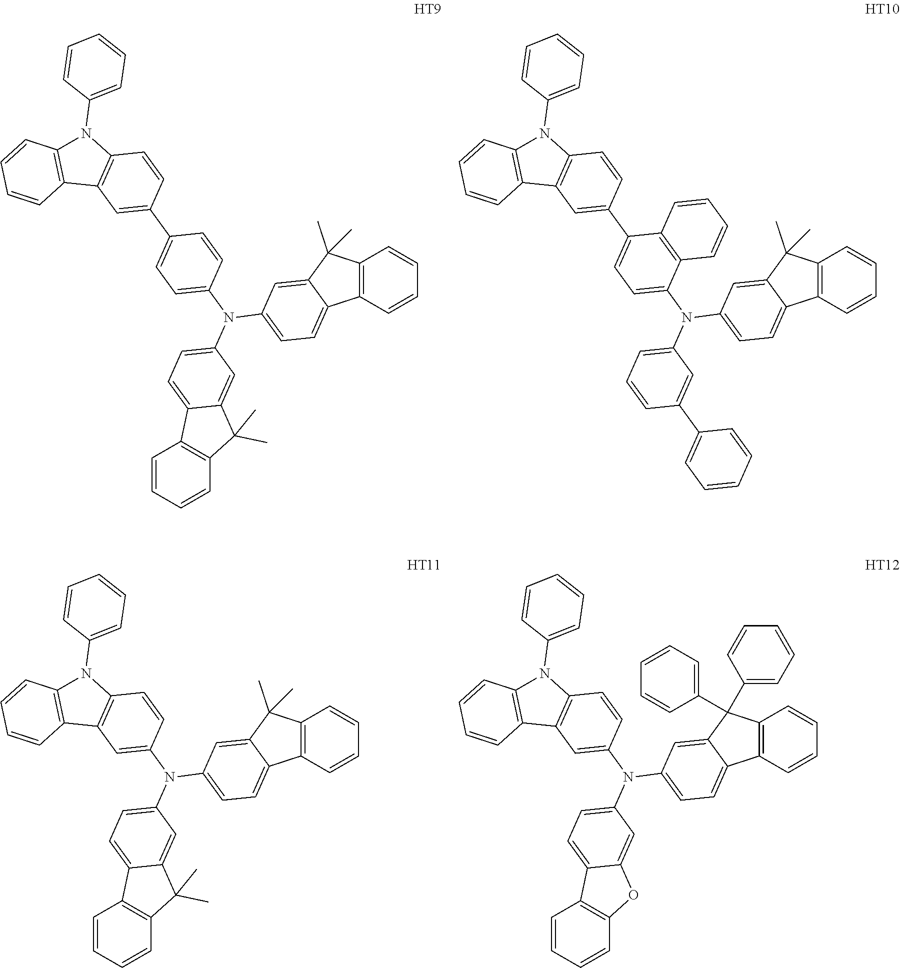 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

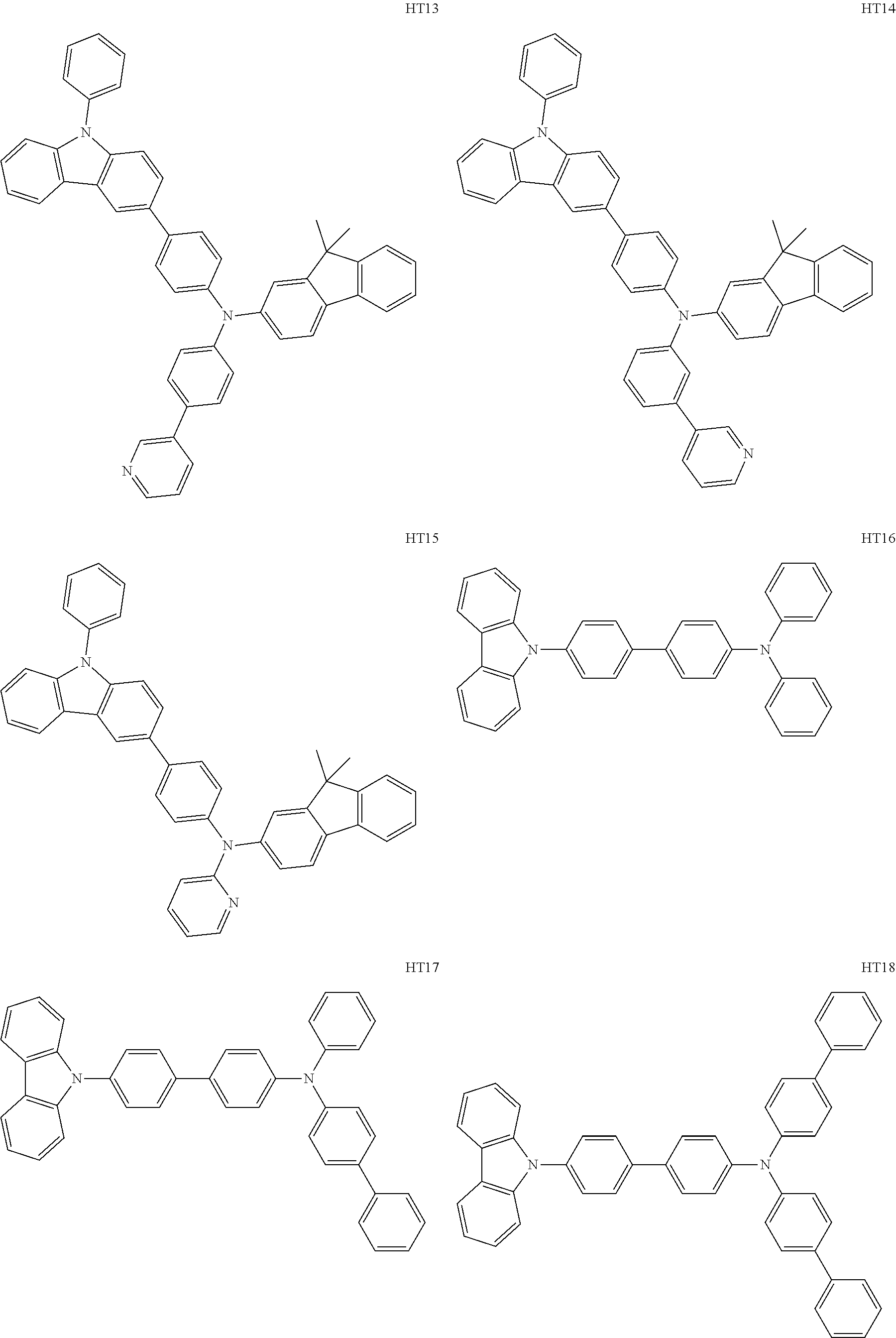

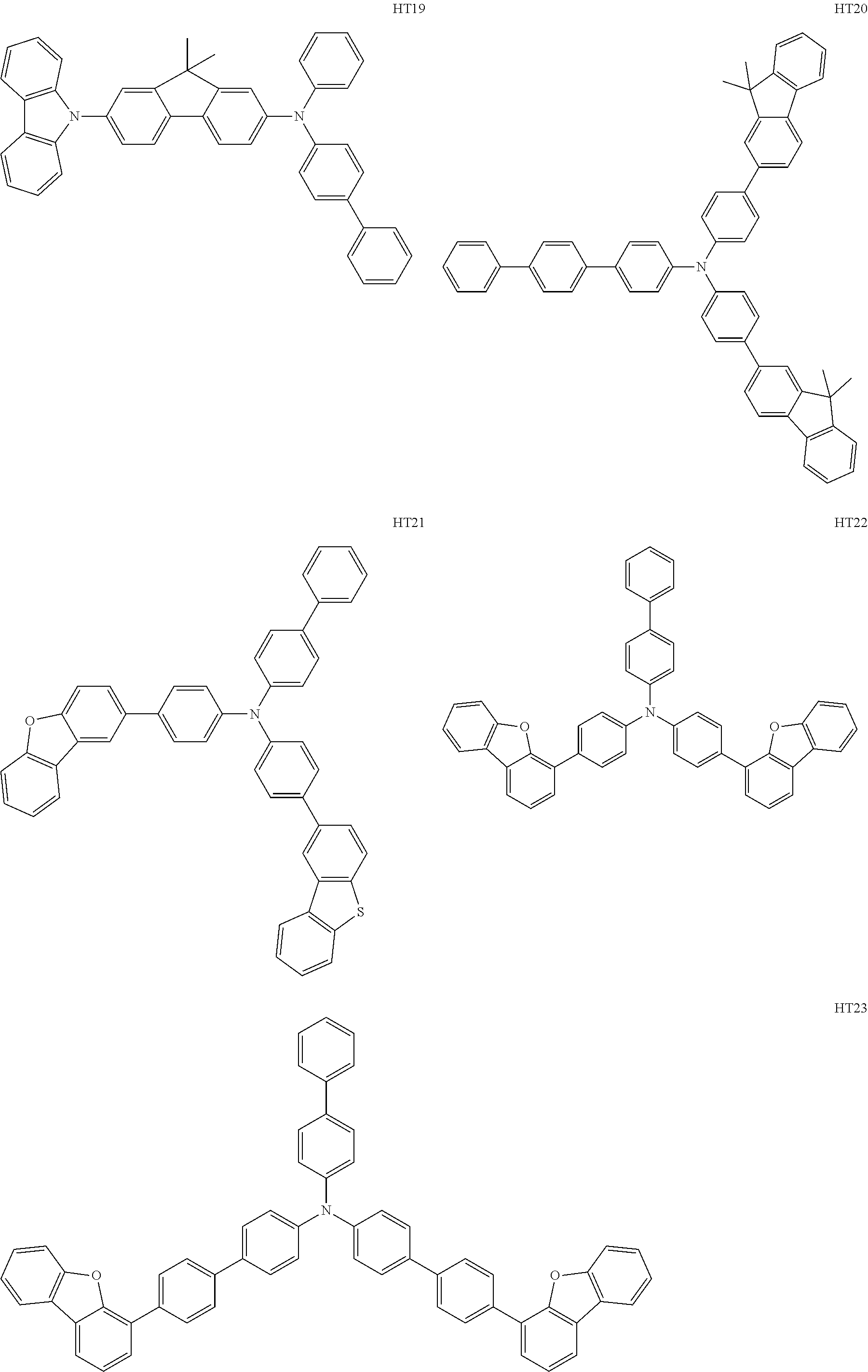

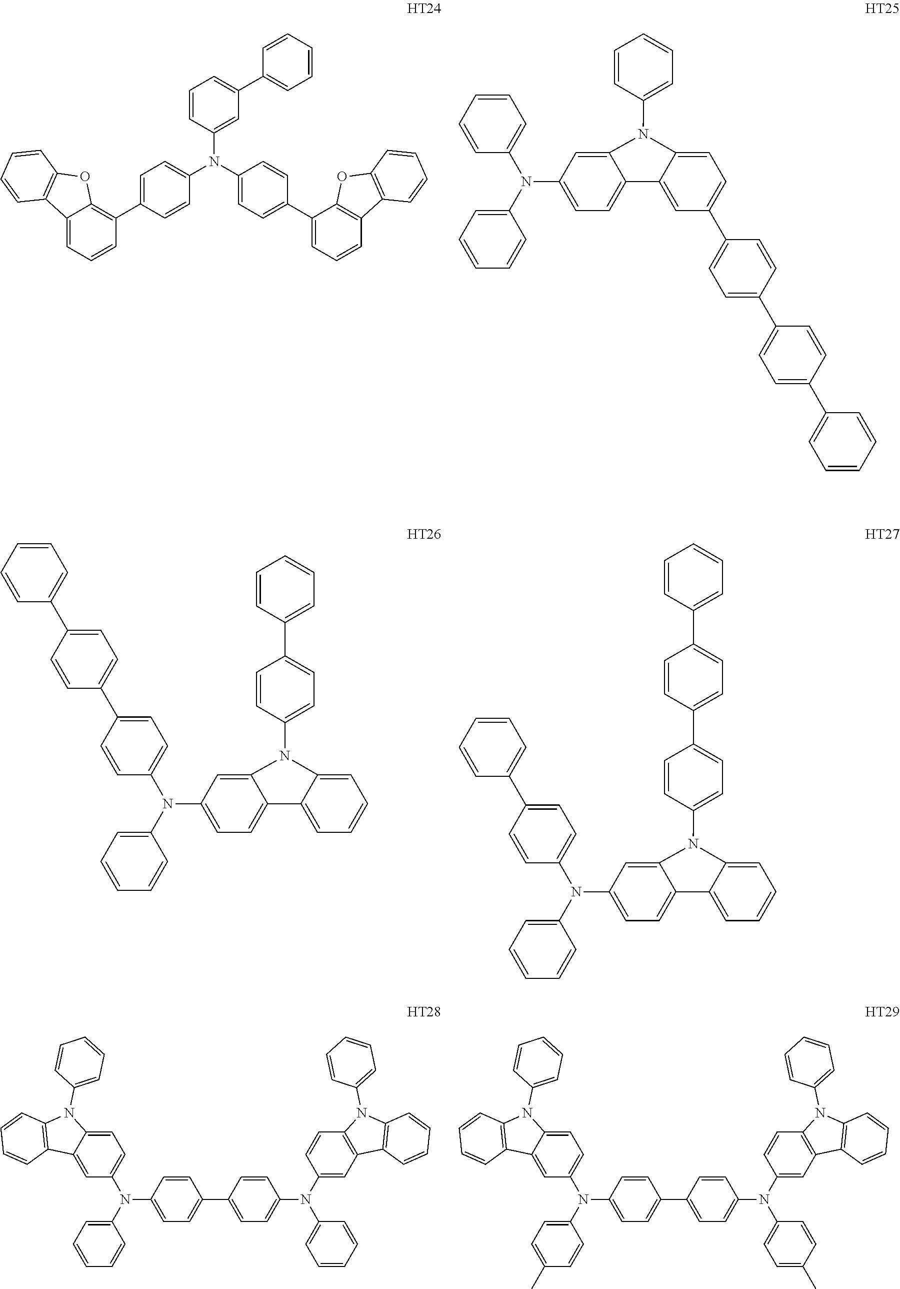

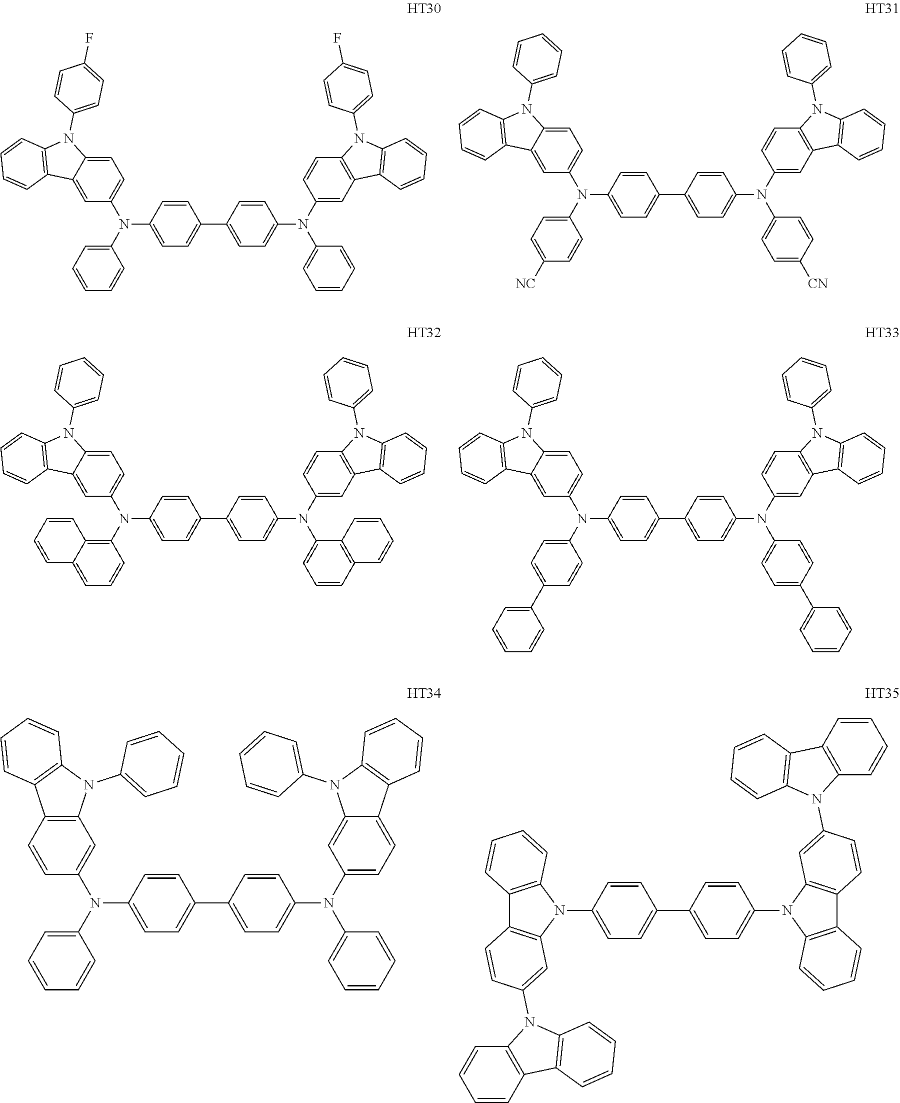

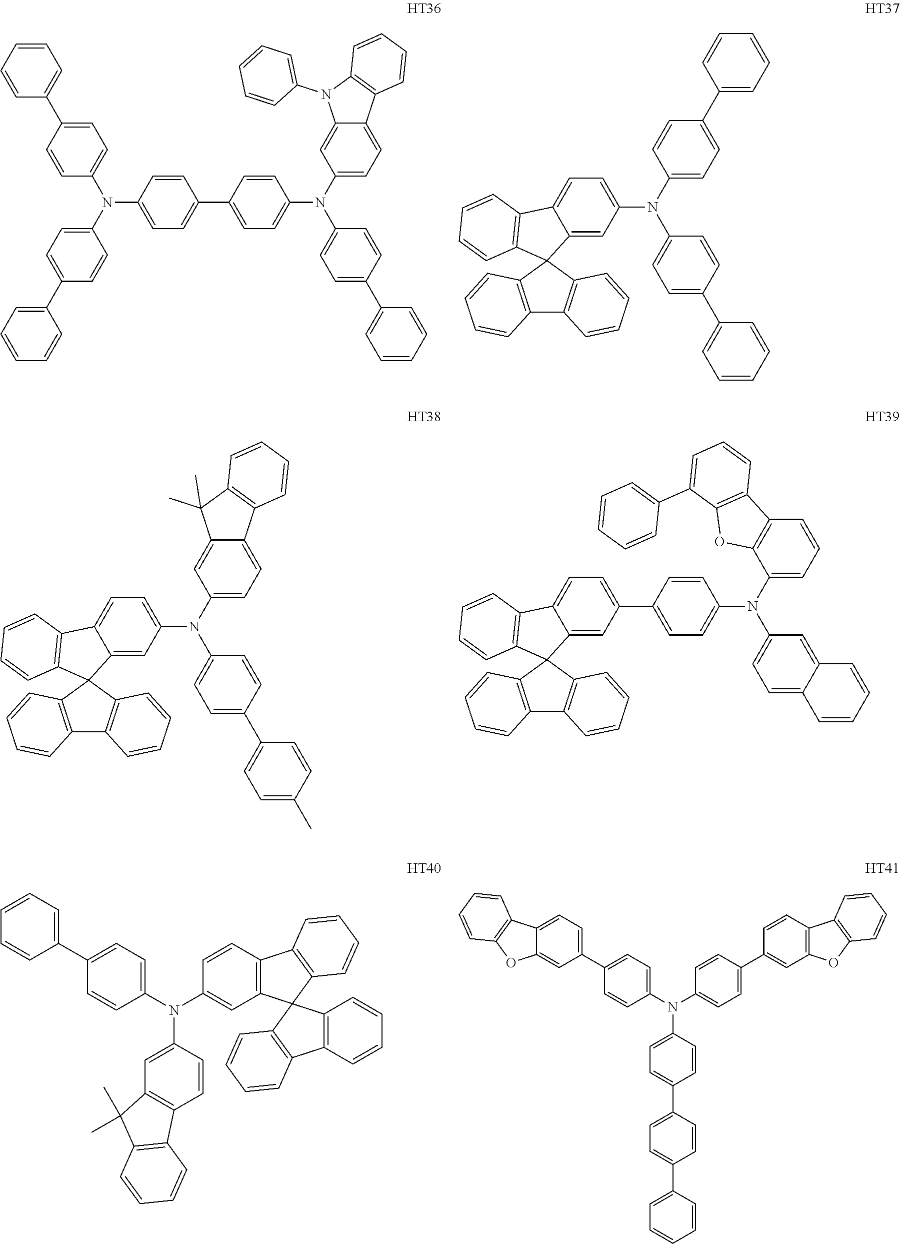

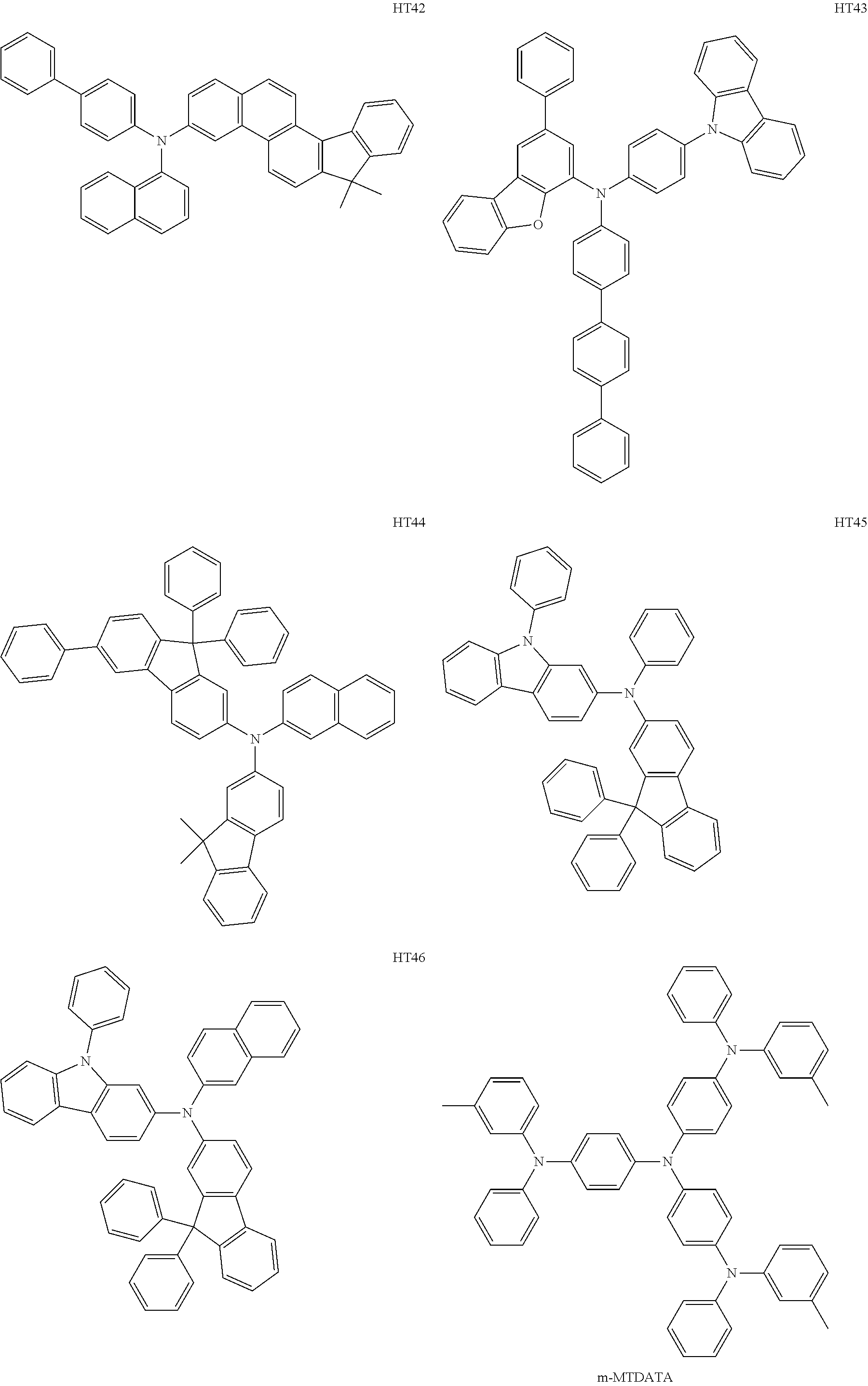

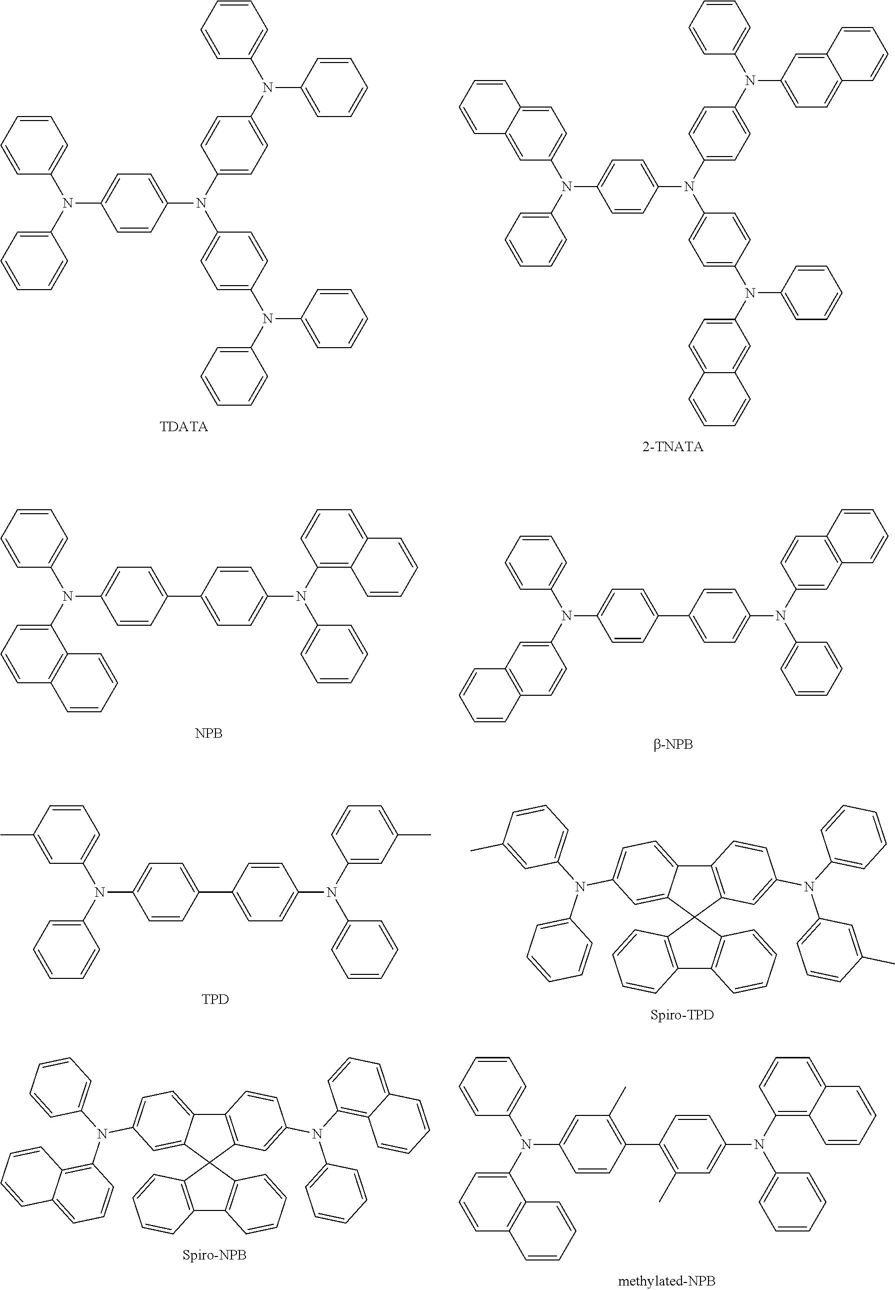

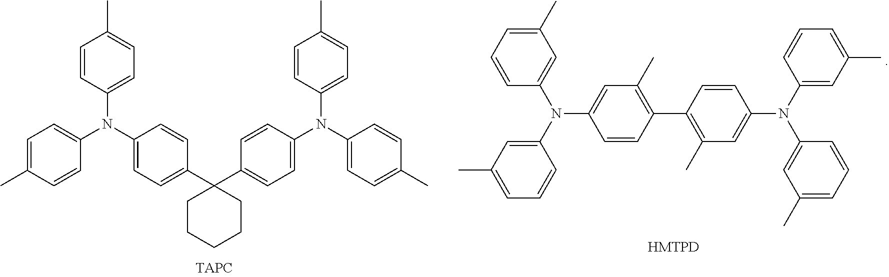

A thickness of the hole transport region may be in a range of about 50 Å to about 10,000 Å, for example, about 100 Å to about 4,000 Å. When the hole transport region includes a hole injection layer, a hole transport layer, or any combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region, the hole injection layer, and the hole transport layer are within these ranges, suitable or satisfactory hole transporting characteristics may be obtained without a substantial increase in driving voltage.

The emission auxiliary layer may increase light-emission efficiency by compensating for an optical resonance distance according to the wavelength of light emitted by an emission layer, and the electron-blocking layer may block or reduce the leakage of electrons from an emission layer to a hole transport region. Materials that may be included in the hole transport region may be included in the emission auxiliary layer and the electron-blocking layer.

p-Dopant

The hole transport region may further include, in addition to these materials, a charge-generation material for the improvement of conductive properties (e.g., electrically conductive properties). The charge-generation material may be uniformly or non-uniformly dispersed in the hole transport region (for example, in the form of a single layer consisting of a charge-generation material).

The charge-generation material may be, for example, a p-dopant.

For example, the lowest unoccupied molecular orbital (LUMO) energy level of the p-dopant may be −3.5 eV or less.

In one or more embodiments, the p-dopant may include a quinone derivative, a cyano group-containing compound, a compound including element EL1 and element EL2, or any combination thereof.

Examples of the quinone derivative are TCNQ, F4-TCNQ, etc.

Examples of the cyano group-containing compound are HAT-CN, and a compound represented by Formula 221 below.

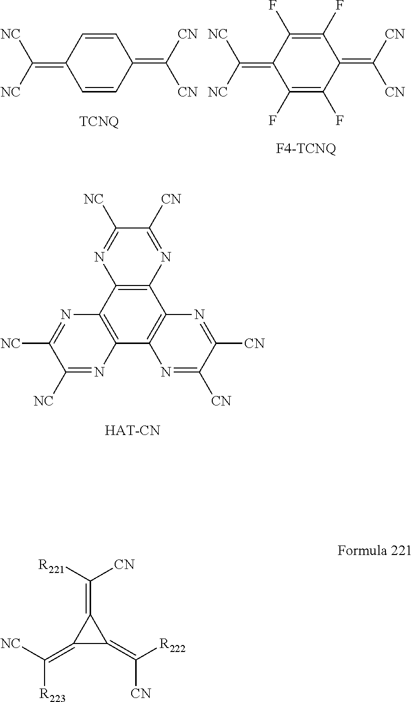

In Formula 221,

R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and

at least one selected from R₂₂₁ to R₂₂₃ may each independently be a C₃-C₆₀ carbocyclic group or a C₁-C₆₀ heterocyclic group, each substituted with a cyano group; —F; —Cl; —Br; —I; a C₁-C₂₀ alkyl group substituted with a cyano group, —F, —Cl, —Br, —I, or any combination thereof; or any combination thereof.

In the compound including element EL1 and element EL2, element EL1 may be metal, metalloid, or any combination thereof, and element EL2 may be non-metal, metalloid, or any combination thereof.

Examples of the metal include an alkali metal (for example, lithium (Li), sodium (Na), potassium (K), rubidium (Rb), cesium (Cs), etc.); alkaline earth metal (for example, beryllium (Be), magnesium (Mg), calcium (Ca), strontium (Sr), barium (Ba), etc.); transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); post-transition metal (for example, zinc (Zn), indium (In), tin (Sn), etc.); and lanthanide metal (for example, lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.).

Examples of the metalloid include silicon (Si), antimony (Sb), and tellurium (Te).

Examples of the non-metal include oxygen (O) and halogen (for example, F, Cl, Br, I, etc.).

Examples of the compound including element EL1 and element EL2 include metal oxide, metal halide (for example, metal fluoride, metal chloride, metal bromide, and/or metal iodide), metalloid halide (for example, metalloid fluoride, metalloid chloride, metalloid bromide, and/or metalloid iodide), metal telluride, or any combination thereof.

Examples of the metal oxide include tungsten oxide (for example, WO, W₂O₃, WO₂, WO₃, W₂O₅, etc.), vanadium oxide (for example, VO, V₂O₃, VO₂, V₂O₅, etc.), molybdenum oxide (MoO, Mo₂O₃, MoO₂, MoO₃, Mo₂O₅, etc.), and rhenium oxide (for example, ReO₃, etc.).

Examples of the metal halide include alkali metal halide, alkaline earth metal halide, transition metal halide, post-transition metal halide, and lanthanide metal halide.

Examples of the alkali metal halide include LiF, NaF, KF, RbF, CsF, LiCl, NaCl, KCl, RbCl, CsCl, LiBr, NaBr, KBr, RbBr, CsBr, LiI, NaI, KI, RbI, and CsI.

Examples of the alkaline earth metal halide include BeF₂, MgF₂, CaF₂, SrF₂, BaF₂, BeCl₂, MgCl₂, CaCl₂, SrCl₂, BaCl₂, BeBr₂, MgBr₂, CaBr₂, SrBr₂, BaBr₂, BeI₂, MgI₂, CaI₂, SrI₂, and BaI₂.

Examples of the transition metal halide include titanium halide (for example, TiF₄, TiCl₄, TiBr₄, TiI₄, etc.), zirconium halide (for example, ZrF₄, ZrCl₄, ZrBr₄, ZrI₄, etc.), hafnium halide (for example, HfF₄, HfCl₄, HfBr₄, HfI₄, etc.), vanadium halide (for example, VF₃, VCl₃, VBr₃, VI₃, etc.), niobium halide (for example, NbF₃, NbCl₃, NbBr₃, NbI₃, etc.), tantalum halide (for example, TaF₃, TaCl₃, TaBr₃, TaI₃, etc.), chromium halide (for example, CrF₃, CrCl₃, CrBr₃, CrI₃, etc.), molybdenum halide (for example, MoF₃, MoCl₃, MoBr₃, Mol₃, etc.), tungsten halide (for example, WF₃, WCl₃, WBr₃, WI₃, etc.), manganese halide (for example, MnF₂, MnCl₂, MnBr₂, MnI₂, etc.), technetium halide (for example, TcF₂, TcCl₂, TcBr₂, TcI₂, etc.), rhenium halide (for example, ReF₂, ReCl₂, ReBr₂, ReI₂, etc.), iron halide (for example, FeF₂, FeCl₂, FeBr₂, FeI₂, etc.), ruthenium halide (for example, RuF₂, RuCl₂, RuBr₂, RuI₂, etc.), osmium halide (for example, OsF₂, OsCl₂, OsBr₂, OsI₂, etc.), cobalt halide (for example, CoF₂, CoCl₂, CoBr₂, Cole, etc.), rhodium halide (for example, RhF₂, RhCl₂, RhBr₂, RhI₂, etc.), iridium halide (for example, IrF₂, IrCl₂, IrBr₂, IrI₂, etc.), nickel halide (for example, NiF₂, NiCl₂, NiBr₂, NiI₂, etc.), palladium halide (for example, PdF₂, PdCl₂, PdBr₂, PdI₂, etc.), platinum halide (for example, PtF₂, PtCl₂, PtBr₂, PtI₂, etc.), copper halide (for example, CuF, CuCl, CuBr, CuI, etc.), silver halide (for example, AgF, AgCl, AgBr, AgI, etc.), and gold halide (for example, AuF, AuCl, AuBr, AuI, etc.).

Examples of the post-transition metal halide include zinc halide (for example, ZnF₂, ZnCl₂, ZnBr₂, ZnI₂, etc.), indium halide (for example, InI₃, etc.), and tin halide (for example, SnI₂, etc.).

Examples of the lanthanide metal halide include YbF, YbF₂, YbF₃, SmF₃, YbCl, YbCl₂, YbCl₃ SmCl₃, YbBr, YbBr₂, YbBr₃ SmBr₃, YbI, YbI₂, YblI₃, and SmI₃.

An example of the metalloid halide includes antimony halide (for example, SbCl₅, etc.).

Examples of the metal telluride include alkali metal telluride (for example, Li₂Te, a Na₂Te, K₂Te, Rb₂Te, Cs₂Te, etc.), alkaline earth metal telluride (for example, BeTe, MgTe, CaTe, SrTe, BaTe, etc.), transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), post-transition metal telluride (for example, ZnTe, etc.), and lanthanide metal telluride (for example, LaTe, CeTe, PrTe, NdTe, PmTe, EuTe, GdTe, TbTe, DyTe, HoTe, ErTe, TmTe, YbTe, LuTe, etc.).

### Emission Layer in Interlayer 130

When the light-emitting device 10 is a full-color light-emitting device, the emission layer may be patterned into a red emission layer, a green emission layer, and/or a blue emission layer, according to a sub-pixel. In one or more embodiments, the emission layer may have a stacked structure of two or more layers of a red emission layer, a green emission layer, and a blue emission layer, in which the two or more layers contact (e.g., physically contact) each other or are separated from each other to emit white light. In one or more embodiments, the emission layer may include two or more materials of a red light-emitting material, a green light-emitting material, and a blue light-emitting material, in which the two or more materials are mixed together with each other in a single layer to emit white light.

The emission layer may include a host and a dopant. The dopant may include a phosphorescent dopant, a fluorescence dopant, or any combination thereof.

The amount of the dopant in the emission layer may be from about 0.01 part by weight to about 15 parts by weight based on 100 parts by weight of the host.

In one or more embodiments, the emission layer may include a quantum dot.

In some embodiments, the emission layer may include a delayed fluorescence material. The delayed fluorescence material may act as a host or a dopant in the emission layer.

A thickness of the emission layer may be in a range of about 100 Å to about 1,000 Å, for example, about 200 Å to about 600 Å. When the thickness of the emission layer is within these ranges, excellent light-emission characteristics may be obtained without a substantial increase in driving voltage.

### Host

In one or more embodiments, the host may include a compound represented by Formula 301 below:

Formula 301

[Ar₃₀₁]_{xb11}−[(L₃₀₁)_{xb1}−R₃₀₁]_{xb21}

In Formula 301,

Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xb11 may be 1, 2, or 3,

xb1 may be an integer from 0 to 5,

R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),

xb21 may be an integer from 1 to 5, and

Q₃₀₁ to Q₃₀₃ are each the same as described herein with respect to Q₁.

For example, when xb11 in Formula 301 is 2 or more, two or more of Ar₃₀₁(s) may be linked to each other via a single bond.

In one or more embodiments, the host may include a compound represented by Formula 301-1, a compound represented by Formula 301-2, or any combination thereof:

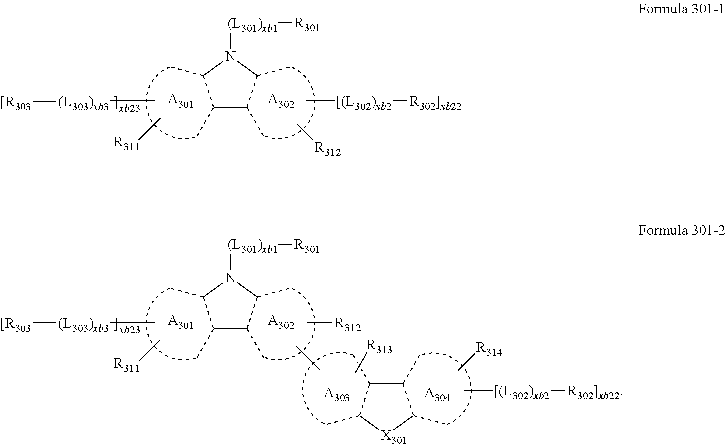

In Formulae 301-1 and 301-2,

rings A₃₀₁ to A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

X₃₀₁ may be O, S, N-[(L₃₀₄)_{xb4}-R₃₀₄], C(R₃₀₄)(R₃₀₅), or Si(R₃₀₄)(R₃₀₅),

xb22 and xb23 may each independently be 0, 1, or 2,

L₃₀₁, xb1, and R₃₀₁ may each be the same as described herein,

L₃₀₂ to L₃₀₄ may each independently be the same as described herein with respect to with L₃₀₁,

xb2 to xb4 may each independently be the same as described herein with respect to xb1, and

R₃₀₂ to R₃₀₅ and R₃₁₁ to R₃₁₄ may each be the same as described herein with respect to R₃₀₁.

In one or more embodiments, the host may include an alkali earth metal complex, a post-transition metal complex, or any combination thereof. For example, the host may include a Be complex (for example, Compound H55), an Mg complex, a Zn complex, or any combination thereof.

In an embodiment, the host may include one of Compounds H1 to H124, 9,10-di(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di-9-carbazolylbenzene (mCP), 1,3,5-tri(carbazol-9-yl)benzene (TCP), or any combination thereof:

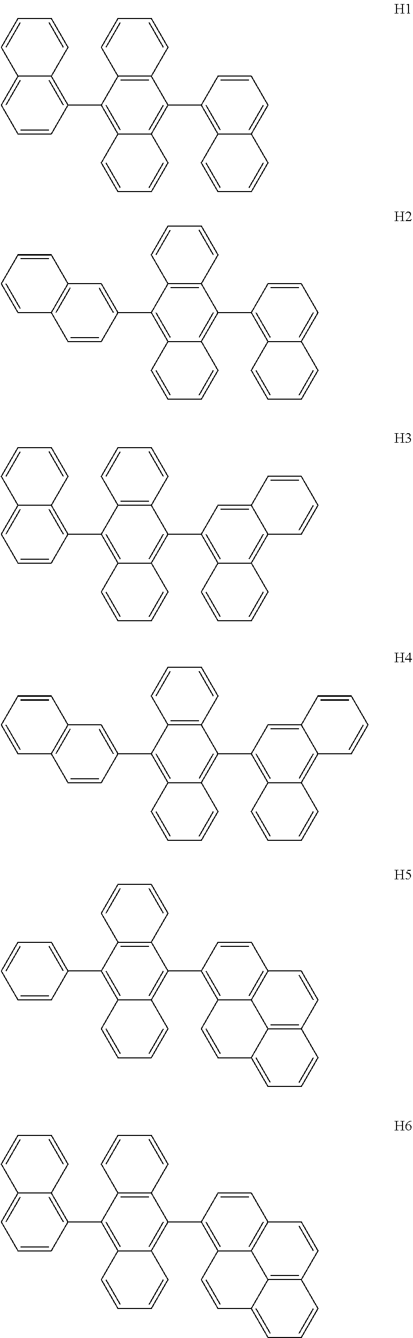

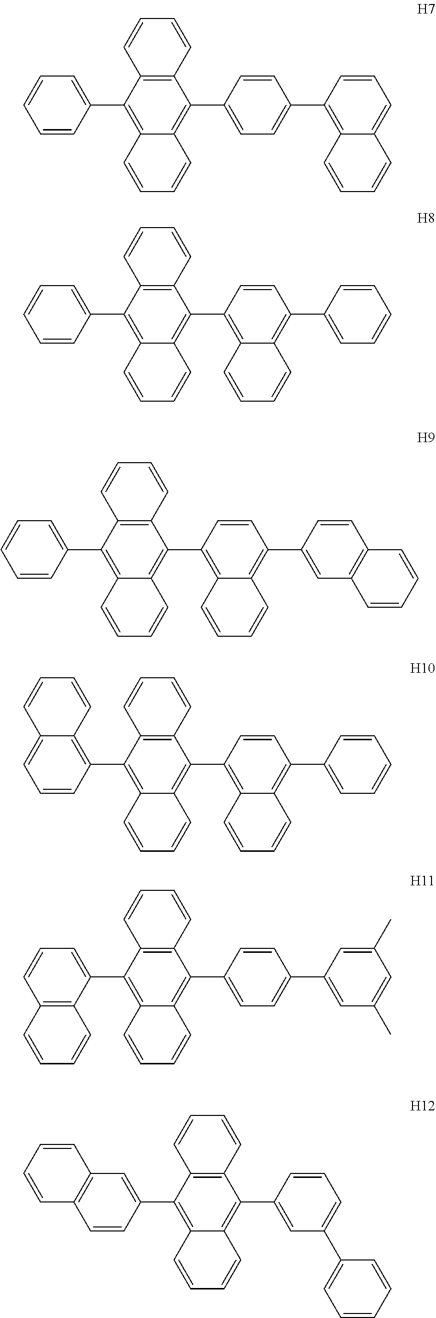

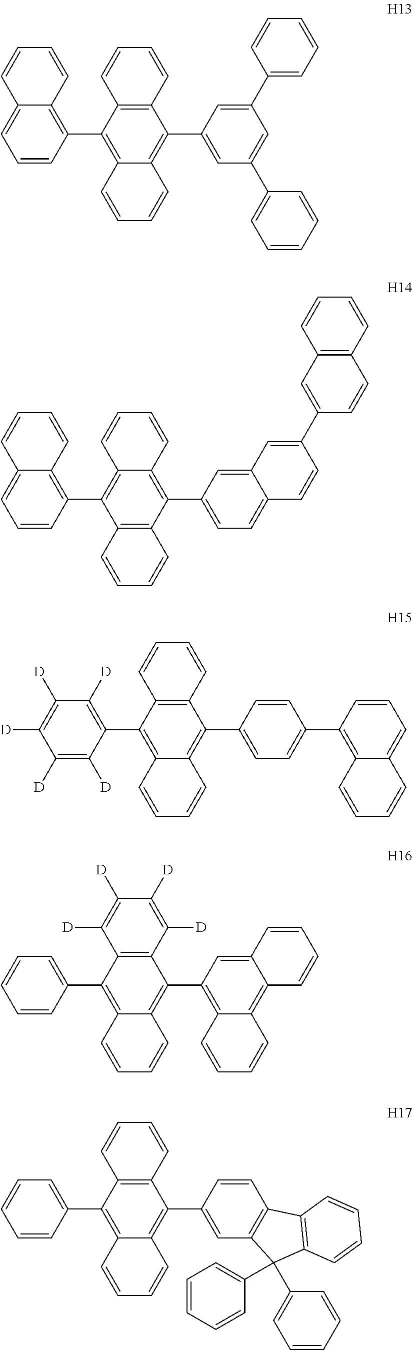

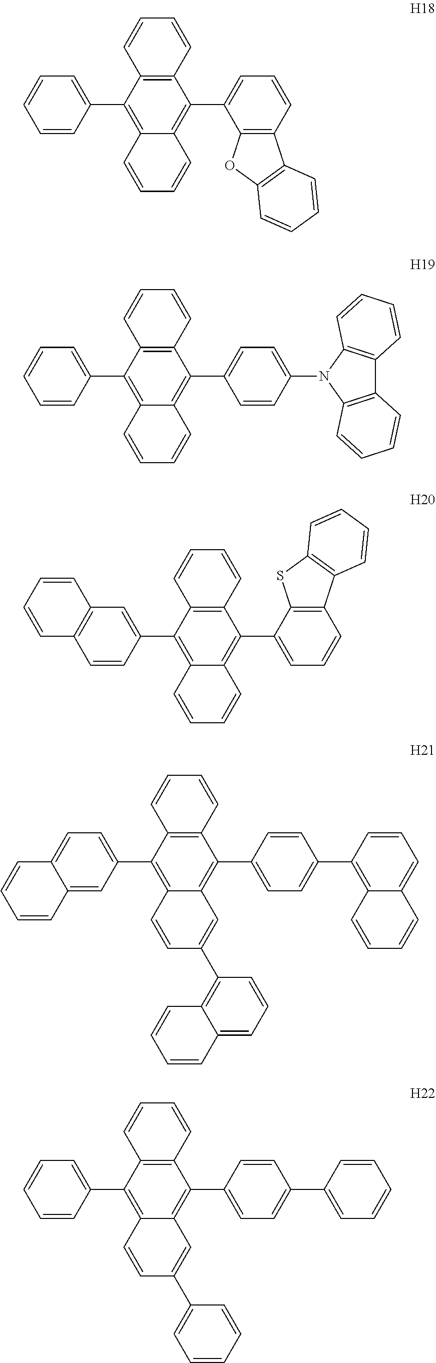

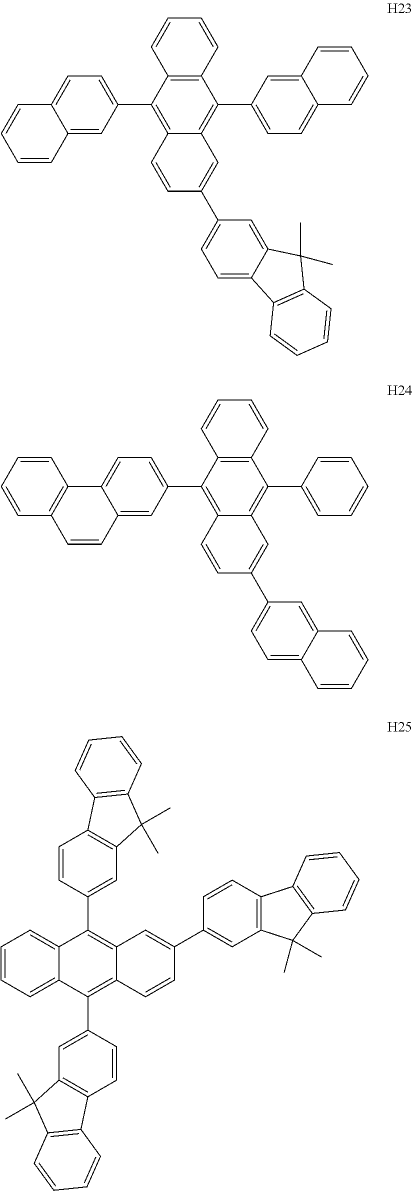

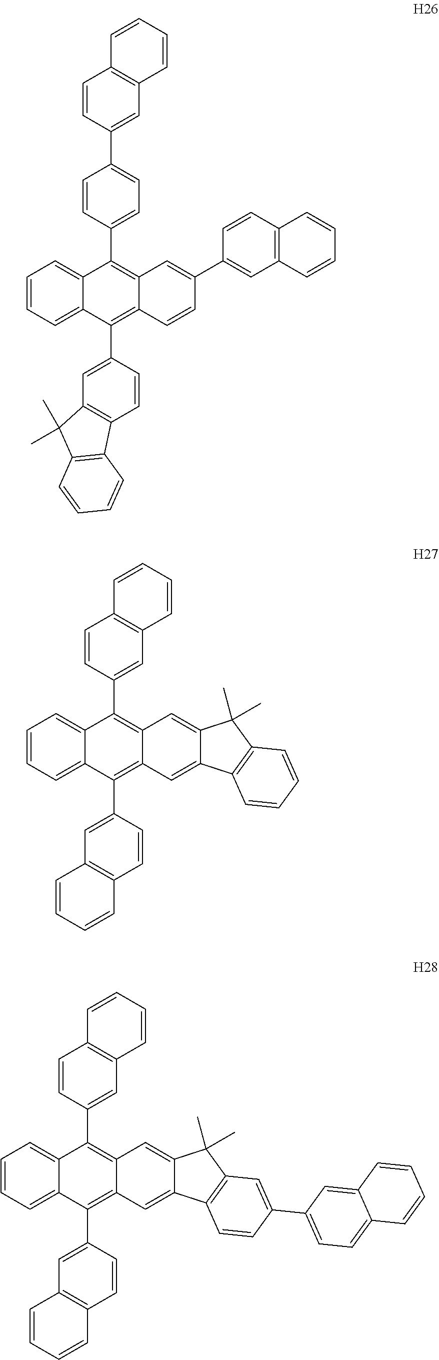

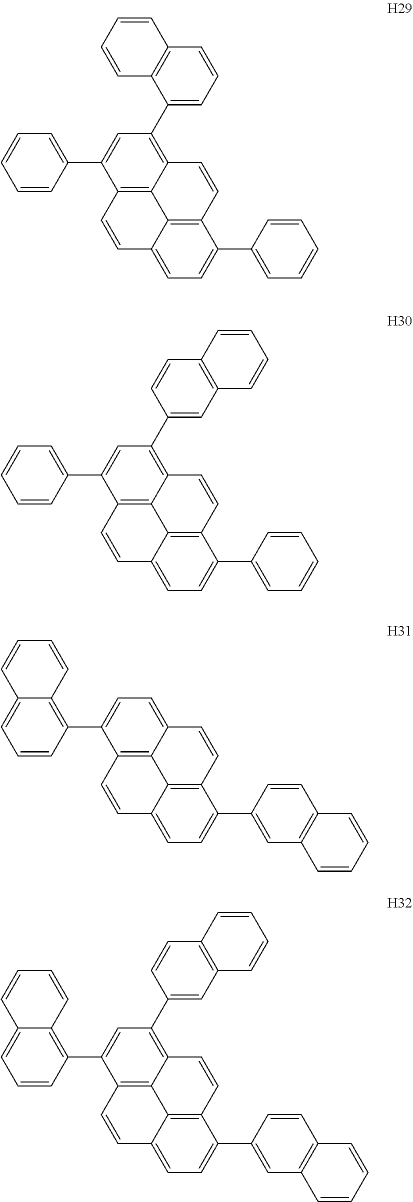

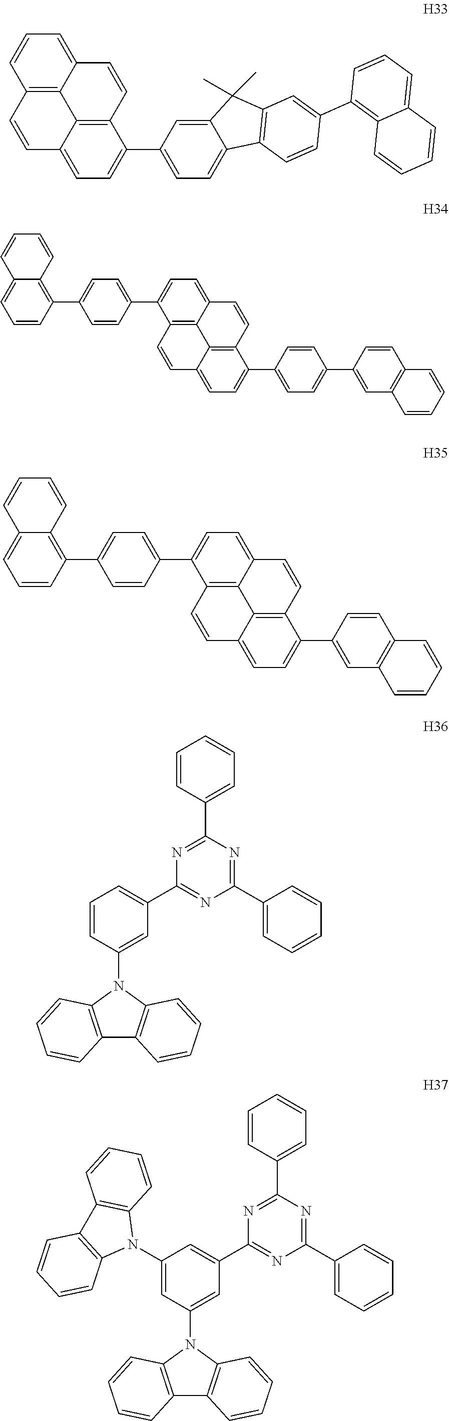

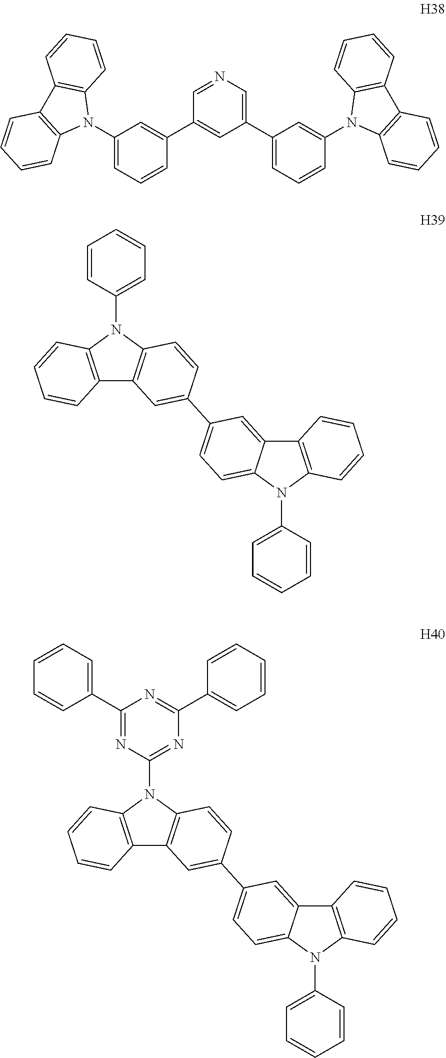 [SMILES: c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1]

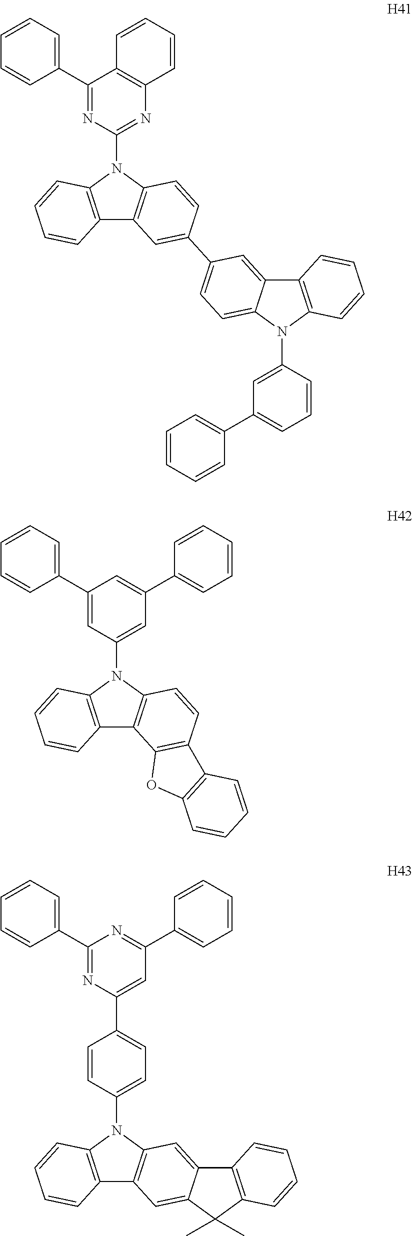

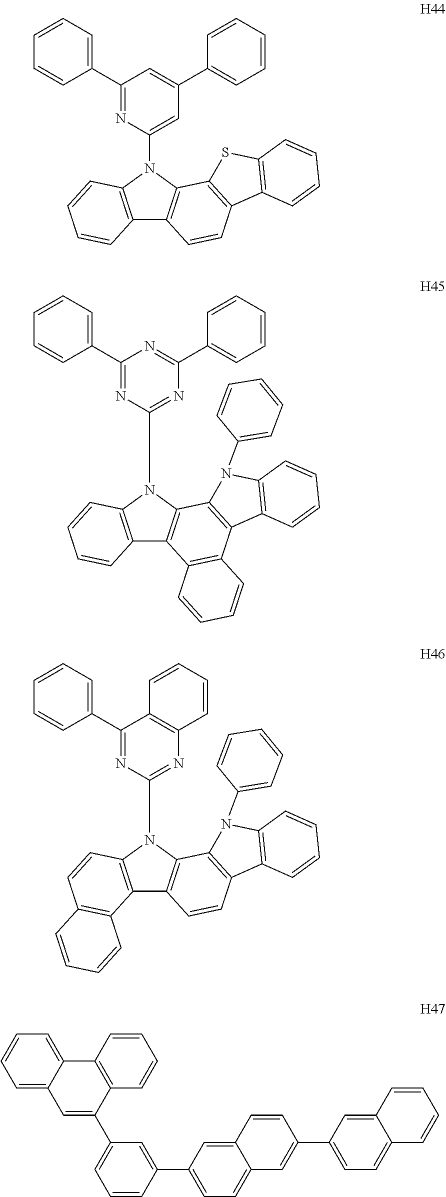

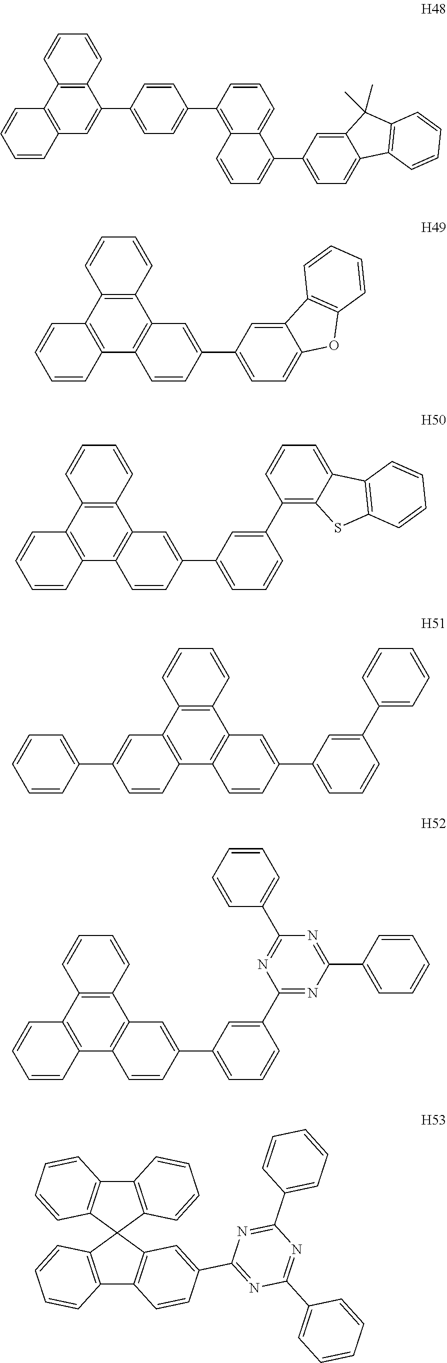

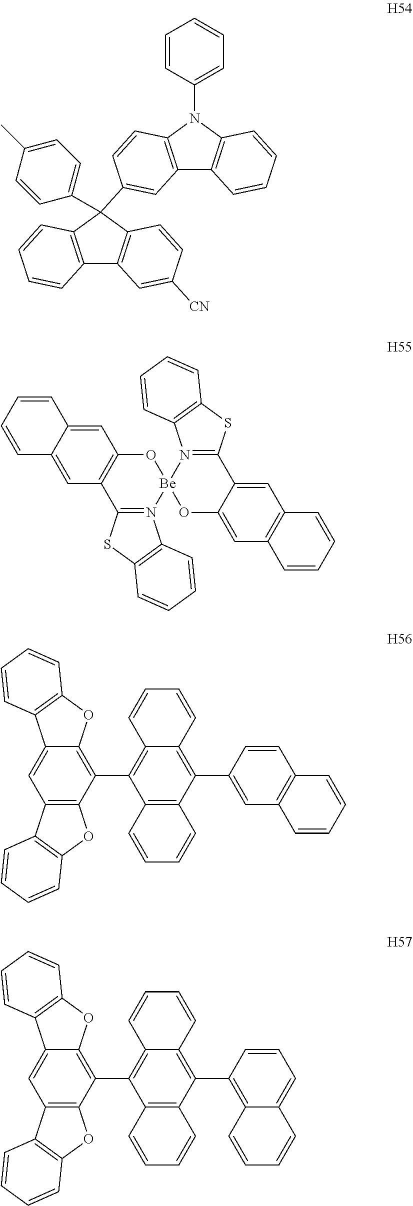

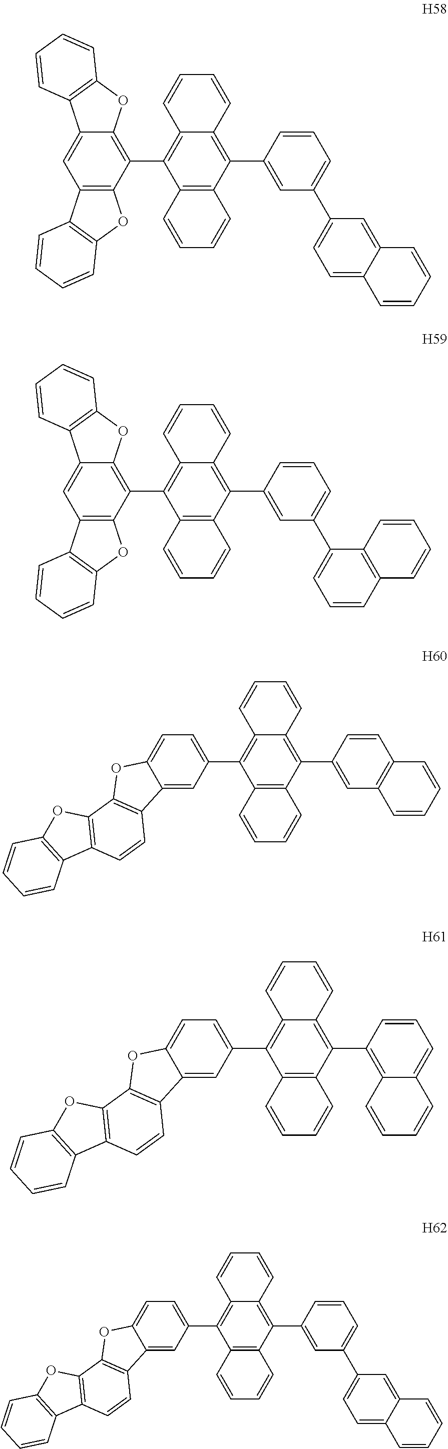 [SMILES: c1cc(-c2ccc3ccccc3c2)cc(-c2c3ccccc3c(-c3c4oc5ccccc5c4cc4c3oc3ccccc34)c3ccccc23)c1 ; c1cc(-c2cccc3ccccc23)cc(-c2c3ccccc3c(-c3c4oc5ccccc5c4cc4c3oc3ccccc34)c3ccccc23)c1 ; c1ccc2cc(-c3c4ccccc4c(-c4ccc5oc6c(ccc7c8ccccc8oc76)c5c4)c4ccccc34)ccc2c1 ; c1ccc2c(-c3c4ccccc4c(-c4ccc5oc6c(ccc7c8ccccc8oc76)c5c4)c4ccccc34)cccc2c1 ; c1cc(-c2ccc3ccccc3c2)cc(-c2c3ccccc3c(-c3ccc4oc5c(ccc6c7ccccc7oc65)c4c3)c3ccccc23)c1]

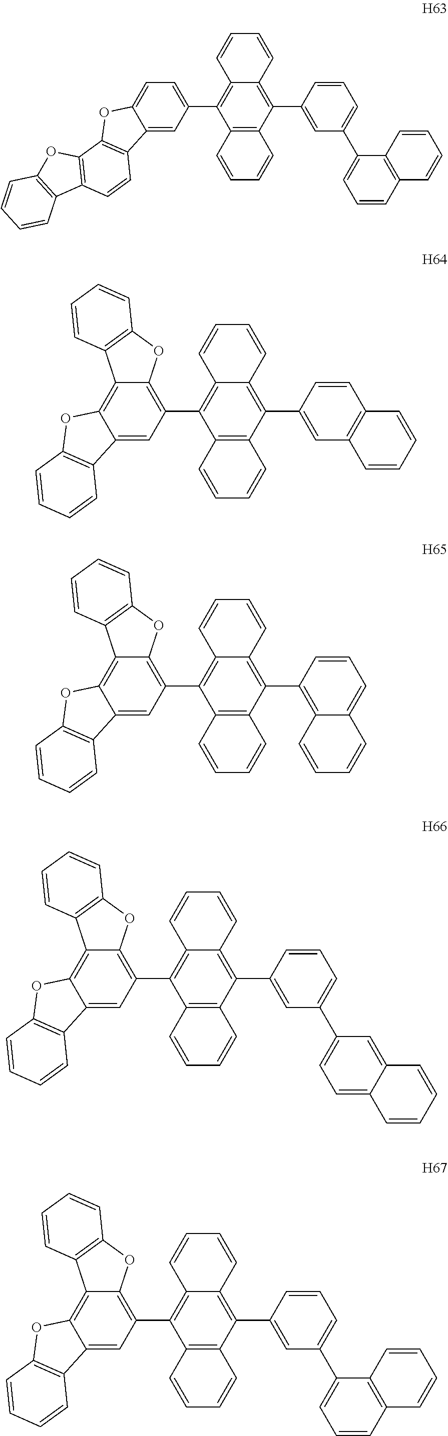 [SMILES: c1cc(-c2cccc3ccccc23)cc(-c2c3ccccc3c(-c3ccc4oc5c(ccc6c7ccccc7oc65)c4c3)c3ccccc23)c1 ; c1ccc2cc(-c3c4ccccc4c(-c4cc5c6ccccc6oc5c5c4oc4ccccc45)c4ccccc34)ccc2c1 ; c1ccc2c(-c3c4ccccc4c(-c4cc5c6ccccc6oc5c5c4oc4ccccc45)c4ccccc34)cccc2c1 ; c1cc(-c2ccc3ccccc3c2)cc(-c2c3ccccc3c(-c3cc4c5ccccc5oc4c4c3oc3ccccc34)c3ccccc23)c1 ; c1cc(-c2cccc3ccccc23)cc(-c2c3ccccc3c(-c3cc4c5ccccc5oc4c4c3oc3ccccc34)c3ccccc23)c1]

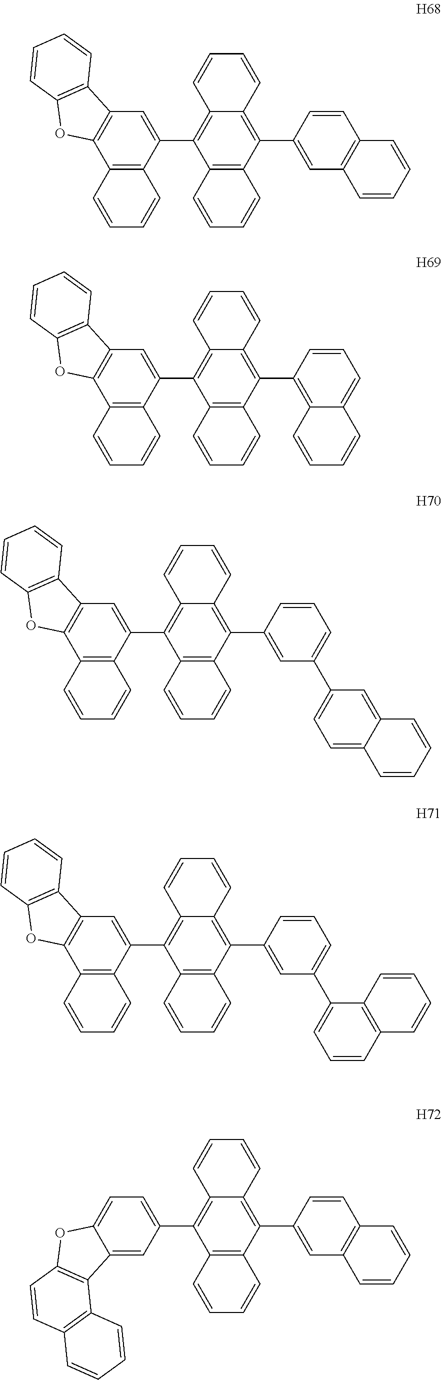 [SMILES: c1ccc2cc(-c3c4ccccc4c(-c4cc5c6ccccc6oc5c5ccccc45)c4ccccc34)ccc2c1 ; c1ccc2c(-c3c4ccccc4c(-c4cc5c6ccccc6oc5c5ccccc45)c4ccccc34)cccc2c1 ; c1cc(-c2ccc3ccccc3c2)cc(-c2c3ccccc3c(-c3cc4c5ccccc5oc4c4ccccc34)c3ccccc23)c1 ; c1cc(-c2cccc3ccccc23)cc(-c2c3ccccc3c(-c3cc4c5ccccc5oc4c4ccccc34)c3ccccc23)c1 ; c1ccc2cc(-c3c4ccccc4c(-c4ccc5oc6ccc7ccccc7c6c5c4)c4ccccc34)ccc2c1]

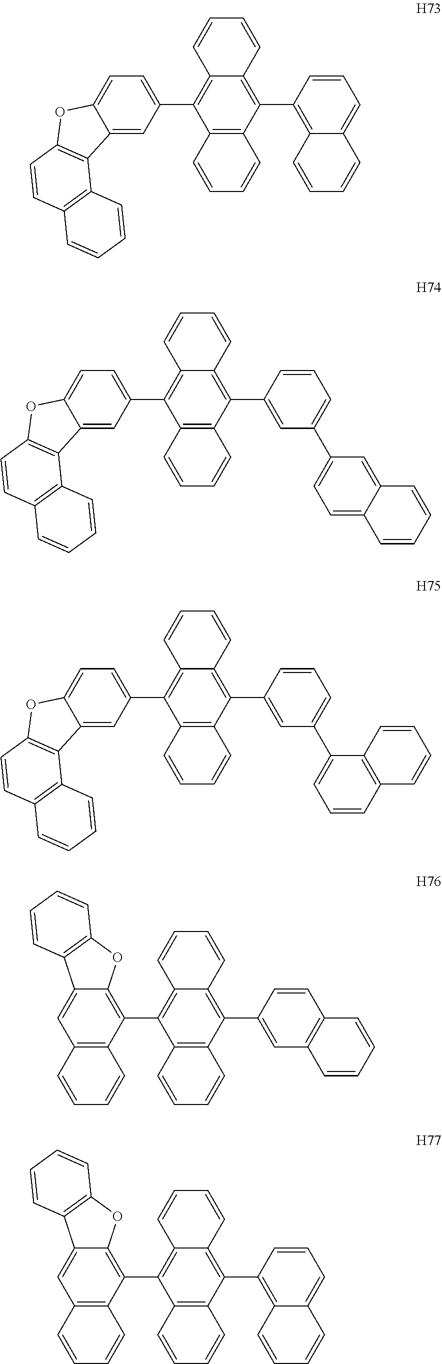

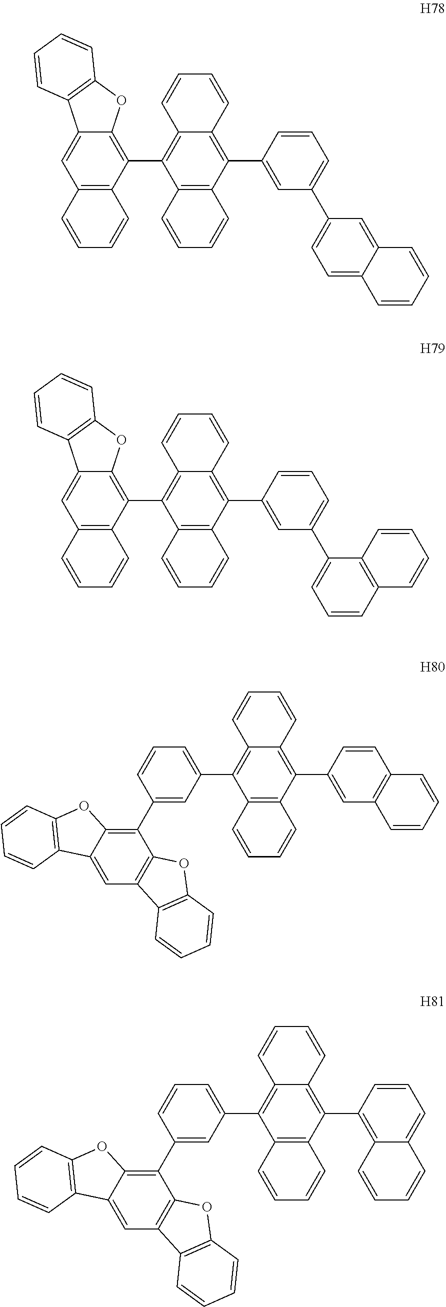 [SMILES: c1cc(-c2ccc3ccccc3c2)cc(-c2c3ccccc3c(-c3c4ccccc4cc4c3oc3ccccc34)c3ccccc23)c1 ; c1cc(-c2cccc3ccccc23)cc(-c2c3ccccc3c(-c3c4ccccc4cc4c3oc3ccccc34)c3ccccc23)c1 ; c1cc(-c2c3ccccc3c(-c3ccc4ccccc4c3)c3ccccc23)cc(-c2c3oc4ccccc4c3cc3c2oc2ccccc23)c1 ; c1cc(-c2c3ccccc3c(-c3cccc4ccccc34)c3ccccc23)cc(-c2c3oc4ccccc4c3cc3c2oc2ccccc23)c1]

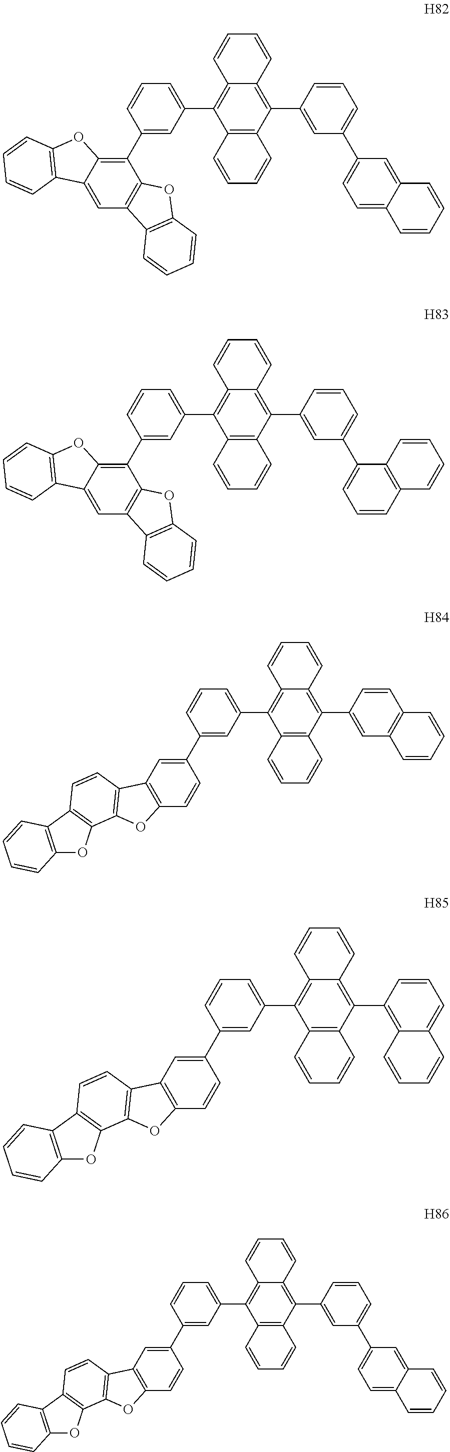 [SMILES: c1cc(-c2ccc3ccccc3c2)cc(-c2c3ccccc3c(-c3cccc(-c4c5oc6ccccc6c5cc5c4oc4ccccc45)c3)c3ccccc23)c1 ; c1cc(-c2cccc3ccccc23)cc(-c2c3ccccc3c(-c3cccc(-c4c5oc6ccccc6c5cc5c4oc4ccccc45)c3)c3ccccc23)c1 ; c1cc(-c2ccc3oc4c(ccc5c6ccccc6oc54)c3c2)cc(-c2c3ccccc3c(-c3ccc4ccccc4c3)c3ccccc23)c1 ; c1cc(-c2ccc3oc4c(ccc5c6ccccc6oc54)c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6c(ccc7c8ccccc8oc76)c5c4)c3)c3ccccc23)c1]

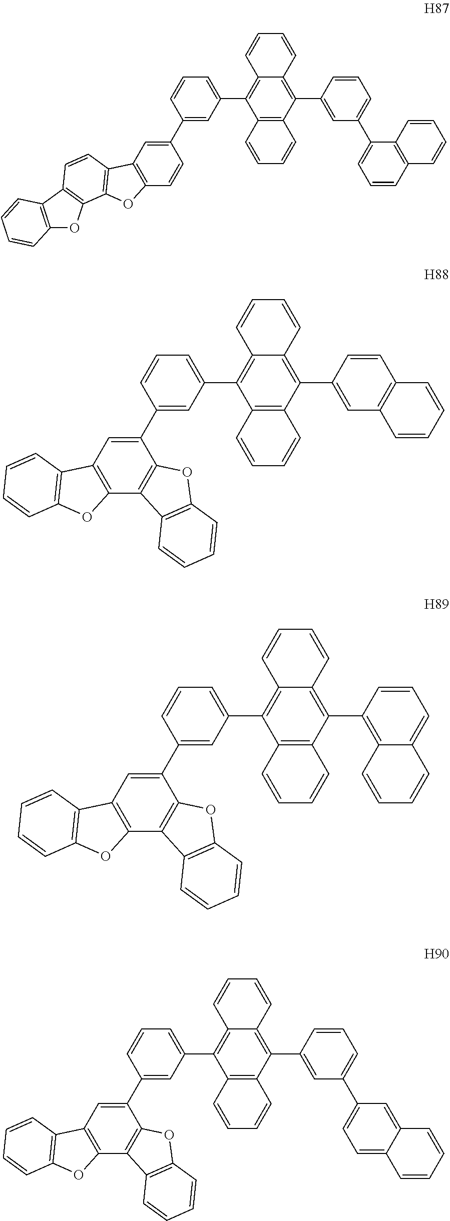 [SMILES: c1cc(-c2ccc3oc4c(ccc5c6ccccc6oc54)c3c2)cc(-c2c3ccccc3c(-c3cccc(-c4cccc5ccccc45)c3)c3ccccc23)c1 ; c1cc(-c2c3ccccc3c(-c3ccc4ccccc4c3)c3ccccc23)cc(-c2cc3c4ccccc4oc3c3c2oc2ccccc23)c1 ; c1cc(-c2c3ccccc3c(-c3cccc4ccccc34)c3ccccc23)cc(-c2cc3c4ccccc4oc3c3c2oc2ccccc23)c1 ; c1cc(-c2ccc3ccccc3c2)cc(-c2c3ccccc3c(-c3cccc(-c4cc5c6ccccc6oc5c5c4oc4ccccc45)c3)c3ccccc23)c1]

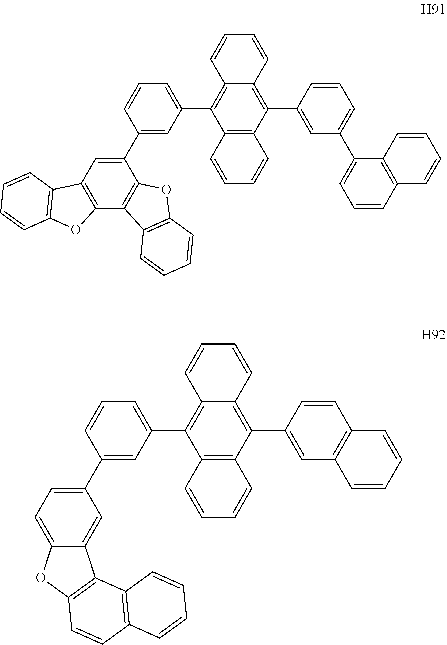 [SMILES: c1cc(-c2cccc3ccccc23)cc(-c2c3ccccc3c(-c3cccc(-c4cc5c6ccccc6oc5c5c4oc4ccccc45)c3)c3ccccc23)c1 ; c1cc(-c2ccc3oc4ccc5ccccc5c4c3c2)cc(-c2c3ccccc3c(-c3ccc4ccccc4c3)c3ccccc23)c1]

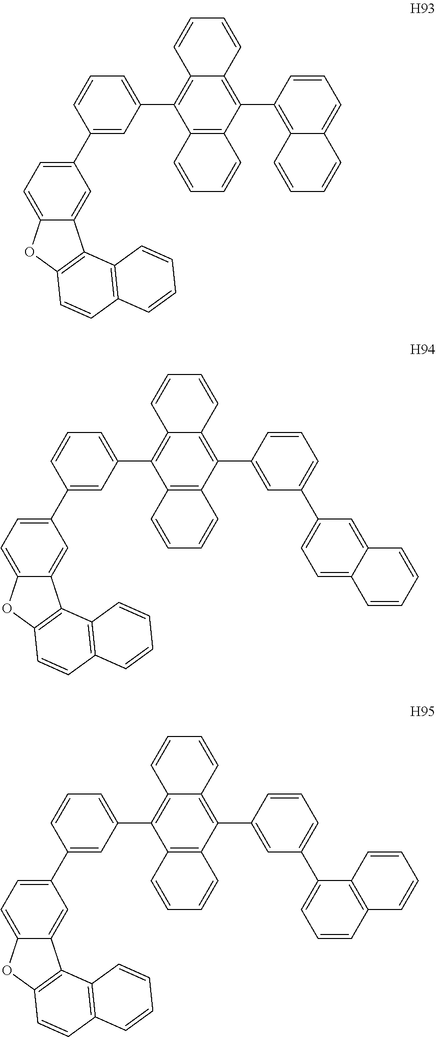 [SMILES: c1cc(-c2ccc3oc4ccc5ccccc5c4c3c2)cc(-c2c3ccccc3c(-c3cccc4ccccc34)c3ccccc23)c1 ; c1cc(-c2ccc3ccccc3c2)cc(-c2c3ccccc3c(-c3cccc(-c4ccc5oc6ccc7ccccc7c6c5c4)c3)c3ccccc23)c1 ; c1cc(-c2ccc3oc4ccc5ccccc5c4c3c2)cc(-c2c3ccccc3c(-c3cccc(-c4cccc5ccccc45)c3)c3ccccc23)c1]

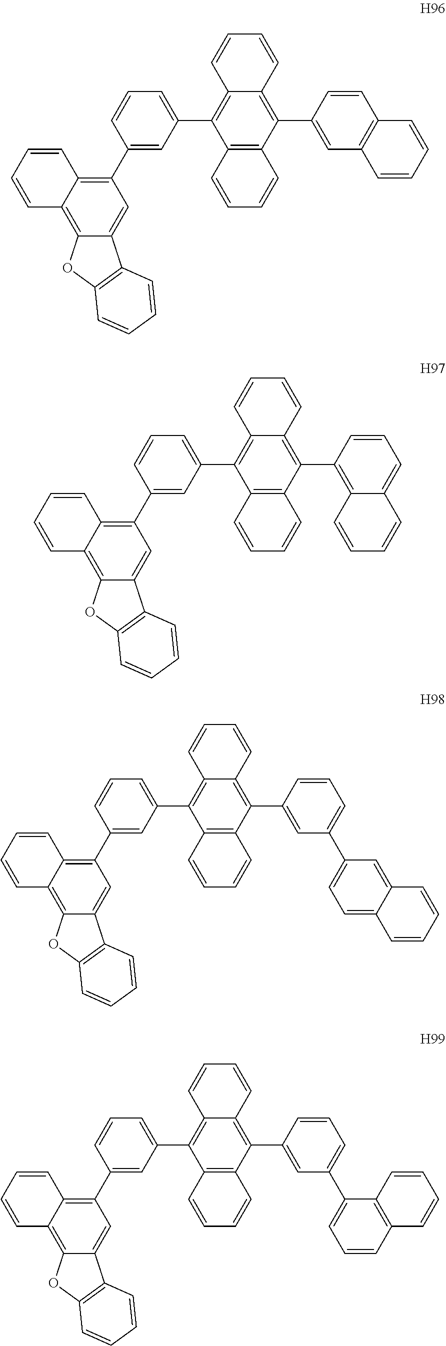 [SMILES: c1cc(-c2c3ccccc3c(-c3ccc4ccccc4c3)c3ccccc23)cc(-c2cc3c4ccccc4oc3c3ccccc23)c1 ; c1cc(-c2c3ccccc3c(-c3cccc4ccccc34)c3ccccc23)cc(-c2cc3c4ccccc4oc3c3ccccc23)c1 ; c1cc(-c2ccc3ccccc3c2)cc(-c2c3ccccc3c(-c3cccc(-c4cc5c6ccccc6oc5c5ccccc45)c3)c3ccccc23)c1 ; c1cc(-c2cccc3ccccc23)cc(-c2c3ccccc3c(-c3cccc(-c4cc5c6ccccc6oc5c5ccccc45)c3)c3ccccc23)c1]

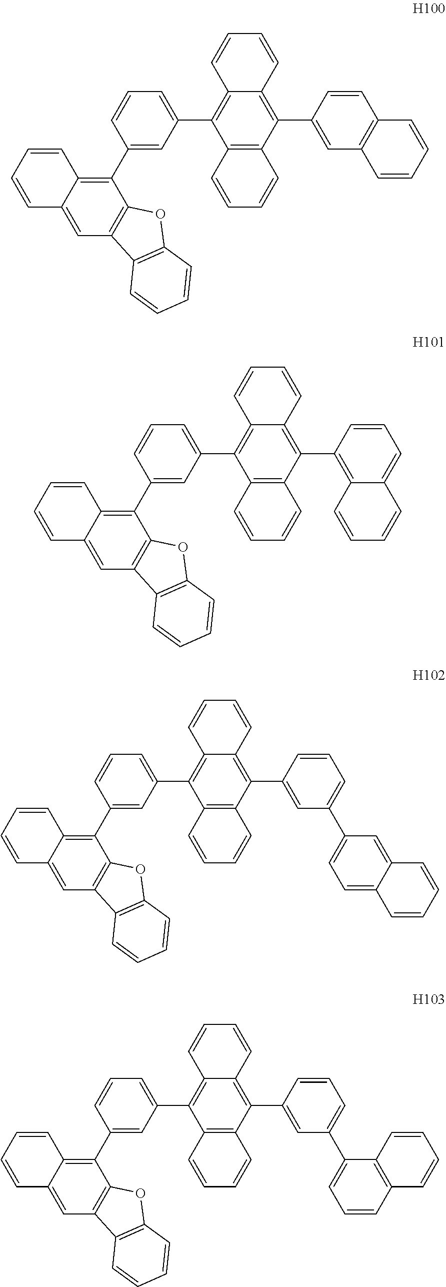 [SMILES: c1cc(-c2c3ccccc3c(-c3ccc4ccccc4c3)c3ccccc23)cc(-c2c3ccccc3cc3c2oc2ccccc23)c1 ; c1cc(-c2c3ccccc3c(-c3cccc4ccccc34)c3ccccc23)cc(-c2c3ccccc3cc3c2oc2ccccc23)c1 ; c1cc(-c2ccc3ccccc3c2)cc(-c2c3ccccc3c(-c3cccc(-c4c5ccccc5cc5c4oc4ccccc45)c3)c3ccccc23)c1 ; c1cc(-c2cccc3ccccc23)cc(-c2c3ccccc3c(-c3cccc(-c4c5ccccc5cc5c4oc4ccccc45)c3)c3ccccc23)c1]

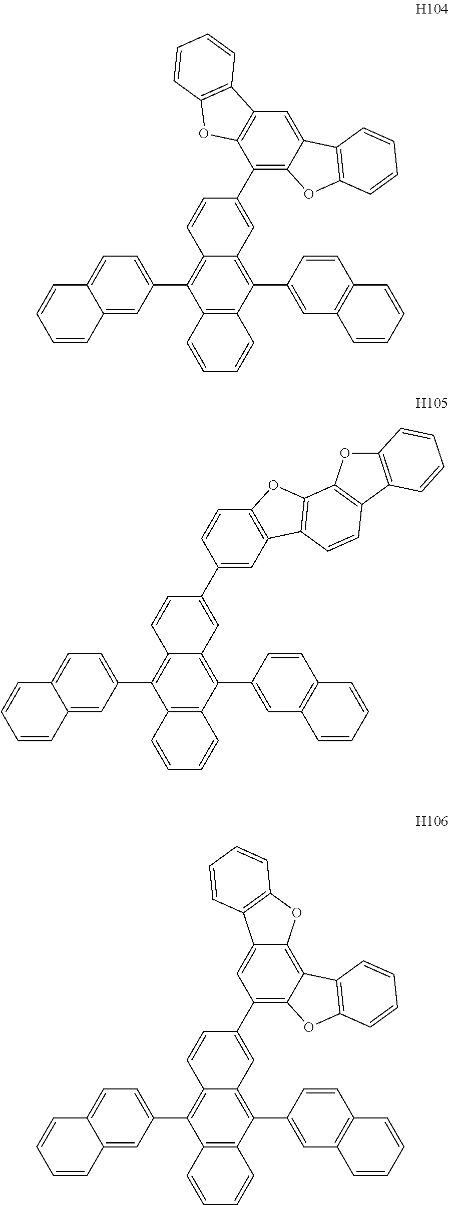 [SMILES: c1ccc2cc(-c3c4ccccc4c(-c4ccc5ccccc5c4)c4cc(-c5c6oc7ccccc7c6cc6c5oc5ccccc56)ccc34)ccc2c1 ; c1ccc2cc(-c3c4ccccc4c(-c4ccc5ccccc5c4)c4cc(-c5ccc6oc7c(ccc8c9ccccc9oc87)c6c5)ccc34)ccc2c1 ; c1ccc2cc(-c3c4ccccc4c(-c4ccc5ccccc5c4)c4cc(-c5cc6c7ccccc7oc6c6c5oc5ccccc56)ccc34)ccc2c1]

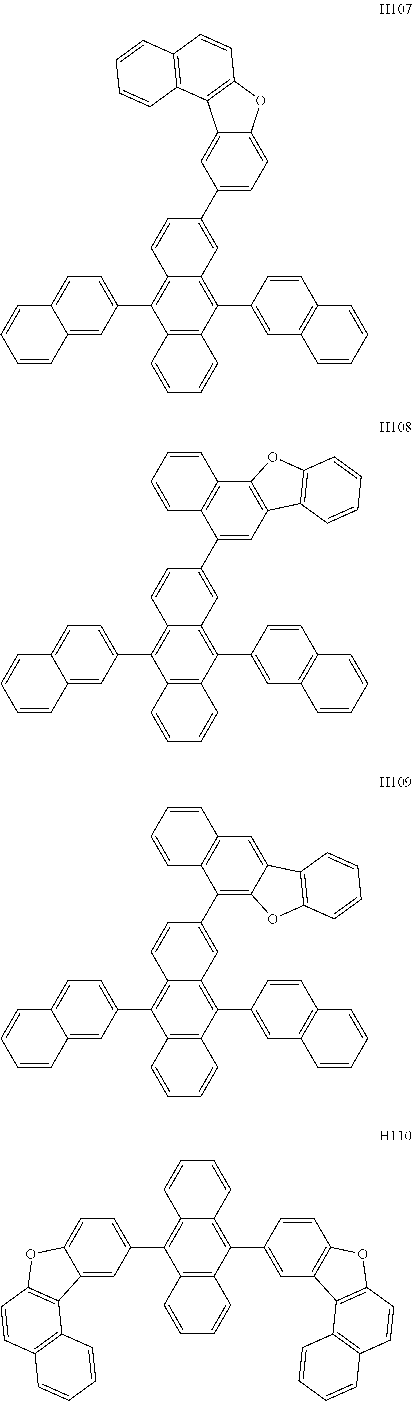 [SMILES: c1ccc2cc(-c3c4ccccc4c(-c4ccc5ccccc5c4)c4cc(-c5ccc6oc7ccc8ccccc8c7c6c5)ccc34)ccc2c1 ; c1ccc2cc(-c3c4ccccc4c(-c4ccc5ccccc5c4)c4cc(-c5cc6c7ccccc7oc6c6ccccc56)ccc34)ccc2c1 ; c1ccc2cc(-c3c4ccccc4c(-c4ccc5ccccc5c4)c4cc(-c5c6ccccc6cc6c5oc5ccccc56)ccc34)ccc2c1 ; c1ccc2c(c1)ccc1oc3ccc(-c4c5ccccc5c(-c5ccc6oc7ccc8ccccc8c7c6c5)c5ccccc45)cc3c12]

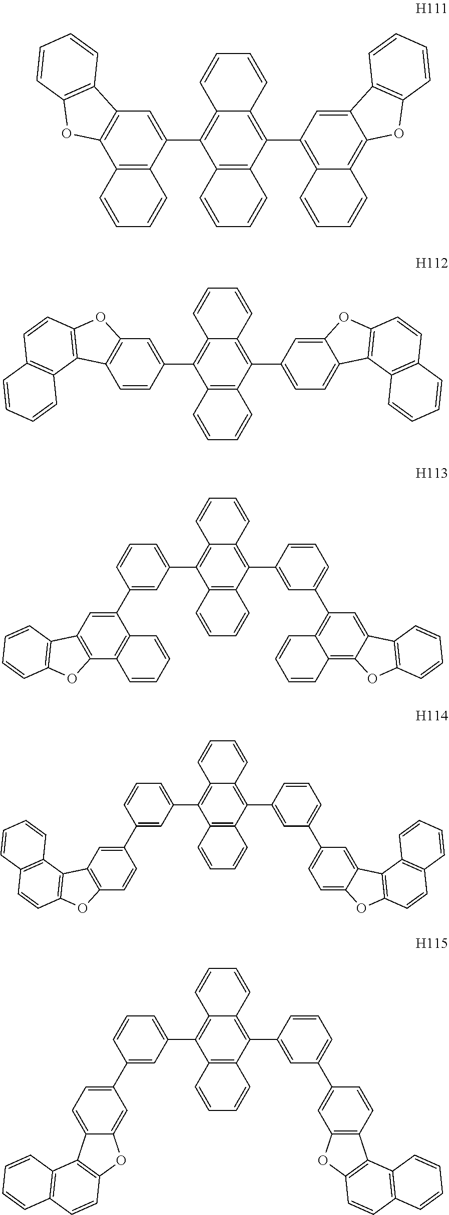 [SMILES: c1ccc2c(c1)oc1c3ccccc3c(-c3c4ccccc4c(-c4cc5c6ccccc6oc5c5ccccc45)c4ccccc34)cc21 ; c1ccc2c(c1)ccc1oc3cc(-c4c5ccccc5c(-c5ccc6c(c5)oc5ccc7ccccc7c56)c5ccccc45)ccc3c12 ; c1cc(-c2c3ccccc3c(-c3cccc(-c4cc5c6ccccc6oc5c5ccccc45)c3)c3ccccc23)cc(-c2cc3c4ccccc4oc3c3ccccc23)c1 ; c1cc(-c2ccc3oc4ccc5ccccc5c4c3c2)cc(-c2c3ccccc3c(-c3cccc(-c4ccc5oc6ccc7ccccc7c6c5c4)c3)c3ccccc23)c1 ; c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1]

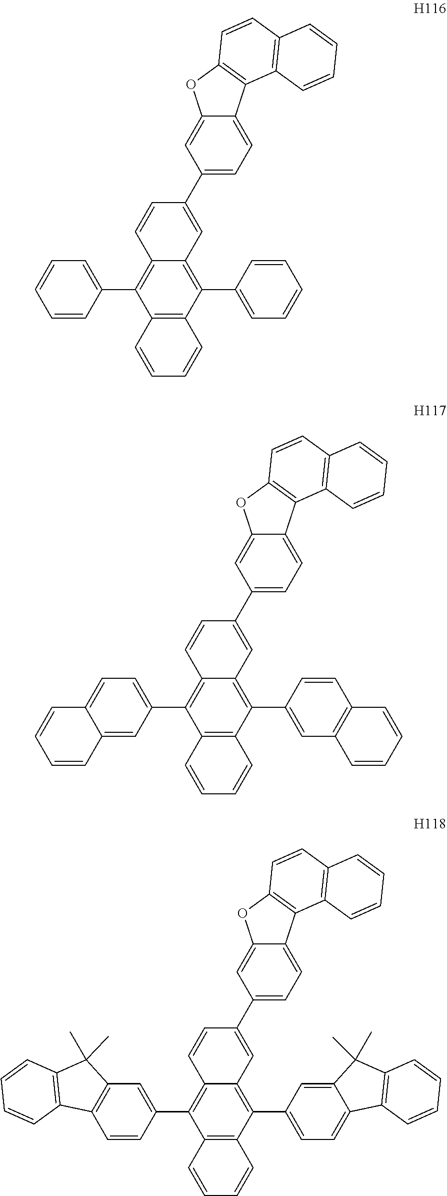 [SMILES: c1ccc(-c2c3ccccc3c(-c3ccccc3)c3cc(-c4ccc5c(c4)oc4ccc6ccccc6c45)ccc23)cc1 ; c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21]

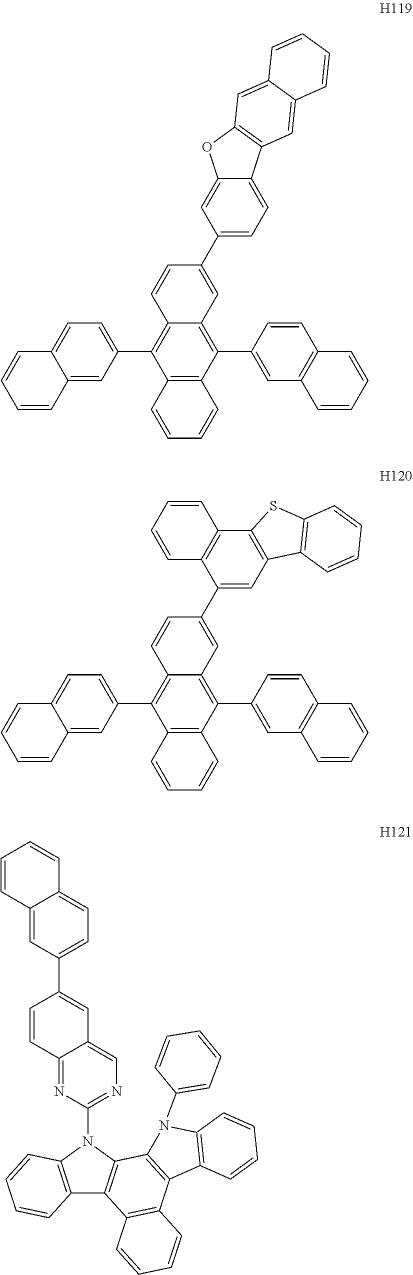 [SMILES: c1ccc2cc(-c3c4ccccc4c(-c4ccc5ccccc5c4)c4cc(-c5ccc6c(c5)oc5cc7ccccc7cc56)ccc34)ccc2c1 ; c1ccc2cc(-c3c4ccccc4c(-c4ccc5ccccc5c4)c4cc(-c5cc6c7ccccc7sc6c6ccccc56)ccc34)ccc2c1 ; c1ccc(-n2c3ccccc3c3c4ccccc4c4c5ccccc5n(-c5ncc6cc(-c7ccc8ccccc8c7)ccc6n5)c4c32)cc1]

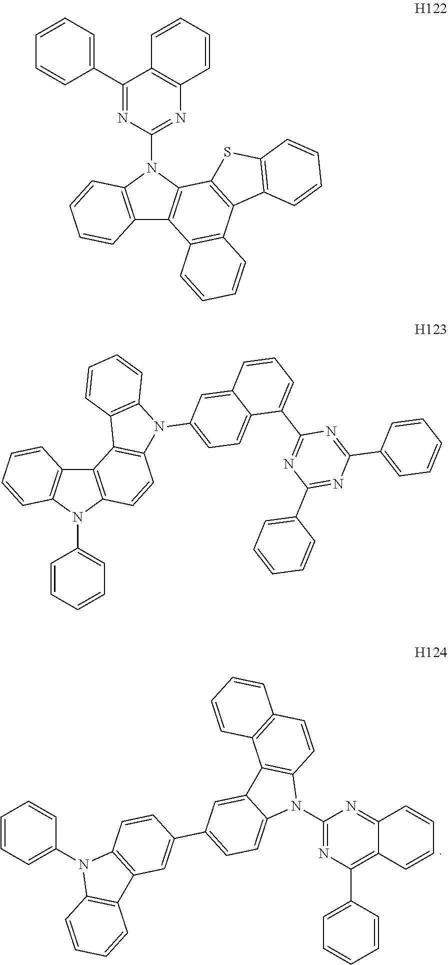 [SMILES: c1ccc(-c2nc(-n3c4ccccc4c4c5ccccc5c5c6ccccc6sc5c43)nc3ccccc23)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

### Phosphorescent Dopant

In one or more embodiments, the phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

For example, the phosphorescent dopant may include an organometallic compound represented by Formula 401:

M(L₄₀₁)_{xc1}(L₄₀₂)_{xc2} Formula 401

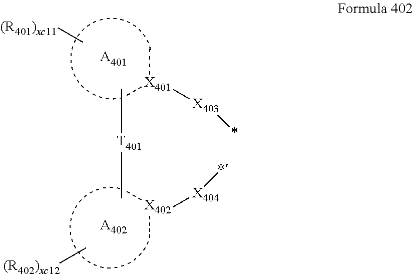

wherein, in Formulae 401 and 402,

M may be a transition metal (for example, iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),

L₄₀₁ may be a ligand represented by Formula 402, and xc1 may be 1, 2, or 3, wherein when xc1 is two or more, two or more of L₄₀₁(s) may be identical to or different from each other,

L₄₀₂ may be an organic ligand, and xc2 may be 0, 1, 2, 3, or 4, and when xc2 is 2 or more, two or more of L₄₀₂(s) may be identical to or different from each other,

X₄₀₁ and X₄₀₂ may each independently be nitrogen or carbon,

ring A₄₀₁ and ring A₄₀₂ may each independently be a C₃-C₆₀ carbocyclic group or a C₁-C₆₀ heterocyclic group,

T₄₀₁ may be a single bond, *—O—*′, *—S—*′, *—C(═O)—*′, *—N(Q₄₁₁)-*′, *—C(Q₄₁₁)(Q₄₁₂)-*′, *—C(Q₄₁₁)=C(Q₄₁₂)-*′, *—C(Q₄₁₁)=*′, or *═C(Q₄₁₁)=*′,

X₄₀₃ and X₄₀₄ may each independently be a chemical bond (for example, a covalent bond or a coordinate covalent bond), O, S, N(Q₄₁₃), B(Q₄₁₃), P(Q₄₁₃), C(Q₄₁₃)(Q₄₁₄), or Si(Q₄₁₃)(Q₄₁₄),

Q₄₁₁ to Q₄₁₄ may each be the same as described herein with respect to Q₁,

R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),

Q₄₀₁ to Q₄₀₃ may each be the same as described herein with respect to Q₁,

xc11 and xc12 may each independently be an integer from 0 to 10, and

* and *′ in Formula 402 each indicate a binding site to M in Formula 401.

For example, in Formula 402, i) X₄₀₁ may be nitrogen, and X₄₀₂ may be carbon, or ii) each of X₄₀₁ and X₄₀₂ may be nitrogen.

In one or more embodiments, when xc1 in Formula 402 is 2 or more, two ring A₄₀₁(s) in two or more of L₄₀₁(s) may be optionally linked to each other via T₄₀₂, which is a linking group, and two ring A₄₀₂(s) may be optionally linked to each other via T₄₀₃, which is a linking group (see Compounds PD1 to PD4 and PD7). T₄₀₂ and T₄₀₃ may each be the same as described herein with respect to T₄₀₁.

L₄₀₂ in Formula 401 may be an organic ligand. For example, L₄₀₂ may include a halogen group, a diketone group (for example, an acetylacetonate group), a carboxylic acid group (for example, a picolinate group), —C(═O), an isonitrile group, —CN group, a phosphorus group (for example, a phosphine group, a phosphite group, etc.), or any combination thereof.

The phosphorescent dopant may include, for example, one of compounds PD1 to PD39, or any combination thereof:

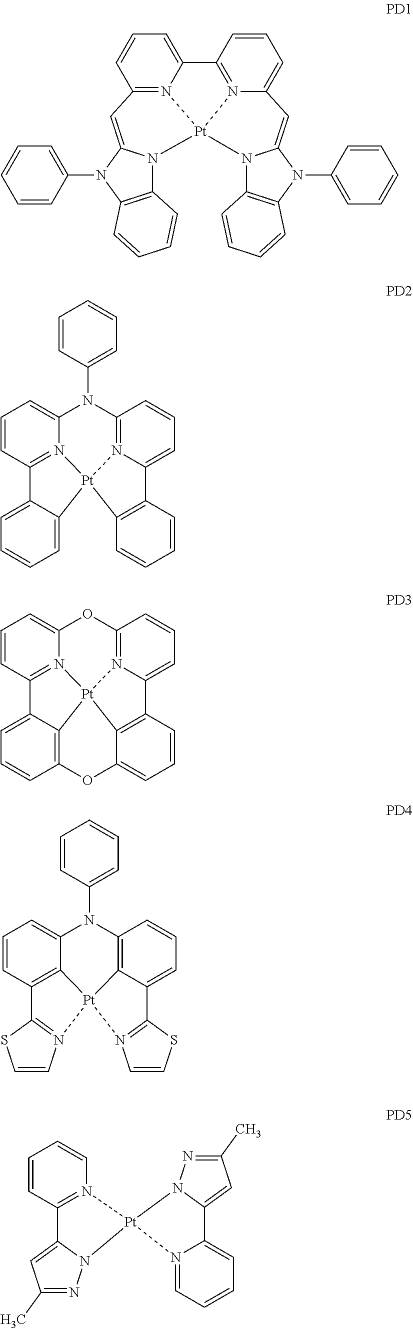

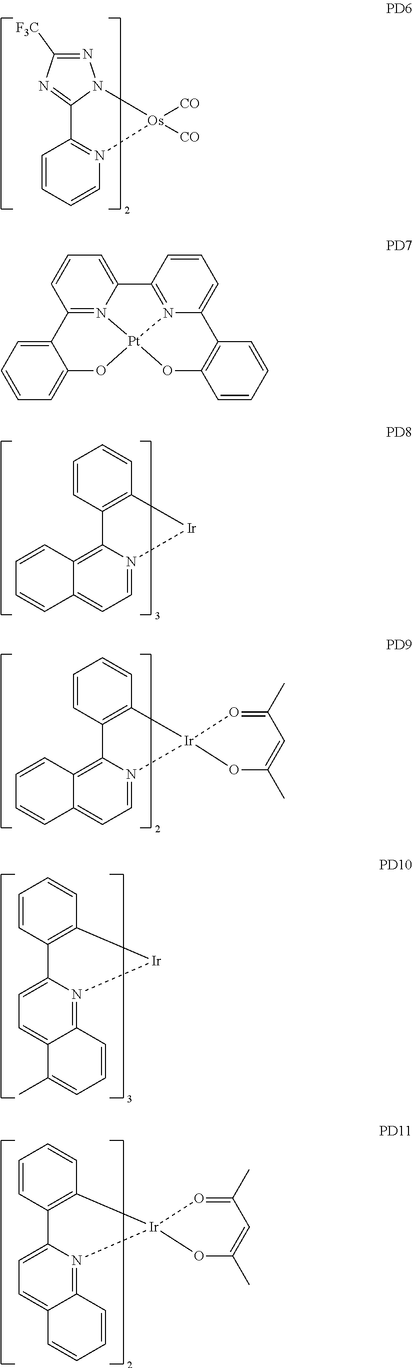

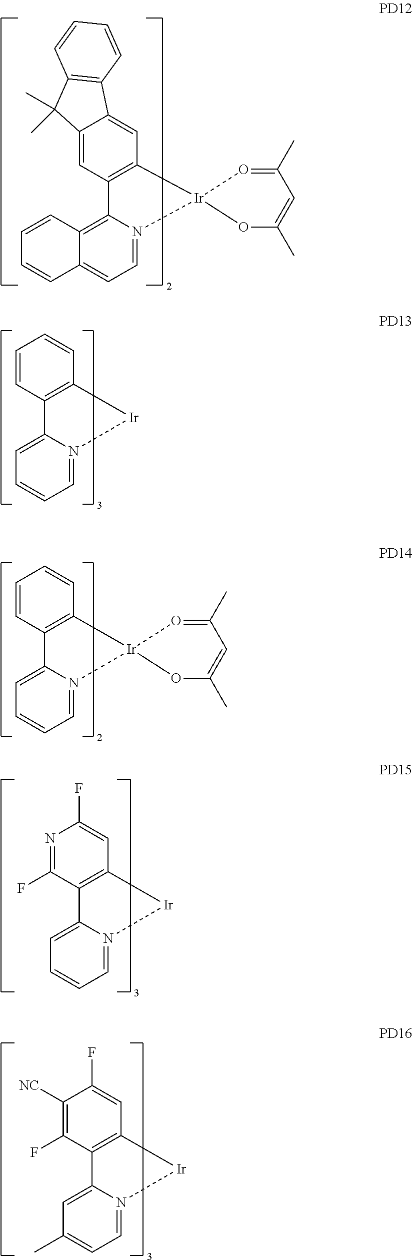

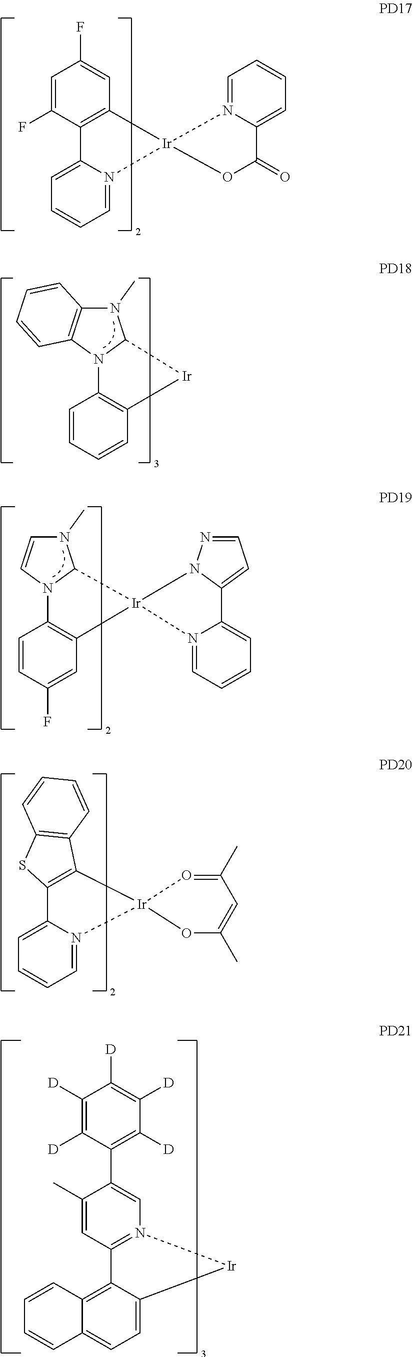

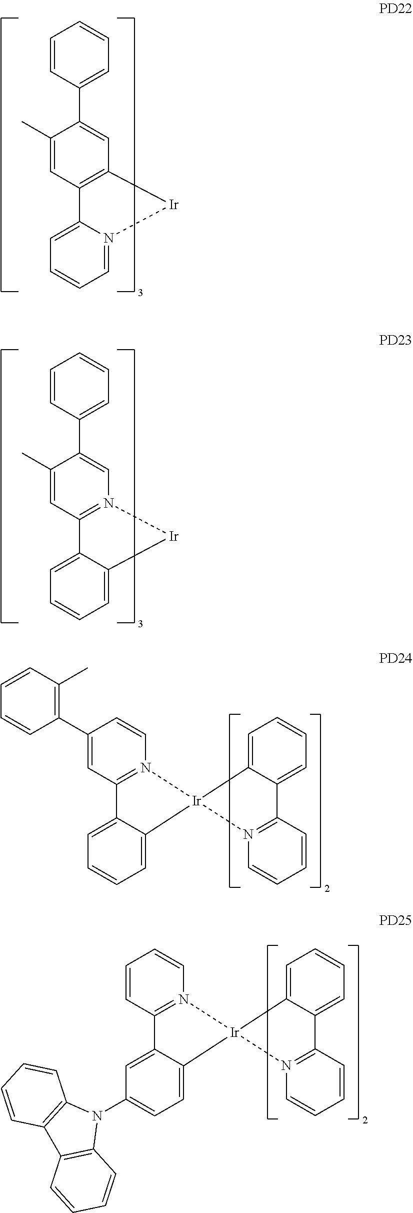

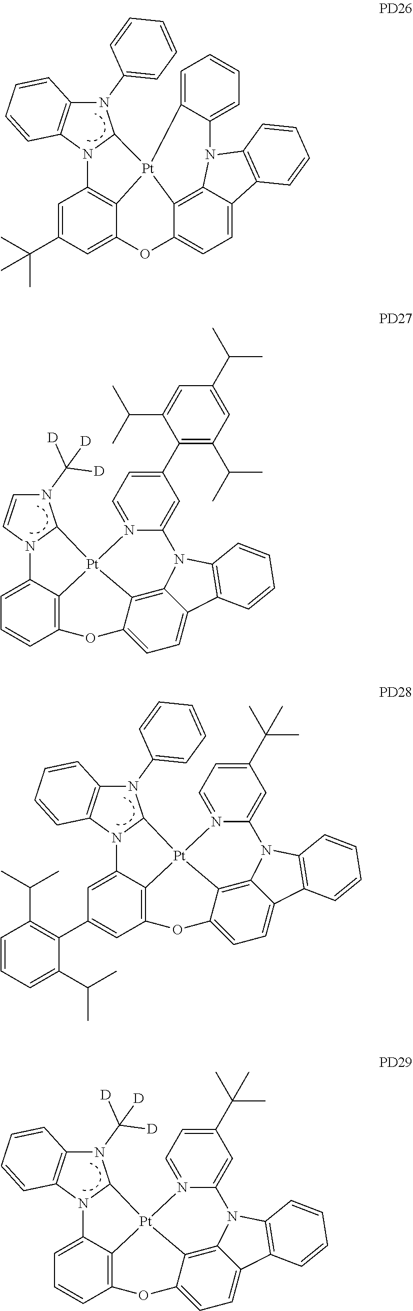

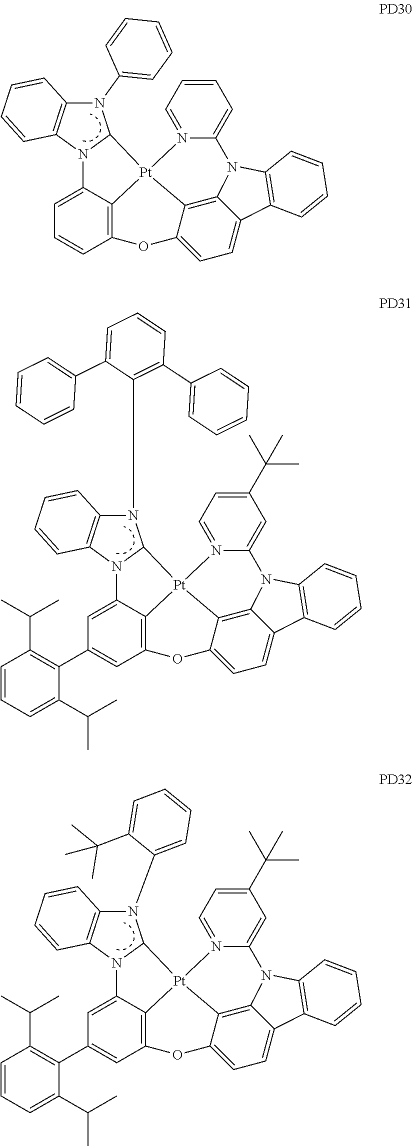

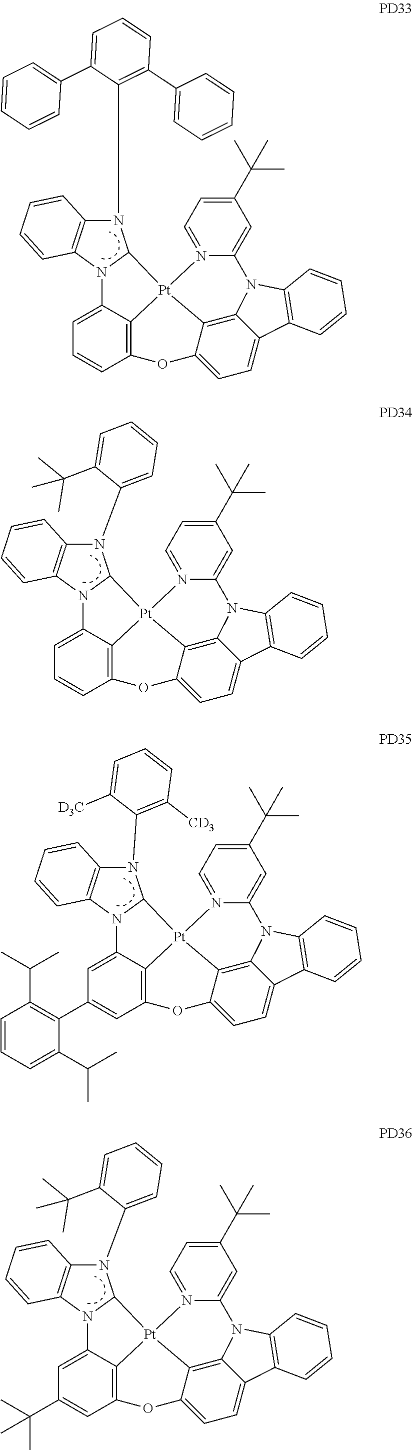

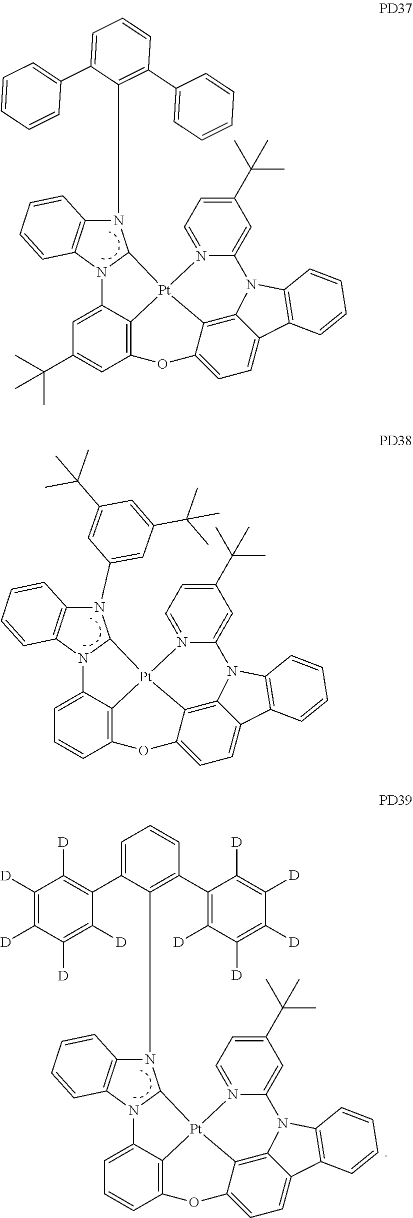

### Fluorescence Dopant

The fluorescence dopant may include an amine group-containing compound, a styryl group-containing compound, or any combination thereof.

For example, the fluorescence dopant may include a compound represented by Formula 501:

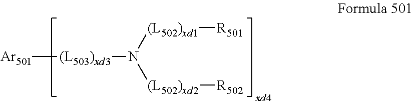

wherein, in Formula 501,

Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xd1 to xd3 may each independently be 0, 1, 2, or 3, and

xd4 may be 1, 2, 3, 4, 5, or 6.

For example, Ar₅₀₁ in Formula 501 may be a condensed cyclic group (for example, an anthracene group, a chrysene group, or a pyrene group) in which three or more monocyclic groups are condensed together.

In one or more embodiments, xd4 in Formula 501 may be 2.

For example, the fluorescence dopant may include: one selected from Compounds FD1 to FD36; DPVBi; DPAVBi; or any combination thereof:

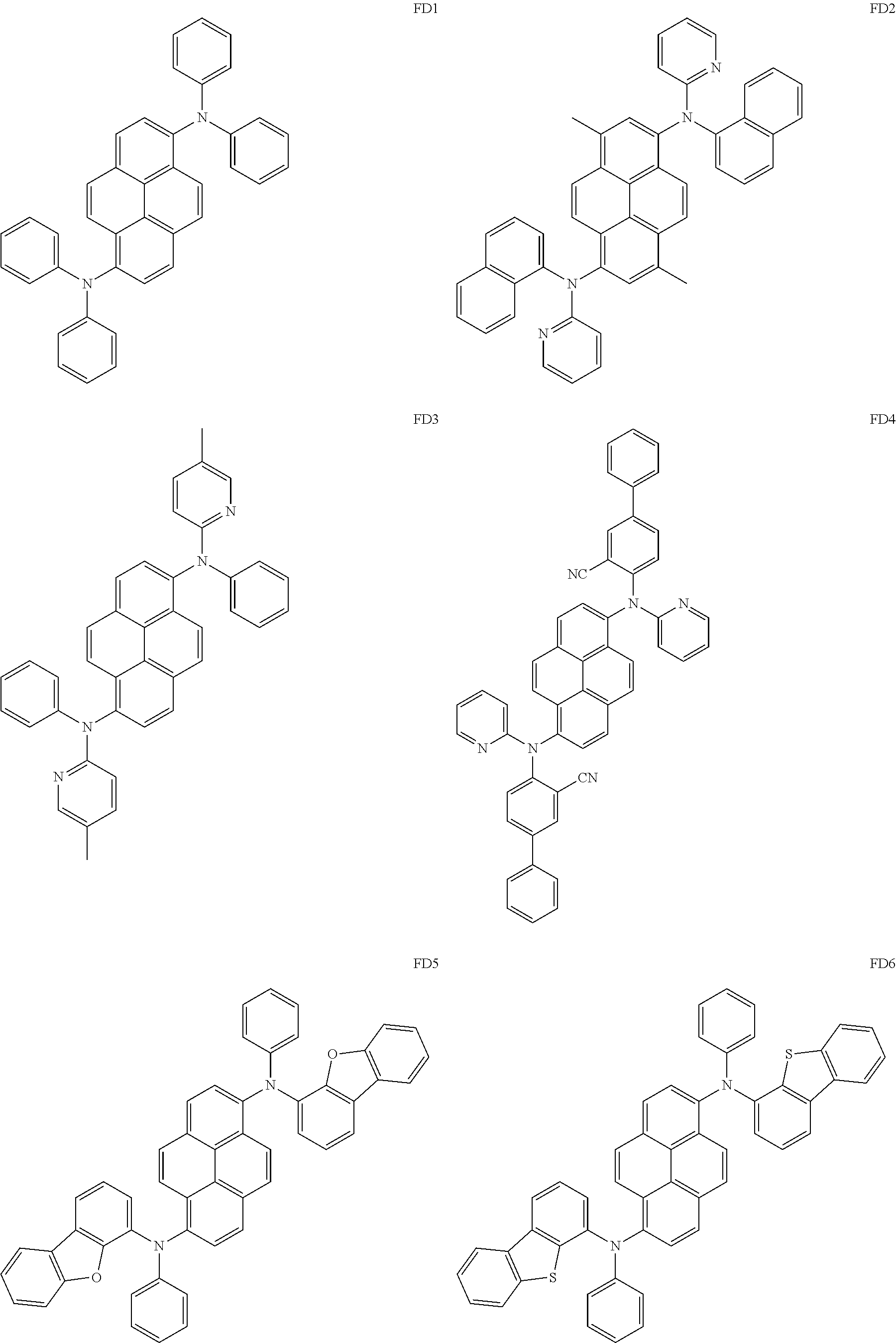

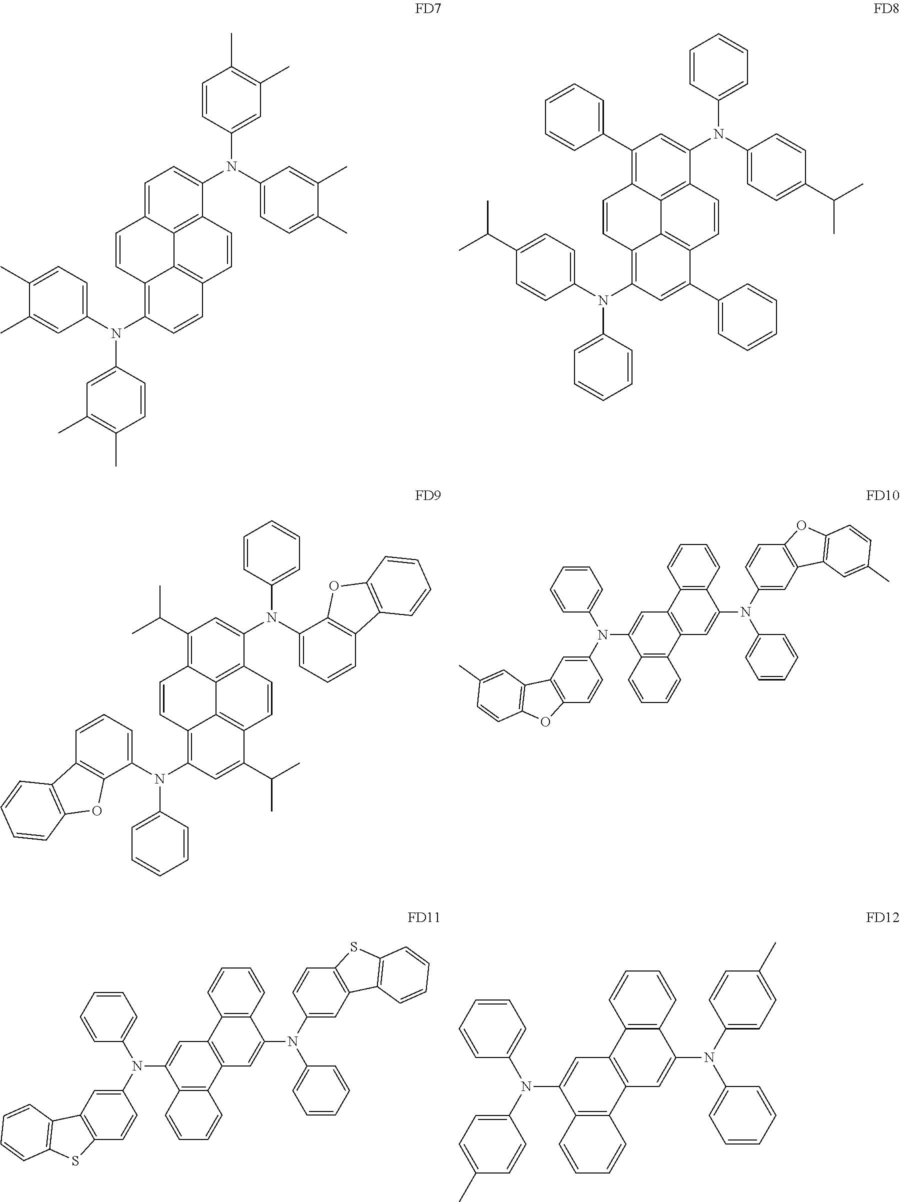

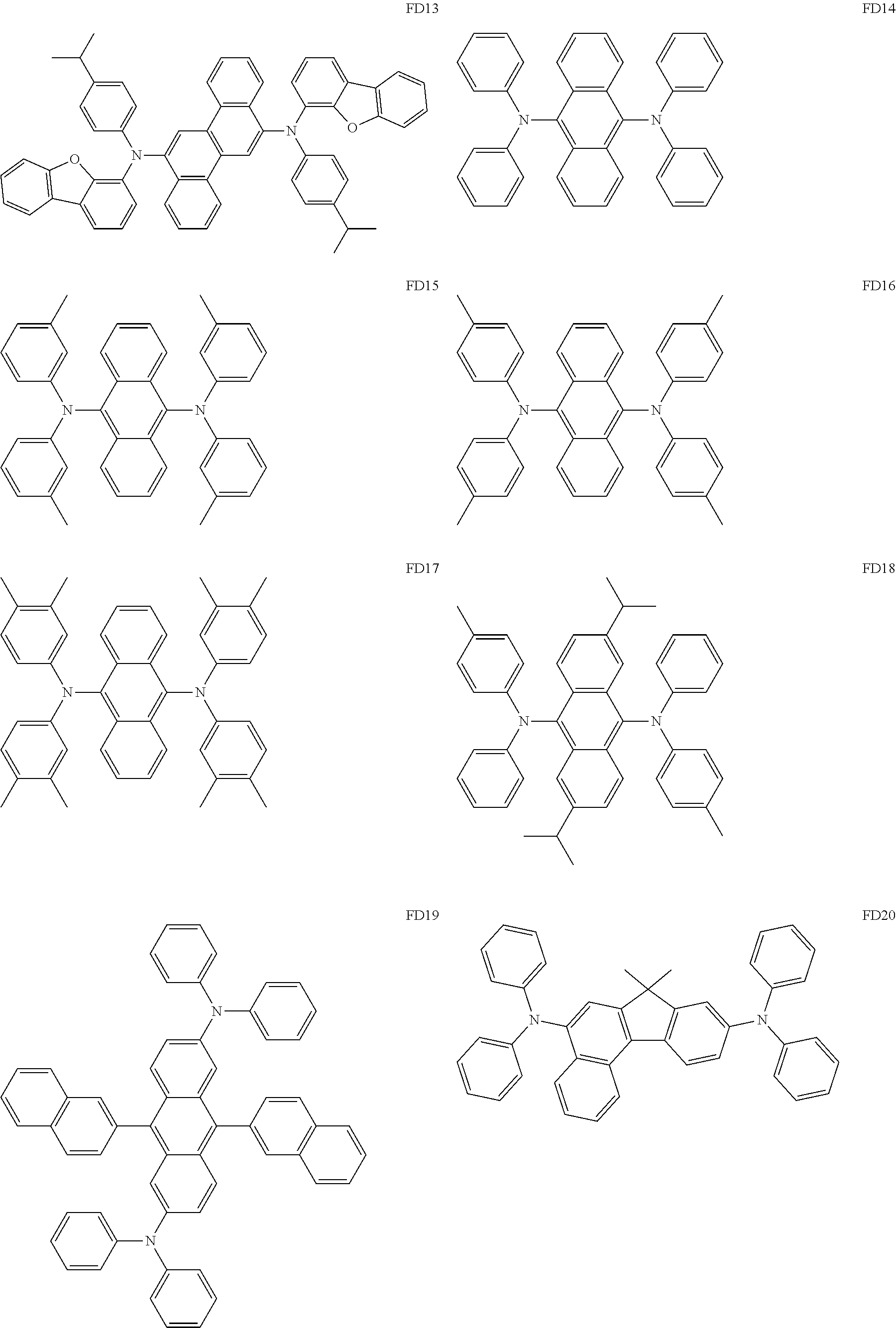

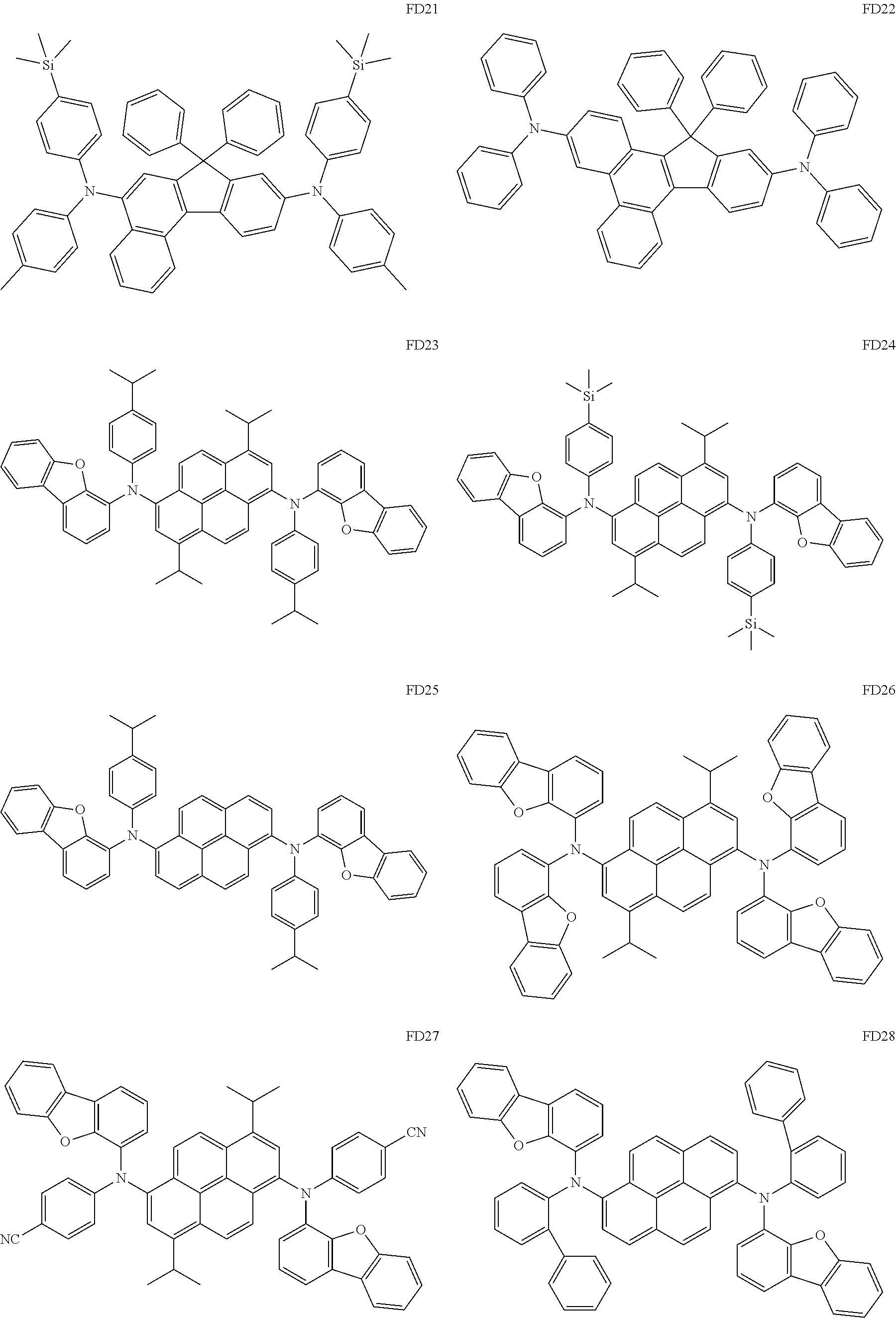

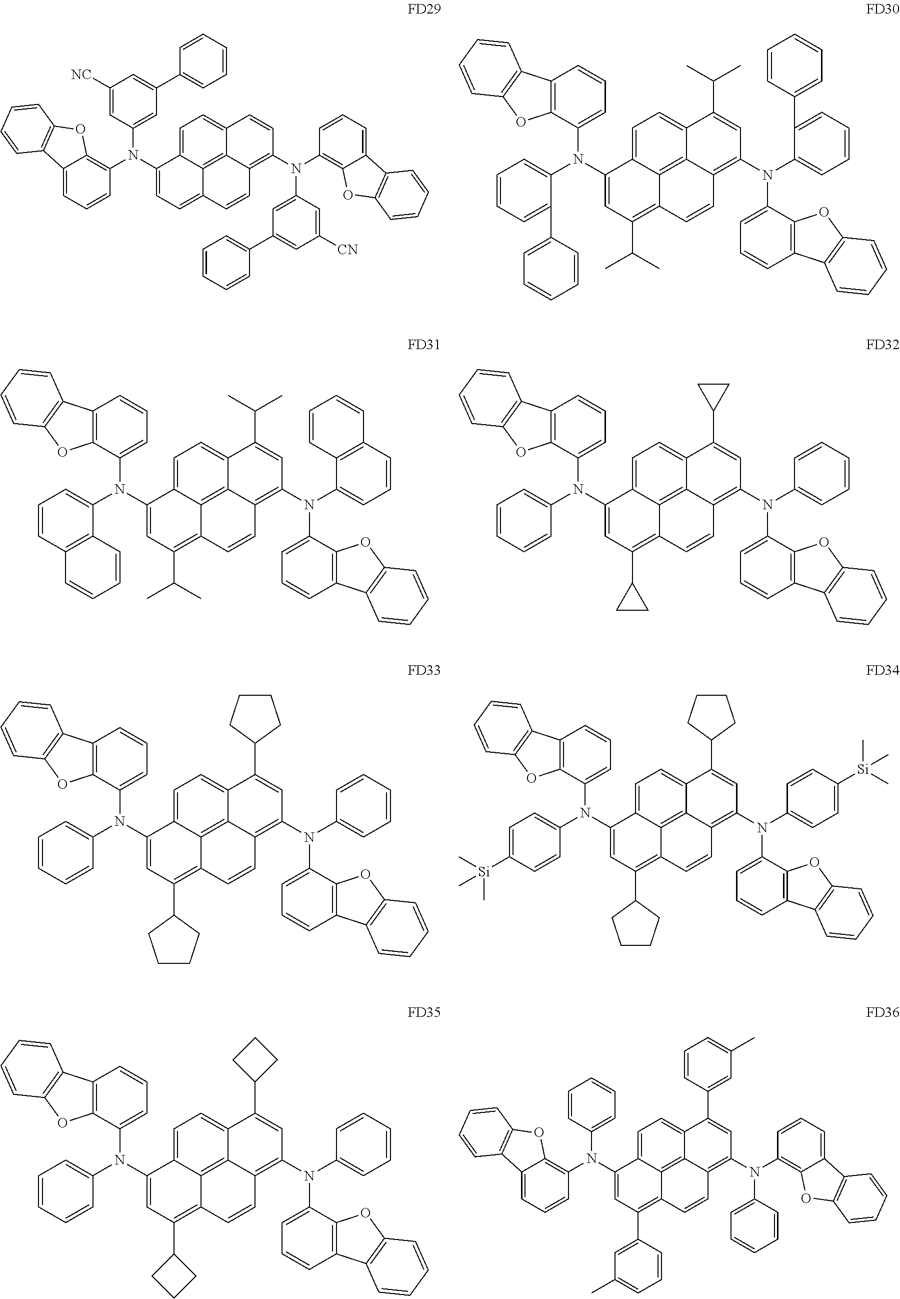

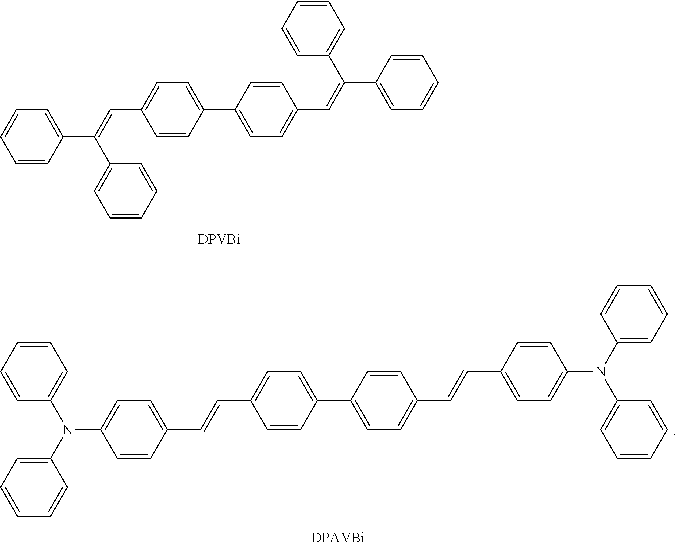

### Delayed Fluorescence Material

The emission layer may include a delayed fluorescence material.

In the present specification, the delayed fluorescence material may be selected from compounds capable of emitting delayed fluorescent light based on a delayed fluorescence emission mechanism.

The delayed fluorescence material included in the emission layer may act as a host or a dopant depending on the type (or kind) of other materials included in the emission layer.

In one or more embodiments, the difference between the triplet energy level (eV) of the delayed fluorescence material and the singlet energy level (eV) of the delayed fluorescence material may be greater than or equal to 0 eV and less than or equal to 0.5 eV. When the difference between the triplet energy level (eV) of the delayed fluorescence material and the singlet energy level (eV) of the delayed fluorescence material satisfies the above-described range, up-conversion from the triplet state to the singlet state of the delayed fluorescence materials may effectively occur, and thus, the luminescence efficiency of the light-emitting device 10 may be improved.

For example, the delayed fluorescence material may include i) a material including at least one electron donor (for example, a π electron-rich C₃-C₆₀ cyclic group, such as a carbazole group) and at least one electron acceptor (for example, a sulfoxide group, a cyano group, or a π electron-deficient nitrogen-containing C₁-C₆₀ cyclic group), and ii) a material including a C₈-C₆₀ polycyclic group in which two or more cyclic groups are condensed together while sharing boron (B).

Examples of the delayed fluorescence material may include at least one selected from the following compounds DF1 to DF9:

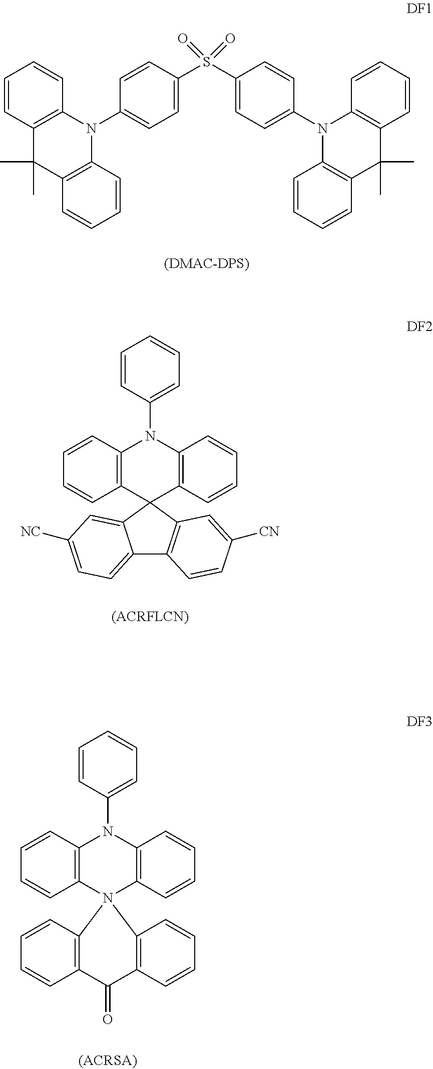

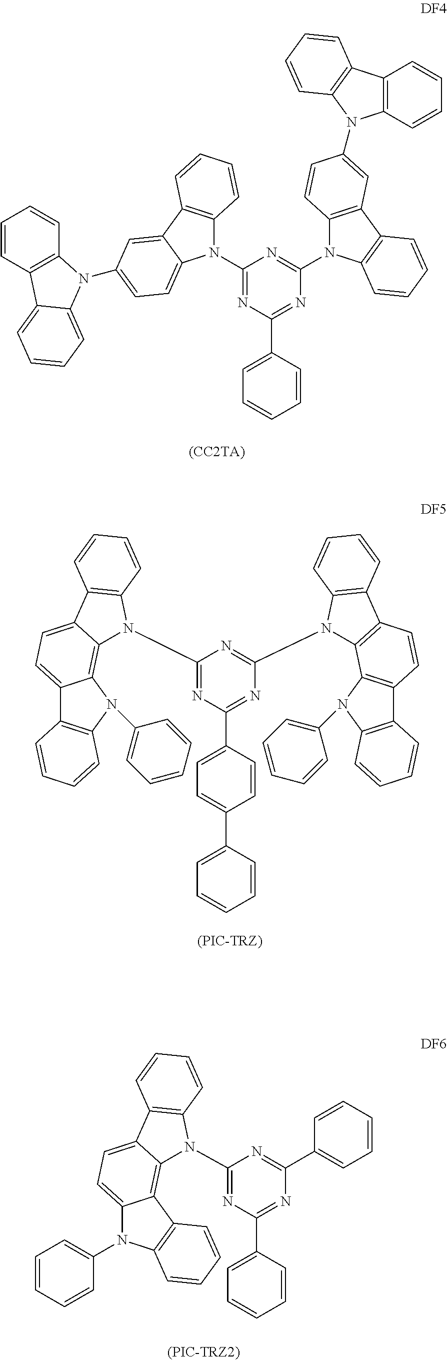 [SMILES: c1ccc(-c2nc(-n3c4ccccc4c4cc(-n5c6ccccc6c6ccccc65)ccc43)nc(-n3c4ccccc4c4cc(-n5c6ccccc6c6ccccc65)ccc43)n2)cc1 ; c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4ccc5c(c6ccccc6n5-c5ccccc5)c43)n2)cc1]

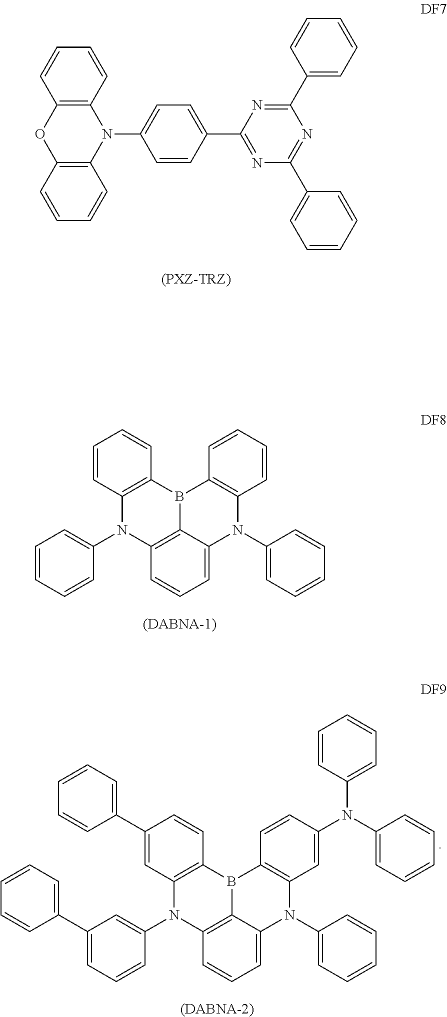

### Quantum Dot

The emission layer may include a quantum dot.

The term “quantum dot,” as used herein, refers to a crystal of a semiconductor compound, and may include any suitable material capable of emitting light of various suitable emission wavelengths according to the size of the crystal.

A diameter of the quantum dot may be, for example, in a range of about 1 nm to about 10 nm.

The quantum dot may be synthesized by a wet chemical process, a metal organic chemical vapor deposition process, a molecular beam epitaxy process, and/or any suitable process similar thereto.

The wet chemical process is a method including mixing a precursor material together with an organic solvent and then growing a quantum dot particle crystal. When the crystal grows, the organic solvent naturally acts as a dispersant coordinated on the surface of the quantum dot crystal and controls the growth of the crystal so that the growth of quantum dot particles can be controlled through a process having lower costs, and is easier than vapor deposition methods, such as metal organic chemical vapor deposition (MOCVD) or molecular beam epitaxy (MBE),

The quantum dot may include Group II-VI semiconductor compounds, Group III-V semiconductor compounds, Group III-VI semiconductor compounds, Group I-III-VI semiconductor compounds, Group IV-VI semiconductor compounds, a Group IV element or compound, or any combination thereof.

Examples of the Group II-VI semiconductor compound include a binary compound, such as CdSe, CdTe, ZnS, ZnSe, ZnTe, ZnO, HgS, HgSe, HgTe, MgSe, and/or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, and/or MgZnS; a quaternary compound, such as CdZnSeS, CdZnSeTe, CdZnSTe, CdHgSeS, CdHgSeTe, CdHgSTe, HgZnSeS, HgZnSeTe, and/or HgZnSTe; or any combination thereof.

Examples of the Group III-V semiconductor compound may include: a binary compound, such as GaN, GaP, GaAs, GaSb, AlN, AlP, AlAs, AlSb, InN, InP, InAs, and/or InSb; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, and/or InPSb; a quaternary compound, such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, and/or InAlPSb; or any combination thereof. In some embodiments, the Group III-V semiconductor compound may further include a Group II element. Examples of the Groups III-V semiconductor compound further including a Group II element include InZnP, InGaZnP, InAlZnP, etc.

Examples of the Group III-VI semiconductor compound include: a binary compound, such as GaS, GaSe, Ga₂Se₃, GaTe, InS, InSe, In₂S₃, In₂Se₃, and/or InTe; a ternary compound, such as InGaS₃, and/or InGaSe₃; and any combination thereof.

Examples of the Group I-III-VI semiconductor compound include: a ternary compound, such as AgInS, AgInS₂, CuInS, CuInS₂, CuGaO₂, AgGaO₂, and/or AgAlO₂; or any combination thereof.

Examples of the Group IV-VI semiconductor compound include: a binary compound, such as SnS, SnSe, SnTe, PbS, PbSe, and/or PbTe; a ternary compound, such as SnSeS, SnSeTe, SnSTe, PbSeS, PbSeTe, PbSTe, SnPbS, SnPbSe, and/or SnPbTe; a quaternary compound, such as SnPbSSe, SnPbSeTe, and/or SnPbSTe; or any combination thereof.

The Group IV element or compound may include: a single element compound, such as Si and/or Ge; a binary compound, such as SiC and/or SiGe; or any combination thereof.

Each element included in a multi-element compound such as the binary compound, the ternary compound, and the quaternary compound may be present at a uniform concentration or non-uniform concentration in a particle.

In some embodiments, the quantum dot may have a single structure in which the concentration of each element in the quantum dot is uniform (e.g., substantially uniform), or a core-shell dual structure. For example, the material included in the core and the material included in the shell may be different from each other.

The shell of the quantum dot may act as a protective layer that prevents or reduces chemical degeneration of the core to maintain semiconductor characteristics, and/or as a charging layer that imparts electrophoretic characteristics to the quantum dot. The shell may be a single layer or a multi-layer. The interface between the core and the shell may have a concentration gradient in which the concentration of an element existing in the shell decreases a long a direction toward the center of the core.

Examples of the shell of the quantum dot may include an oxide of metal, metalloid, or non-metal, a semiconductor compound, and any combination thereof. Examples of the oxide of metal, metalloid, or non-metal are a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, or NiO; a ternary compound, such as MgAl₂O₄, CoFe₂O₄, NiFe₂O₄, or CoMn₂O₄; and any combination thereof. Examples of the semiconductor compound include, as described herein, Group II-VI semiconductor compounds; Group III-V semiconductor compounds; Group III-VI semiconductor compounds; Group I-III-VI semiconductor compounds; Group IV-VI semiconductor compounds; and any combination thereof. For example, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or any combination thereof.

A full width at half maximum (FWHM) of the emission wavelength spectrum of the quantum dot may be about 45 nm or less, for example, about 40 nm or less, for example, about 30 nm or less, and within these ranges, color purity and/or color reproducibility may be increased. In addition, because the light emitted through the quantum dot is emitted in all directions (e.g., substantially all directions), the wide viewing angle may be improved.

In addition, the quantum dot may be in the form of a spherical particle, a pyramidal particle, a multi-arm particle, a cubic nanoparticle, a nanotube particle, a nanowire particle, a nanofiber particle, and/or a nanoplate particle.

Because the energy band gap may be adjusted by controlling the size of the quantum dot, light having various suitable wavelength bands may be obtained from the quantum dot emission layer. Accordingly, by using quantum dots of different sizes, a light-emitting device that emits light of various suitable wavelengths may be implemented. In one or more embodiments, the size of the quantum dot may be selected to emit red, green and/or blue light. In addition, the size of the quantum dot may be configured to emit white light by combination of light of various suitable colors.

Electron Transport Region in Interlayer 130

The electron transport region may have: i) a single-layered structure consisting of a single layer consisting of a single material, ii) a single-layered structure consisting of a single layer consisting of a plurality of different materials, or iii) a multi-layered structure including a plurality of layers including different materials.

The electron-transporting region may include a buffer layer, a hole-blocking layer, an electron control layer, an electron-transporting layer, an electron injection layer, or any combination thereof.

For example, the electron transport region may have an electron transport layer/electron injection layer structure, a hole-blocking layer/electron transport layer/electron injection layer structure, an electron control layer/electron transport layer/electron injection layer structure, or a buffer layer/electron transport layer/electron injection layer structure, the constituting layers of each structure being sequentially stacked from an emission layer.

In an embodiment, the electron transport region (for example, the buffer layer, the hole-blocking layer, the electron control layer, and/or the electron transport layer in the electron transport region) may include a metal-free compound including at least one π electron-deficient nitrogen-containing C₁-C₆₀ cyclic group.

For example, the electron transport region may include a compound represented by Formula 601 below:

[Ar₆₀₁]ₓₑ₁₁−[(L₆₀₁)ₓₑ₁−R₆₀₁]ₓₑ₂₁ Formula 601

wherein, in Formula 601,

Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xe11 may be 1, 2, or 3,

xe1 may be 0, 1, 2, 3, 4, or 5,

R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂ (Q₆₀₁), or —P(═O)(Q₆₀₁)(Q₆₀₂),

Q₆₀₁ to Q₆₀₃ may each be the same as described herein with respect to Qi,

xe21 may be 1, 2, 3, 4, or 5,

at least one of Ar₆₀₁, L₆₀₁, and R₆₀₁ may each independently be air electron-deficient nitrogen-containing C₁-C₆₀ cyclic group unsubstituted or substituted with at least one R₁₀ₐ.

For example, when xe11 in Formula 601 is two or more, two or more of Ar₆₀₁(s) may be linked to each other via a single bond.

In other embodiments, Ar₆₀₁ in Formula 601 may be a substituted or unsubstituted anthracene group.

In other embodiments, the electron transport region may include a compound represented by Formula 601-1:

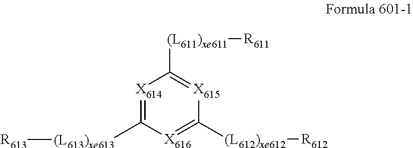

wherein, in Formula 601-1,

X₆₁₄ may be N or C(R₆₁₄), X₆₁₅ may be N or C(R₆₁₅), X₆₁₆ may be N or C(R₆₁₆), and at least one of X₆₁₄ to X₆₁₆ may be N,

L₆₁₁ to L₆₁₃ may each be the same as described herein with respect to L₆₀₁,

xe611 to xe613 may each be the same as described herein with respect to xe1,

R₆₁₁ to R₆₁₃ may each be the same as described herein with respect to R₆₀₁, and

R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, xe1 and xe611 to xe613 in Formulae 601 and 601-1 may each independently be 0, 1, or 2.

The electron transport region may include one of Compounds ET1 to ET45, 2,9-dimethyl-4,7-diphenyl-1,10-phenanthroline (BCP), 4,7-diphenyl-1,10-phenanthroline (Bphen), Alq3, BAlq, TAZ, NTAZ, or any combination thereof:

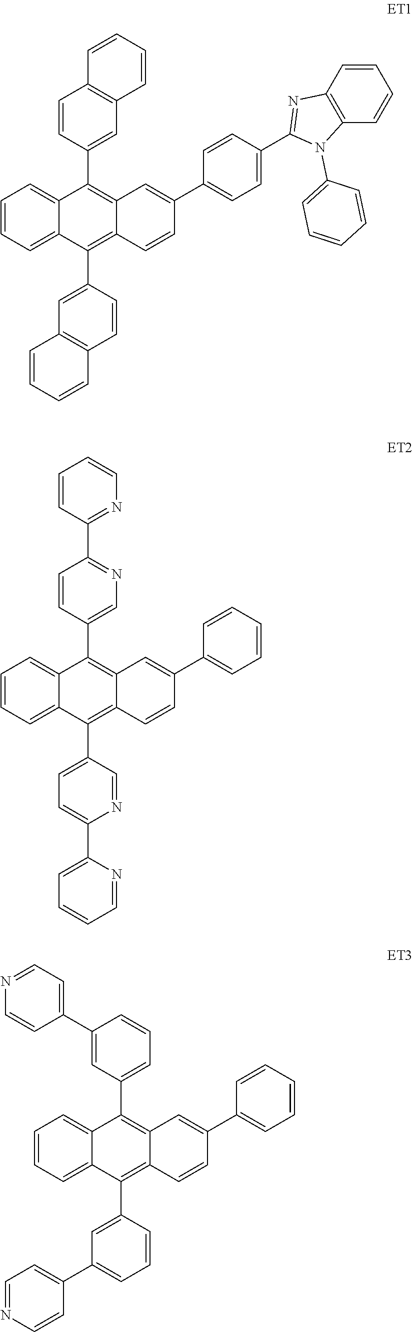 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

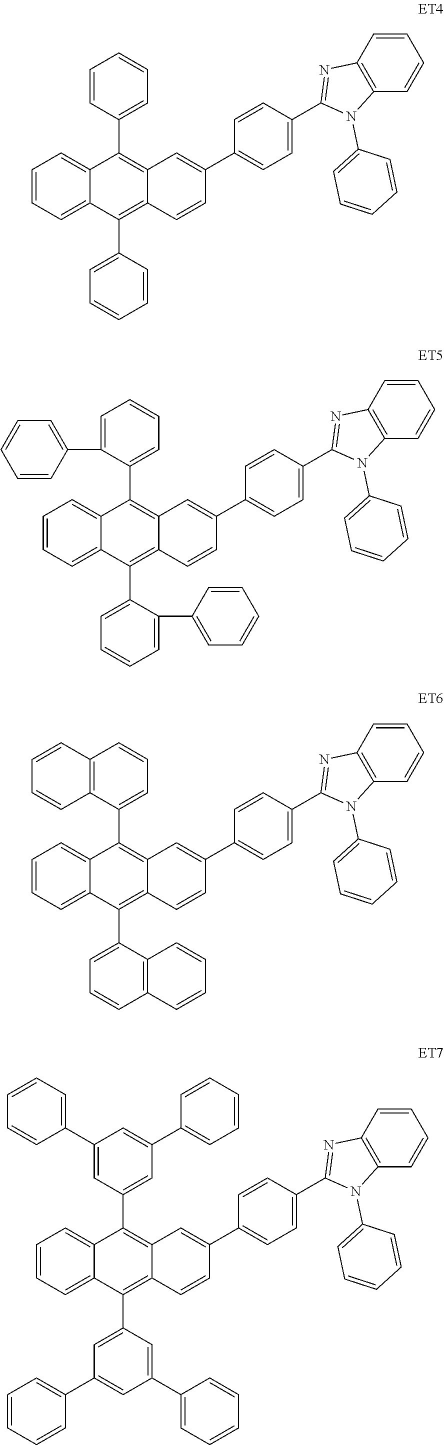 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

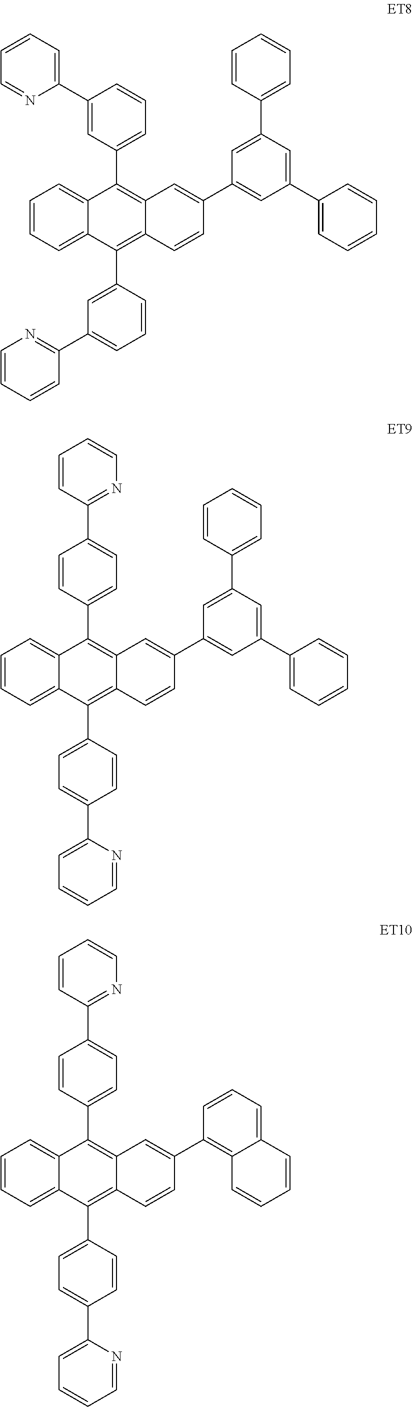

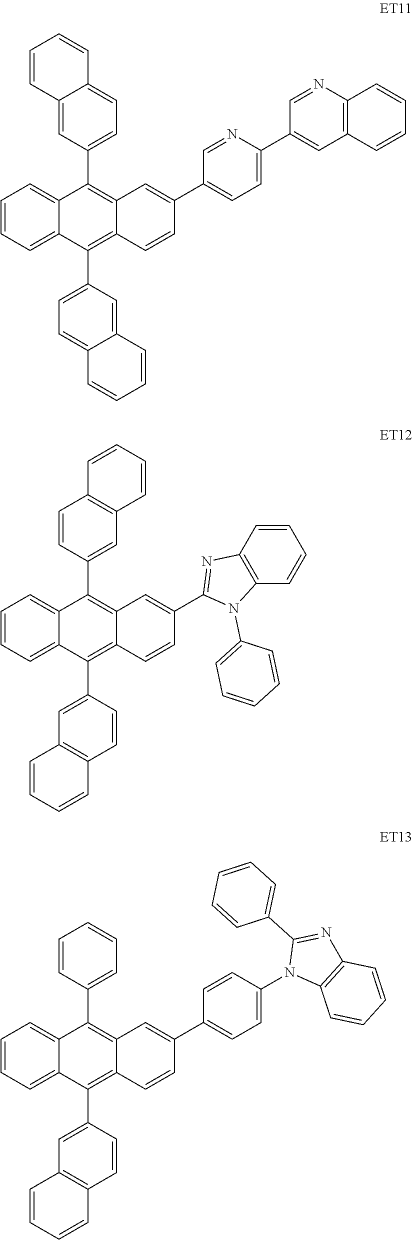

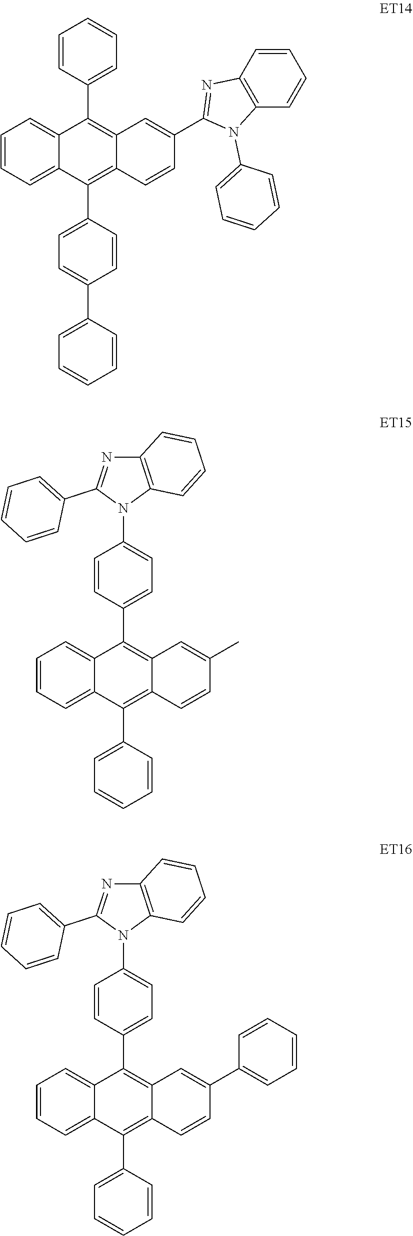 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

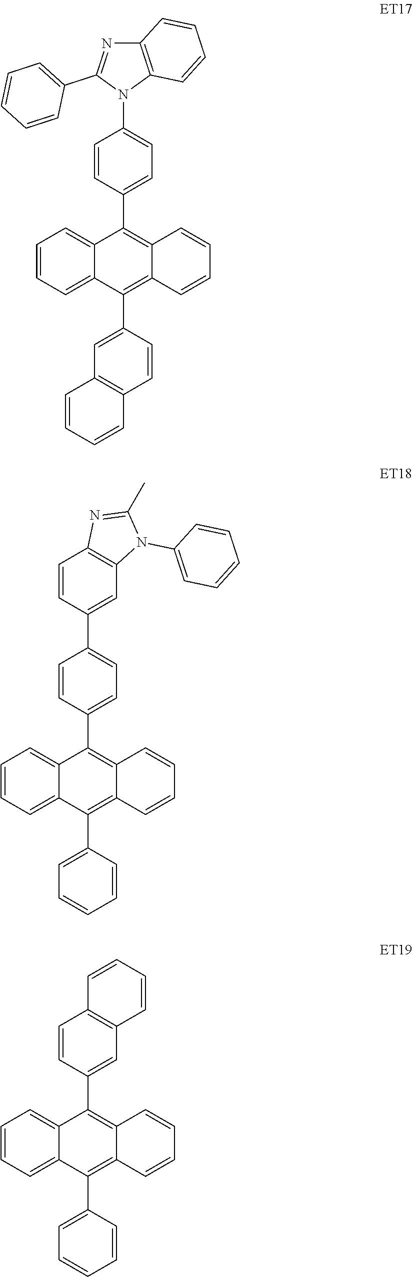

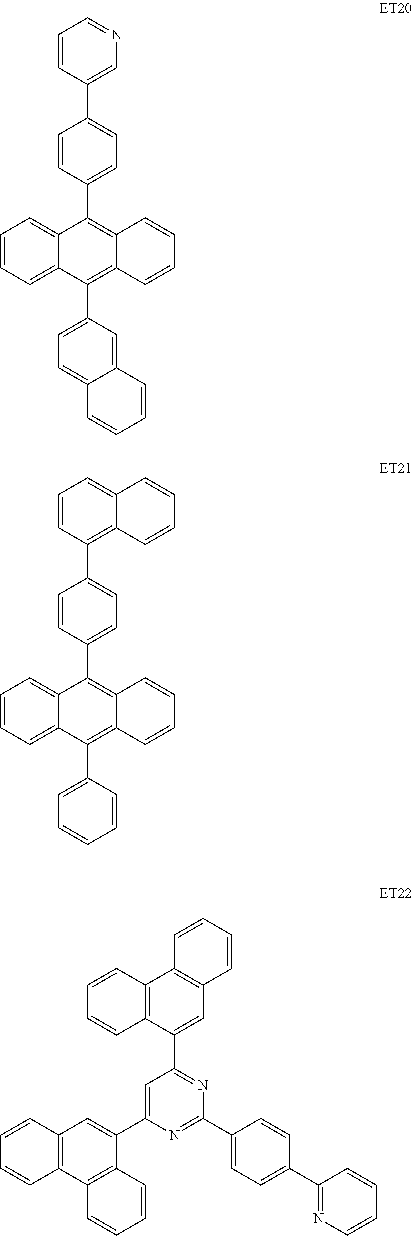

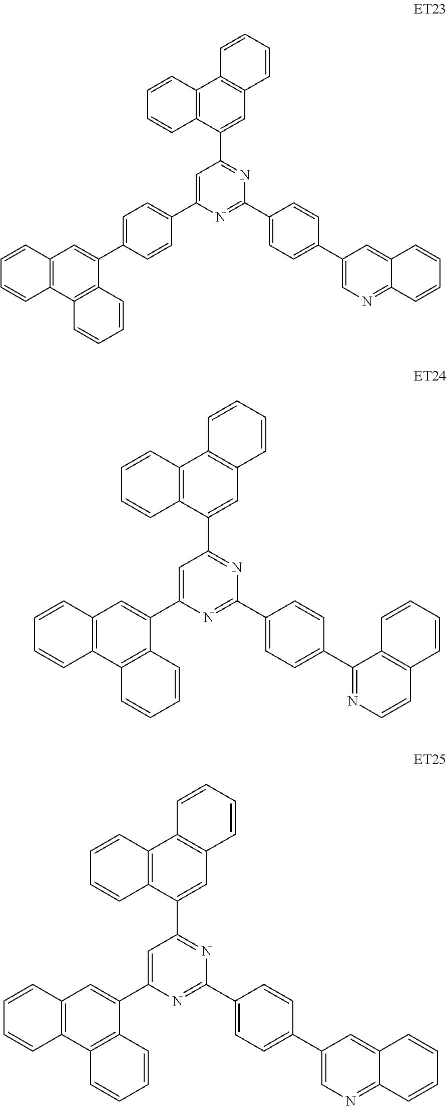 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)cc(-c5cc6ccccc6c6ccccc56)n4)cc3)cc2c1]

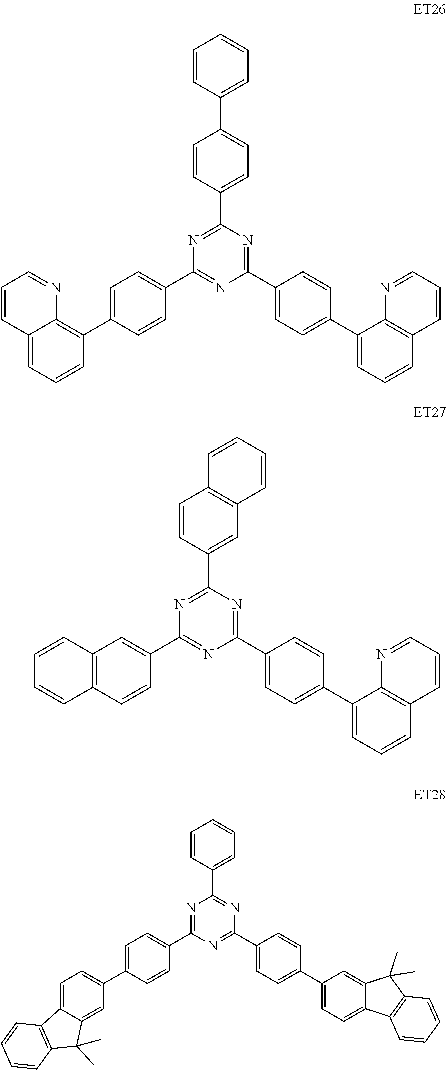 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21]

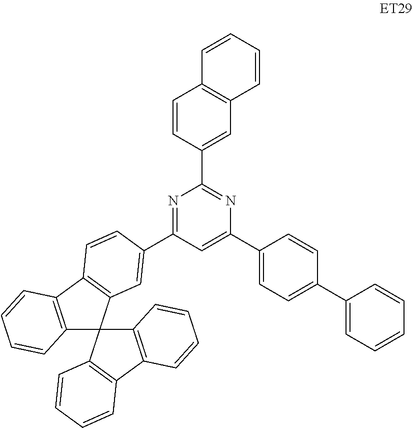 [SMILES: c1ccc(-c2ccc(-c3cc(-c4ccc5c(c4)C4(c6ccccc6-c6ccccc64)c4ccccc4-5)nc(-c4ccc5ccccc5c4)n3)cc2)cc1]

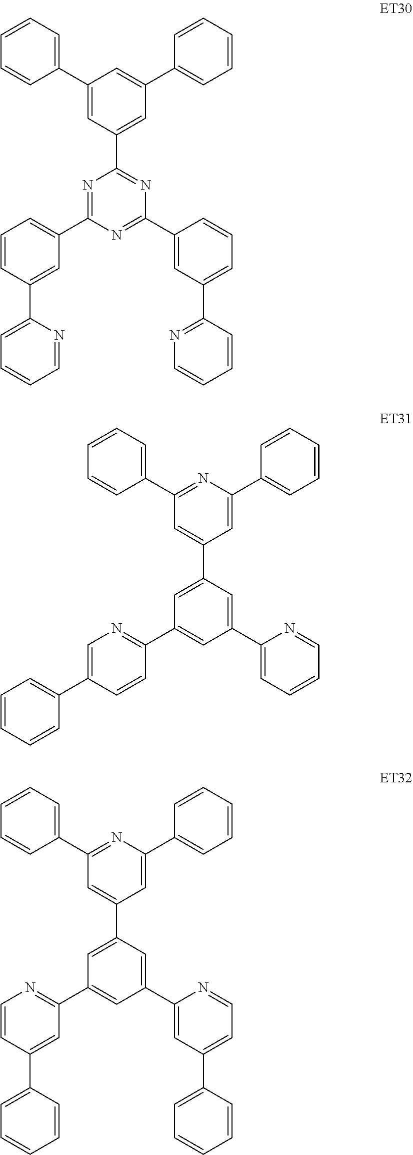

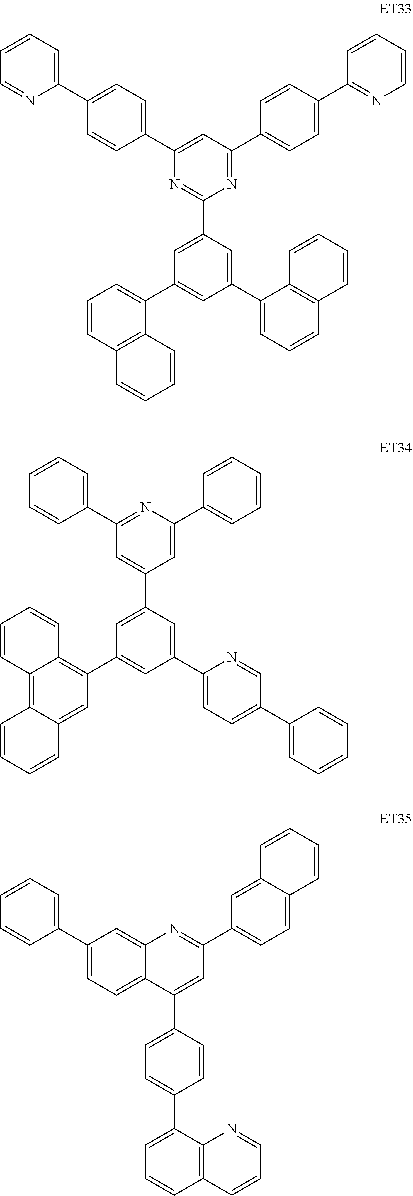 [SMILES: c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1]

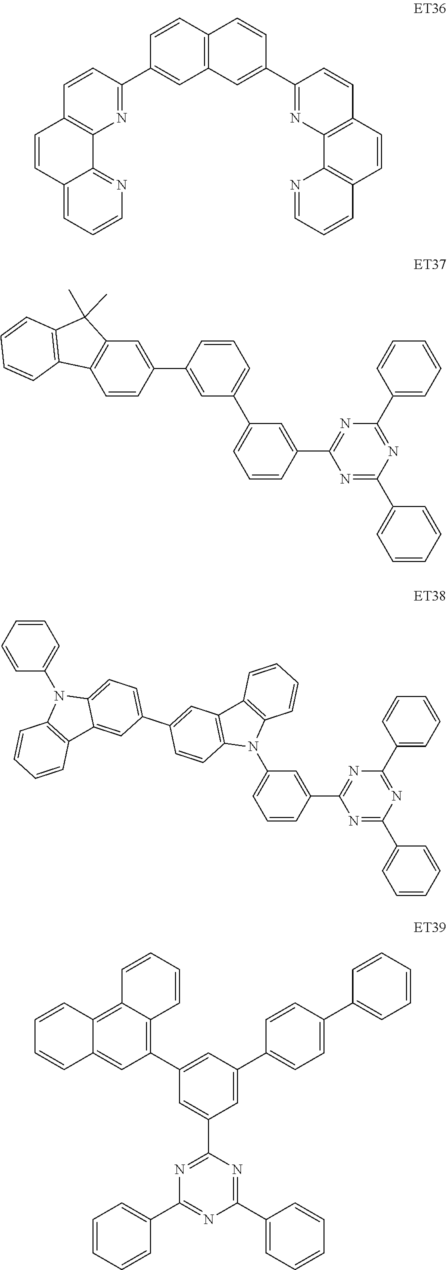 [SMILES: c1cnc2c(c1)ccc1ccc(-c3ccc4ccc(-c5ccc6ccc7cccnc7c6n5)cc4c3)nc12 ; CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1]

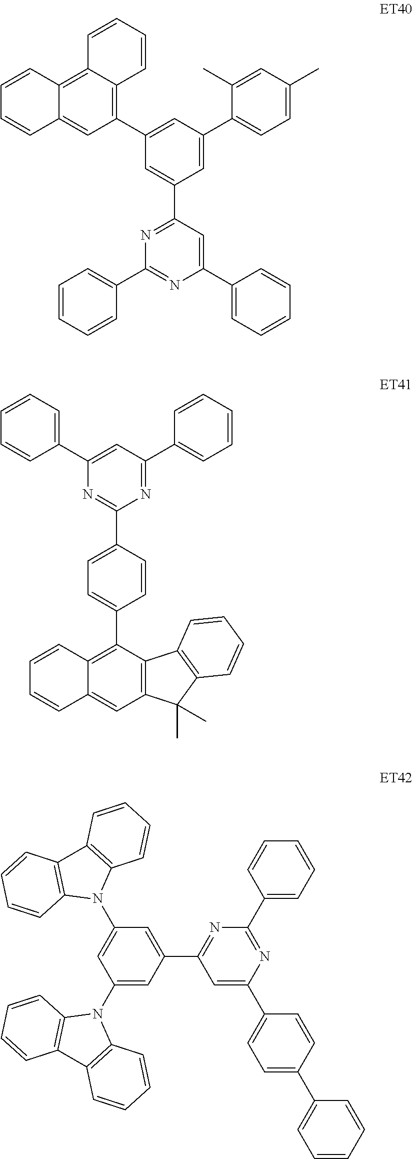 [SMILES: Cc1ccc(-c2cc(-c3cc(-c4ccccc4)nc(-c4ccccc4)n3)cc(-c3cc4ccccc4c4ccccc34)c2)c(C)c1 ; CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1]

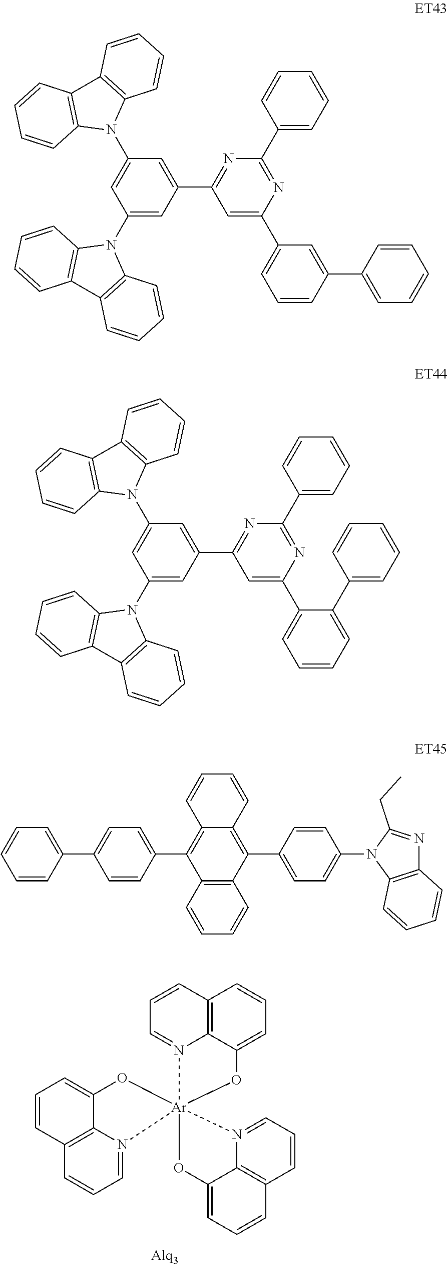

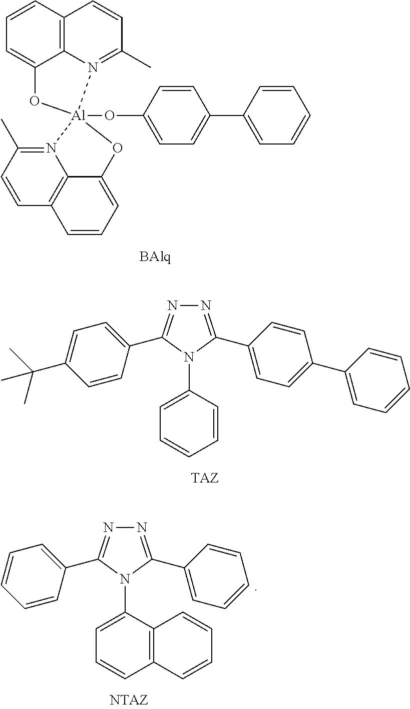

A thickness of the electron transport region may be from about 100 Å to about 5,000 Å, for example, about 160 Å to about 4,000 Å. When the electron transport region includes a buffer layer, a hole-blocking layer, an electron control layer, an electron transport layer, or any combination thereof, the thickness of the buffer layer, the hole-blocking layer, or the electron control layer may each independently be from about 20 Å to about 1000 Å, for example, about 30 Å to about 300 Å, and the thickness of the electron transport layer may be from about 100 Å to about 1000 Å, for example, about 150 Å to about 500 Å. When the thickness of the buffer layer, the hole blocking layer, the electron control layer, the electron transport layer, and/or the electron transport region are within these ranges, suitable or satisfactory electron transporting characteristics may be obtained without a substantial increase in driving voltage.

The electron transport region (for example, the electron transport layer in the electron transport region) may further include, in addition to the materials described above, a metal-containing material.

The metal-containing material may include an alkali metal complex, an alkaline earth metal complex, or any combination thereof. The metal ion of an alkali metal complex may be a Li ion, a Na ion, a K ion, a Rb ion, or a Cs ion, and the metal ion of an alkaline earth metal complex may be a Be ion, a Mg ion, a Ca ion, a Sr ion, or a Ba ion. A ligand coordinated with the metal ion of the alkali metal complex or the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

For example, the metal-containing material may include a Li complex. The Li complex may include, for example, Compound ET-D1 (LiQ) and/or ET-D2:

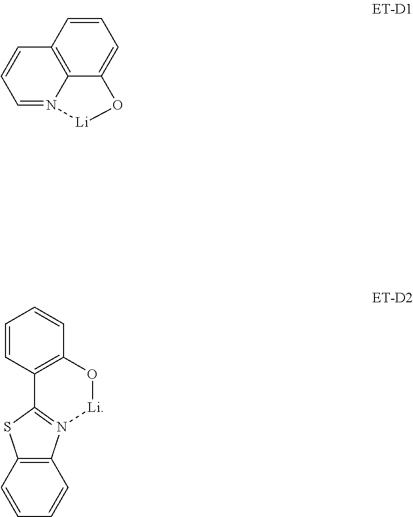

The electron transport region may include an electron injection layer that facilitates the injection of electrons from the second electrode 150. The electron injection layer may directly contact (e.g., physically contact) the second electrode 150.

The electron injection layer may have: i) a single-layered structure consisting of a single layer consisting of a single material, ii) a single-layered structure consisting of a single layer consisting of a plurality of different materials, or iii) a multi-layered structure including a plurality of layers including different materials.

The electron injection layer may include an alkali metal, alkaline earth metal, a rare earth metal, an alkali metal-containing compound, alkaline earth metal-containing compound, a rare earth metal-containing compound, an alkali metal complex, an alkaline earth metal complex, a rare earth metal complex, or any combination thereof.

The alkali metal may include Li, a Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may be oxides, halides (for example, fluorides, chlorides, bromides, and/or iodides), and/or tellurides of the alkali metal, the alkaline earth metal, and the rare earth metal, or any combination thereof.

The alkali metal-containing compound may include: alkali metal oxides, such as Li₂O, Cs₂O, and/or K₂O; alkali metal halides, such as LiF, NaF, CsF, KF, LiI, NaI, CsI, and/or KI; or any combination thereof. The alkaline earth metal-containing compound may include an alkaline earth metal compound, such as BaO, SrO, CaO, BaₓSr₁₋ₓO (wherein x is a real number satisfying the condition of 0<x<1), BaₓCa₁₋ₓO (wherein x is a real number satisfying the condition of 0<x<1), and/or the like. The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In one or more embodiments, the rare earth metal-containing compound may include lanthanide metal telluride. Examples of the lanthanide metal telluride include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, and Lu₂Te₃.

The alkali metal complex, the alkaline earth-metal complex, and the rare earth metal complex may include i) one of ions of the alkali metal, the alkaline earth metal, and the rare earth metal and ii), as a ligand bonded to the metal ion, for example, a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenyl benzimidazole, a hydroxyphenylbenzothiazole, a bipyridine, a phenanthroline, a cyclopentadiene, or any combination thereof.

The electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In one or more embodiments, the electron injection layer may further include an organic material (for example, a compound represented by Formula 601).

In one or more embodiments, the electron injection layer may include (e.g., consist of): i) an alkali metal-containing compound (for example, an alkali metal halide); or ii) a) an alkali metal-containing compound (for example, an alkali metal halide), and b) an alkali metal, an alkaline earth metal, a rare earth metal, or any combination thereof. For example, the electron injection layer may be a KI:Yb co-deposited layer, an RbI:Yb co-deposited layer, and/or the like.

When the electron injection layer further includes an organic material, alkali metal, alkaline earth metal, rare earth metal, an alkali metal-containing compound, an alkaline earth metal-containing compound, a rare earth metal-containing compound, alkali metal complex, alkaline earth-metal complex, rare earth metal complex, or any combination thereof may be uniformly or non-uniformly dispersed in a matrix including the organic material.

A thickness of the electron injection layer may be in a range of about 1 Å to about 100 Å, and, for example, about 3 Å to about 90 Å. When the thickness of the electron injection layer is within the ranges described above, suitable or satisfactory electron injection characteristics may be obtained without a substantial increase in driving voltage.

### Second Electrode 150

The second electrode 150 may be on the interlayer 130 having a structure as described above. The second electrode 150 may be a cathode, which is an electron injection electrode, and as the material for the second electrode 150, a metal, an alloy, an electrically conductive compound, or any combination thereof, each having a low-work function, may be used.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layered structure or a multi-layered structure including a plurality of layers.

### Capping Layer

A first capping layer may be outside the first electrode 110, and/or a second capping layer may be outside the second electrode 150. For example, the light-emitting device 10 may have a structure in which the first capping layer, the first electrode 110, the interlayer 130, and the second electrode 150 are sequentially stacked in the stated order, a structure in which the first electrode 110, the interlayer 130, the second electrode 150, and the second capping layer are sequentially stacked in the stated order, or a structure in which the first capping layer, the first electrode 110, the interlayer 130, the second electrode 150, and the second capping layer are sequentially stacked in the stated order.

Light generated in an emission layer of the interlayer 130 of the light-emitting device 10 may be extracted toward the outside through the first electrode 110 which is a semi-transmissive electrode or a transmissive electrode, and the first capping layer. Light generated in an emission layer of the interlayer 130 of the light-emitting device 10 may be extracted toward the outside through the second electrode 150 which is a semi-transmissive electrode or a transmissive electrode, and the second capping layer.

The first capping layer and the second capping layer may increase external emission efficiency according to the principle of constructive interference. Accordingly, the light extraction efficiency of the light-emitting device 10 is increased, so that the luminescence efficiency of the light-emitting device 10 may be improved.

Each of the first capping layer and the second capping layer may include a material having a refractive index of 1.6 or more (at a wavelength of 589 nm).

The first capping layer and the second capping layer may each independently be an organic capping layer including an organic material, an inorganic capping layer including an inorganic material, or an organic-inorganic composite capping layer including an organic material and an inorganic material.

At least one selected from the first capping layer and the second capping layer may each independently include carbocyclic compounds, heterocyclic compounds, amine group-containing compounds, porphyrin derivatives, phthalocyanine derivatives, a naphthalocyanine derivatives, alkali metal complexes, alkaline earth metal complexes, or any combination thereof. Optionally, the carbocyclic compound, the heterocyclic compound, and the amine group-containing compound may be substituted with a substituent including O, N, S, Se, Si, F, Cl, Br, I, or any combination thereof. In one or more embodiments, at least one selected from the first capping layer and the second capping layer may each independently include an amine group-containing compound.

For example, at least one selected from the first capping layer and the second capping layer may each independently include a compound represented by Formula 201, a compound represented by Formula 202, or any combination thereof.

In one or more embodiments, at least one selected from the first capping layer and the second capping layer may each independently include one of Compounds HT28 to HT33, one of Compounds CP1 to CP6, β-NPB, or any combination thereof:

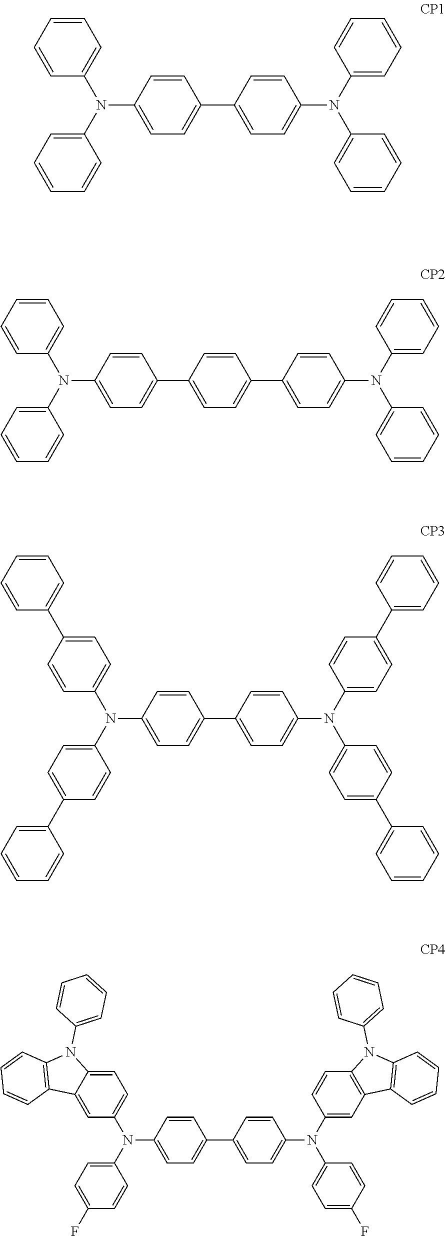 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1]

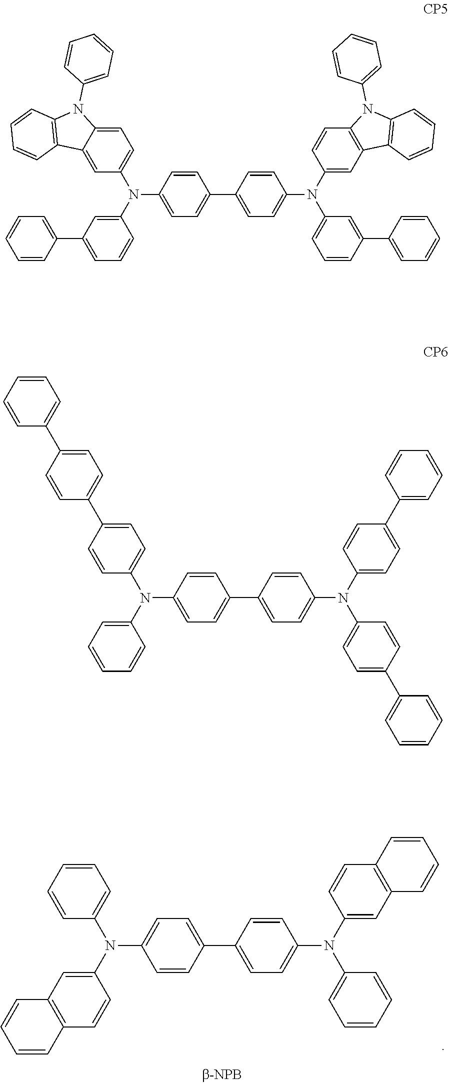 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

### Film

The diamine compound represented by Formula 1 may be included in various suitable films. Accordingly, another aspect of embodiments provides a film including the diamine compound represented by Formula 1. The film may be, for example, an optical member (and/or a light control means) (for example, a color filter, a color conversion member, a capping layer, a light extraction efficiency enhancement layer, a selective light absorbing layer, a polarizing layer, a quantum dot-containing layer, and/or the like), a light-blocking member (for example, a light reflective layer, a light absorbing layer, and/or the like), a protective member (for example, an insulating layer, a dielectric layer, and/or the like).

### Electronic Apparatus

The light-emitting device may be included in various suitable electronic apparatuses. For example, the electronic apparatus including the light-emitting device may be a light-emitting apparatus, an authentication apparatus, and/or the like.

The electronic apparatus (for example, a light-emitting apparatus) may further include, in addition to the light-emitting device, i) a color filter, ii) a color conversion layer, or iii) a color filter and a color conversion layer. The color filter and/or the color conversion layer may be in at least one traveling direction of light emitted from the light-emitting device. For example, the light emitted from the light-emitting device may be blue light or white light. For additional details of the light-emitting device, related description provided above may be referred to. In one or more embodiments, the color conversion layer may include a quantum dot. The quantum dot may be, for example, a quantum dot as described herein.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel-defining film may be located among the subpixel areas to define each of the subpixel areas.

The color filter may further include a plurality of color filter areas and light-shielding patterns located among the color filter areas, and the color conversion layer may further include a plurality of color conversion areas and light-shielding patterns located among the color conversion areas.

The plurality of color filter areas (or the plurality of color conversion areas) may include a first area that emits a first color light, a second area that emits a second color light, and/or a third area that emits a third color light, wherein the first color light, the second color light, and/or the third color light may have different maximum emission wavelengths from one another. For example, the first color light may be red light, the second color light may be green light, and the third color light may be blue light. For example, the plurality of color filter areas (or the plurality of color conversion areas) may include quantum dots. For example, the first area may include a red quantum dot, the second area may include a green quantum dot, and the third area may not include a quantum dot. For additional details of the quantum dot, related descriptions provided herein may be referred to. The first area, the second area, and/or the third area may each include a scatterer (e.g., a light scatterer).

For example, the light-emitting device may emit a first light, the first area may absorb the first light to emit a first-first color light, the second area may absorb the first light to emit a second-first color light, and the third area may absorb the first light to emit a third-first color light. In this regard, the first-first color light, the second-first color light, and the third-first color light may have different maximum emission wavelengths. For example, the first light may be blue light, the first-first color light may be red light, the second-first color light may be green light, and the third-first color light may be blue light.

The electronic apparatus may further include a thin-film transistor, in addition to the light-emitting device as described above. The thin-film transistor may include a source electrode, a drain electrode, and an activation layer, wherein any one selected from the source electrode and the drain electrode may be electrically connected to any one selected from the first electrode and the second electrode of the light-emitting device.

The thin-film transistor may further include a gate electrode, a gate insulating film, and/or the like.

The activation layer may include crystalline silicon, amorphous silicon, an organic semiconductor, an oxide semiconductor, and/or the like.

The electronic apparatus may further include a sealing portion for sealing the light-emitting device. The sealing portion may be between the color conversion layer and/or color filter and the light-emitting device. The sealing portion allows light from the light-emitting device to be extracted to the outside, and concurrently (e.g., simultaneously) prevents or reduces penetration of ambient air and/or moisture into the light-emitting device. The sealing portion may be a sealing substrate including a transparent glass substrate and/or a plastic substrate. The sealing portion may be a thin-film encapsulation layer including at least one layer of an organic layer and/or an inorganic layer. When the sealing portion is a thin film encapsulation layer, the electronic apparatus may be flexible.

Various suitable functional layers may be additionally on the sealing portion, in addition to the color filter and/or the color conversion layer, according to the use of the electronic apparatus. Examples of the functional layers may include a touch screen layer, a polarizing layer, and the like. The touch screen layer may be a pressure-sensitive touch screen layer, a capacitive touch screen layer, and/or an infrared touch screen layer. The authentication apparatus may be, for example, a biometric authentication apparatus that authenticates an individual by using biometric information of a living body (for example, fingertips, pupils, etc.).

The authentication apparatus may further include, in addition to the light-emitting device as described above, a biometric information collector.

The electronic apparatus may be applied to various suitable displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, and/or endoscope displays), fish finders, various suitable measuring instruments, meters (for example, meters for a vehicle, an aircraft, and/or a vessel), projectors, and/or the like.

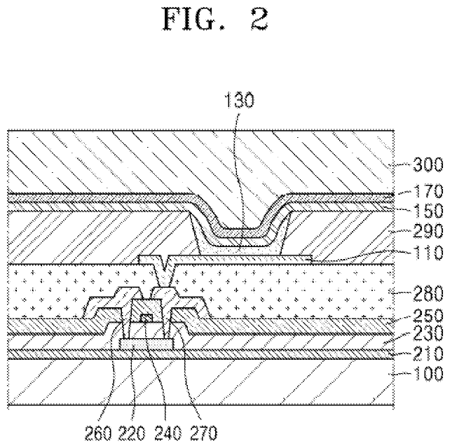

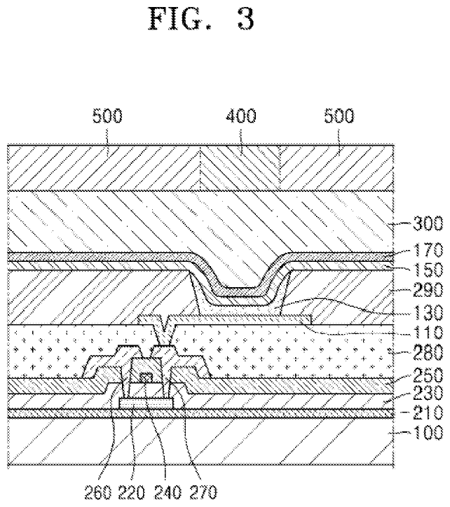

### Description of FIGS. 2 and 3

FIG. 2 is a cross-sectional view showing a light-emitting apparatus according to an embodiment of the present disclosure.

The light-emitting apparatus of FIG. 2 includes a substrate 100, a thin-film transistor (TFT), a light-emitting device, and an encapsulation portion 300 that seals the light-emitting device.

The substrate 100 may be a flexible substrate, a glass substrate, and/or a metal substrate. A buffer layer 210 may be on the substrate 100. The buffer layer 210 may prevent or reduce penetration of impurities through the substrate 100 and may provide a flat surface on the substrate 100.

A TFT may be on the buffer layer 210. The TFT may include an activation layer 220, a gate electrode 240, a source electrode 260, and a drain electrode 270.

The activation layer 220 may include an inorganic semiconductor such as silicon or polysilicon, an organic semiconductor, and/or an oxide semiconductor, and may include a source region, a drain region, and a channel region.

A gate insulating film 230 for insulating the activation layer 220 from the gate electrode 240 may be on the activation layer 220, and the gate electrode 240 may be on the gate insulating film 230.

An interlayer insulating film 250 may be on the gate electrode 240. The interlayer insulating film 250 may be between the gate electrode 240 and the source electrode 260 and between the gate electrode 240 and the drain electrode 270, to insulate from one another.

The source electrode 260 and the drain electrode 270 may be on the interlayer insulating film 250. The interlayer insulating film 250 and the gate insulating film 230 may expose the source region and the drain region of the activation layer 220, and the source electrode 260 and the drain electrode 270 may be in contact (e.g., physical contact) with the exposed portions of the source region and the drain region of the activation layer 220.

The TFT is electrically connected to a light-emitting device to drive the light-emitting device, and is covered and protected by a passivation layer 280. The passivation layer 280 may include an inorganic insulating film, an organic insulating film, or any combination thereof. A light-emitting device is provided on the passivation layer 280. The light-emitting device may include a first electrode 110, an interlayer 130, and a second electrode 150.

The first electrode 110 may be on the passivation layer 280. The passivation layer 280 may expose a portion of the drain electrode 270, not fully covering the drain electrode 270, and the first electrode 110 may be connected to the exposed portion of the drain electrode 270.

A pixel defining layer 290 including an insulating material may be on the first electrode 110. The pixel defining layer 290 may expose a certain region of the first electrode 110, and an interlayer 130 may be formed in the exposed region of the first electrode 110. The pixel defining layer 290 may be a polyimide and/or polyacrylic organic film. In some embodiments, at least some layers of the interlayer 130 may extend beyond the upper portion of the pixel defining layer 290 in the form of a common layer.

The second electrode 150 may be on the interlayer 130, and a capping layer 170 may be additionally on the second electrode 150. The capping layer 170 may cover the second electrode 150.

The encapsulation portion 300 may be on the capping layer 170. The encapsulation portion 300 may be on a light-emitting device to protect the light-emitting device from moisture and/or oxygen. The encapsulation portion 300 may include: an inorganic film including silicon nitride (SiNx), silicon oxide (SiOx), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, and/or the like), an epoxy-based resin (for example, aliphatic glycidyl ether (AGE), and/or the like), or any combination thereof; or any combination of the inorganic films and the organic films.

FIG. 3 shows a cross-sectional view showing a light-emitting apparatus according to an embodiment of the present disclosure.

The light-emitting apparatus of FIG. 3 is substantially the same as the light-emitting apparatus of FIG. 2, except that a light-shielding pattern 500 and a functional region 400 are additionally on the encapsulation portion 300. The functional region 400 may be i) a color filter area, ii) a color conversion area, or iii) a combination of the color filter area and the color conversion area. In an embodiment, the light-emitting device included in the light-emitting apparatus of FIG. 3 may be a tandem light-emitting device.

### Manufacturing Method

The layers included in the hole transport region, the emission layer, and the layers included in the electron transport region may be formed in a certain region by using various suitable methods such as vacuum deposition, spin coating, casting, Langmuir-Blodgett (LB) deposition, ink-jet printing, laser-printing, laser-induced thermal imaging, and/or the like.

When layers constituting the hole transport region, an emission layer, and layers constituting the electron transport region are formed by vacuum deposition, the deposition may be performed at a deposition temperature of about 100° C. to about 500° C., a vacuum degree of about 10⁻⁸ torr to about 10⁻³ torr, and a deposition speed of about 0.01 Å/sec to about 100 Å/sec, depending on a material to be included in a layer to be formed and the structure of a layer to be formed.

### DEFINITION OF TERMS

The term “C₃-C₆₀ carbocyclic group,” as used herein, refers to a cyclic group consisting of carbon only as a ring-forming atom and having three to sixty carbon atoms, and the term “C₁-C₆₀ heterocyclic group,” as used herein, refers to a cyclic group that has one to sixty carbon atoms and further has, in addition to carbon, a heteroatom as a ring-forming atom. The C₃-C₆₀ carbocyclic group and the C₁-C₆₀ heterocyclic group may each be a monocyclic group consisting of one ring or a polycyclic group in which two or more rings are condensed together with each other. For example, the C₁-C₆₀ heterocyclic group has 3 to 61 ring-forming atoms.

The term “cyclic group,” as used herein, may include the C₃-C₆₀ carbocyclic group, and the C₁-C₆₀ heterocyclic group.

The term “π electron-rich C₃-C₆₀ cyclic group,” as used herein, refers to a cyclic group that has three to sixty carbon atoms and does not include *—N═*′ as a ring-forming moiety, and the term “π electron-deficient nitrogen-containing C₁-C₆₀ cyclic group,” as used herein, refers to a heterocyclic group that has one to sixty carbon atoms and includes *—N═*′ as a ring-forming moiety.

For example,

the C₃-C₆₀ carbocyclic group may be i) a T1 group or ii) a condensed cyclic group in which two or more T1 groups are condensed together with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),

the C₁-C₆₀ heterocyclic group may be i) a T2 group, ii) a condensed cyclic group in which two or more T2 groups are condensed together with each other, or iii) a condensed cyclic group in which at least one T2 group and at least one T1 group are condensed together with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),

the 7 electron-rich C₃-C₆₀ cyclic group may be i) a T1 group, ii) a condensed cyclic group in which two or more T1 groups are condensed together with each other, iii) a T3 group, iv) a condensed cyclic group in which two or more T3 groups are condensed together with each other, or v) a condensed cyclic group in which at least one T3 group and at least one T1 group are condensed together with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, etc.),

the π electron-deficient nitrogen-containing C₁-C₆₀ cyclic group may be i) a T₄ group, ii) a condensed cyclic group in which two or more T₄ groups are condensed together with each other, iii) a condensed cyclic group in which at least one T₄ group and at least one T1 group are condensed together with each other, iv) a condensed cyclic group in which at least one T₄ group and at least one T3 group are condensed together with each other, or v) a condensed cyclic group in which at least one T₄ group, at least one T1 group, and at least one T3 group are condensed together with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.),

the T1 group may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or a bicyclo[2.2.1]heptane) group, a norbornene group, a bicyclo[1.1.1]pentane group, a bicyclo[2.1.1]hexane group, a bicyclo[2.2.2]octane group, or a benzene group,

the T2 group may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group,

the T3 group may be a furan group, a thiophene group, a 1H-pyrrole group, a silole group, or a borole group, and

the T4 group may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “the cyclic group,” “the C₃-C₆₀ carbocyclic group,” “the C₁-C₆₀ heterocyclic group,” “the π electron-rich C₃-C₆₀ cyclic group,” or “the π electron-deficient nitrogen-containing C₁-C₆₀ cyclic group,” as used herein, refer to a group condensed to any cyclic group, a monovalent group, or a polyvalent group (for example, a divalent group, a trivalent group, a tetravalent group, etc.) according to the structure of a formula for which the corresponding term is used. For example, the “benzene group” may be a benzo group, a phenyl group, a phenylene group, or the like, which may be easily understood by one of ordinary skill in the art according to the structure of a formula including the “benzene group.”

Examples of the monovalent C₃-C₆₀ carbocyclic group and the monovalent C₁-C₆₀ heterocyclic group are a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group. Examples of the divalent C₃-C₆₀ carbocyclic group and the monovalent C₁-C₆₀ heterocyclic group are a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a substituted or unsubstituted divalent non-aromatic condensed heteropolycyclic group.

The term “C₁-C₆₀ alkyl group,” as used herein, refers to a linear or branched aliphatic hydrocarbon monovalent group that has one to sixty carbon atoms, and examples thereof include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group. The term “C₁-C₆₀ alkylene group,” as used herein, refers to a divalent group having substantially the same structure as the C₁-C₆₀ alkyl group.

The term “C₂-C₆₀ alkenyl group,” as used herein, refers to a monovalent hydrocarbon group having at least one carbon-carbon double bond at a main chain (e.g., in the middle) or at a terminal end (e.g., the terminus) of the C₂-C₆₀ alkyl group, and examples thereof are an ethenyl group, a propenyl group, and a butenyl group. The term “C₂-C₆₀ alkenylene group,” as used herein, refers to a divalent group having substantially the same structure as the C₂-C₆₀ alkenyl group.

The term “C₂-C₆₀ alkynyl group,” as used herein, refers to a monovalent hydrocarbon group having at least one carbon-carbon triple bond at a main chain (e.g., in the middle) or at a terminal end (e.g., the terminus) of the C₂-C₆₀ alkyl group, and examples thereof include an ethynyl group and a propynyl group. The term “C₂-C₆₀ alkynylene group,” as used herein, refers to a divalent group having substantially the same structure as the C₂-C₆₀ alkynyl group.

The term “C₁-C₆₀ alkoxy group,” as used herein, refers to a monovalent group represented by —OA₁₀₁ (wherein A₁₀₁ is the C₁-C₆₀ alkyl group), and examples thereof include a methoxy group, an ethoxy group, and an isopropyloxy group.

The term “C₃-C₁₀ cycloalkyl group,” as used herein, refers to a monovalent saturated hydrocarbon cyclic group having 3 to 10 carbon atoms, and examples thereof are a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, and a bicyclo[2.2.2]octyl group. The term “C₃-C₁₀ cycloalkylene group,” as used herein, refers to a divalent group having substantially the same structure as the C₃-C₁₀ cycloalkyl group.

The term “C₁-C₁₀ heterocycloalkyl group,” as used herein, refers to a monovalent cyclic group of 1 to 10 carbon atoms, further including, in addition to carbon atoms, at least one heteroatom, as ring-forming atoms, and examples thereof include a 1,2,3,4-oxatriazolidinyl group, a tetrahydrofuranyl group, and a tetrahydrothiophenyl group. The term “C₁-C₁₀ heterocycloalkylene group,” as used herein, refers to a divalent group having substantially the same structure as the C₁-C₁₀ heterocycloalkyl group.

The term “C₃-C₁₀ cycloalkenyl group,” as used herein, refers to a monovalent cyclic group that has three to ten carbon atoms and at least one carbon-carbon double bond in the ring thereof or has no aromaticity (e.g., is not aromatic), and examples thereof include a cyclopentenyl group, a cyclohexenyl group, and a cycloheptenyl group. The term “C₃-C₁₀ cycloalkenylene group,” as used herein, refers to a divalent group having substantially the same structure as the C₃-C₁₀ cycloalkenyl group.

The term “C₁-C₁₀ heterocycloalkenyl group,” as used herein, refers to a monovalent cyclic group of 1 to 10 carbon atoms, further including, in addition to carbon atoms, at least one heteroatom, as ring-forming atoms, and having at least one carbon-carbon double bond in the cyclic structure thereof. Examples of the C₁-C₁₀ heterocycloalkenyl group include a 4,5-dihydro-1,2,3,4-oxatriazolyl group, a 2,3-dihydrofuranyl group, and a 2,3-dihydrothiophenyl group. The term “C₁-C₁₀ heterocycloalkenylene group,” as used herein, refers to a divalent group having substantially the same structure as the C₁-C₁₀ heterocycloalkenyl group.

The term “C₆-C₆₀ aryl group,” as used herein, refers to a monovalent group having a carbocyclic aromatic system of 6 to 60 carbon atoms, and the term “C₆-C₆₀ arylene group,” as used herein, refers to a divalent group having a carbocyclic aromatic system of 6 to 60 carbon atoms. Examples of the C₆-C₆₀ aryl group are a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, and an ovalenyl group. When the C₆-C₆₀ aryl group and the C₆-C₆₀ arylene group each include two or more rings, the rings may be condensed together with each other.

The term “C₁-C₆₀ heteroaryl group,” as used herein, refers to a monovalent group having a heterocyclic aromatic system of 1 to 60 carbon atoms, further including, in addition to carbon atoms, at least one heteroatom, as ring-forming atoms. The term “C₁-C₆₀ heteroarylene group,” as used herein, refers to a divalent group having a heterocyclic aromatic system of 1 to 60 carbon atoms, further including, in addition to carbon atoms, at least one heteroatom, as ring-forming atoms. Examples of the C₁-C₆₀ heteroaryl group are a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group. When the C₁-C₆₀ heteroaryl group and the C₁-C₆₀ heteroarylene group each include two or more rings, the rings may be condensed together with each other.

The term “monovalent non-aromatic condensed polycyclic group,” as used herein, refers to a monovalent group (for example, having 8 to 60 carbon atoms) having two or more rings condensed to each other, only carbon atoms as ring-forming atoms, and no aromaticity in its entire molecular structure (e.g., is not aromatic when considered as a whole). Examples of the monovalent non-aromatic condensed polycyclic group are an indenyl group, a fluorenyl group, a spiro-bifluorenyl group, a benzofluorenyl group, an indenophenanthrenyl group, and an indeno anthracenyl group. The term “divalent non-aromatic condensed polycyclic group,” as used herein, refers to a divalent group having substantially the same structure as the monovalent non-aromatic condensed polycyclic group described above.

The term “monovalent non-aromatic condensed heteropolycyclic group,” as used herein, refers to a monovalent group (for example, having 1 to 60 carbon atoms) having two or more rings condensed to each other, further including, in addition to carbon atoms, at least one heteroatom, as ring-forming atoms, and having no aromaticity in its entire molecular structure (e.g., is not aromatic when considered as a whole). Examples of the monovalent non-aromatic condensed heteropolycyclic group include a pyrrolyl group, a thiophenyl group, a furanyl group, an indolyl group, a benzoindolyl group, a naphtho indolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indenocarbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, and a benzothienodibenzothiophenyl group. The term “divalent non-aromatic condensed heteropolycyclic group,” as used herein, refers to a divalent group having substantially the same structure as the monovalent non-aromatic condensed heteropolycyclic group described above.

The term “C₆-C₆₀ aryloxy group,” as used herein, indicates —OA₁₀₂ (wherein A₁₀₂ is a C₆-C₆₀ aryl group), and the term “C₆-C₆₀ arylthio group,” as used herein, indicates —SA₁₀₃ (wherein A₁₀₃ is a C₆-C₆₀ aryl group).

The term “C₇-C₆₀ aryl alkyl group,” as used herein, refers to -A₁₀₄A₁₀₆ (where A₁₀₄ may be a C₁-C₅₄ alkylene group, and A₁₀₅ may be a C₆-C₅₉ aryl group), and the term C₂-C₆₀ heteroaryl alkyl group,” as used herein, refers to -A₁₀₆A₁₀₇ (where A₁₀₆ may be a C₁-C₆₀ alkylene group, and A₁₀₇ may be a C₁-C₆₀ heteroaryl group).

The term “R₁₀ₐ,” as used herein, refers to:

deuterium, —F, —Cl, —Br, —I, a hydroxyl group, a cyano group, or a nitro group,

a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof,

a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, or a C₂-C₆₀ heteroaryl alkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ aryl alkyl group, a C₂-C₆₀ heteroaryl alkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),

wherein Q₁ to Q₃, Q₁₁ to Q₁₃, Q₂₁ to Q₂₃ and Q₃₁ to Q₃₃ used herein may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof; a C₇-C₆₀ aryl alkyl group; or a C₂-C₆₀ heteroaryl alkyl group.

The term “heteroatom,” as used herein, refers to any atom other than a carbon atom. Examples of the heteroatom include O, S, N, P, Si, B, Ge, Se, and any combinations thereof.

The term “third-row transition metal,” as used herein, includes hafnium (Hf), tantalum (Ta), tungsten (W), rhenium (Re), osmium (Os), iridium (Ir), platinum (Pt), gold (Au), and the like.

“Ph,” as used herein, refers to a phenyl group, “Me,” as used herein, refers to a methyl group, “Et,” as used herein, refers to an ethyl group, “tert-Bu” or “But,” as used herein, refers to a tert-butyl group, and “OMe,” as used herein, refers to a methoxy group.

The term “biphenyl group,” as used herein, refers to “a phenyl group substituted with a phenyl group.” In other words, the “biphenyl group” is a substituted phenyl group having a C₆-C₆₀ aryl group as a substituent.

The term “terphenyl group,” as used herein, refers to “a phenyl group substituted with a biphenyl group”. In other words, the “terphenyl group” is a substituted phenyl group having, as a substituent, a C₆-C₆₀ aryl group substituted with a C₆-C₆₀ aryl group.

* and *′, as used herein, unless defined otherwise, each refer to a binding site to a neighboring atom in a corresponding formula or moiety.

Hereinafter, compounds according to embodiments and light-emitting devices according to embodiments will be described in more detail with reference to the following synthesis examples and examples. The wording “B was used instead of A” used in describing Synthesis Examples means that an identical molar equivalent of B was used in place of A.

### EXAMPLES

### Synthesis Example 1

### Synthesis Example 1: Synthesis of Compound 1

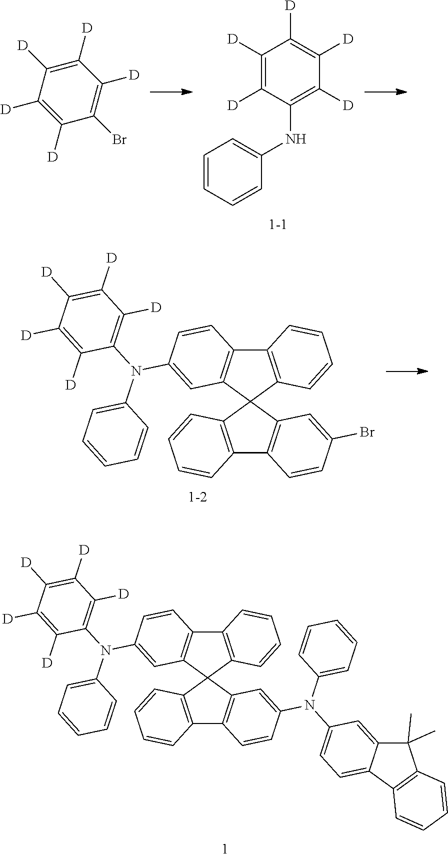 [SMILES: [2H]c1c([2H])c([2H])c(Br)c([2H])c1[2H] ; [2H]c1c([2H])c([2H])c(Nc2ccccc2)c([2H])c1[2H] ; [2H]c1c([2H])c([2H])c(N(c2ccccc2)c2ccc3c(c2)C2(c4ccccc4-c4ccc(Br)cc42)c2ccccc2-3)c([2H])c1[2H] ; [2H]c1c([2H])c([2H])c(N(c2ccccc2)c2ccc3c(c2)C2(c4ccccc4-c4ccc(N(c5ccccc5)c5ccc6c(c5)C(C)(C)c5ccccc5-6)cc42)c2ccccc2-3)c([2H])c1[2H]]

### Synthesis of Intermediate 1-1

1.62 g (10.0 mmol) of 1-bromobenzene-2,3,4,5,6-d₅, 1.2 ml (13 mmol) of aniline, 0.46 g (0.5 mmol) of Pd₂dba₃ (tris(dibenzylideneacetone)dipalladium(0)), 2.00 g (1 mmol) of P(t-Bu)₃, and 2.90 g (30 mmol) of sodium tert-butoxide were dissolved in 120 ml of toluene and then stirred at 80° C. for 3 hours. The resultant reaction solution was cooled to room temperature, 40 ml of water was added to the reaction solution, followed by extraction three times with 50 ml of ethyl ether. The collected ethyl ether was dried with MgSO₄, the solvent was evaporated, and the obtained residue was separated and purified by silica gel column chromatography to obtain 0.96 g of Intermediate 1-1 (yield 55%). The formation of Intermediate 1-1 was confirmed by LC-MS. C12H6D5N: M+: 174.1

### Synthesis of Intermediate 1-2

0.96 g (5.5 mmol) of Intermediate 1-1, 3.16 g (6.7 mmol) of 2,2′-dibromo-9,9′-spirobi[fluorene], 0.31 g (0.55 mmol) of dppf (1,1′-bis(diphenylphosphino)ferrocene), 0.25 g (0.28 mmol) of Pd₂dba₃ (tris(dibenzylideneacetone)dipalladium(0)), and sodium 1.6 g (16.6 mmol) of tert-butoxide were dissolved in 100 ml of toluene and then stirred at 80° C. for 3 hours. The resultant reaction solution was cooled to room temperature, 30 ml of water was added to the reaction solution, followed by extraction three times with 40 ml of ethyl ether. The collected ethyl ether was dried with MgSO₄, the solvent was evaporated, and the obtained residue was separated and purified by silica gel column chromatography to obtain 1.05 g of Intermediate 1-2 (yield 28%). The formation of Intermediate 1-2 was confirmed by LC-MS. C37H19D5BrN M+: 566.1

### Synthesis of Compound 1

10.5 g (18.5 mmol) of Intermediate 1-2, 6.9 g (24 mmol) of 9,9-dimethyl-N-phenyl-9H-fluoren-2-amine, 0.85 g (0.93 mmol) of Pd₂dba₃ (tris(dibenzylideneacetone)dipalladium(0)), 0.40 g (1.85 mmol) of P(t-Bu)₃, and 5.4 g (55 mmol) of sodium tert-butoxide were dissolved in 120 ml of toluene and then stirred at 80° C. for 3 hours. The resultant reaction solution was cooled to room temperature, 40 ml of water was added to the reaction solution, followed by extraction three times with 50 ml of ethyl ether. The collected ethyl ether was dried with MgSO₄, the solvent was evaporated, and the obtained residue was separated and purified by silica gel column chromatography to obtain 8.6 g of Compound 1 (yield 60%). The formation of Compound 1 was confirmed by fast atom bombardment mass spectrometry (MS/FAB) and proton nuclear magnetic resonance (1H NMR) spectroscopy.

### Synthesis Example 2: Synthesis of Compound 2

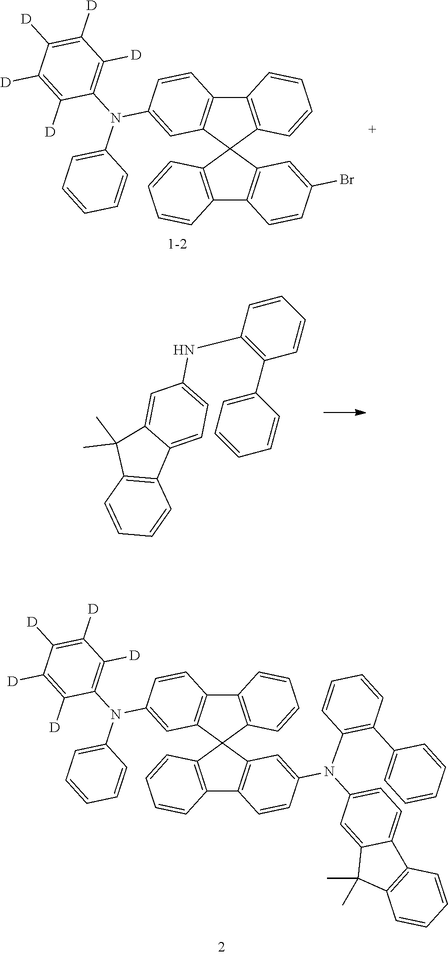 [SMILES: [2H]c1c([2H])c([2H])c(N(c2ccccc2)c2ccc3c(c2)C2(c4ccccc4-c4ccc(Br)cc42)c2ccccc2-3)c([2H])c1[2H] ; CC1(C)c2ccccc2-c2ccc(Nc3ccccc3-c3ccccc3)cc21 ; [2H]c1c([2H])c([2H])c(N(c2ccccc2)c2ccc3c(c2)C2(c4ccccc4-c4ccc(N(c5ccc6c(c5)C(C)(C)c5ccccc5-6)c5ccccc5-c5ccccc5)cc42)c2ccccc2-3)c([2H])c1[2H]]

Compound 2 was synthesized using substantially the same method as in synthesizing Compound 1, except that N-([1,1′-biphenyl]-2-yl)-9,9-dimethyl-9H-fluoren-2-amine was used instead of 9,9-dimethyl-N-phenyl-9H-fluoren-2-amine. The formation of Compound 2 was confirmed by MS/FAB and 1H NMR.

### Synthesis Example 3: Synthesis of Compound 33

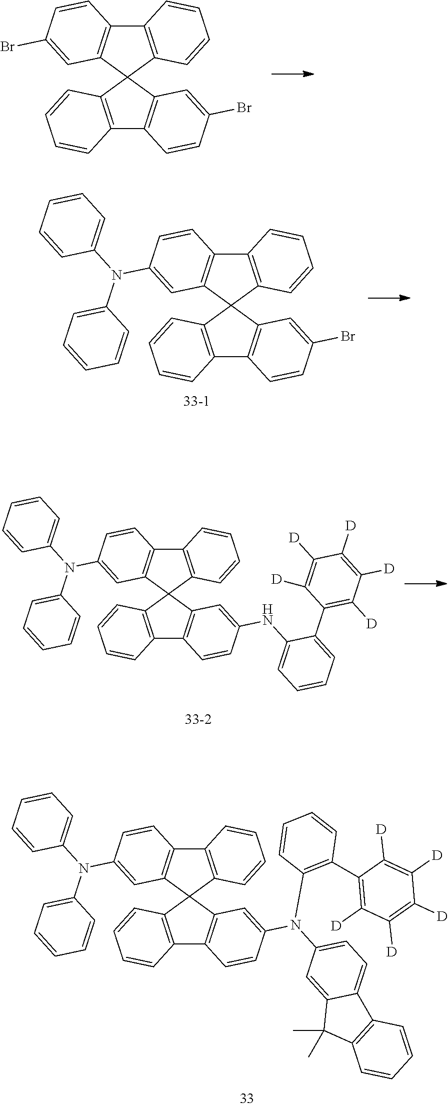

### Synthesis of Intermediate 33-1

21.5 g (45 mmol) of 2,2′-dibromo-9,9′-spirobi[fluorene], 6.34 g (37.5 mmol) of diphenylamine, 1.7 g (1.9 mmol) of Pd₂dba₃ (tris(dibenzylideneacetone)dipalladium(0)), 2.10 g (3.75 mmol) of dppf (1,1′-bis(diphenylphosphino) ferrocene) and 11.0 g (111 mmol) of sodium tert-butoxide were dissolved in 500 ml of toluene and then stirred at 80° C. for 3 hours. The resultant reaction solution was cooled to room temperature, 100 ml of water was added to the reaction solution, followed by extraction three times with 120 ml of ethyl ether. The collected ethyl ether was dried with MgSO₄, the solvent was evaporated, and the obtained residue was separated and purified by silica gel column chromatography to obtain 8.70 g of Intermediate 33-1 (yield 34%). The formation of Intermediate 33-1 was confirmed by LC-MS. C37H24BrN M+: 561.1

### Synthesis of Intermediate 33-2

8.70 g (15.5 mmol) of Intermediate 33-1, 3.5 g (20.2 mmol) of [1,1′-biphenyl]-2′,3′,4′,5′,6′-d5-2-amine, 0.71 g (0.78 mmol) of Pd₂dba₃ (tris(dibenzylideneacetone)dipalladium(0)), 0.31 g (1.55 mmol) of P(t-Bu)₃, and 4.5 g (46.5 mmol) of sodium tert-butoxide were dissolved in 120 ml of toluene and then stirred at 80° C. for 3 hours. The resultant reaction solution was cooled to room temperature, 40 ml of water was added to the reaction solution, followed by extraction three times with 50 ml of ethyl ether. The collected ethyl ether was dried with MgSO₄, the solvent was evaporated, and the obtained residue was separated and purified by silica gel column chromatography to obtain 3.40 g of Intermediate 33-2 (yield 33%). The formation of Intermediate 33-2 was confirmed by LC-MS. C49H29D5N2 M+: 655.3

### Synthesis of Compound 33

3.4 g (5.2 mmol) of Intermediate 33-2, 1.6 g (5.8 mmol) of 2-bromo-9,9-dimethyl-9H-fluorene, 0.24 g (0.26 mmol) of Pd₂dba₃ (tris(dibenzylideneacetone)dipalladium(0)), 0.11 g (0.52 mmol) of P(t-Bu)₃, and 1.5 g (15.6 mmol) of sodium tert-butoxide were dissolved in 100 ml of toluene and then stirred at 80° C. for 3 hours. The resultant reaction solution was cooled to room temperature, 40 ml of water was added to the reaction solution, followed by extraction three times with 50 ml of ethyl ether. The collected ethyl ether was dried with MgSO₄, the solvent was evaporated, and the obtained residue was separated and purified by silica gel column chromatography to obtain 3.4 g of Compound 33 (yield 77%). The formation of Compound 33 was confirmed by MS/FAB and 1H NMR.

### Synthesis Example 4: Synthesis of Compound 73

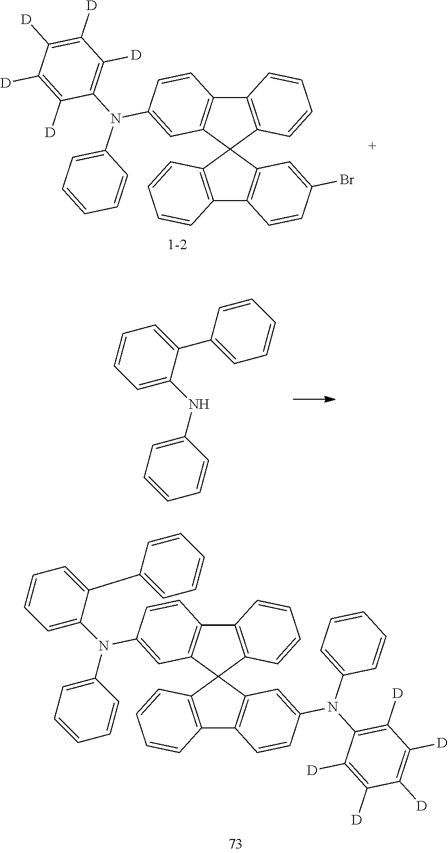

Compound 73 was synthesized using substantially the same method as in synthesizing Compound 1, except that N-phenyl-[1,1′-biphenyl]-2-amine was used instead of 9,9-dimethyl-N-phenyl-9H-fluoren-2-amine. The formation of Compound 73 was confirmed by MS/FAB and 1H NMR.

### Synthesis Example 5: Synthesis of Compound 84

Compound 84 was synthesized using substantially the same method as in synthesizing Compound 1, except that N,9-diphenyl-9H-carbazol-2-amine was used instead of 9,9-dimethyl-N-phenyl-9H-fluoren-2-amine. The formation of Compound 84 was confirmed by MS/FAB and 1H NMR.

### Synthesis Example 6: Synthesis of Compound 121

Compound 121 was synthesized using substantially the same method as in synthesizing Compound 1, except that N-phenyl-[1,1′:3′,1″-terphenyl]-2′-amine was used instead of 9,9-dimethyl-N-phenyl-9H-fluoren-2-amine. The formation of Compound 121 was confirmed by MS/FAB and 1H NMR.

### Synthesis Example 7: Synthesis of Compound 156

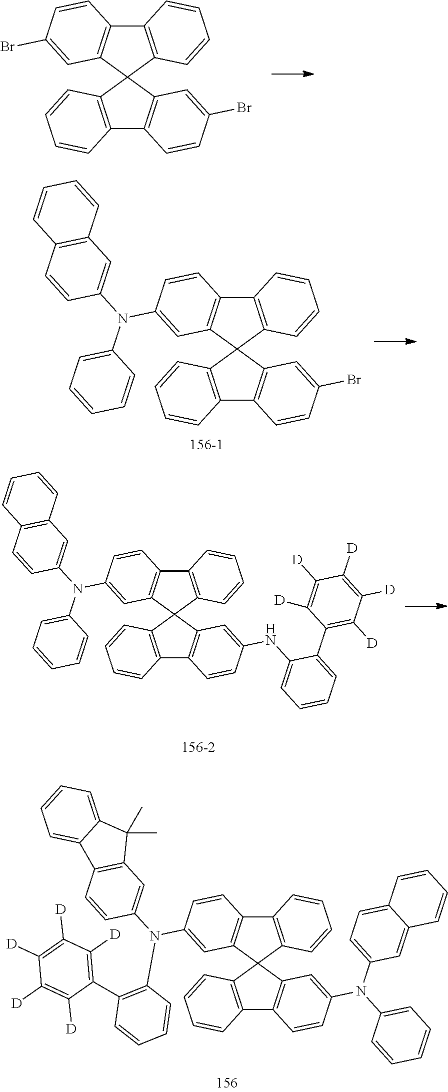

### Synthesis of Intermediate 156-1

21.5 g (45 mmol) of 2,2′-dibromo-9,9′-spirobi[fluorene], 8.22 g (37.5 mmol) of N-phenylnaphthalen-2-amine, 1.7 g (1.9 mmol) of Pd₂dba₃ (tris(dibenzylideneacetone)dipalladium(0)), 2.10 g (3.75 mmol) of dppf (1,1′-bis(diphenylphosphino) ferrocene) and 11.0 g (111 mmol) of sodium tert-butoxide were dissolved in 500 ml of toluene and then stirred at 80° C. for 3 hours. The resultant reaction solution was cooled to room temperature, 100 ml of water was added to the reaction solution, followed by extraction three times with 120 ml of ethyl ether. The collected ethyl ether was dried with MgSO₄, the solvent was evaporated, and the obtained residue was separated and purified by silica gel column chromatography to obtain 9.35 g of Intermediate 156-1 (yield 34%). The formation of Intermediate 156-1 was confirmed by LC-MS. C37H24BrN M+: 611.1

### Synthesis of Intermediate 156-2

9.35 g (15.5 mmol) of Intermediate 156-1, 3.5 g (20.2 mmol) of [1,1′-biphenyl]-2′,3′,4′,5′,6′-d5-2-amine, 0.71 g (0.78 mmol) of Pd₂dba₃ (tris(dibenzylideneacetone)dipalladium(0)), 0.31 g (1.55 mmol) of P(t-Bu)₃, and 4.5 g (46.5 mmol) of sodium tert-butoxide were dissolved in 120 ml of toluene and then stirred at 80° C. for 3 hours. The resultant reaction solution was cooled to room temperature, 40 ml of water was added to the reaction solution, followed by extraction three times with 50 ml of ethyl ether. The collected ethyl ether was dried with MgSO₄, the solvent was evaporated, and the obtained residue was separated and purified by silica gel column chromatography to obtain 3.6 g of Intermediate 156-2 (yield 33%). The formation of Intermediate 156-2 was confirmed by LC-MS. C49H29D5N2 M+: 705.3

### Synthesis of Compound 156

3.6 g (5.2 mmol) of Intermediate 156-2, 1.6 g (5.8 mmol) of 2-bromo-9,9-dimethyl-9H-fluorene, 0.24 g (0.26 mmol) of Pd₂dba₃ (tris(dibenzylideneacetone)dipalladium(0)), 0.11 g (0.52 mmol) of P(t-Bu)₃, and 1.5 g (15.6 mmol) of sodium tert-butoxide were dissolved in 100 ml of toluene and then stirred at 80° C. for 3 hours. The resultant reaction solution was cooled to room temperature, 40 ml of water was added to the reaction solution, followed by extraction three times with 50 ml of ethyl ether. The collected ethyl ether was dried with MgSO₄, the solvent was evaporated, and the obtained residue was separated and purified by silica gel column chromatography to obtain 3.6 g of Compound 156 (yield 77%). The formation of Compound 156 was confirmed by MS/FAB and 1H NMR.

### Synthesis Example 8: Synthesis of Compound 221

Compound 221 was synthesized using substantially the same method as in synthesizing Compound 1, except that diphenylamine was used instead of 9,9-dimethyl-N-phenyl-9H-fluoren-2-amine. The formation of Compound 221 was confirmed by MS/FAB and 1H NMR.

### Synthesis Example 9: Synthesis of Compound 396

Compound 396 was synthesized using substantially the same method as in synthesizing Compound 1, except that 2,2′-dibromo-3-phenyl-9,9′-spirobi[fluorene] was used instead of 2,2′-dibromo-9,9′-spirobi[fluorene]. The formation of Compound 396 was confirmed by MS/FAB and 1H NMR.

### Synthesis Example 10: Synthesis of Comparative Compound 1

Comparative Compound 1 was synthesized using substantially the same method as in synthesizing Compound 33, except that 2,2′-diphenylamine was used instead of [1,1′-biphenyl]-2′,3′,4′,5′,6′-d5-2-amine. The formation of Comparative Compound 1 was confirmed by MS/FAB and 1H NMR.

### Synthesis Example 11: Synthesis of Comparative Compound 2

Comparative Compound 2 was synthesized using substantially the same method as in synthesizing Compound 1, except that 2,2′-2,2′-dibromo-9,9′-spirobi[fluorene]-toluene-2,3,4,5,6-d5 was used instead of 2,2′-dibromo-9,9′-spirobi[fluorene]. The formation of Comparative Compound 2 was confirmed by MS/FAB and 1H NMR.

### Synthesis Example 12: Synthesis of Comparative Compound 3

Comparative Compound 3 was synthesized using substantially the same method as in synthesizing Compound 33, except that 9,9-dimethyl-N-phenyl-9H-fluoren-3-amine was used instead of [1,1′-biphenyl]-2′,3′,4′,5′,6′-d5-2-amine. The formation of Comparative Compound 3 was confirmed by MS/FAB and 1H NMR.

Table 1 shows the results of analyzing the Compounds synthesized according to the Synthesis Examples and the Comparative Compounds with MS/FAB and ¹H NMR.

TABLE 1

| | | MS/FAB | |
|---|---|---|---|
| Compound | ¹H NMR (CDCl₃, 400 MHz) | found | calc. |
| Compound | 7.90-7.85 (m, 6H), 7.55 (d, 2H), 7.38-7.00 (m, | 771.50 | 771.37 |
| 1 | 23H), 1.69 (s, 6H) | | |
| Compound | 8.10 (d, 1H), 7.90-7.85 (m, 6H), 7.55 (d, 2H), | 847.65 | 847.40 |
| 2 | 7.43-7.00 (m, 26H), 1.69 (s, 6H) | | |
| Compound | 8.10 (d, 1H), 7.90-7.85 (m, 6H), 7.55 (d, 2H), | 847.72 | 847.40 |
| 33 | 7.43-7.00 (m, 26H), 1.69 (s, 6H) | | |
| Compound | 8.10 (d, 1H), 7.90-7.85 (m, 4H), 7.55 (d, 1H), | 731.56 | 731.33 |
| 73 | 7.45-7.00 (m, 27H) | | |
| Compound | 8.55 (d, 1H), 8.24 (d, 1H), 7.94-7.85 (m, 5H), | 820.75 | 820.36 |
| 84 | 7.62-7.00 (m, 29H) | | |
| Compound | 8.20 (d, 2H), 7.90-7.85 (m, 4H), 7.55 (d, 1H), | 807.65 | 807.37 |
| 121 | 7.45-7.00 (m, 30H) | | |
| Compound | 8.10 (d, 1H), 7.90-7.85 (m, 6H), 7.55 (d, 1H), | 847.91 | 847.40 |
| 156 | 7.45-7.00 (m, 33H) | | |
| Compound | 7.90-7.85 (m, 4H), 7.55 (d, 1H), 7.45-7.00 (m, | 655.87 | 655.30 |
| 221 | 24H) | | |
| Compound | 8.14 (s, 1H), 7.90-7.85 (m, 5H), 7.55-7.45 (m, | 847.56 | 847.40 |
| 396 | 3H), 7.45-7.00 (m, 32H) | | |
| NPB | 8.22 (t, 2H), 8.15 (t, 2H), 7.81 (d, 2H), 7.63-7.49 | 588.42 | 588.26 |
| | (m, 12H), 7.37 (d, 4H), 7.24 (m, 4H), 7.08-7.00 | | |
| | (m, 6H) | | |
| Comparative | 7.89-7.85 (m, 4H), 7.50 (d, 2H), 7.33-7.24 (m, | 650.82 | 650.27 |
| Compound | 14H), 7.11-7.00 (m, 14H) | | |
| 1 | | | |
| Comparative | 7.89-7.85 (m, 4H), 7.50 (d, 2H), 7.33-7.24 (m, | 731.94 | 731.33 |
| Compound | 14H), 7.08-7.00 (m, 13H) | | |
| 2 | | | |
| Comparative | 7.90-7.85 (m, 4H), 7.62 (s, 1H), 7.55-7.00 (m, | 766.42 | 766.33 |
| Compound | 37H) | | |
| 3 | | | |

### Evaluation Example 1

The LUMO and HOMO values of the Compounds of the Synthesis Example and the Comparative Compounds were measured using the method described in Table 2. The results are shown in Table 3.

TABLE 2

| | |
|---|---|
| HOMO energy level | By using cyclic voltammetry (CV) (electrolyte: 0.1M |
| evaluation method | Bu₄NPF₆/solvent: dimethylforamide (DMF)/electrode: 3- |
| | electrode system (working electrode: GC, reference |
| | electrode: Ag/AgCl, and auxiliary electrode: Pt)), the |
| | potential (V)-current (A) graph of each compound was |
| | obtained, and then, from the oxidation onset of the graph, |
| | the HOMO energy level of each compound was calculated. |
| LUMO energy level | By using cyclic voltammetry (CV) (electrolyte: 0.1M |
| evaluation method | Bu₄NPF₆/solvent: dimethylforamide (DMF)/electrode: 3- |
| | electrode system (working electrode: GC, reference |
| | electrode: Ag/AgCl, and auxiliary electrode: Pt)), the |
| | potential (V)-current (A) graph of each compound was |
| | obtained, and then, from the reduction onset of the graph, |
| | the LUMO energy level of each compound was calculated. |

TABLE 3

| Compound | HOMO | LUMO |
|---|---|---|
| No. | (eV) | (eV) |
| Compound | −5.18 | −1.84 |
| 1 | | |
| Compound | −5.16 | −1.85 |
| 2 | | |
| Compound | −5.20 | −1.90 |
| 33 | | |
| Compound | −5.15 | −1.87 |
| 73 | | |
| Compound | −5.13 | −1.88 |
| 84 | | |
| Compound | −5.19 | −1.95 |
| 121 | | |
| Compound | −5.12 | −1.82 |
| 156 | | |
| Compound | −5.13 | −1.86 |
| 221 | | |
| Compound | −5.14 | −1.90 |
| 396 | | |
| NPB | −5.11 | −1.84 |
| Comparative | −5.12 | −1.82 |
| Compound | | |
| 1 | | |
| Comparative | −5.14 | −1.85 |
| Compound | | |
| 2 | | |
| Comparative | −5.17 | −1.85 |
| Compound | | |
| 3 | | |

The structures of Comparative Compounds 1 to 3 of Table 3 are shown below.

Comparative Compound 1:

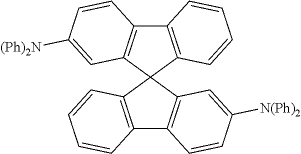

Comparative Compound 2:

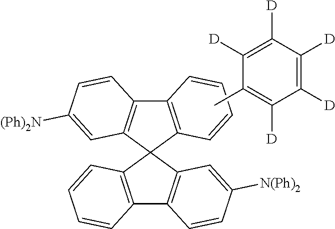

Comparative Compound 3:

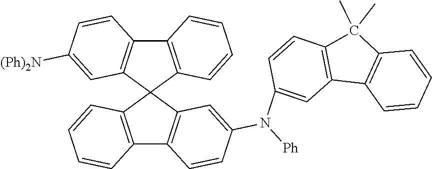

### Example 1

As an anode, a glass substrate with an ITO deposited thereon was cut to a size of 50 mm×50 mm×0.7 mm, sonicated with isopropyl alcohol and pure water, each for 5 minutes, and then cleaned by exposure to ultraviolet rays and ozone for 30 minutes. Then, the glass substrate was provided to a vacuum deposition apparatus.

Compound 2-TNATA was vacuum-deposited on the ITO substrate to form a hole injection layer having a thickness of 600 Å, and then, Compound 1 was vacuum-deposited on the hole injection layer to form a hole transport layer having a thickness of 300 Å.

9,10-di(naphthalen-2-yl)anthracene (hereinafter, referred to as DNA) which is a blue fluorescence host and 4,4′-bis[2-(4-(N,N-diphenylamino)phenyl)vinyl]biphenyl (hereinafter, referred to as DPAVBi) which is a blue phosphorescence dopant compound were co-deposited on the hole transport layer to a weight ratio of 98:2 to form an emission layer having a thickness of 300 Å.

Alq3 was deposited on the emission layer to form an electron transport layer having a thickness of 300 Å, LiF, which is a halogenated alkali metal, was deposited on the electron transport layer to form an electron injection layer having a thickness of 10 Å, and Al was vacuum-deposited on the electron injection layer to form a LiF/AI electrode having a thickness of 3,000 Å (cathode), thereby completing the manufacture of a light-emitting device.

### Examples 2 to 9

A light-emitting device was manufactured in substantially the same manner as in Example 1, except that a different hole transport material as in Table 4 was used.

### Comparative Examples 1 to 4

A light-emitting device was manufactured in substantially the same manner as in Example 1, except that a different hole transport material as in Table 4 was used.

Voltage was applied to the light-emitting device manufactured according to Examples 1 to 9 and Comparative Examples 1 to 3 such that the light-emitting devices have a current density of 50 mA/cm². The driving voltage (V), luminance (cd/m²), luminescence efficiency (cd/A), emission color, emission wavelength (nm), and half lifespan (hr@100 z mA/cm²) were each measured by using a Keithley MU 236 and a luminance meter PR650, and the result thereof is shown in Table 4.

TABLE 4

| | | Driving | Current | | | | Half lifespan |
|---|---|---|---|---|---|---|---|
| | Hole transport | voltage | density | Luminance | Efficiency | Emission | (hr @ 100 |
| | material | (V) | (mA) | (cd/m²) | (cd/A) | color | mA/cm²) |
| Example 1 | Compound 1 | 4.98 | 50 | 3205 | 6.50 | Blue | 650 |
| Example 2 | Compound 2 | 5.01 | 50 | 3130 | 6.26 | Blue | 670 |
| Example 3 | Compound 33 | 5.04 | 50 | 3175 | 6.35 | Blue | 550 |
| Example 4 | Compound 73 | 4.99 | 50 | 3215 | 6.43 | Blue | 580 |
| Example 5 | Compound 84 | 5.10 | 50 | 3265 | 6.53 | Blue | 490 |
| Example 6 | Compound 121 | 5.20 | 50 | 3180 | 6.36 | Blue | 580 |
| Example 7 | Compound 156 | 4.95 | 50 | 3260 | 6.52 | Blue | 530 |
| Example 8 | Compound 221 | 5.13 | 50 | 3255 | 6.51 | Blue | 540 |
| Example 9 | Compound 396 | 5.07 | 50 | 3140 | 6.28 | Blue | 590 |
| Comparative | NPB | 7.01 | 50 | 2645 | 5.29 | Blue | 258 |
| Example 1 | | | | | | | |
| Comparative | Comparative | 5.30 | 50 | 2800 | 5.60 | Blue | 520 |
| Example 2 | Compound 1 | | | | | | |
| Comparative | Comparative | 5.53 | 50 | 2960 | 5.92 | Blue | 510 |
| Example 3 | Compound 2 | | | | | | |
| Comparative | Comparative | 5.42 | 50 | 3020 | 6.04 | Blue | 490 |
| Example 4 | Compound 3 | | | | | | |

From Table 4, it can be seen that the light-emitting device including the diamine compound according to each embodiment has excellent driving voltage (V), luminance (cd/m²), efficiency (cd/A), and half lifespan (hr@100 mA/cm²) compared to those of the light-emitting devices including hole transport materials according to Comparative Examples 1 to 4.

According to the one or more embodiments, the use of the diamine compound may enable the manufacture of a light-emitting device having high efficiency and a long lifespan and a high-quality electronic apparatus including the light-emitting device.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope of the present disclosure as defined by the following claims, and equivalents thereof.

What is claimed is:

1. A light-emitting device comprising:
a first electrode,
a second electrode facing the first electrode; and
an interlayer between the first electrode and the second electrode and including an emission layer, wherein:
the light-emitting device further comprises a diamine compound represented by Formula 1: 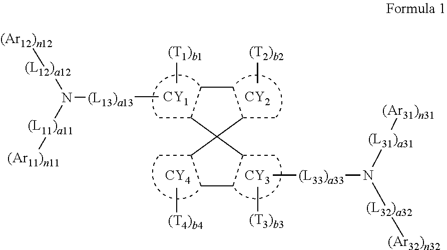
wherein, in Formula 1,
ring CY₁ to ring CY₄ are each independently a C₃-C₃₀ carbocyclic group or a C₁-C₃₀ heterocyclic group,
b1 to b4 are each independently an integer from 0 to 20,
L₁₁ to L₁₃ and L₃₁ to L₃₃ are each independently a C₃-C₃₀ carbocyclic group unsubstituted or substituted with at least one R₁₀ₐ or a C₁-C₃₀ heterocyclic group unsubstituted or substituted with at least one R₁₀ₐ,
a11 to a13 and a31 to a33 are each independently an integer from 0 to 3,
when a11 is 0, *-(L₁₁)ₐ₁₁-*′ is a single bond,
when a12 is 0, *-(L₁₂)ₐ₁₂-*′ is a single bond,
when a13 is 0, *-(L₁₃)ₐ₁₃-*′ is a single bond,
when a31 is 0, *-(L₃₁)ₐ₃₁-*′ is a single bond,
when a32 is 0, *-(L₃₂)ₐ₃₂-*′ is a single bond,
when a33 is 0, *-(L₃₃)ₐ₃₃-*′ is a single bond,
Ar₁₁, Ar₁₂, Ar₃₁, and Ar₃₂ are each independently a C₃-C₆₀ carbocyclic group unsubstituted or substituted with at least one R₁₀ₐ or a C₁-C₆₀ heterocyclic group unsubstituted or substituted with at least one R₁₀ₐ,
at least one of Ar₁₁, Ar₁₂, Ar₃₁, and Ar₃₂ is substituted with four or more deuterium atoms,
n11, n12, n31, and n32 are each independently an integer from 1 to 3,
R₁₀ₐ is:
deuterium, —F, —Cl, —Br, —I, a hydroxyl group, a cyano group, or a nitro group,
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group; or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —B(Q₃₁)(Q₃₂), —C(═O)(Q₃₁), —S(═O)₂(Q₃₁), or —P(═O)(Q₃₁)(Q₃₂);
wherein Q₁ to Q₂, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof, and
T₁ to T₄ are each defined the same as R₁₀ₐ.

2. The light-emitting device of claim 1, wherein the interlayer comprises a diamine compound represented by Formula 1.

3. The light-emitting device of claim 1, wherein:
the first electrode is an anode,
the second electrode is a cathode,
the interlayer further comprises a hole transport region between the first electrode and the emission layer and an electron transport region between the emission layer and the second electrode,
the hole transport region comprises a hole injection layer, a hole transport layer, an emission auxiliary layer, an electron blocking layer, or any combination thereof, and
the electron transport region comprises a hole blocking layer, an electron transport layer, an electron injection layer, or any combination thereof.

4. The light-emitting device of claim 3, wherein the hole transport region comprises the diamine compound represented by Formula 1.

5. The light-emitting device of claim 3, wherein the hole transport layer comprises the diamine compound represented by Formula 1.

6. The light-emitting device of claim 3, further comprising:
a first capping layer and/or a second capping layer, wherein:
the first capping layer is on a surface of the first electrode, and
the second capping layer is on a surface of the second electrode.

7. The light-emitting device of claim 6, wherein at least one selected from the first capping layer and the second capping layer comprises the diamine compound represented by Formula 1.

8. The light-emitting device of claim 1, wherein the emission layer emits blue light.

9. An electronic apparatus comprising the light-emitting device of any one of claim 1.

10. The light-emitting device of claim 9, further comprising a thin-film transistor, wherein:
the thin-film transistor comprises a source electrode and a drain electrode, and
the first electrode of the light-emitting device is electrically connected to at least one selected from the source electrode and the drain electrode of the thin-film transistor.

11. The electronic apparatus of claim 9, further comprising a color filter, a color conversion layer, a touch screen layer, a polarizing layer, or any combination thereof.

12. A diamine compound represented by Formula 1: 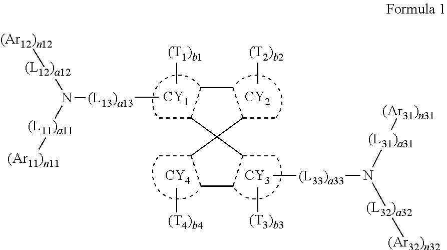
wherein, in Formula 1,
rings CY₁ to CY₄ are each independently a C₃-C₃₀ carbocyclic group or a C₁-C₃₀ heterocyclic group,
b1 to b4 are each independently an integer from 0 to 20, L₁₁ to L₁₃ and L₃₁ to L₃₃ are each independently a C₃-C₃₀ carbocyclic group unsubstituted or substituted with at least one R₁₀ₐ or a C₁-C₃₀ heterocyclic group unsubstituted or substituted with at least one R₁₀ₐ,
a11 to a13 and a31 to a33 are each independently an integer from 0 to 3,
when a11 is 0, *-(L₁₁)ₐ₁₁-*′ is a single bond,
when a12 is 0, *-(L₁₂)ₐ₁₂-*′ is a single bond,
when a13 is 0, *-(L₁₃)ₐ₁₃-*′ is a single bond,
when a31 is 0, *-(L₃₁)ₐ₃₁-*′ is a single bond,
when a32 is 0, *-(L₃₂)ₐ₃₂-*′ is a single bond,
when a33 is 0, *-(L₃₃)ₐ₃₃-*′ is a single bond,
Ar₁₁, Ar₁₂, Ar₃₁, and Ar₃₂ are each independently a C₃-C₆₀ carbocyclic group unsubstituted or substituted with at least one R₁₀ₐ or a C₁-C₆₀ heterocyclic group unsubstituted or substituted with at least one R₁₀ₐ,
at least one of Ar₁₁, Ar₁₂, Ar₃₁, and Ar₃₂ is substituted with four or more deuterium atoms,
n11, n12, n31, and n32 are each independently an integer from 1 to 3,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, —Si(Q₁₁)(Q₁₂)(Q₁₃), B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, —Si(Q₂₁)(Q₂₂)(Q₂₃), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —B(Q₃₁)(Q₃₂), —C(═O)(Q₃₁), —S(═O)₂(Q₃₁), or —P(═O)(Q₃₁)(Q₃₂);
wherein Q₁ to Q₂, Q₁₁ to Q₁₃, Q₂₁ to Q₂₃, and Q₃₁ to Q₃₃ are each independently: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof, and
T₁ to T₄ are each defined the same as R₁₀ₐ.

13. The diamine compound of claim 12, wherein rings CY₁ to CY₄ are each independently a benzene group or a naphthalene group.

14. The diamine compound of claim 12, wherein,
in Formula 1,
a group represented by 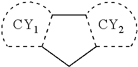
is a group represented by one of Formulae 1-5-1 to 1-5-4: 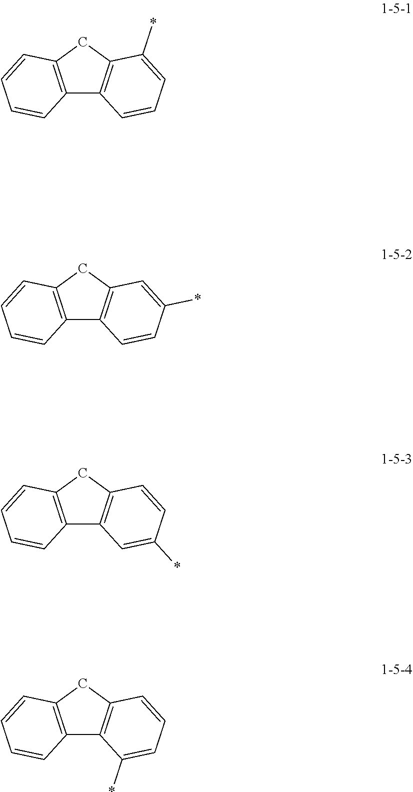
wherein, in Formulae 1-5-1 to 1-5-4,
* indicates a binding site to an atom included in a group represented by 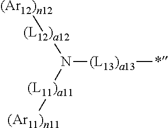
in Formula 1, and
*″ indicates a binding site to a neighboring atom.

15. The diamine compound of claim 12, wherein,
in Formula 1,
a group represented by 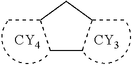
is a group represented by one of Formulae 1-5-1 to 1-5-4: 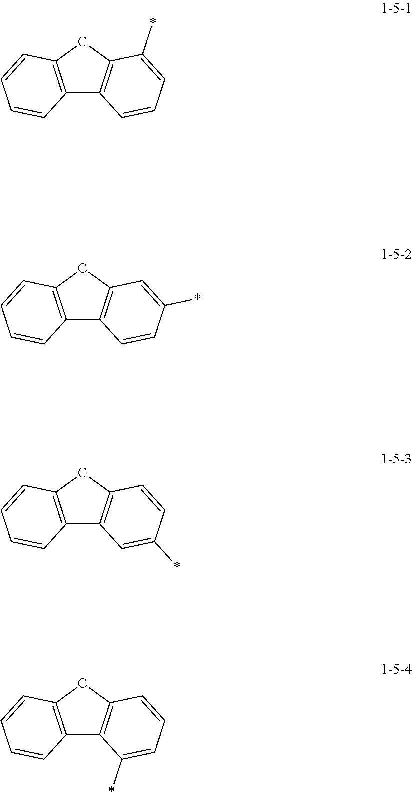
wherein, in Formulae 1-5-1 to 1-5-4,
* indicates a binding site to an atom included in a group represented by 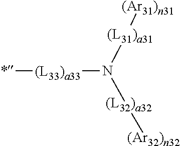
in Formula 1, and
*″ indicates a binding site to a neighboring atom.

16. The diamine compound of claim 12, wherein at least one of L₁₁, L₁₂, L₁₃, L₃₁, L₃₂, and L₃₃ is a benzene group unsubstituted or substituted with at least one R₁₀ₐ, a naphthalene group unsubstituted or substituted with at least one R₁₀ₐ, a carbazole group unsubstituted or substituted with at least one R₁₀ₐ, a fluorene group unsubstituted or substituted with at least one R₁₀ₐ, a dibenzofuran group unsubstituted or substituted with at least one R₁₀ₐ, or a dibenzothiophene group unsubstituted or substituted with at least one R₁₀ₐ.

17. The diamine compound of claim 12, wherein at least one of L₁₁, L₁₂, L₁₃, L₃₁, L₃₂, and L₃₃ is a group represented by one of Formulae 1-6-1 to 1-6-6: 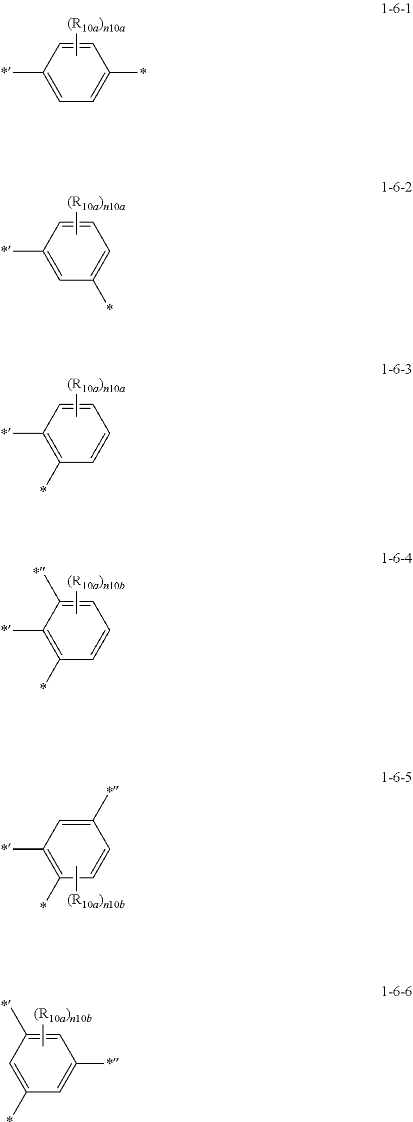
wherein, in Formulae 1-6-1 to 1-6-6,
R₁₀ₐ is defined the same as R₁₀ₐ as described with respect to Formula 1,
n10a is an integer from 0 to 4,
n10b is an integer from 0 to 3, and
*, *′, and *″ each indicate a binding site to a neighboring atom.

18. The diamine compound of claim 12, wherein at least one of Ar₁₁, Ar₁₂, Ar₃₁, and Ar₃₂ is a benzene group substituted with four or more deuterium atoms, a naphthalene group substituted with four or more deuterium atoms, an anthracene group substituted with four or more deuterium atoms, a phenanthrene group substituted with four or more deuterium atoms, a pyrene group substituted with four or more deuterium atoms, or a chrysene group substituted with four or more deuterium atoms.

19. The diamine compound of claim 12, wherein at least one selected from Ar₁₁, Ar₁₂, Ar₃₁, and Ar₃₂ is a group represented by Formula 1-1 or a group represented by Formula 1-2: 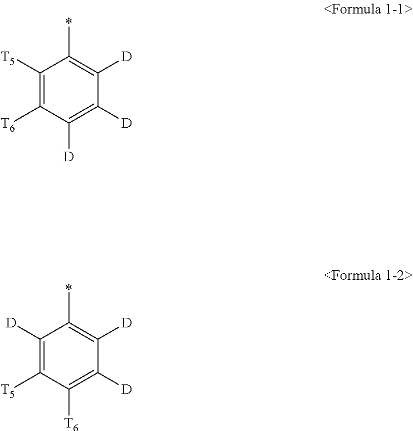
wherein, in Formulae 1-1 and 1-2,
T₅ to T₆ are each defined the same as described with respect to R₁₀ₐ, T₅ and T₆ are connected to each other to form a C₃-C₃₀ carbocyclic group unsubstituted or substituted with at least one R₁₀ₐ or a C₁-C₃₀ heterocyclic group unsubstituted or substituted with at least one R₁₀ₐ, and
* indicates a binding site to a neighboring atom.

20. The diamine compound of claim 12, wherein at least one selected from Ar₁₁, Ar₁₂, Ar₃₁, and Ar₃₂ is a group represented by Formula 1-3 or a group represented by Formula 1-4: 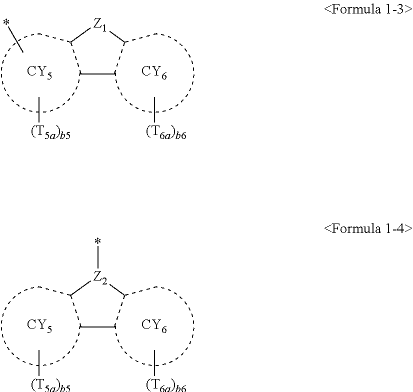
wherein, in Formulae 1-3 and 1-4,
Z₁ is O, S, N(T₇), P(T₇), C(T₇)(T₈), or Si(T₇)(T₈),
Z₂ is N, P, C(T₇), or Si(T₇),
CY₅ and CY₆ are each independently a C₃-C₃₀ carbocyclic group or a C₁-C₃₀ heterocyclic group,
T₅ₐ and T₆ₐ are each defined the same as described with respect to R₁₀ₐ, T₅ₐ and T₆ₐ are connected to each other to form a C₃-C₃₀ carbocyclic group unsubstituted or substituted with at least one R₁₀ₐ or a C₁-C₃₀ heterocyclic group unsubstituted or substituted with at least one R₁₀ₐ,
b5 and b6 are each independently an integer from 0 to 5,
T₇ to T₈ are each defined the same as described with respect to R₁₀ₐ, T₇ and T₈ are connected to each other to form a C₃-C₃₀ carbocyclic group unsubstituted or substituted with at least one R₁₀ₐ or a C₁-C₃₀ heterocyclic group unsubstituted or substituted with at least one R₁₀ₐ, and
* indicates a binding site to a neighboring atom.
